SEQUENCE LISTING (49 sequences)

SEQ ID NO: 1 - DNA, 814 bp, Mus musculus
agtacagacaaagaccagatttgtatcagcacacaattttaagagatacatggatgtgggtatggcagtgaaatttatcttgttattgtttgttgctgcctttgttcctgttgtgactatcagacctctcaaactggatagagctctctataataaactgatcacggaatcacaaactgaaccacaagaggaaattgtcaatacccataacgccttcaggagaaaagtgtccccgccagccaggaacatgctgaaagtgagttggagttcagctgctgcagagaatgctagaatcttggcgaggtactgtgacaagtcagacagtgactcacttgagagacgactaccaaatacattttgtggagaaaatatgcttatggaacattatccctcttcctggtcaaaagttattgaaatctggttcaatgagtccaaatacttcaaatatggagaatggccatccacagatgatgacatcgaaactgatcactacactcagatggtctgggcctctacctacctcgttggctgtgatgttgcagcttgtcgcagacaaaaggcagctacatatctttatgtgtgtcactattgtcatgagggaaatcatcaagatacactaaatatgccttataaggagggctcaccatgtgacgattgtccaaataactgcgaagatggactgtgcactaacccctgcatttattatgatgaatacaacaactgtgatacgcaggtaaaactttatgggtgctcacatccagctgtccagccattctgcaaggcttcttgtctgtgtacaactgaaataaaataa

SEQ ID NO: 2 - DNA, 753 bp, Rattus norvegicus
atggcaatgaaactcatcttgttattgtttcttgctgctactcttactgtctttgttcctgttgtgactctcagacatctcaaactggatagagctctctataatcaactgatcacggaatcacaaactgaaccacaggaagaaattgttgatacacacaatgccttcaggagaaacgtgtccccgccagccaggaacatgctgaaaatggtaagtttaaacatatcatcagtggagatgcatggggcactgggggcatttcctacatcgttacttagtcctatgtactatacattttgtggagaaaatatgcatatggaaaactatccctcttcctggtcaaatgtaattgaaatctggtacaatgagtcgaaatatttcaaatacggagaatggccatccacagatgatgacattgaaacttatcactacacccagatggtctgggcctcttcctacctcattggctgtgatgttgcatcatgccgcaggcaaaaggcagctacgtatctctatgtgtgtcactattgccatgaggggaatagtcaagacacattaaacatgccttataaggaggggccgccatgtcaagactgtccaaacaactgtgaagatggactgtgtaccaacccctgcctttattacgatgaatataacaactgtgataagcaagtaaaacttcttggatgctcacatccagcagtccggccattctgtaaggctttttgtctgtgtaaaactgaaataaaataa

SEQ ID NO: 3 - DNA, 33201 bp, Mus musculus
  misc_feature (30964)..(31071): n is unknown
gatacttggttttcatttcttgcaggctatttccaggcattcattacaacttccatatcatggccagaaaattctgtattaaaagcctcattttttttcattttaattttcatttaactcctatgtgagtactaacacaaagtaactctttactacctactttgtattttcttcccttattgatagtcaagtcaccctcccttttaaaacatgtataaataaatattttaattaaatgttttattattctacaatggaaaatagaaaaaaggcatggagagaatgttttcaatataatttatgcataaaggtgtacatgtataaatggatatatatatgtatgtattgaaatgttatttgtatataagcagaacatagatcggtagatcaatagatagatagatagatagatagatagatagatagatagatagatagacagacagacagacagatagatgacagggagatataaataaggtcaaataagcatttgtattttttatatccttttctaaagtgtcctggctttaatcatatatgtgttaaatgatgcatagtcaaaagatgatcaataggacaggctccctaaaaattcaattgagagcagtttttctttcttttcttttcttttcttttcttttcttttctttttttttttttttttttttttttttttgtatgcaatgagaaccatgtcatacaattcctggccctcttctttaatgtttctggaatagtaagcaaaattaaaagcaattgtttgacaaactatgagcactcattcaagagtaagttttctatttttatatgtgctttcagtccattggcaagaattgccattcttttcttttaaaagctctttaacttcttatcctcaaactcaagagatatctggattcttagtttttccttactgacatttagtacaaacctacttaatatttccagatttttcaaggcagtgtgtcaactccatacacttctgatttttttttatcagaattttatctttttgtagatatttgttttctgtaatcactgagatctttatgagagtggactagcttcttttgcatctttggatcatcctaagctaactagaataacattgtatcatctcactaatgcactgcaagcactacacagcaggcactaaagaaattcctcagcagatttcactctgtgtgtgtctgtttatgataatcagccaaacatgcagaaaagaacatccttaatactacaattttaatacaagactggagaaatgatgcagtagttaaagtcttttccagaccatattaggaccagaattcaatccaatacagaggacccaaataaataggtaggaactcctttcactctgttttaatctttgctctgcggctgtgagatcaggaggatttctaggatatgctggcccagtagtatatactaggttcagtgagaaaactcatctcaaaaaaaaaaaaaaaatgaggtgaggagcaattaaggacaataccctacattgaccctcgacatctggcctccattcacatataaacctgagtgtacacacatgtgaagacaagtatatagacacataaacataatatttgtaccacacgcatatgaatgcacaaacacacacacatacacacacacacttaaataaacattgatttttaagttagagaaaaaaataaaactgataaaatatttcctttcccactattgagactttcaggaatctttctattcttttgtgagtctatgtatcctgcagctaagttttacagtttgttctcagtgcaaaaatttaacgctgataaagttgtctaggtcattgtatattctagaatatgctcatgatcgtgacttgaaatctaattttgttgtgaacatgtgagaaagcatatctgctgctcttccttgatctgctgaagcagttccccttgtactaattgcctcattgagcatatcctccttttgaaatgtcatttcactcgttagtatgcagtacagacaaagaccagatttgtatcagcacacaattttaagagatacatggatgtgggtaagttatacatcatatttcagttcatttaattgtaagtgaaatgcatatataaaaattcagcagcactgttataaaatcaattacatgcaaaatctattaatgttgacataatctaaaagcaaaggtccaccatttccacaattttagtaggatttatgatcaatacattctgtccaagattatgtcacttttcgagttcattctcataaactactgaagtaagatttaataactccaatttggacatctttacttaagttttaaattttatagatgtatttttgagtatattcccctacttcatgcttctttcccttccctatctctccttttcccttcaattaatctcttttcacaaaagacagttttgatgttacttgtatgccatatatctatacatgattttatgtacctatattatatcaaagaatcacaaaagagagaaaagatgtaacgtttgtcttatgagattgtcttaactcatttaatatctatagttgcttgcattcccatctgaaagacatgactatgttcttctttctgtctggaaaggattagattatgtagttttatatctatgtgtgtatagatagaagaaatatcacacaggtttataaatgtctctgaaatatattgtcttgacactctttgagtaaatactcaggagtggtatagttgtttcatatgataaacctattgttagacttttgagaacccttcatactgacttccagagaggctggactagttcacatccccaccagaagtttaaagcatacaactgtggattagtggagtctggacacattttatttcacatgaatctatttgaaactgtggtcaaaataagcagttagtagagaaacattataatactgtgcatattttacatttgcagtttatcttggggaatcatgaattattttgtggataataggataaacactttactttacataagaaaagcaattcccagaaatcgtagtcttggggaaggagggataattttagctgttcaaatttctttctgcagatcttctagttgctgttctttgatctttcctcatgggagatctcatagtctgaaagatcacacatttcgcagcctttctcagcactgtctcttctctgtgccttctcctttcacatttctcttgtggtgctttctttgttctacttatgtttcctgttgtgtatgctgttcttctgttctctgatttatgaaaatattgtcttcagtacatattctatagtgtttgtgagtacatgtttatttctgtactggacattagtacagaagataatgaagcaaatttaaattaaaagggagtaaaagtaaattaaaagtgaaagtaaaaagatgatcatttatttcaaaagttatgactagacactaatacagtagattattatcaacatatgtgtggatttatgtcaaacaactgtaatctgcagtaaataatacaattactattaattatatggcatgtaaaacataatttttgtctcaaatgtgagacagagttcgaattcattatttttgtattagatacttcactgaattttctttatttgatttcatgttagcctttgtcttaaacgagatgatcatatgtagaagtttattttgtatcctataatattcccttgatccgtttttctaaacttatgacaccaatgctgtccatgcccacaattatagtacattttaatatttagtagtgcaagtgttcaaatttagatgctattccttaatattttatagttttaatcaacttttagaagaaaaatgctttcataatttgtctattcaggtaacaaaaattatcaaacattcatatacacagagacacacaaacacaaaattagctattgtaagtaaaactaaaacctaatatctacttttaaactatttgctaaataagatatagagatatgggtcagctatttcatatacactacttctaaggctagagattattaagaaagagggtgaaataattatatgaataaaggacattgtctataagaaaattatcttctgaagatagcagggatactgcagatatgaacttacagggattgtgacataacacataagatacaggcactaacatgtagtctgctgaatgtaaggattaaggtatttgccaaagcaaagaagattaaatgtgggaaatggtcttttggcaaggtatgtttttatcggttattcagtcaaggcaaggaagagcctgttcctctttttagttgattatgaaaggtcatgttctaacaattcccagatgggggcgttttctctaaggcttgccacagtgtactgcttgatcctcctcagctttttcccacatcctctatctttcctcccacaaaatattaatttttaattgttcatggcccatttaataaagcttaaagttaggacaataagtttatgtagtcggtttttcaagttcaggtactgttcattttttaaaaagaaagttgactaagagtcgcacattatgaacaaaacatgtgtgttcaaacagaaatgctgattttattctcctttgtctctgttttaactgagttcaggaaactagttagcatctcagataaaaggtaagctgtgtttacagatttgcagcctgtggtcaatggagaatcatgcaagatactctggatgctttgccctggtcagtccatggattacatcaagtctcagtgatctagtgaacaaagctcagacaaggcctctaccctgagactatttttcctctaagcctgaatagctgaatagcttcgatttatgctgtctaatatttatctttagaccagcatctttgttgatatcaggctcaattttctgctgaaaccacaacctctttctgatccttctctctacccttgactcctgattttgagttgtgtttacaaacactacccaagagatccaaagtctgtaatctctcatactcatatctagaataatagcaattatatattatatattctggcaattatatggattctattttcattctaaaatccttaatgcattcctggtggtaaggtattaaatagtatcttcctgtttcaacttgattgctctaagtgctatttgagttttaattaacctaaaataatcacctatgttctttgtacttttgattatttaaggaacattaacatgagtggtcacattctacctgctcacaagtcctgaagccaaagcaggattaaggatttgagagatttttttcttagcagatctccttccttctgttcagtcaaactgatctttgctaaaccaaagttcttcgtatgagaccaatgtgctaaattaaccataatctacctgaaaccagtggtcccatgatcttatatcagaggtcagataggagtttttttgtggtttatgtctattgttcacagataaaaagtcacaggcatatattatagtgttcagtcatttatgtatgctacataaaacaaatatttttcacctatttgtgacagaagcctttgtatttattttttacatgaatatgtacatatacatgtttcagttaatctttatgtagttagatatattttccaacaatgcccttatcattaactaaaggcaatatatagtatattatatgccttcacattatgatatgaggcaatctaacagattgtagcattctgggctgcccacttgggttagatgtctactaaagattttggctgtttcttttgatgcaggctgctcctcttatttgagcagatatgtctgaaattttcagaagaccctctgttgtaataaagttttggtggtataatctatttaactaatattctaaactcctggctctatctctactctgtctctttgggctgggtaatatctgcagcaagttactattcagacattagtaaccatgatgcagacacttattaagtactgattaatttatggaataagaggccctcatgtctattattaggttagctattgtagtgataagtagaaaagaactgaagagtaggtaagcaattatagtaagaatagtggttaatatcagattgttagatggaagtaaaggaagggagtatacatgggaataggaataagagaagagaaaaaaacaggatgaatacataggggtttgagttaatgacaaactaaaaagaagagagaaagatgttttagatatcacaaaccaagtctgtctctaggctttagggaaataactgtaagaaaaatgggcagaagctaacagcttctgggacaggcgggagccacagagcttctaaggcagcccccttttccggccccagacatccagccactttcccggccagaggataggtgtccgcccagcccaggagcgctttgcctgagcatcagcagtggacatcttggttccaggactcctccaagtgtattctgcacaggtaagagtgtagaatacagaagctaacagcttctgggacaggctgaaacaactcagcttcggggacaggtcctgttttgggccttcatcttcggccatgagggaggtccaaaagccagatatctgtgcaccttccctgtaagaggagagcttgcctgcagagagtgctctgaccactgaaactcagaggagagagctactctcccaggtctgctgatagaggctaacagaatcacctgaggaacaagctctaaccagagacaactaaatcaactaactccagagattaccagatggagagggttaaacgtaagaatcttactaacagaaaccaagaccactcaccatcagcagaaaccagctctcccacctcacccagtccagggcaccccaacacacccgaaaagctagacccggatttaaaagcatatctcatgatgatggtagaggacatcaagaaggaatcactcaaagaaataaagtagaacatggctaaacaggtataagatcttaaagaggaagcacaaaaaccccttaaagaattacaggaatacatgaccaaacaggtgatggaattgaataaaaccatctaagacctaaagagggaagtaaacacaataaagaaaacccaaagtgatgcaacgctggagatagaaaccatagaaaagaaatctggaaccatagatgcaagcatcagcaacagaatacaagagatagaagagataatctcaggtacagaagattccatagagaacattggaacaccaatcaaagaaaatacaaaatgcaaaacaaccctaactcaaaacatccaggaaatgaaggacaaaatgagaagaccaaacctacagataatagaagtagatgagaatgaagattttcaactcaaaggcccagcaaataacttagacaaacttatagaagaaaacttcccaaacctaaagaatgacatgcccatgaacatacaagaagcctacagaactccaaatagactggaccagaaaagaaattcctcccaacatataataatcagaacaacaaatgcactaattaaagatagaatattaaaagcagaaaggaaaaagggtcaagtaacatataaaggcaagcctatcagaattacaccagatttttcaccagatactaattaagcaagaagagcttggacagaggttatacagacactaagaaaacacaaatgccaacccaggctactatacccagccaaactttcaattaccttagatggagaaaccaaagtattccatgacagaaccaaattcacacattatttttccacaaatctagcccttcaaaggataataacaggaaaaaagaaaatccaatacaaggacagaaaccatgtcctagaaaaagaaagaaggtaatccctcaacaaacctaaaataagacagccacaagaacagaatgccaactttaacaacaaaaaataggaagcaacaattacttttccttaatatctcttaatatcaatggactcaactccccaataaaaaacatagactaacagactggctacaaaaacaggacccaacattctgctgcttacaagaaacacatctcagggaaaaggattgacactaccttaaaatgaaaggctggaaaacaattttccaagcaaatggtctgaagaaacaagctggagtagccattctaatatctaataaaatcgacttccaacccaaggtcatcaaaaaagacaaggaggggcacttcatactcatcaatggtaaaatcttccaagaggaactctcaattctgaatatctatgctccaaatacaagggcagccacattcattaaataaactttagtaaagctcaaagcacacattgcaattcacacaataatagtgggagacttcaacacaccactttctccaatagacagatcatggaaacagaaactaaacagggacacagtgaaactaacagaagttatgaaacaaatggatctaacagatatctatataacatttcatcctaaaacaaaaggatataccttcttctcatcacctcatggtaccttctccaaaattggccacataattggacacaaaacaagtctcaacagatacaataatattgaaattgtcccatgcatcctatcagatcaccatggactaaggccgatcttcaataacaaaataaataatagaaagccaactttcatgtggaaactgaacaatactcttctcaatgataccttggtcaaggaaggaataaagaaaaaaattaaggactttttggagtttaatgaaaatgaagccacaacatacccaaacttatgggacacaatgaaagcattttaagaggaaaactcatagctctgagtgcctccaaaagaaactagagagagcacacattagcagcttgacaacacacctaaaagctctagaacaaaacaaagcaaattcacccaagaggagtagaagacaggaaataatcaaactcaggggtgaaatcaaccaagtggaaacaagaagaactattcagagaatcaaccaaaggaggagctggttcttgagaaaatcaacaagatagataaacccttagccagactcactagagggcacagggaaagcatcctaattaacaaaatcagaaatgaaaagggagacataacaacagatcctgaagaaatccaaaacaccatcagatccttctacaaaaggctatactcaacaaaactggagaacctggatgaaatggacaagtttctagacagataccaggttccaaagttaaatcaagatcaggttaatgatctaaacagtcctatatccctaaagaaatcaaagcagttattaatagtctcccaaccaaaaaaagcccaggactagatgggtttagtgcagagttctatcagaccttcaaagaagatctaattccagtccttcacaaactattccacaaaatagaaacagaaggtaatctacccagctcattctatgaagccacaattactctgatacctaaactacaaaaagacccaacaaagatagagaacttcagaccaatttcccttatgaatatcgatgcaaaagtcctcaataaagttctcactaaccgaatccaagaacacatcaaaacaatcatccatcctgaccaagtagatttcatcccagggatgcagggacggttcaatatacagaaatccatcaaagtactccagtatataaacaaactcaaagacaaaaaccacatgatcatctccttagatgcggagaaaacatttgacaaaatccaacactcattcatgataaaagtcttggaaagatcaggaattcaaggcccatacctaaacatgataaaagcaatctacagcaaaccaatagccaacatcaaagtaaatggtgagaagctggaagaaatcccactaaaatcatgtggttgattgtgaggatgtgggaagcactggagaaaatgagctgtagagagtagaggtgaagaaaaaggcagagcaatgctacatggttcatttaatgaagaatccctcaggttatacttttttttttcagaggacatgggacatctgagagcaatgacaggacagccatgtaactatggaaacaagcataagtactcctgtttactatttaattcttcataaattgattaggttgcttttaaagattagaaatttctgtaaaagttaactagtaggtttatacaaatacacataacaggttgttagaaaaagtgccacctcattagtatcttagaattgactaactgtactaaaaatgaataaagacccattagtatttattttgcataacacaaagacaattattattagccaagtaatggaaatgtacagaatatctttcaattaaatgtatagaaatctttaaattacacacaaacaacagtagtaacatgatcacctgccttctatgtactctttcaaatatacatggctgatggagtctatttcatgttatattctacaaatgttgtctcctgactttttctttcacaattaggcacctttgttattaacttaaatcaatgaagaatatataggataatctcttttaagctttagtcacttggtgagaaacctaaatgctatctgctattttaataccccataaacacacaattgtgtatatttagactctggaagttaaggagtccatatcatttaagttaagtccatatcatctaaatcaactgaatattattttatgaggccccttagctaaggcttaatattgaactttattttcatgtcccattaacaaaaatctaacaaagtaaaaccttgctcaaggcttcttagaaaataattttttacacactaagagtaacctaaatttctgttacttatatttttcaggtatggcagtgaaatttatcttgttattgtttgttgctgcctttgttcctgttgtgactatcagagtatgtaaatattatatgttacataactatattttatttaagggtaggagaagggtgtatcagttattcacatacttcatataatgatgcagttaaacaagtcttattctgttttataaaaagaattatttatttattctatgtatatgattacactatagctatctccagacatatcagaagaggacattggattcctttacagatggttgtgagccatgtggttcctgtgtattgaactcagaacatcttgaagagcaatcattgctcttaactactgagctatttcttctgcccaaacaagttctattcttaatacagcagaatcacgaatgaaaccaaaaggagaaggcctaagtgaactttcaagtttagaagaattgatgaggattaaagttcaactttcactttaactcattggatccatgtctatgtctttttttataaacaataagaaaacaaacaaacaaataaataaaggcaagctagcaaaggatgtgataaggtagagaagggatggaggaagagaaagaaaaaaatccataggcaagaacttgtttacaatttacccatgtgttttaataaccagatcgaagaaattagataaatagtattatttttaattttcctcccacataccctttttgattttaagaatgttataaatcaaaattaaatatgaaattgaaagttacaactgcaaagatgtgtgagagattcagcaatggaatcaaggtatgatgcttggatctaattgctcataaaaataaaacctttgggctagtgatatagctcagtattatagcacttgaattgtgtgtgtgtgtgatgtccagaatagatccccaggatcacaaagtaagataatgatggtaatgctgatgttgttaatgataatgatgacttcaatatttatagaacaaaaccaacatcttcagtggtaaaataggtagagtgtcctacaaatttgtgatcattgagtgacaattcatcattagcaaccaaaataaggcatggaaaactgatcttaatttactttatctaattaaaaaatgacttaatgtcaatagatttttcttttgtagaactagattttttgcgtgactgtgtatgtgtgtttctcatgagtcacagggagatattcacagtgggaaaaattacataatattattgttgaaagattattgtcatcagctactggagtggtggattggtatcagtataccatgatgtccatgtccccatgatcttctaggaagctgaatattgaataccaagctttatggaaggtttcctttaacttccctttgattctctgcttgtttcacatcttttttgtggaagaacatcacaccaccaaaactagagctaacataaagtataggctgcattacaaaggaaaaaggaaagttgggtagtcagtaaccaacatgtgcctttgtgacccggtagtctacgataatttagcccaattttggagcacaaataaaagaaagtatgagcactaccagagttcagagaaaagttcactgaaggaactaaggaactaagatatcagaggtttccagacagatatatggatactagaccactggactgattaaaggaatctagatgtgcaaggtgaatttacataagagatttgatttttgtgaaagtaaatgaacaatctcagaaaaaaaatagacattcctttactgttttattatagaggttgtaaatatttgtggatgaacagagagtcaaatgttatgtttttcattgaaatcaaatgctcaaaggaggaagtctgtgcattcttgtgagcctacaaatttttctttaatataaactagagctgattgcaatgatggaagaaaccagcctgtgagcatgttccaaggaaaggagttggtgtctgtagaggttccatgggaatgtactcttgacctgaatgacttctgttttgtcaaagctccattctggattcttaccattcttaaatttactcaataatccagtagtagctaaggaaaaaacatacaccattcaaattacatattaattttgataactgataaaagaaaccttgggaaatatatgggttttaaaccacacatattaaagactcattatggcaactaatgcgaacacagactattttagaaaactatttttttcttaatgaattacaaagtaaaatgtataactttttaaaaaaggagtcatacattcgaaactacaacggtagtttgaattttacttacacttttaaatgcacccacttgttttaaagcccaccgggtttttaagatataaatataaaagagctataaacctggagagtgaaaagaacatgtagtcacatgctcaaaatacactcttaattttggttcttacacacacatgcacacacacttacatgcacacacacacacttggactcaagtattcattgttcatttgtagagtacaggcattgctttcaacagctttatatagagagaatgagctaaggcagagagtcattccgtgaacaagagatgtcaagccatggcttttgtgattcctattgttcaataacaagaacttctacttgcctctaaatatgaattataaatttgaaaatttcgaagaacagtataatttgtgtactgttttcaacccaaaaatgtagaaatccttaaaacttgaatactaataatttctaaatactaaacataattgttcactgttaagttctaatttcacattgcctataggtctctatgtaatagactttgtgtgacaagggctacttcaaagaagtgatattaatgaataattttctggtagcctctcaaactggatagagctctctataataaactgatcacggaatcacaaactgaaccacaagaggaaattgtcaatacccataacgccttcaggagaaaagtgtccccgccagccaggaacatgctgaaagtggtaagtctagagcacagcatcagtgtagaactggtggcattccctacattgttacttagtcttatgtactgtgactatgccaattgtttgatcatcgcaacaaatctatgtcataacagcaaaagaaactgagtctaagagaatgtgactaactctctcatgtatatgatagaataacaattctgactcagcctcaaatcagtcttaggtgccttcagacatgtgacatgcatttgagaatccggatgttcaaattcatagctaaaaaaatttgacagattttgaaaaaaaaaaactagtagtgaatttcattaaattccatagaaatttagagatctgaccttggcagtgcaaaaactgatggtttctatcacaagaaatatttagggactcaagacagtcaagagtgatgaagttcagtcaacagtgcaggccctgagcagtagttttcaatctataaatatcaaccccttggggggtcacaaagcagttatgcattatggcttataacagtaacaaaattacagttgtgaagtagaaacagaataacttcatggttagtggtcaccacaataggaggaactgtactaaagggtcacagcaccaggaaggttgagaaatactgccctattatcatatgataataactggcagtacactcctgtaaatgatggagaggaagcaacaattgaaaacgaatgtagtttctgaaaaattatctctagaccaccttaaaaattaacagtattttctgatacactattttaaaaccatcagttgtcataagtatccatgtttgtgctactgttaataaatgttctcgagctaaacatagtgatttatatctagactcccagcattcagatagactccgaggctgaggcagaggatccctaggtgttcaaatccaacacaggccacatgctgcgctccagaaaatgtattcttatagtttctaaaagtaatctagtttatttaattttgcactacaatggtttccactgcaaggagtctcaattatatgatcaattttactctaaatccttaattcaagagggacatacccagtaaatctgatcctaatcatattaagataatttccaaattcctatctaccagatgaacaaataactatttctaggaagggctctctactgagcagtctaattatatactgaaagcaattttggtcttttcaattccttggttagacacacacatcaaaaagtccccaaaggcccttatacctttcctactccctgctccccaatccaccctctcctgcttcctggccatggcattctcctgtactggggcatatgatcttcgcaagaccaagggcctctcctcccattgatggccgaatgggccatcctctgctacatatgcaactagagacacagctctggggggtactggctagttcatattgttgttccacctatagggttgcccacccctttagctccttgggtactttctctagcttcttcattaggggccctgtattccatccaatagatgactgtgagcatccacttctgtatttgccaggaattggcatagcctcacaaaagagagctatgtcagggtcctgtcagcaaaatcttgctggcatatgcaatagtgtctgggtttggtggttgaacatgggatggatccctgggtggggcagtctctggatggtccttccttccatctcagctctgaactttgtctctgtaattccttcccatgggtattttgttccccattctaaaaaggagtgaaatatccacactttggtcttccttcttcttgagtttcatgtgttttgcaaatagtgtcttggctataagtttctggcctaatatccacttatcagtgagtgcatatcatgtgggttcttttatgattgggttacctcactcagaatgatatcctccagatgcatccacttgcctaagaatttcatgaattcattgtttttaattgctgagtagtactccattgtgtaaatgtatcacattttctgtatccattcctctgttgaggtacatctgagttctttgatgtccctcaaaagtattatttcttatttgaaaaacggaaacaaataacatgttttggatgcagtcaactagaagagtaattctacacaatagccactagatggtaccgtacacacatacagtaattatcttttcactgtcatgctgagaaaattgacctttactctgaagagtgatggcaataattctaatgttgcctcctacattattactaagtccagtgaacataatgtgttctaaaaatttcgttaaaatgccaatctcaaaccacagaagtagggtaaaacgagtctaaaaagagttgtaactgtctgtacatccttagaaaatttatataaaagtctgggaacataccaaggaaatcatgttattgttgacagtgtaacacacaggctgtgagcacactctgacgatgctgggtgcaggattgtcaggatgtcactgctactcctacatacgtgtgtctgagtggtttattaaatgatgcttcaactgagtatatgctacacttagcccacccaaatgaagagttcaggctaatctcgagtaactgattcaaattctctcttgtatgacccttaaaagctttagaacctggttttgggctggagaaatggctctgcagataagagtactggctgctcttcctgagaatcccggttcaatttccaacatccacagggcagctcacaactgtctgtaattcctgttccaggtgatccaacatgtgctcacagatatacatgcaggcaaaacaccaatgaactttaaaaaataaaatattttttaaaattcacacacagtacactgagttactgcaagagtggaagatttatggttaataacagtgtctaggaatgttaactcctttgctttaatcactacttgaggcagggatctagaactctgctttcattcacagaagccatttgtgtgctaagtgcatattttgagactcaaaatatcacaggttgattgtaaggactgccgactggacaaagcttttggcataacttccatatcatctttacagaagctgtgactcaaagaggtgaaatgacaagccagggttttcttgtttgcatctctgttgcagtgatgaaacactgacccaagggaacatagaggagggggtttatttggtctaaaatgcctgatgacaataaattgttgaggaaaggcaaggcaaggtctagcagaggccatagaagaatgcctcttactggcttattcttcatgacttctcccatctgttttcttatatcacttagaaccacatacaatctctgatcaagaatatgtcaccaaagcatgttcccaagtcactatgatgtagccaattccttagggacagttctttctttccaagtgactctagtttgtatcaaattgacaaaaactagccagcactgagataaaacaaccgataagttgctgggttagttttgtgattgtttgtttcctgaaactctttacataaattataagaaacaagttatggtctgaaggttataacctaattttgaatttgtaatatcacaagaaactgaaatgaattacacacttgttttctttctatgttcctattagaatatttaaggaaaatgtcaccatgtaaaagatcagtttccacaagaaaggacataaggaattgtacatttttttttatatagaacacaagaatcttaggttgagaaagctaatttcattgtgtttctggatattctcttgccttacagagttggagttcagctgctgcagagaatgctagaatcttggcgaggtactgtgacaagtcagacagtgactcacttgagagacgactaccaagtatgtatgggtctgcatgacacaacttgcattaaatagttttcccaacactatagtgaaatgcctgggggtggggctgatcaattcaaagggaggggtttgtttttaaatcatggcttcagaaagttcagcccgtggatttttagcttgaatgatttggaacagaacggaggccacataagatagtggattgaaaagtggaggatgctgttcacctcaaatagaaaagacgtaggagagagaaagagagagaatgccttcagagatccaactctgtcatataggctccaattctcaaagcacctacaacccactcagtcccagtatctagagaacaagccttcaacaggtgagaccttgtgatacagttaaggtgcaaataacttttaaacaaacaaaaatgttaatgaataaaataaatctcaaaaaagaacgtgcctacaaacaactggacaatagtatgctaacagatagaatctacagggaccctcacctatccttgctggcagattgtagcttgatatcatcatttttaatttttaaaatttggatacttttatttttgtagtctaagttctcatcaacaggaggaagctatgatcagcaacattaaagaatgaagcacatcaggctctctaggaggtttctattcaaacatctctcagttgtccacaattttaggatgtgacatttcctcagtttggggacactaggatatgaggaactctagaactaccctaataaacaattttgagtgctcaaaggtaagctcagttttgtcatataaactaaacaagtactacccaatttttagtgaatcctacagaattacgtaacattttaagtaatgctttcttccaaactaactttaagaaatacaaatgtaatttatacttatagattattggaactcttatgccactgtgttaaacaatgcgggtatcatggtattctaaagaaagacagaattattcctcaatatcaaatcctacataaagcatataggacaaatggatccaccatcttttgagcatatgtatcctctttgtaactgattttacctccagagctggtttactttcagctctcatgtgctcaaacagacttttaccatcatcctttacctgataaaagacagagattgatatctgcagtcatgattttaaagctttctcaacaactgtcagtaccatcgcttgccagacttgagcacaacatgatcattcagtgaaacagataaacaactgagtttatttcctgttttaaaacaatgcctaggtgatcatgaaaacatttgggaaataaaatgaggagatcctctgtgttctaacataaaaacatactacacaggctttttcctcatttcagatacattttgtggagaaaatatgcttatggaacattatccctcttcctggtcaaaagttattgaaatctggttcaatgagtccaaatacttcaaatatggagaatggccatccacagatgatgacatcgaaactgatcactacactcaggtaatctgtattcttctagatatacacatgactggatatatgtgtcagtaaaggataactactatttctcttctcaggcctaaggaaaataacccaggaaatcccaaacatctacttcctagattgtggccaggataatgataataccacttgactgacagtgcaaacgggaaattccacctgagcccaccttcttgtctaagaacaagtagtcaataacttctcagctaaagagtgtcagtcttgaacatatttacatatgagtaatcgtaaaggatctcagtaggttcaatacacaacctatatgtgtctagatctatatgtaataataatgattaaagaaggaacaaggagctcacgtttgaaatggaagatgcaaagtgatatagatagggctcatgtatgaagttctcagttaacagagttttttgtttgtatttttttcaaaaacaatttttattagatatttttttcatttacatttcaaatgctatcctgaaagttctctatatgctccccctgctctgcgccccaacccacccacccctgcttctggccctggcattcccctgtactggggcatatgatcttcacaagatcaagggcctctcctcccaatgatggctgactaggccatcttctcttacatatgtaactagagacacaatcaataaacagatttttttttggtttgtatttttatgattaagtggaagcaaaaggcaatcagtaagtttatgggtaatatacctgctggagtcttataaaggatctgaagatctgtactctggtccttatgaagaaaggcaaatattttgtctaatgaggcattttcccagcccacttagcttcattagcatgttttagtttgagtaagttttagttaaagtaagttttattatgagatttcaacatgcaatgtatgtgcagattataatcactgcttttatttgctatttactgtccctgatccatttccacatccatcacctcattgtccctctattgccatgacacatgaaattcatgtgtgacagaaaacctaatccttgacttaaggagactatctatattctctcaataaaaaagatccatagttccgtacattttcctgaaaatggcatattttattgttcttattgtttgaaaaactgtcttttgtgtctttctaacacattcatatggaaacagatacctagactgtcttgtctattgttaatggtacccttgtaaagacaagagcgcatgtcagtattgtatgttgatcaggatttcctcatatatacatccagaaaaaataaaacttaaggaagttctacttctaataatttaagtaactccatacagattttcacaagggattcccagtttatgtttccaccagcaccatgcaggaatccttctctttccacagcatcacaaatatttgtacttttatttttctggagagccatcattctaagagcagtgacacggaatcttgcagaaatgttgattaaatttttctaacagctgaatatgttgataatttttcatgtattaattggacattcatatttcttttaagtacaaccaaccaatagttaaactgttcaaaacctgacactgtcaaggaatagtatcattttcaaaccacatcagatctactttaatttctttcatttaatattctacagatctcaccatgaaatatttaaggctctttgctaaagattctaagtagtttattgccattgtgctttttaagtagaacaattttcttaatttaattttcttattattcatttagttttagtgttatagaaaatttgagagtgattaatgggaatagataaggttttcacaagagagacaaagtgatcatattacccagtataatatttacatttatattaaatgcaggatgtaatcagtggaggaaaatagacaaaggattgacttaatttgattaaaattttgaattatcatttgcctgccacacagagtccaagttaattttaaagaaaaatatccctagaatctctaatgtaacaaaatgtatgctagagaaagagtatttaaatatttaaaatgtaaaacattaaaatatagttattgtccacaaaagagaggactcggaagtgttcactggtgattctcataacttacatgaagtaaattttagttaggattgaccatataagttcccaaagatgtacctcaaataatcagttatgaatgtatgcacagaattattaaataaacaacagcaaattatcttttctttaaggatcagtaccactgaacataaacagcatgtgcaaagaatccatgataccttaaaacttagcaggttaatccaatgatttatgatgccaatggattagaagagagcctgagaaagaaaaacagtgaaaactatgatgatgcaaccccagtcttataacccattcatacaaagtcaatccctttcttatatcatatggctttggttctttcagtcttgtgcagcaatttatccctttgactttctatttatcaaaataattcaagccatatcaaataaatttataaaaataactggtcccatttgccaaaggtgctagaaattgtataacattttgtaaattaataacaagagaggccatgaatttaaaaagtagcaaaattatataacagggtttggagaaagaaaatgaaaatactgtaaaattatcataatcttaaagccaaagaaatattttcaaaaatcaaagaaattggtctttatttgcttaaattttaagagaaaaatactgtcattatcgtacaatgataagaatccattaatattcattttttcacattatttgtactttttcttgaatatggactgaatgaataaatggaacaaatacacaaaatagatgttctaatgtatttttattgtaatttttcaaagtatatgttctatattagtcagaggttaacaaagtttcataaggcaaatgtacgtttaggagaaagaatgagaagagaaacttgcaatcattgagtttttgcctcatccaacgtttacatagtgggtggagccacagatttcatgaaagcgtattcttattccccatagataattaaatgctgtctggtgtggagggaaactgactcctggaaaagttgtgaagagaaagtaatactttacaaaatcagtcagagaaaatcactatgaatagaaagaagaagaaagaggaggaggagaagaggaggagaataataggaaatagggagaagaagggagaaggagaggaggaggaagaggaggaggagaagaaagagaggaaggagaaaaagtagaatagaagaaaaggcaaaagaagaagaaacctgattaaagatttcagtgttggctgagggaaatacaagccaggctcctacccaggtcacagaagaaagtgggcgccattgcatcacccttatttccttctccagcttttttaaacattgtaatatttaaccctcactatacaaatgcatgtactgggcagcattattatagtcgtcttacagatgagaaaaatgaaaccctaataaagaaacttctagatccaatccaaacagaaccaaaccaaaactaaaaccagaacaacaaaaacaaaacaaaacaaaaacaaagtgaacaaacaaacaaaattattacccagcttagaaatacagaggtggaatttaagcttggttcatttgcattaaaatccatttgagtattatactctatgcttgaccagtaattaaacactgttcttgggtttgagtcttatagaaataacctttatcatattagcatattcaccgcataggtttgacatggagagaatgagctgaatagatgatatcaattctgggtttcattgggaattaatataacgagattcttgttccactaaggtgtcctaaacacatgagtttttcatatatctgtcttctggataagacatgtagaatgcaaacttaagacatgatttcctatttaagattaacttttagtgtgtgtgtgtgtgtgtgtgtgtgtgtgtgtgtgtgtgtgtgtgtgtttgtgaacgtgcatgtgtgtttgtttataagtgttatacatataggtaataagaaggccttggatacctggtattggatttagagatggttattggcagtctaggtggatggtggagtcactttcatgtccactaaagaatcaagctcttataaccattgagccatcactccatgacgtgcatgcttacatataataacaataacagtgctgatgttgtcgttagggaacatgaaaatctgtcatcaagtcatctcttatctttacagatggtctgggcctctacctacctcgttggctgtgatgttgcagcttgtcgcagacaaaaggcagctacatatctttatgtgtgtcactattgtcatgagtatgtgatgccagtttattattctttctaaatagcgtatcttcaaaaaataaccttgtctaaggtttcaaatattgtaaattaatgttctagggtttttccccagttattttgttaacaaaatgtcacaaatataccccaaatgtttgatttttcatcatgtttttactaagttaacagagatatacacatttggaaaatttattggtgattctagtcattcactggaaagtgttatcatacagggaagtgaatttgaactgggttaggctaagaaaggagatgcaggcaaatgccactccctgtgctaggctttgggatccctatgttgacttgtgtatttcctttagaaataaaatttatgttactctgagtgacataaagacaatgttttatttttaacagagaaacaattttaaaggcaatatatgtatgcatatatattatgtatgcatatattacttgcatacaaacatatgcatacatacaatgcaaatataaaaaccttttaaaataaattaaatgcacatattctaaatgctaccaacacaggtactagtcacaaaatctagaacatataacccaattatttcagagaaatcagatgtacttccttggaccagtgagtatatttttgtaactttaaaaatggcactaagagggctggtgagatggctcagtgggtaagagcaccgactgctcttctgaaggtcccgagttcaaatctcagcaaacacatggtgactcacaagcacccataatgagattggaagccctcttctggcatgtctgaagacagttacagagtacttatgtataataataaataaatcttttaaaaaagtggcactaagaactgcatattatctaaataaacatttagatattgatagtcaagaagttttgcatttcctgaaacttcaaatatttcatatctatggcttttttctacaatctttcccacacccggtactttctttagttttttttttttcatgtttatgtgcttttatgatttatatactgtaaaatgaggtaaaaagatgctaatgaattgtgtacttttgccactaaactctatgttttctaccacaattttccaagttcttgattaaagcagtacttgccaaatttgactgtacaaatatccagagccctgttagaacatgactgctcaaagatagggagaaatgagtccaagtgtacatacacagaactgctatgggatgagtgcaaaggcagtgctcctagccatgaatctatatcacaaaacccttgtaactagtagctctgtttgcaaatacacttaaacaccatcttttgaattggctgctggcagtagatgtaagttagtattttcccgtgacagttttcactatatctcgacttcttttcttttttaaaggggaaatcatcaagatacactaaatatgccttataaggagggctcaccatgtgacgattgtccaaataactgcgaagatggactgtgcagtgagttgtctttgttgtttaatttaggttgcctgaagttggcataccaaaatggggaacacacatcctttaagtgaagcattgacctgcggtctatatttctgtgactttcatcttaatatatacattatttctgcttcccttctaacacccagagtaatccaacctctttgactttatcactggagggcagaattagaatatctatatatgaaaacttaccttcttgcccaaaagtttaaaattatgaatatagaagcattctaggtaaattattaatatgcatggctacttttcccagacacttcctgagataccttattgctgaccacaaactattcctgtaaggttaccagtctctctgaaatgcttactgctctataaccctcaacttgtctgtaatagatcagtgtggaattttttttattatcacttttattttttttaaagtcaagtcatggcatttccccttcaacagttcctcatcccgtttctcctccccccatcaccaacaggatgtccatccctgtggcaaaccttcccagtccctgggtctcaagtctctggaggtttaggtgcatcttctcccactgaggccagactaggcagtcctctgctgtatatgtgtcgggggccttggaccagctcatgtatggtgcctggatggctcaatctgagagatctcaagggtccagattagttgagactgctggtcttgctatgggaccaccctctacctcagctccatccagccttcccctaattcaaccacggggttccctgacttcaatccaatgtctgttctaagtgtttgcctctgtttcactcagctgcttgtcaggcctctcagaggacagccatgctttgcacaccatagcatcagtaagcgtcaggtcttggaaccttcccataaaacggattctgagttgaaatggtcactggaccttgtttctctgactcttctctgtttttgtccctgcagttcttttagacaggaacagtcctgattagaaattataactgtgggttagtaaccctatccctccacttgatgccctgtctttctgctggaggtggactctacaagttctcttttcccattgtagggcatttcatctaaggtactttcctttgattcctgacagtctctaacctcgcatgtctctggtactttctagatcccccccccccccacctcctacacaccatggtcgcatatttccattcattatgctggtcctcagagtttctcttctctttcccccacctcataccttatcacattcccttttcctctttcccctccttttccacccaggttctctccctttgccatccatggtgattttcatctccctctcaagtgggattgaaacattctcacttggtcccttctgcttgttaacctccttgaattctgtgaattgaattctctacttttttttttaaccaatatccacttattagtgagtacataccatgcatgtttttaggtctgagtttcctcactcaggatgatattttctagttccatccacttgcctgcaaaactcatgatgtccttgttcttaacaactgaatagtattcaattgtgtgcatctggattgtttccagcttctggctattataaataagaccacaatgaacatagtgaagcaagtgtctttgtgctatggtagggcatatttttttgtacattcccagtagtggtatagctgggtcttcaggtagagatatttccaattttctgaggaacctccagattgatatccagagtggttgtaccatttacaatcagtgtggaatttttgtaagctgaccattattcttcttcattagctcagaatctatccaatactggatttttctgatctctcttatattgccatccagatatactactccttagcatatgcctaaggaacttggcatccttctccacatattcttatgtaatagttgtcattttcactccactcacaataactaggaaatgaaaatatgaacctcttcaatagattaatggatagtgaaaatatggtatgtgtatgcactgtggaattctgttcatcctgaaaaaaaaactggaaataaaatatttttcaaagaaattggtgaaactggaaaatactatgctgattaaggtaactcaggcccagaaaataaatgccacatgttctctcctatttgtgaatcttagctccaaagttttggatgcatgtacctcctggggtaacctacagaaataaggaatgtaagaaggaccacagtgtggggctgacagctctagaaaagcgaatagtaggacacaggcacgatgaaggtgaaaatggcaaatatagaaaggagactataactggggataaaggaatatgggcaatatatagggagaaagaggaccgaggaatatataacagtaaggttgtttaaaaaatatgttgaaatattttatgtttagttcaaaatatacacacagtttaaatctaattatatgacttggagtaataatgcttccctcaatgcttctccccaaatcatagactatctaacaaaatcctgagagccaggaatgtgaaaccttccttccaatcgttggtcaggagtgtctaagagactcaaacacaagaaagtctatttccaatgccttgcctgccagccagaactttaagggaagaccctgatgctgaagatagtacagactttggacacaaaaatcaaagaaattaagcttggattgtgaatgtcacaatctgacaaagctccaactctatagacaaaaacctataggcaattagtgattgttttaaaagggagtatagtactgcaacttggagacctctccttttctaaagagaaacaggggagcaaaagtagacgagagattgcagggaaaggaagttgtagagggagagaggaaaaatgggagaagaggaggatagaaagtagaaacattagttgagatgtaaagtaaataataaattaattaaaaatagccaaagcaatcttgagaaaatgtgagggaccaggtcttgcaatactaagctttaatatatcctacaaagctatattagtcaaaacaggacagtattaatagaaagatacgcctctagagcatatgggcagaatccaatgggcagaatacaagtaaacttgtgcagctataaagagaaggtaataaaaaatttaaaaagaaaaaaaaaaagaaaaggaaataaggggaaagggaaggaaagaaggagggaaagagagagagagaatgaatgaatgaatgaatgaatgaggaagaaaatcagtttttttagggatgagacccttaatagattatccaagtcttgacacatatataaaatctaaagaagagtcatcaattaaagagagacagaggaagaaacattggtgggatcctatgaggaagagtaaaggataaaaatgatataaatactatatataagtatcaaattttaaaatattttattattaaatactttattaatttaaattataagttaaaatattataacatataattcatgccacttatgaaactagcactcctaaagagagtcccatttttcccccattctctcggtcattcataaataaagaatgtgtgtttggtctacatcttaaatttactcctctgtttgttgaatttcttcatggacaatatgtccgttgatgacaatggagtgttaaagtcctcatgcatcacttcttggaaagctactaatatttggtttgtgtacttagaagcttttatgcatatgcacttagaatttttattattttacccacttactgtttatcattacacaatgaccttctttgatttttcctgaagtctattttatctgtaattaatggaatgacacttgctttcatctgaattttattaacatggaatatcttgattcattcatatactatctgctagtgtgaatccccagagattttcttgcaaacaacatctatttaaatattgtcttgttttctgtttgttcatttcctatgtctttcaggtggaaaatgaagattaatttacttacattcaaagacatcaactaatgatgtcattgttcaaacttgctttttagttttctttctttttttttaaagatttatttattatttattatatgtaagtacactgtagctgtcttcagacacaccagaagagggcatcagatctcattacagatggttgtgagccaccatgtggttgctgggatttgaactctggacctttgaaagagcagtcgggtactcttacccactgagccatctcaccagcccactttttagttttcaaataacccattttcttcctctcacacagccttcttgcagttagtttacttaatataatagctacattttgatatctgacttacttttttgttttctattaaagatgttttcttagcaataagttgtcaaaggaacttagcaccagatgtgaggtaccccctgttacattttgtattatagaatttagaaataacagaaacttagataaaataataaaaataaatggagaatactttgatttttaaaagactatatttgcagattgttttctcttctgttagattcctattacatgtcaaactttacatctttctaaatctcttacttcagtatattaaagaacctgttattaattttattattttagttttagtatttatactaaagagatgggtagcttatgcattacatttgtaatattggagtatcctgaatttgtgtgtagaattactattttcactgaattttatacttcccaggatttccactgttcatattataggtaaatttgttcagcttaaagaatcacctgcagcatttttttctataggacaggcatagtaaaaattccctttgacctaattttttctgattttgcatgtatctttccttcatttccaaacataacgttggcagggacagaaaccctgcttcacagtttctttccttccacactgaaaaaatgttcaagtccctgttttcctaagccgtggagcatggactatgtcagcacactgctcttaagaatctcctttctaactttccattttgagttgctgattatacaacacatcacgatatatgttgagctaaataaggagggcaaacgtgactttcctatacctggatattattactttgtacagattaaagaatgaacactgtcttttgcgatgtcagagggatggtggagcgtgtttataatgacctgaatgtcacaatgtcacaggtctagaggctcagttcatgaacagtgatttagagggaggagctggacaattcctgatactgaatttctctatgttgagtttctttttttttattaaggcatttttattagatattttcttcatttacatttcaaatgctatccccaaggccccctataccctctcgcgccctgctcccaaacccctccactcctgcttcctggccctgacattcccctgtactggggcatataatcttcacaaaagcaaggtcctctcctcccattgatggccgactacatcatcctctgctacatatgcaactagagacatagctctagggagtaatggttagtccatattgttcctcctctaaggttgcagaccccttcagctccttgggtactttctctagctcctccattcggggcactgtgttccattcaatagatgactgtgagcatccgcttctgtgtttgccaggcattggcatagcctcccaagagagagctatatcagggtcctgtcagcaaaatctttctggcatatgcaataaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntcacgagcaggaggatctctctttaagctgtatttttatgttgaggaagaactatcatggcagtttttcagttagtttgactgctttccctattttcataccataaaaccaagtactgagatctcacagatggtgaatttcttgtgatgatagtttcatgcctgactaggcactgaaatgaacgtgtctttagagagagttggttggagagcactttttcttttttctttttcttggatctatgggagtcatgtggaagcatagggagaagtattgcaggccacaaggggaatagggagtccacaagaagaccaaaagagtcaattcacctggacccttgggggctcccagagactgaaccaccaaccaaagagtgaggatgggctggatctagccccctggaaatatagcaaatgtgtaacttggtcttcatgcaggtccccgaacaactggagcaggggctgtccttgaacctgttgcttggatatagttcccccagcagcgctgccttgtctggcctcagtgggagaagatgagcctaatcctgtagagacttgatctgccagagtgcagtgataccaatgggaggcatctaccctctcatcagagaaagggatgggagaaggaggaggagggaggatctgtggaaaggggatactggcaggagagggggacagatatagggaggtaaagtaaataaagaaatcaattagtggaaaaagagtatatccttcaatgataaacctcaaatacattttaaaaagtgactgaaatgccagtgtcttatcataattgtcatggtaatattgttctcaaaatgacagagaaattgcaaagatcacttatctccagtctgttcaggtgcagtgtcaccaattacaaaactgatattgaataaacatgcttcatagcatgacaagcactttctcctttacttcaaggggatctgattcaacatgtcaattgttttgcattaacctaaaaagctaaccttaatcaagttaatcctttattatatagaacttttctcttgacgtcataaaaagatgatgtctttccttaatggtcacaactgttttaagcacattgttgcataccattctttttgctttccagctaacccctgcatttattatgatgaatacaacaactgtgatacgcaggtaaaactttatgggtgctcacatccagctgtccagccattctgcaaggcttcttgtctgtgtacaactgaaataaaataacctaaagtttgctactatgctctgaagataaagtgtgttttcaggctcaattatttcaccttaatgtaatgtactgtaagattttgtactgaatttcatttttttaaattatgctcaagaacttaatttttaaccatcatttatagacatcaatatattttcatcaattctgtgcccagtgcatgtaataacaaagatttaaatttcattgtttaatgaaagggcaagtcaaatgcttgccccaaatttaatacatgtgatttgtgatatgtagctaaacttaatccagctaatgaatattcttcatctgtaacttgtagtaaaatagcttttacctaacccttttcccaatatctccacttcccaacaaccctaattttttttgttttaatgtttaattttcattctattttaaatcagacattaaaaatattcaacaaacaaacaagagtcaggcttcaacttgaagtatagatatgactcaactaagagtggtcaaaatgtctcccaaactcattagtatctcagcatgcatgtttgtgatgtaggaaatgagagccaggctgtctttgggttccttgtcccttcacctagatttctcctgtgccctcctacacacccctgttgccctgagcaccccattcctagattgtcgtgtatctaaaaccataatgaaatgaccataacaccaggctgccaaattatttgaaatatctaatcctttaaattatattgctttactagaaaaattatgtcctaagatagttctgtatacagttctaataaaaggaaaaaagttctgtatacagttctaatcaaaggaaaaaaagtgtgtgtgtgtgtgtgtgtgtgtgtgtgtgtgtgtgaaaggtgtgtgtttgtatgt

SEQ ID NO: 4 - DNA, 20590 bp, Rattus norvegicus
  misc_feature (6299)..(6300): n is unknown
gttttctaacatttccctttatcattaattaaagacaatttatagttttttatatgccttcacaatagggacatgaggaaagttagcaggttgtagcattctgtgctaggctctgcccacctgggtaagaattctgataaagattttggttgtttcttttgatttaggcttctcctcttatttgagcaaatgtttttgaagttttcagtagatctttggttgtattaaaattatggtgatgttgtcatctgttcaactaatattctaaactctcggctctatctctactctgtctttatgggctgggtaatgtctgcagcaagttactattcagacatccataactatgaagcaggcacttgttaagtattgatagatttaaggaataaaacgtcctcatgtctgttatcaggttagctattgtagtgataagtagaaaagaatagaatagtaggcaatcagtcttggtgataataaaggatagcatcagattgttatgtggaagtagaagaagggagcataagttgggatagaaaaggagagaaacatggtggatacatgggggcttgtattaatgacaaactgaaaagatgttttagatatcacaaagcaagtccatctctaagctttgggtatataatagtgaggaaaatgagcatgggaaaaagaaacaaaagaaagagacagagagatatagagacagagagagagagagagagagagagagagagagagagacagagacagagagagacagagagaggagagagagagagagacccaagctaggacaagaatgagattaagagagggaaacaggaaagaggtaaacagtataagacagtgtaggccaactttgctgacctaatgtactctataaataagaagcagctgctgcttggagcagagctggacagaattgtaagataatcaaatggctggcttgaatgttgttttcttctgggcagctctgttgcattgttttgatttcctggtctgggaaatgctctgagcttgttgttaatggccctactcctcttatacagtggatcctcagttcctttatatgtgattagtattgtgagtgtttgttaggatgtggaaaacactggaaaaagtggtgctaatgagagtggtgttgatgaaaaaggcagaataatgttacatggtttatttaataaagcatccctcatgttacacatcttttcagaggacttgggacatctaagagcaatgacaggacatccatgcgactacagaaacaagcataagtactcctgtttactatgaaatttttcacaaactgattaggttgcttttcaagatcataaatttctataaaagataactagtaggtttatagaaatacacataatagattgttagcaaaagtgtcaccctattaagatcttagaattgacaaactgtattaaataagaataaagatccattagtactgattttgttacaacacaaacacaagtatttgtagccaagtaatagagagagatgtgcagaatatctttaaaccacatacatatatacattagtagtaacaggatcacctgcttctgtgtactctttcaaataatgcctggctgatggagcctatctcgtgttataggctatagatgttgtctcttgacttttacaattaggcacctttgggattaacttgaatcaatgaagaaaacgtagttctatctctgttcatcacctggtgagccatgtaaataccatttgctactttccttccgtattaacaaacagttgtgcctattcagactctggaaattaagtagtttacattttgagtccatatcatttaaatcagtggtatattattttctgagtccccttagctaagacataatattggactttattttcatgtcctattataaaaagatagccatgtaaagttagttcaaggttccttagaaaatagtttcccgcacataaagaataatctaaaattctgttacttttgtttttcaggtatggcaatgaaactcatcttgttattgtttcttgctgctactcttactgtctttgttcctgttgtgactctcagagtatgtaaagatcatatgttacatacctaattttagtttaagaatacccaagaaccctttgatgggatgtagaggtaggagaagggtgtatcaattattcacatacttcatctaatgatgcagataaacatgtcctattctgttttagaaaaagaatcatttgtttattttatgttttcgagtacactgttggtgtcttcagacataccagatgagggcattggatccctttacagatggttgtgagccatgtgattcgtgggaattgaactcaggacctttggaagagcgttcattgctcttaaccactaacccatctctctacccccaataagtcctattaatacagcagaagcaagggcacaacaaaagaatagaaggcctaagtgaactgtcaagtttagaggactagaatcgaacaagaaaatatcctgagtgaggtaatccaatcacagaaaaacacacttggtatgcacttattgataagtggatattatcccaaaagtctgaatacccaagatgcaaaccacagaccacaggaagctgaagaagaaggatgaccaaaatgcagatgctgccactccttcttaagaggaaaaaaatatctataggaagggatatggaggcaaagtttagagtagagactgaaggaatggccattcagagcctgcccgacatgtggcccatatatatacagccaccaaaactagataagattgatgaagctaaaaagtgcatgctgaaagggaccggatatagatctctcctgagagacacattcagagcatgtccaatagagaagcgaatgccagcagcaaaccactgaactgagaacagggccctttttggggggaattagaggaagaattgagagagctgaaggggcttgtaatcccttaagaacaacaatttcaaccaaccagagcttctagggactaaaagactagtgaaagagtttacatggactgacccagggctccaactgcatatgtagcagagaatagccttgttggaacatcggtggaagggggagtccttggtccttccaaagttgaacccccagtggaagggaatgtcggcgggggcagtaagggggtggatggggggaataagaaaaaaataaagtgcatggagtgcttggattttaattttaaaaaagtagagaactggaattgatgatgcttaaaattccacttttattttaatgcattgggccccagtctatgtcatttttataaacacgaaagaaaggaagaaagaaagaaagaaagaaaggaaggaagaaagaaagaaagaaaaaaaaagaaaggaagaaaggaagaaaaaagaaagaaagaaagaaagaaaggaaggaagaaagaaaaaagaaagaaagaaaagaaggaaggaaagaaaagaaggaaggaaagaaagaaggctagcaaagaatgcaataaaggggaggagggatggagggaggatgagaaataataaaagcataggcaagaatttctttacaatttatccatgtgttttcataactaaatcaaagaaaaatagacaaatactgttattgctacttttcctcccacataaccatatcttcaaatgaggtatattctttttgattttaagaatattataaattgggctggagagaatgtgtactttttcttaaagcggagtcacacatttgaagctgaaatctgagtttgaattttctttatgctttctagtatacccgcttgtttttaaaaatccatggagtttttaaatctaagcaataaaaagagatataaacctggagcatgaaaagaccacttaatcatgtgctcaaaatacactcttaattttggttcttacataaatgtgcacataaacaaacacacacacacacacacacactttgacaaggactcaagtgtgcattgttcatttgtcaattacacaggcattgcttttagcagctgtgcagagaatgagctaaggcagagagttgttcagtgaacaagagacaccaagccatggcctctgtgtttcccattgtccaataacaagaacttctacttgactataagtatgaatttgaaaaaaaattgtatactcttttcaacacaaaatgtagaaaaacttgtaaatcaataattcctgaatactgaccattattgtttgttcactgtaagttctaatttcacattgcctatacatctacatgcaatagacttcatgtgacaagagccacttcaaagaagtgatattaattaataattttctggtagcatctcaaactggatagagctctctataatcaactgatcacggaatcacaaactgaaccacaggaagaaattgttgatacacacaatgccttcaggagaaacgtgtccccgccagccaggaacatgctgaaaatggtaagtttaaacatatcatcagtggagatgcatggggcactgggggcatttcctacatcgttacttagtcctatgtactgttactatgccaattgttcggtcattgcaacaaatctatgacatagcagcagaagaacctgagtctaagaggatgtgagtaactctcccatgtacatgacagagcagttcagatatgtgacaaggatttgagaatcaggatgttcgaattcatagctaaaaaattagacagacattaaacactagtattgaatttcattgaattccatagaaatctagagacccagcgtggcagtgcaaaacctgatggctactatcacaagacaaatttagggaccttggtcatcatccttgggcacaagagagccaagagtgatggagttcagataataatgcaggccctagagcaagagttttcaacctatggtttttagtcactttggagggccacatagcatgtttgtattatagtttacaacattaaaaaattacagctatgaagtagaaacagagtaaatttatggttagtggtcaccactataggaggaactttactaaagagtcacagcattagggaggttaggaaatactgccctagagggggatcctaaatgacagtactctcttgtgaacaatggagaggaagcaacaagagaaaaaaacatgtaatttctgaaaaattatctctggaccactttaaaaagtaatagtattatttgataaactatcttaaaagggttagttgccgtaggaatccatgtttgtgctactcttaataaatgttcttgagccaaccatagtggttcatatccagaatcccagtattcagatagactaccaggctgaggctaaggataagggtatgtacaaatctaaactaggcggttggtgagctcctgaagaaatattcttataattagtaaaagtaatttagtttatttaattttgcactacagctgtctccatttcaaggagtctcaagaatataatctgttttatgctaaatccttcattcaagggaagatcgccagagactctgatcttaattatattaaagataatttccatctcctttctgccagatgaacaaacaactatttctaggattgcctctctattgcgaagtcctattgtatacagaaagtaattttggcgttttatatttcctcagctggccacacacacacacatgaaagagtaatctttcttaacttgaaaaatggaaaaaaaatttgttttgtaggggctggggatttagctcagtggtagagcgcttgcttaggaagcacaaggccctgggttcggtccccagctccgaaaaaagacccccaaaaaaaaaaaaaaaaaaaaaaaaagaatagtcttacagccagaatacagcaaattcagaggcgaatgccagcagcaaaccactgaactgagaataggtccccgttgaaggaatcagagaaagaactggaagagcttgaaggggctcgagaccccaaaagtacaacaatgtcaagcaaccagagcttccagggactaagccactacctaaagactatacatggactgaccctggactctgacctcataggtagcaatgaatatcctagtaagagcaccagtggaaggggaagccctgggtcctgctaagactgaacccccagtgaactagactgtgggtgggagggcggcaatggggggagggttgggaggggaacacccataaggaaggggaggggggaggggtagggggatgactgcccggaaaccggaaagggaataacactcgaaatgtataagaaatactcaagttaactgaaaaaannttctcntggactgtttgaataattcatatatactggatattggtcccagttatacatactgtttgctactcattttcctatgatatacattgctttgttaactccattaatttttctnnctgctatttatgagagaggctttttgttantttccacctctgtttnntccangtgttttcttcgttgttgttggtggtgtgtctgactttgcatttgatacagaaactanggcttagaccaagatgtcactcgatgtaggtctcttccccaaagaccttacatttaagccccccattttgagctcagtnnnnnnnggttaannnctgcacaccnnnnnnnntnnnnnnnnnnnnnnaannnnnntttnnatgnnnnnnnnnnnnnnnnnnnnttnnnnnnnnnnnnnnngtnnnnnnnnnnnnnnnnnnnnnnnnnnnnnnnnnnnnnnnnnnnnnnnnnnnnnnnngnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggtgagagcttgtgggacagtttnnntaaaactgaattttaaataaataaagatattaatgaatagattacacctcaaaaaaaaaagcattcacacaagcaactggacaatagaaagctaacagatttaatctacaataaccttcaactatccttgatggtagattgtaccttgatacacattgcttttgatttttaaagtttggatattcttaattttggagtttaagctctcatccatgggaggaagcaatcatgacaagcagcattaaagcattgattacatctggatagctgggaagtttccatccaatgctatcttagttgtccacaatttcagagtggggatttccttagttttggagcacagagacatgaggaactgtggaactacccagataaacaattttgagttctcaaagacaggctcagttttgtcatataaactgaacaactactacccaatatttaatgcatcatatggaatttcacaacattttaaggaatgctttcttccaaactaactttaaaaaatacacatacaactttatgcctacagattattggtactcttatgatatggtgttcaacaatggggctgtctttatattctagagaaagacagattattcttcagtaccatatctacctgaagcatgtatgataaatgtgtctaatgtctttaagaatgtgttccctctttgtaactgcttttatctctagagcttgttcacttacagctaccacagctcatacagacttttaccatcattttagacctgttaggagacaggttgagagatctgaaggcttgaaggggctcgagaccccatatgtacaacaatgccaagcaaccagagcttccagggactaagccactacctaaagactatacatggactgaccctggattctgaccccataggttgcaatgaatatcctagtaagagcaccagtggaaggggaagcccttggtcctgttaagactgaacccccagtgaactagattgttggggggagggcggcaatggggggaggatgggggaggggaacacccataaagaaggggaggggggagggggatgtttgcccggaaaccgggaaagggaataacactcgaaatgtatataagaaatactcaagttaataaaaataaataaataaagctttctcatcatctgtctcaccagacttgacacaccatactcattgagcaaaacagatagacaaccgagtttattttctgttttaaaacaatgcctaggtgacccttaaaacatttttgaagtaaaatgaggagaacatctgtgttctactttataaaaatattatacagccatctttcctcatttcagatacattttgtggagaaaatatgcatatggaaaactatccctcttcctggtcaaatgtaattgaaatctggtacaatgagtcgaaatatttcaaatacggagaatggccatccacagatgatgacattgaaacttatcactacacccaggtaatctgtatccttccagatacacacatgactggatacatgtgtagttaaaaaataagtattggttttcttctcagaagtaaggaaaatggcccatgaaatcctacccatctagttcctgaattgtgaccaggacaatgataacaccgcttgtcttgacagtgccaacaggggaaattttacctgatcccgccttttagttgaagtacagggccaatgactgctcagctaaagacagtgtcagcattggacatatgagaaagcaaatagtaaagaaactcagtaggttgaatatacaatgtatatgtacatatatatatacatatatgtatatatatatatatatacatataatgattaaagaagtaacaagaagctcatgggaggagtttgaagtgggagagccaaagtgatatagatacacatgtatgatgttctcagtaaaagatactttgtttgtatttttctgattaaatgaagataaaagacaagcaataaaattacaaatggctaccacacctgctgaagtctttatataggttctggagttctatactctggtccccagtggaaaaggcaaatatttcgtcacttccccaccccacttagcttctttattagcatatattaattgtataaaataatgtttgttatgagagttcttacatgcatgttatactcacttcttttattttctatttattttccctgtcctatctccacattcatcattctaatggtccctttattaccatgtctccttttgacacatggacttcatatgtgagagaaaatctgatccttccttgattaaggaggttatccatattcactcaataaaaagatccatagctctatatattttcctgcaaatggcataatttattgttcttagtgtttaaaaaaaaactctctttttattctttctaacaaattcatcaaataacagctacctaggctggcttgtctattgtgaatggtactatggtaaagacaagagtgcctatctccaaactgacctcctcagaagatgcacagtttatattcctaccagccaccattgaagaatccttctctccccacatcattgcaaacatctctcctcttattcttccagagggcaatcattctaagagcagtgacatagaattttactgaaacattgattaaatttttctaacggttctaaataagttgaacatttatcatttatctattggacatgcatatttctttcaaaaaacaaccaattgatggttaaaatgttcaaaacctgacactgtctgggaatggtattagtttcaaaccacatcagatctactttaattatttttaattagtgttctgcaagtctcaccttgaaatgtttaagactccttgctagggattctaagtagtttattgttattgtgcttcttaaacagaacaattttcttaatttattttcttcttattcattcagtcttagtgttatagaaagtgagagagtggttaataggaatagagaaggtgttcacaacagaaacaaagtgatgatattacccagtgtaaatttacaattattttaaatgccagaatcaatcggtggaggaaaatagacaagggattgacttaatttgattaacaggttgaatgatcatttgcctgccacacagagtccaagttaattttaaagaaaaaaaaacttaaatctctaaatttgaagtaacaaagtatatactagaagaaagaagatttgaatatttaaaatggaaaacattagaaacagtgattgttcataaagcacaagactcaaaagcattcattcatgagtctcataacttacatgaagtaaattttagggaagattgaggttaaacgtttctaaagatataactcaaaataaccagtcatgaatgcatgcacggaattgttaaataaacatcagcacatcatattttctttcaaggccaatagcacagaacatgaatggtgtgtacaaagaatccaagatgccttcagacttaacactttaatccggtgatttatgatgccaatggattagaaggaaacctgagagagaaagacagtaaatccaggatgatgtagcctccatcttataacccattcataaaaagtcaacctcatcctgtttccctatcataatgcttttggctctttcagtcttgtgcagcaatttaccccatgaactttttatttatcaaaaagaagcactatactaaaaagttataaaaatgactgctcccatttgccaaaggttctagaaaatgtataaaaattgtatacatgtaagaaacatcaattaacaaaaagaagggccatacatttgaaaaaaagtagaacaattatatatgaggatttggagaaagaaaatgtaacagaaaaatgcaaatgctgtaaaattattataatctcaaaaaccaaagaaatagtttaaaaaaaaacaagaaattagtctttacttgcttaccttttaagagaaaaatagtctcattatttgtagaatatagggatggattaatattaatttttcacattatttgtactttttcttggattaggaactgaatgaataaatggaagaattacactcactacttttaagactaatatatagtgtgaatacaatgcaatagatattctaatgcattttgttgcatttttcctaagtatatgttctatatcattcagaggttaacaaaattttataagacatatttaggagaaagaataagcagagaaacttgctatcattgagtggcatcacccaaagtttgcatagtggatggagcctcagatttggtgaaagcatattcttattttcaatagatacttaagtgctgcatggtgtgcagggagacagacacctaggaaagttgtgaacagaaaataatactttacaaaatcagccagagaaaactactatgatggaaagaataaggaggagcgagaggaggaagaggaggagaaagggagaaagagggagagagagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntattacatgcatattacagatgagaaaatgaaactgctacatccaaaccaaatcaaaccaaactgaccaaacaaacaaaattataaaattattacctagcatacaagtacagagaaggaatttaagcttggctcatttgaattaaaaaccatttgagtattatacccaatcttgaccagtgagaaaacactgttcctgggtttgagttgtgtgaaaacaaccattactgtattagcatattcattacagaggtttaacatgtagaggttgagctgagtagatgaatccgatcctggttttcagtgggaatgaatataacaggacacttgttcccctaagatgacataaacgcatgagtgtttcatgtctgtcttctgtgtgtgaatgtatgtgtgtgtgtccccatacgtgtgcacacatgtgcttgcacttgagtgtgcatgtgaatgtgcgtgtgtgtgtgtgtgtgtgtgtgtgtgtgtgtgtgtgtaagtgtaaccatagaggtaataaaaaggccttggatacccttgttctggatttagagatgctattggtagtccaggtggacggtggagtcactgtcatgtccactaaaggagaagtaagttcttgtagccactgaagtatcactccatgccctgtatgcttatgtgtaaagatgatggcgttgatgttttagtcattgggaaacatgaaagtctgttgagtcatctcttatctttgcagatggtctgggcctcttcctacctcattggctgtgatgttgcatcatgccgcaggcaaaaggcagctacgtatctctatgtgtgtcactattgccatgagtatgtaacgccagtttattactctttctaaatatattgtcttcctacaataaccttgtataatgtttcaaatattataaatgaacgttctgaatttctcccagttactttgttagcgaaatgtcacaaatataccccaaatgtttcgcttttcatcatgtttttattacataagttattagagatacacacatttggacaatttgttggtgactctgttctttcacttgaaagtgttacgacacagggaagggaatgtgactgggttaggctaagaaaggatattcagataaatgataccctctgcgctcagcttggggatcccaatagtgacttgtgtgtttccttgtgaaatttatgtggcactggggagatggctcagagatttaaagaacctgttacaaagagcaagaattgtttccaacacccaagaactggagtctcaggactgccaataacctcagcttcaggaattctatgccttaaccaggcttccactgacacctacacacagacacaaacacacacacatacacatacacacacacatacacacacacttacacatacatatatatttatatataatactatttggttttaatggggagataattttaaaggcaatatgtacatacatatattacattcatatatactacatgcatacaaacattcatatatacaatataaacacaaaccaaaacaaaatttttttaaataaatgtgcatattctaaatgttaccatcacagcaactagtcacaaaatctaggccatagaacccaattgcttcagggagagcagatgtacttccttgtcctagtgaaagcatttttataactttaaaagtgacactaaccaaagactgcagattatctaaacaacatttagatactgatagtcaagactttttgcatttcctgaaatttcaaatatttcctatcttatggctttcttctatactctttctcacatccagtactttccttagttcttttttttcatgcttatgtgcttttctgattttatgatagttttcccatgggacacaatatcagatgctatactttgcatcactggtacaaaatgagataaaaagatgccaattaattgtgtactttttccctagactttctattttccacaacactttccaagacattgactaaaacttaaaaaactaactacctaatgccccattatcagatttgactatacacatacatggagccctggtagcacatgactcatagcagcattgagtccaagtgtacttaggcagaactcccaaaggatgagggcaacagtggcgcttggagccatcaatctagatcacgtaaccattttaaatattagttctgtttgtaatgtccctgaaaatccatcttttgattcgactgttgacagtggacacatatgtaattaaggaataaataatttccaggggtggttttcattatacctctacttcattcctgttttaaagggggaatagtcaagacacattaaacatgccttataaggaggggccgccatgtcaagactgtccaaacaactgtgaagatggactgtgtagtaagttgtgtttcttgttaaatttaagtcacctgacgttggcgtattgacgtggggaacacatgccctttaagtgaagcattaaccaacgtagaatattcctgtgattttcatcttaatatttcaccaacatagatctatattctttctgcttccattcccctcacatccagagtaatcaaacctctttgactttatcactggatggtagaactagaatatctaaatatgaaagctcagcttcccatttaaatggttaaaattatgagttagaggcagtctaggtaaaatattaatatgggtgactacctttcccctcaactacctgagataccttattgtaaactacaaactactcctttaaggttaccagtctttgcaaagtgcttgatgtcccatcaccttcaacctgcctcactgtggaaatttttttactttacttttatttttatacagtccagtcgtagcccttccacagttcctcatcctattactcctccccttgtccccaagaagatgatcctccccacactagacctctccactccctttgcttcaaagagacgcacatcttctattctggagtgttaggtgcattttctcccactgaggtcagaccaggctgttctctgccggatgtgtcatgggcctttggacctgctcacgcatggtacctggttggtagctcagtgtctgagagatctcaagggtccaggttagttgagattgctggttttcctatggggttgccctcctcctgagcttcttcctgcctttccctaattcgaccactgtggtccctgacttcagtccaatggttggttgggtgtaagtatctgcttctgtttcagtcagctgcttgttatgccgctaagagaactgccatgctaggcacaggtctgtaagcacaccaacatccataatagtgtcagcccttggagacttcccttgagatggatcccaaattggaccggttactggactgcgtttctctcagactcttcttcatttttgtccctgtagttctttcagacaggaacaattttgggtcagaatatttgactatgggatggcaactttatccctccacttgatgcactgtctttgtgctggagtttcctctacccaccttagggcacttcacctaagatccctccctttgagccctgagagtttctcacctacctcccttcaccatacaatcatattccccctttcttcttctcctcacctcttccactcaggtactttcttccctctgtctcctgcgattgttgtcttctccctccaaagtgggattgaagcatccccatcctcacttgggcccttctgcttgttcgctatgtggacctgagtccctgtggtatggtggggcatcttttgagcaaatgcccaagagcagtatagctgagtcttcaggtagatatatttctaattttctgaggaacccttgaattgatttgcagagtagttgtaccagtttgcaatcattgtgtaatgtttttaagttgttcatcattcttcattagctcaggttctatctgttactggatttatctgatctctctgatattgctatccattataccactccttagtctatgcctaaagacttggcatccttctccatgtatttttacttagtatgctcattggcactccactcacaataactaggaattgaaaacaatgtgaataaaactagcactagtaaagacagtccccaatttttctccttccattggttattcataaataatgtgtgtttggcttacattttaaatttactcttttgtttgttgagtttcttcatggacaatatgtccattggtagcagtggagtgttaaagttttcatgcatcacttttggggaggtgactaatgtttagtttgtatatttagaagcttttatggatatgtagttagcatttttattattttacccgcttaatgtttatcattatataatagccttctggatttttttttctgaagtccattttatttgctattaacttagtgactcttgtggaaaactagggaaaaatattccaggccacaaatgcaatagggactccacagtaatacaaagtcaactaacctggacccttggggactcccagagactgaaccaccaaccaaagagtggatgggctggccctaggccctagaacatacatagccaatgaatcttcatgcaggttccccaacaactagagcagaggctgtccctgaatctgttacttgtctgtggatatagtttcccaagctggtcttacttgtctgacctcactgggagaggatgtgcctaatcctgcaaaaacttgatatgccagagtggatggatactcaagggaggtctccttcctctcagaagagaaagggacagggaagagggagcggagagaggatctgtgagaaggggatactggtaggagaggggctcagatataaggatttaaataaagaaattaatggaaaaagattatgtctatccttcaatgacaaacttcgcattcattttaaaaagtggctgaaatgtcagtgatacatcattatttttacggcaatattgttttcaaaatgacagagtaattgaaaagactgtttatctccagcctgttcaggtgtggtatcaccaattaaaaaactgagattgagtaaaagagctttagagaatgagaagcattttgtcctttacttcaagaggatctgattcaacaagttaatttcattcagacctaataatctacgttaatcaaattaatcctctattataagaatatttctcagagttcaaagctagcagcaaatcactgaactgagaacaggacccctttgagggaattagaggaaggattgaaagagctgaaggagcttgcaaccccgtaagaacaacaatgccaaccaaccagagcttccatggactaagccactaccaaaagactgtacatggactgacacaggggtccaactgcatgtgtggcagagaatagcctcgttggggtgccaggggaagggcaagcccttggtcctgccaaggttggattcccagggcaagggaatatgggagggaggggcggtaagggaggtggaggggatgcgggtttatggacaggaaaccgggaaagggaataacatttgaaatgtaagtaaagaaatatatctaataaaaaaaatttttaaaagaacatttctcttgacctcttaggaagttgatgtctttcctcagtgttcacaactgttttaatcacaatgtttcataccattcttttttttttccagccaacccctgcctttattacgatgaatataacaactgtgataagcaagtaaaacttcttggatgctcacatccagcagtccggccattctgtaaggctttttgtctgtgtaaaactgaaataaaataacctattgcttgctgctatgctgtggggtaaaagggtgttttcaagatcatctatttcacctaatgtaatggactataggaccttctactgaatttcacttgtttttaaatttgtgctcaataacttaatttttaagcatcatttacagacatcagcatatttcatcaagactgtgccctttccaggcagtaaaataactttaaacttccttgtttaatgaaagggcaagtcaaattcttgccccaatttaatacatgtgatttgtgttgtgtagttcgacataaccccactaatgcaggagctcttcatctatttactggcagtaaaacagtcttttacataaccccttttcctaatatccccatttcccctacaatcctaatgtttttgtttaatgtttcaattttcattctattttgaatcaagcattaaaaatattcaacaagtgaataagaacaagagttggacctctacttgatgtgtagatatgactcaactaggatttaaataactttgtgtctttgtatgcatgtttgtgacatgggggaatgagggtcaggctgtctctgggtttcttgttccttctcttctacttctctagtgacttcccacattcttcccttgccctgagcatcccattcccacattgttgtgtttctaacaccataactgaatgcacataacaccaggatgctaaattatttgaaacacctaatcctttaagttggtcgcattttattacttcattagacaaaccgtgtcctataaattgttctgtacagcattctaatcaaaggaaactgaaagaggaacataaaaaacggtgacactgtggtgtgttcttcacatactcgttcacttagaactgtgaaccttgccagatcaaatggaatataaagatctacatgcctgaatatgaaagcgacgttgaaaggcacacagatatgtacaaagtctgatgtttggtatttttagttgttctggaatttcttttttcaaaaagttcacctgcactttctgaaaatggtcccagtgcaaagttcctcaagctcctgtttcgctctttcttttttcgtacacgtagtaagtggaagtgcacacaagcctgtttagcttcgaaagatgggaattttatgttatccaaatgtgatttcctgcgatgttttctcataaaatgtgaggcaaagagtaggacacgtttctacatccttccagagccaaagctcatgaaggacgtttgtgtactcagcagcacacttacgttataacatgacaaaggatataagtgcttcaggagaagatcgccgtcctctgattccagcatccaacacatatgcatagttatgtgtgcctgtaagcacattaatgaatgtgaaaggtactctgattcctgttcgagacatgttgaatccccgggggaccctaaaagggatggcaggtgtatttccatcctcccagatcagggcctcactagttgcagcccccacaaactcctagacagaggagagaggtttgagacagacaagtcacatagagcaatgccccaaacccctcctccacgggtgaggaaatgaccacaggttactaggctcaagacagatcagtcaatagaaacaggaccacgcccccgaatatgtagatgagatctgactcatccaaaaacctatttaactgtcatgttcagaatgaaaggtgtgcaggaattaatccattgtctgagagcttctgtcttacgagctataacaccgccgcttggggaagagatctgctcttcctgaaacttctttcagaaacccctctgcacccctggtaggttagtcagtctcctgctggctcagctcgggtcctagccctggaggaaacagaggtggagcagggtccgcagctggggttggtggaagcagtcccctctctctgcctgagttctctctccctctcacctaacccaaagagctaagcaggtaagagatctccatgggagagctccgaggcatcagag

SEQ ID NO: 5 - protein, 254 aa, Mus musculus
MDVGMAVKFILLLFVAAFVPVVTIRPLKLDRALYNKLITESQTEPQEEIVNTHNAFRRKVSPPARNMLKVSWSSAAAENARILARYCDKSDSDSLERRLPNTFCGENMLMEHYPSSWSKVIEIWFNESKYFKYGEWPSTDDDIETDHYTQMVWASTYLVGCDVAACRRQKAATYLYVCHYCHEGNHQDTLNMPYKEGSPCDDCPNNCEDGLCTNPCIYYDEYNNCDTQVKLYGCSHPAVQPFCKASCLCTTEIK

SEQ ID NO: 6 - protein, 250 aa, Rattus norvegicus
MAMKLILLLFLAATLTVFVPVVTLRHLKLDRALYNQLITESQTEPQEEIVDTHNAFRRNVSPPARNMLKMVSLNISSVEMHGALGAFPTSLLSPMYYTFCGENMHMENYPSSWSNVIEIWYNESKYFKYGEWPSTDDDIETYHYTQMVWASSYLIGCDVASCRRQKAATYLYVCHYCHEGNSQDTLNMPYKEGPPCQDCPNNCEDGLCTNPCLYYDEYNNCDKQVKLLGCSHPAVRPFCKAFCLCKTEIK

SEQ ID NO: 7 - DNA, 21 bp, Artificial
  Mouse homologue of hCRISP1 TaqMan forward primer
actcagatggtctgggcctct

SEQ ID NO: 8 - DNA, 20 bp, Artificial
  Mouse homologue of hCRISP1 TaqMan reverse primer
ctgccttttgtctgcgacaa

SEQ ID NO: 9 - DNA, 27 bp, Artificial
  Mouse homologue of hCRISP1 TaqMan probe
cctacctcgttggctgtgatgttgcag

SEQ ID NO: 10 - DNA, 21 bp, Artificial
  Rat homologue of hCRISP1 TaqMan forward primer
attggctgtgatgttgcatca

SEQ ID NO: 11 - DNA, 21 bp, Artificial
  Rat homologue of hCRISP1 TaqMan reverse primer
cccctcatggcaatagtgaca

SEQ ID NO: 12 - DNA, 26 bp, Artificial
  Rat homologue of hCRISP1 TaqMan probe
ccgcaggcaaaaggcagctacgtatc

SEQ ID NO: 13 - protein, 16 aa, Mus musculus
RPLKLDRALYNKLITE

SEQ ID NO: 14 - protein, 14 aa, Mus musculus
DEYNNCDTQVKLYG

SEQ ID NO: 15 - protein, 16 aa, Rattus norvegicus
RHLKLDRALYNQLITE

SEQ ID NO: 16 - protein, 14 aa, Rattus norvegicus
DEYNNCDKQVKLLG

SEQ ID NO: 17 - DNA, 980 bp, Mus musculus
agtacagacaaagaccagatttgtatcagcacacaattttaagagatacatggatgtggacatccagccactttcccggccagaggataggtgtccgcccagcccaggagcgctttgcctgagcatcagcagtggacatcttggttccaggactcctccaagtgtattctgcacaggtatggcagtgaaatttatcttgttattgtttgttgctgcctttgttcctgttgtgactatcagacctctcaaactggatagagctctctataataaactgatcacggaatcacaaactgaaccacaagaggaaattgtcaatacccataacgccttcaggagaaaagtgtccccgccagccaggaacatgctgaaagtgagttggagttcagctgctgcagagaatgctagaatcttggcgaggtactgtgacaagtcagacagtgactcacttgagagacgactaccaaatacattttgtggagaaaatatgcttatggaacattatccctcttcctggtcaaaagttattgaaatctggttcaatgagtccaaatacttcaaatatggagaatggccatccacagatgatgacatcgaaactgatcactacactcagatggtctgggcctctacctacctcgttggctgtgatgttgcagcttgtcgcagacaaaaggcagctacatatctttatgtgtgtcactattgtcatgagggaaatcatcaagatacactaaatatgccttataaggagggctcaccatgtgacgattgtccaaataactgcgaagatggactgtgcactaacccctgcatttattatgatgaatacaacaactgtgatacgcaggtaaaactttatgggtgctcacatccagctgtccagccattctgcaaggcttcttgtctgtgtacaactgaaataaaataacctaaagtttgctactatgctctgaaaaaaaaaaaaaaaaaaaaaaaaa

SEQ ID NO: 18 - DNA, 981 bp, Mus musculus
agtacagacaaagaccagatttgtatcagcacacaattttaagagatacatggatgtggacatccagccactttcccggccagaggataggtgtccgcccagcccaggagcgctttgcctgagcatcagcagtggacatcttggttccaggactcctccaagtgtattctgcacaggtatggcagtgaaatttatcttgttattgtttgttgctgcctttgttcctgttgtgactatcagacctctcaaactggatagagctctctataataaactgatcacggaatcacaaactgaaccacaagaggaaattgtcaatacccataacgccttcaggagaaaagtgtccccgccagccaggaacatgctgaaagtgagttggagttcagctgctgcagagaatgctagaatcttggcgaggtactgtgacaagtcagacagtgactcacttgagagacgactaccaaatacattttgtggagaaaatatgcttatggaacattatccctcttcctggtcaaaagttattgaaatctggttcaatgagtccaaatacttcaaatatggagaatggccatccacagatgatgacatcgaaactgatcactacactcagatggtctgggcctctacctacctcgttggctgtgatgttgcagcttgtcgcagacaaaaggcagctacatatctttatgtgtgtcactattgtcatgagggaaatcatcaagatacactaaatatgccttataaggagggctcaccatgtgacgattgtccaaataactgcgaagatggactgtgcactaacccctgcatttattatgatgaatacaacaactgtgatacgcaggtaaaactttatgggtgctcacatccagctgtccagccattctgcaaggcttcttgtctgtgtacaactgaaataaaataacctaaagtttgctactatgctctgaagataaaaaaaaaaaaaaaaaaaaa

SEQ ID NO: 19 - DNA, 898 bp, Mus musculus
atccttttgaaatgtcatttcactcgttagtatgcagtacagacaaagaccagatttgtatcagcacacaattttaagagatacatggatgtgggtatggcagtgaaatttatcttgttattgtttgttgctgcctttgttcctgttgtgactatcagacctctcaaactggatagagctctctataataaactgatcacggaatcacaaactgaaccacaagaggaaattgtcaatacccataacgccttcaggagaaaagtgtccccgccagccaggaacatgctgaaagtgagttggagttcagctgctgcagagaatgctagaatcttggcgaggtactgtgacaagtcagacagtgactcacttgagagacgactaccaaatacattttgtggagaaaatatgcttatggaacattatccctcttcctggtcaaaagttattgaaatctggttcaatgagtccaaatacttcaaatatggagaatggccatccacagatgatgacatcgaaactgatcactacactcagatggtctgggcctctacctacctcgttggctgtgatgttgcagcttgtcgcagacaaaaggcagctacatatctttatgtgtgtcactattgtcatgagggaaatcatcaagatacactaaatatgccttataaggagggctcaccatgtgacgattgtccaaataactgcgaagatggactgtgcactaacccctgcatttattatgatgaatacaacaactgtgatacgcaggtaaaactttatgggtgctcacatccagctgtccagccattctgcaaggcttcttgtctgtgtacaactgaaataaaataacctaaagtttgctactatgctctgaaaaaaaaaaaaaaaaaaaaaaaaa

SEQ ID NO: 20 - DNA, 899 bp, Mus musculus
atccttttgaaatgtcatttcactcgttagtatgcagtacagacaaagaccagatttgtatcagcacacaattttaagagatacatggatgtgggtatggcagtgaaatttatcttgttattgtttgttgctgcctttgttcctgttgtgactatcagacctctcaaactggatagagctctctataataaactgatcacggaatcacaaactgaaccacaagaggaaattgtcaatacccataacgccttcaggagaaaagtgtccccgccagccaggaacatgctgaaagtgagttggagttcagctgctgcagagaatgctagaatcttggcgaggtactgtgacaagtcagacagtgactcacttgagagacgactaccaaatacattttgtggagaaaatatgcttatggaacattatccctcttcctggtcaaaagttattgaaatctggttcaatgagtccaaatacttcaaatatggagaatggccatccacagatgatgacatcgaaactgatcactacactcagatggtctgggcctctacctacctcgttggctgtgatgttgcagcttgtcgcagacaaaaggcagctacatatctttatgtgtgtcactattgtcatgagggaaatcatcaagatacactaaatatgccttataaggagggctcaccatgtgacgattgtccaaataactgcgaagatggactgtgcactaacccctgcatttattatgatgaatacaacaactgtgatacgcaggtaaaactttatgggtgctcacatccagctgtccagccattctgcaaggcttcttgtctgtgtacaactgaaataaaataacctaaagtttgctactatgctctgaagataaaaaaaaaaaaaaaaaaaaa

SEQ ID NO: 21 - DNA, 860 bp, Mus musculus
acagacaaagaccagatttgtatcagcacacaattttaagagatacatggatgtgggtatggcagtgaaatttatcttgttattgtttgttgctgcctttgttcctgttgtgactatcagacctctcaaactggatagagctctctataataaactgatcacggaatcacaaactgaaccacaagaggaaattgtcaatacccataacgccttcaggagaaaagtgtccccgccagccaggaacatgctgaaagtgagttggagttcagctgctgcagagaatgctagaatcttggcgaggtactgtgacaagtcagacagtgactcacttgagagacgactaccaaatacattttgtggagaaaatatgcttatggaacattatccctcttcctggtcaaaagttattgaaatctggttcaatgagtccaaatacttcaaatatggagaatggccatccacagatgatgacatcgaaactgatcactacactcagatggtctgggcctctacctacctcgttggctgtgatgttgcagcttgtcgcagacaaaaggcagctacatatctttatgtgtgtcactattgtcatgagggaaatcatcaagatacactaaatatgccttataaggagggctcaccatgtgacgattgtccaaataactgcgaagatggactgtgcactaacccctgcatttattatgatgaatacaacaactgtgatacgcaggtaaaactttatgggtgctcacatccagctgtccagccattctgcaaggcttcttgtctgtgtacaactgaaataaaataacctaaagtttgctactatgctctgaaaaaaaaaaaaaaaaaaaaaaaaa

SEQ ID NO: 22 - DNA, 861 bp, Mus musculus
acagacaaagaccagatttgtatcagcacacaattttaagagatacatggatgtgggtatggcagtgaaatttatcttgttattgtttgttgctgcctttgttcctgttgtgactatcagacctctcaaactggatagagctctctataataaactgatcacggaatcacaaactgaaccacaagaggaaattgtcaatacccataacgccttcaggagaaaagtgtccccgccagccaggaacatgctgaaagtgagttggagttcagctgctgcagagaatgctagaatcttggcgaggtactgtgacaagtcagacagtgactcacttgagagacgactaccaaatacattttgtggagaaaatatgcttatggaacattatccctcttcctggtcaaaagttattgaaatctggttcaatgagtccaaatacttcaaatatggagaatggccatccacagatgatgacatcgaaactgatcactacactcagatggtctgggcctctacctacctcgttggctgtgatgttgcagcttgtcgcagacaaaaggcagctacatatctttatgtgtgtcactattgtcatgagggaaatcatcaagatacactaaatatgccttataaggagggctcaccatgtgacgattgtccaaataactgcgaagatggactgtgcactaacccctgcatttattatgatgaatacaacaactgtgatacgcaggtaaaactttatgggtgctcacatccagctgtccagccattctgcaaggcttcttgtctgtgtacaactgaaataaaataacctaaagtttgctactatgctctgaagataaaaaaaaaaaaaaaaaaaaa

SEQ ID NO: 23 - DNA, 824 bp, Rattus norvegicus
aagagctacatggatgtgggtatggcaatgaaactcatcttgttattgtttcttgctgctactcttactgtctttgttcctgttgtgactctcagacatctcaaactggatagagctctctataatcaactgatcacggaatcacaaactgaaccacaggaagaaattgttgatacacacaatgccttcaggagaaacgtgtccccgccagccaggaacatgctgaaaatgagttggagttcagctgctgcagaaaatgctagaattctggcgaggtactgcgacaagtcagacagtgactcacttgaaaggagactaccaaatacattttgtggagaaaatatgcatatggaaaactatccctcttcctggtcaaatgtaattgaaatctggtacaatgagtcgaaatatttcaaatacggagaatggccatccacagatgatgacattgaaacttatcactacacccagatggtctgggccccttcctacctcattggctgtgatgttgcatcatgccgcaggcaaaaggcagctacgtatctctatgtgtgtcactattgccatgaggggaatagtcaagacacattaaacatgccttataaggaggggccgccatgtcaagactgtccaaacaactgtgaagatggactgtgtaccaacccctgcctttattacgatgaatataacaactgtgataagcaagtaaaacttcttggatgctcacatccagcagtccggccattctgtaaggctttttgtctgtgtaaaactgaaataaaataacctattgcttgctgctaaaaaaaaaaaaaaaaaaaaaa

SEQ ID NO: 24 - DNA, 1255 bp, Rattus norvegicus
aagagctacatggatgtgggtatggcaatgaaactcatcttgttattgtttcttgctgctactcttactgtctttgttcctgttgtgactctcagacatctcaaactggatagagctctctataatcaactgatcacggaatcacaaactgaaccacaggaagaaattgttgatacacacaatgccttcaggagaaacgtgtccccgccagccaggaacatgctgaaaatgagttggagttcagctgctgcagaaaatgctagaattctggcgaggtactgcgacaagtcagacagtgactcacttgaaaggagactaccaaatacattttgtggagaaaatatgcatatggaaaactatccctcttcctggtcaaatgtaattgaaatctggtacaatgagtcgaaatatttcaaatacggagaatggccatccacagatgatgacattgaaacttatcactacacccagatggtctgggccccttcctacctcattggctgtgatgttgcatcatgccgcaggcaaaaggcagctacgtatctctatgtgtgtcactattgccatgaggggaatagtcaagacacattaaacatgccttataaggaggggccgccatgtcaagactgtccaaacaactgtgaagatggactgtgtaccaacccctgcctttattacgatgaatataacaactgtgataagcaagtaaaacttcttggatgctcacatccagcagtccggccattctgtaaggctttttgtctgtgtaaaactgaaataaaataacctattgcttgctgctatgctgtggggtaaaagggtgttttcaagatcatctatttcacctaatgtaatggactataggaccttctactgaatttcacttgtttttaaatttgtgctcaataacttaatttttaagcatcatttacagacatcagcatatttcatcaagactgtgcccttttcaggcagtaaaataactttaaacttccttgtttaatgaaagggcaagtcaaattcttgccccaatttaatacatgtgatttgtgttgtgtagttcgacataaccccattaatgcaggagctcttcatctatttactggcagtaaaacagtcttttacataaccccttttcctaatatccccatttcccctacaatcctaatgtttttgtttaatgtttcaattttcattctattttgaatcaagcattaaaaatattcaacaagtgaaaaaaaaaaaaaaaaaaaaa

SEQ ID NO: 25 - DNA, 1394 bp, Rattus norvegicus
atatccgttcctgttccttgatctgatgaagctggtccccttgtactaactgcctcattgagcagattgtctttgtgaaatgtcatttaactcattagtacacaatacaaagacctggtttgtagcagcacaaaattttaagagctacatggatgtgggtatggcaatgaaactcatcttgttattgtttcttgctgctactcttactgtctttgttcctgttgtgactctcagacatctcaaactggatagagctctctataatcaactgatcacggaatcacaaactgaaccacaggaagaaattgttgatacacacaatgccttcaggagaaacgtgtccccgccagccaggaacatgctgaaaatgagttggagttcagctgctgcagaaaatgctagaattctggcgaggtactgcgacaagtcagacagtgactcacttgaaaggagactaccaaatacattttgtggagaaaatatgcatatggaaaactatccctcttcctggtcaaatgtaattgaaatctggtacaatgagtcgaaatatttcaaatacggagaatggccatccacagatgatgacattgaaacttatcactacacccagatggtctgggccccttcctacctcattggctgtgatgttgcatcatgccgcaggcaaaaggcagctacgtatctctatgtgtgtcactattgccatgaggggaatagtcaagacacattaaacatgccttataaggaggggccgccatgtcaagactgtccaaacaactgtgaagatggactgtgtaccaacccctgcctttattacgatgaatataacaactgtgataagcaagtaaaacttcttggatgctcacatccagcagtccggccattctgtaaggctttttgtctgtgtaaaactgaaataaaataacctattgcttgctgctatgctgtggggtaaaagggtgttttcaagatcatctatttcacctaatgtaatggactataggaccttctactgaatttcacttgtttttaaatttgtgctcaataacttaatttttaagcatcatttacagacatcagcatatttcatcaagactgtgcccttttcaggcagtaaaataactttaaacttccttgtttaatgaaagggcaagtcaaattcttgccccaatttaatacatgtgatttgtgttgtgtagttcgacataaccccattaatgcaggagctcttcatctatttactggcagtaaaacagtcttttacataaccccttttcctaatatccccatttcccctacaatcctaatgtttttgtttaatgtttcaattttcattctattttgaatcaagcattaaaaatattcaacaagtgaaaaaaaaaaaaaaaaaaaaa

SEQ ID NO: 26 - DNA, 963 bp, Rattus norvegicus
atatccgttcctgttccttgatctgatgaagctggtccccttgtactaactgcctcattgagcagattgtctttgtgaaatgtcatttaactcattagtacacaatacaaagacctggtttgtagcagcacaaaattttaagagctacatggatgtgggtatggcaatgaaactcatcttgttattgtttcttgctgctactcttactgtctttgttcctgttgtgactctcagacatctcaaactggatagagctctctataatcaactgatcacggaatcacaaactgaaccacaggaagaaattgttgatacacacaatgccttcaggagaaacgtgtccccgccagccaggaacatgctgaaaatgagttggagttcagctgctgcagaaaatgctagaattctggcgaggtactgcgacaagtcagacagtgactcacttgaaaggagactaccaaatacattttgtggagaaaatatgcatatggaaaactatccctcttcctggtcaaatgtaattgaaatctggtacaatgagtcgaaatatttcaaatacggagaatggccatccacagatgatgacattgaaacttatcactacacccagatggtctgggccccttcctacctcattggctgtgatgttgcatcatgccgcaggcaaaaggcagctacgtatctctatgtgtgtcactattgccatgaggggaatagtcaagacacattaaacatgccttataaggaggggccgccatgtcaagactgtccaaacaactgtgaagatggactgtgtaccaacccctgcctttattacgatgaatataacaactgtgataagcaagtaaaacttcttggatgctcacatccagcagtccggccattctgtaaggctttttgtctgtgtaaaactgaaataaaataacctattgcttgctgctaaaaaaaaaaaaaaaaaaaaaa

SEQ ID NO: 27 - protein, 250 aa, Mus musculus
MAVKFILLLFVAAFVPVVTIRPLKLDRALYNKLITESQTEPQEEIVNTHNAFRRKVSPPARNMLKVSWSSAAAENARILARYCDKSDSDSLERRLPNTFCGENMLMEHYPSSWSKVIEIWFNESKYFKYGEWPSTDDDIETDHYTQMVWASTYLVGCDVAACRRQKAATYLYVCHYCHEGNHQDTLNMPYKEGSPCDDCPNNCEDGLCTNPCIYYDEYNNCDTQVKLYGCSHPAVQPFCKASCLCTTEIK

SEQ ID NO: 28 - protein, 254 aa, Rattus norvegicus
MAMKLILLLFLAATLTVFVPVVTLRHLKLDRALYNQLITESQTEPQEEIVDTHNAFRRNVSPPARNMLKMSWSSAAAENARILARYCDKSDSDSLERRLPNTFCGENMHMENYPSSWSNVIEIWYNESKYFKYGEWPSTDDDIETYHYTQMVWASSYLIGCDVASCRRQKAATYLYVCHYCHEGNSQDTLNMPYKEGPPCQDCPNNCEDGLCTNPCLYYDEYNNCDKQVKLLGCSHPAVRPFCKAFCLCKTEIK

SEQ ID NO: 29 - DNA, 2000 bp, Mus musculus
gatacttggttttcatttcttgcaggctatttccaggcattcattacaacttccatatcatggccagaaaattctgtattaaaagcctcattttttttcattttaattttcatttaactcctatgtgagtactaacacaaagtaactctttactacctactttgtattttcttcccttattgatagtcaagtcaccctcccttttaaaacatgtataaataaatattttaattaaatgttttattattctacaatggaaaatagaaaaaaggcatggagagaatgttttcaatataatttatgcataaaggtgtacatgtataaatggatatatatatgtatgtattgaaatgttatttgtatataagcagaacatagatcggtagatcaatagatagatagatagatagatagatagatagatagatagatagatagacagacagacagacagatagatgacagggagatataaataaggtcaaataagcatttgtattttttatatccttttctaaagtgtcctggctttaatcatatatgtgttaaatgatgcatagtcaaaagatgatcaataggacaggctccctaaaaattcaattgagagcagtttttctttcttttcttttcttttcttttcttttcttttctttttttttttttttttttttttttttttgtatgcaatgagaaccatgtcatacaattcctggccctcttctttaatgtttctggaatagtaagcaaaattaaaagcaattgtttgacaaactatgagcactcattcaagagtaagttttctatttttatatgtgctttcagtccattggcaagaattgccattcttttcttttaaaagctctttaacttcttatcctcaaactcaagagatatctggattcttagtttttccttactgacatttagtacaaacctacttaatatttccagatttttcaaggcagtgtgtcaactccatacacttctgatttttttttatcagaattttatctttttgtagatatttgttttctgtaatcactgagatctttatgagagtggactagcttcttttgcatctttggatcatcctaagctaactagaataacattgtatcatctcactaatgcactgcaagcactacacagcaggcactaaagaaattcctcagcagatttcactctgtgtgtgtctgtttatgataatcagccaaacatgcagaaaagaacatccttaatactacaattttaatacaagactggagaaatgatgcagtagttaaagtcttttccagaccatattaggaccagaattcaatccaatacagaggacccaaataaataggtaggaactcctttcactctgttttaatctttgctctgcggctgtgagatcaggaggatttctaggatatgctggcccagtagtatatactaggttcagtgagaaaactcatctcaaaaaaaaaaaaaaaatgaggtgaggagcaattaaggacaataccctacattgaccctcgacatctggcctccattcacatataaacctgagtgtacacacatgtgaagacaagtatatagacacataaacataatatttgtaccacacgcatatgaatgcacaaacacacacacatacacacacacacttaaataaacattgatttttaagttagagaaaaaaataaaactgataaaatatttcctttcccactattgagactttcaggaatctttctattcttttgtgagtctatgtatcctgcagctaagttttacagtttgttctcagtgcaaaaatttaacgctgataaagttgtctaggtcattgtatattctagaatatgctcatgatcgtgacttgaaatctaattttgttgtgaacatgtgagaaagcatatctgctgctcttccttgatctgctgaagcagttccccttgtactaattgcctcattgagcatatcctccttttgaaatgtcatttcactcgttagtatgc

SEQ ID NO: 30 - DNA, 2000 bp, Rattus norvegicus
gttttctaacatttccctttatcattaattaaagacaatttatagttttttatatgccttcacaatagggacatgaggaaagttagcaggttgtagcattctgtgctaggctctgcccacctgggtaagaattctgataaagattttggttgtttcttttgatttaggcttctcctcttatttgagcaaatgtttttgaagttttcagtagatctttggttgtattaaaattatggtgatgttgtcatctgttcaactaatattctaaactctcggctctatctctactctgtctttatgggctgggtaatgtctgcagcaagttactattcagacatccataactatgaagcaggcacttgttaagtattgatagatttaaggaataaaacgtcctcatgtctgttatcaggttagctattgtagtgataagtagaaaagaatagaatagtaggcaatcagtcttggtgataataaaggatagcatcagattgttatgtggaagtagaagaagggagcataagttgggatagaaaaggagagaaacatggtggatacatgggggcttgtattaatgacaaactgaaaagatgttttagatatcacaaagcaagtccatctctaagctttgggtatataatagtgaggaaaatgagcatgggaaaaagaaacaaaagaaagagacagagagatatagagacagagagagagagagagagagagagagagagagagagacagagacagagagagacagagagaggagagagagagagagacccaagctaggacaagaatgagattaagagagggaaacaggaaagaggtaaacagtataagacagtgtaggccaactttgctgacctaatgtactctataaataagaagcagctgctgcttggagcagagctggacagaattgtaagataatcaaatggctggcttgaatgttgttttcttctgggcagctctgttgcattgttttgatttcctggtctgggaaatgctctgagcttgttgttaatggccctactcctcttatacagtggatcctcagttcctttatatgtgattagtattgtgagtgtttgttaggatgtggaaaacactggaaaaagtggtgctaatgagagtggtgttgatgaaaaaggcagaataatgttacatggtttatttaataaagcatccctcatgttacacatcttttcagaggacttgggacatctaagagcaatgacaggacatccatgcgactacagaaacaagcataagtactcctgtttactatgaaatttttcacaaactgattaggttgcttttcaagatcataaatttctataaaagataactagtaggtttatagaaatacacataatagattgttagcaaaagtgtcaccctattaagatcttagaattgacaaactgtattaaataagaataaagatccattagtactgattttgttacaacacaaacacaagtatttgtagccaagtaatagagagagatgtgcagaatatctttaaaccacatacatatatacattagtagtaacaggatcacctgcttctgtgtactctttcaaataatgcctggctgatggagcctatctcgtgttataggctatagatgttgtctcttgacttttacaattaggcacctttgggattaacttgaatcaatgaagaaaacgtagttctatctctgttcatcacctggtgagccatgtaaataccatttgctactttccttccgtattaacaaacagttgtgcctattcagactctggaaattaagtagtttacattttgagtccatatcatttaaatcagtggtatattattttctgagtccccttagctaagacataatattggactttattttcatgtcctattataaaaagatagccatgtaaagttagttcaaggttccttagaaaatagtttcccgcacataaagaataatctaaaattctgttacttttgtttttcaggt

SEQ ID NO: 31 - DNA, 27 bp, Artificial
  Mouse CRISPC (mCRISP4) RT-PCR Cloning forward primer
atggcagtgaaatttatcttgttattg

SEQ ID NO: 32 - DNA, 28 bp, Artificial
  Mouse CRISPC (mCRISP4) RT-PCR Cloning full- length reverse primer
ttattttatttcagttgtacacagacaa

SEQ ID NO: 33 - DNA, 22 bp, Artificial
  Mouse CRISPC (mCRISP4) RT-PCR Cloning PR-1 domain truncation reverse primer
tgagccctccttataaggcata

SEQ ID NO: 34 - DNA, 24 bp, Artificial
  Rat CRISPC (rCRISP4) RT-PCR Cloning forward primer
atggcaatgaaactcatcttgtta

SEQ ID NO: 35 - DNA, 30 bp, Artificial
  Rat CRISPC (rCRISP4) RT-PCR Cloning full-length reverse primer
ttattttatttcagttttacacagacaaaa

SEQ ID NO: 36 - DNA, 18 bp, Artificial
  Rat CRISPC (rCRISP4) RT-PCR Cloning PR-1 domain-only truncation reverse primer
cggcccctccttataagg

SEQ ID NO: 37 - DNA, 753 bp, Mus musculus
atggcagtgaaatttatcttgttattgtttgttgctgcctttgttcctgttgtgactatcagacctctcaaactggatagagctctctataataaactgatcacggaatcacaaactgaaccacaagaggaaattgtcaatacccataacgccttcaggagaaaagtgtccccgccagccaggaacatgctgaaagtgagttggagttcagctgctgcagagaatgctagaatcttggcgaggtactgtgacaagtcagacagtgactcacttgagagacgactaccaaatacattttgtggagaaaatatgcttatggaacattatccctcttcctggtcaaaagttattgaaatctggttcaatgagtccaaatacttcaaatatggagaatggccatccacagatgatgacatcgaaactgatcactacactcagatggtctgggcctctacctacctcgttggctgtgatgttgcagcttgtcgcagacaaaaggcagctacatatctttatgtgtgtcactattgtcatgagggaaatcatcaagatacactaaatatgccttataaggagggctcaccatgtgacgattgtccaaataactgcgaagatggactgtgcactaacccctgcatttattatgatgaatacaacaactgtgatacgcaggtaaaactttatgggtgctcacatccagctgtccagccattctgcaaggcttcttgtctgtgtacaactgaaataaaataa

SEQ ID NO: 38 - DNA, 765 bp, Rattus norvegicus
atggcaatgaaactcatcttgttattgtttcttgctgctactcttactgtctttgttcctgttgtgactctcagacatctcaaactggatagagctctctataatcaactgatcacggaatcacaaactgaaccacaggaagaaattgttgatacacacaatgccttcaggagaaacgtgtccccgccagccaggaacatgctgaaaatgagttggagttcagctgctgcagaaaatgctagaattctggcgaggtactgcgacaagtcagacagtgactcacttgaaaggagactaccaaatacattttgtggagaaaatatgcatatggaaaactatccctcttcctggtcaaatgtaattgaaatctggtacaatgagtcgaaatatttcaaatacggagaatggccatccacagatgatgacattgaaacttatcactacacccagatggtctgggccccttcctacctcattggctgtgatgttgcatcatgccgcaggcaaaaggcagctacgtatctctatgtgtgtcactattgccatgaggggaatagtcaagacacattaaacatgccttataaggaggggccgccatgtcaagactgtccaaacaactgtgaagatggactgtgtaccaacccctgcctttattacgatgaatataacaactgtgataagcaagtaaaacttcttggatgctcacatccagcagtccggccattctgtaaggctttttgtctgtgtaaaactgaaataaaataa

SEQ ID NO: 39 - DNA, 582 bp, Mus musculus
atggcagtgaaatttatcttgttattgtttgttgctgcctttgttcctgttgtgactatcagacctctcaaactggatagagctctctataataaactgatcacggaatcacaaactgaaccacaagaggaaattgtcaatacccataacgccttcaggagaaaagtgtccccgccagccaggaacatgctgaaagtgagttggagttcagctgctgcagagaatgctagaatcttggcgaggtactgtgacaagtcagacagtgactcacttgagagacgactaccaaatacattttgtggagaaaatatgcttatggaacattatccctcttcctggtcaaaagttattgaaatctggttcaatgagtccaaatacttcaaatatggagaatggccatccacagatgatgacatcgaaactgatcactacactcagatggtctgggcctctacctacctcgttggctgtgatgttgcagcttgtcgcagacaaaaggcagctacatatctttatgtgtgtcactattgtcatgagggaaatcatcaagatacactaaatatgccttataaggagggctca

SEQ ID NO: 40 - DNA, 594 bp, Rattus norvegicus
atggcaatgaaactcatcttgttattgtttcttgctgctactcttactgtctttgttcctgttgtgactctcagacatctcaaactggatagagctctctataatcaactgatcacggaatcacaaactgaaccacaggaagaaattgttgatacacacaatgccttcaggagaaacgtgtccccgccagccaggaacatgctgaaaatgagttggagttcagctgctgcagaaaatgctagaattctggcgaggtactgcgacaagtcagacagtgactcacttgaaaggagactaccaaatacattttgtggagaaaatatgcatatggaaaactatccctcttcctggtcaaatgtaattgaaatctggtacaatgagtcgaaatatttcaaatacggagaatggccatccacagatgatgacattgaaacttatcactacacccagatggtctgggccccttcctacctcattggctgtgatgttgcatcatgccgcaggcaaaaggcagctacgtatctctatgtgtgtcactattgccatgaggggaatagtcaagacacattaaacatgccttataaggaggggccg

SEQ ID NO: 41 - DNA, 62300 bp, Artificial
  Mouse CRISP4 knockout construct sequence
accaagctcattcctaattgccagaataataccatagtctaaaaaacataaattttagaattatatttcttaaaataatcagtctccccaaaagtaggtctataagctgaaacaaatctaacaacttgtagtttaactagctagaataattgcctagatctttgtaggggagccattaacaaaaatcacaggctgcttggagtgagattctttttgactataaagctatcgaaatgcaggttatgcttgaacatttgtactagacaacacttctgtatatttattattaaaatcagctaattagcataagtggggaattgaagacttgcttgcttatgtgttaatgaggactagagattgtagaggggttttctagcctgagtcaaggttaacatgccttgttgccaattctagttgggaatatgacctgctgcaatgtcatcacctcacatctgctcaacactgacaataacccattttggggctaagcatcctgacaaacagcactttcttgtgattaatttttggcatattttcaagtattcatgaattctgccagaaaaaaatatcatttgttcctacaggaatttgtagaatagaaatcaattatttccttagttatgattattatgtataggcataaggtattggaaaaagaaaattttcaggaatatacacccatattaactaagtagtgttgtaaaattctgttcataaattcatagtattctgttcaaatcatctgacaataaaatctcggatttaaaaaaagacaagggaacaaattattatgcatgactgtgtgttatagaaaaagttataattactattataagccaactgtgattacctcattgtgcttcatttaaccttgcataacaaaggagattcaggttctgttttgtcaactatgacttagtaaagtgaactttaaatctggctcttttttcattgttttgttgttgttgttgtttgtttgtttgttttgttttgttgttaattgtttctacaaaaatttcacatgtatagacaatgcatgctaacaagagctccccaccctccacagcctcccatttttcaccaccctgtctattcccacagttccctattgctctctaattctctacagtttttttccaatttttattatgcatttgcttcatttacatttcaaatgctatcctgaaagtccgctataccctttcccccacccccgctcccctacccacccactcccacttcttggcatttttttaacccatgtataaaagctactctcacccctaatcagaatactggtgtttaaaaaaacaggcagaaaggttaactctctgaactagtcaattcagttgttcatttgttttaagtcaggagtgtcaaatctctatatatcttacatttatgtttgcctataagaaggcaaaataatgtacagtttgctgataaaatatgagtgtgcaaatgtctgcttgctttctttaaaagtctgaaaatgagtactatttagaaagttgtacaccctttggaaactgaagctgactgaagaattgaatcagacagcataaagattcttaagaattggatggacttattcacaccaacaaaaatcctccataattatatatacctttgactggacgtttagaattattccatggatattgtttcacatttgttcactattttattgttttctatgtgtacatgtccatgtatgtacatctgcacatgcatttgggtgccaatgatgccaaaagaggaaatcagattggctacaacaggagttacaattggttatgagacacctgaaatgtatgctagaatgatactcttgcaaagaagcatggctcctaggagccaaagttaggtcccctataagagcagtaagtgcccttaaactctaaatcatatctccagctcctaaacatgtgtctataaatttttctatttatttgtcattcatatgcatatgaatgtgggtacctgtggaagtcctagagggtgaatctcttgaaactggagttagaggtggttgtgagccactaggcatgagtgctgggaacagaactagggtcctcagcaagtgatgcaaatgttctaaatggtggggccatttctctagttcctagataaatatttcaattcaaaacattattctatagtctgatacaatacaaatgatatgcaaaccattgttaaactggattgtttagagggtaatggtaggaaaagagagccaggacttgttcagccagctatgctgtctttgttgagagtatctttcatctggagaggtggaaaggttaggtttgaaattccaactgtacacacaatcaacgccctaaatacacacagacaaaggaactcctttaatgtctaatcattgccctttcagggaacagtatgatgcagtttcagatggtggtttaaatagttgaaccatgacctaaatcaaatgggcagcatctacttgtgaagatagaacaaagtaaagctgtgcaacacatttccattaatcagaggctctcacacttacagcaaggtcatagttcttaaaattggaaagttataatgtgtagccaagtagggtggagagagaaactcaagattgatatctggaagaattcaattgacaaaaagatttgggggtaccttttctaccaggactacatgtaacgaacaaagcattctagataaaaaagacctggatcttcaataagagagaagatataaatgactccttgaggaatttggtagctcaaatcctttgagctaactttccacagctctctggcaaccatcaaccattggaagcattttcttaacgaatatctctatttttaccgctatccattagtccctggcccaatcctttatttcaaataaaggtacatggaagtctgtgaaggtgctcaccagactcaaatcatacacacttaagaataagtcagaaatgaatacagccaatctactgaggtacctccctcagcagaacagtgtatgccacatatcatgtgcttatcctcatggtagcatggctgactagaagcattgttcactgcaggaaccagaatccaaagagagtatcatgggcttgtatactgatagctcaggtaaacatcaaaatttaaaactagaagtacaaatctattctgctaaagctgaaaaacgcatagtcaaaccatggcaaatcaagaaccatctctactgtgattctactacacagctatgctatagatgtaagtaacttcaagagttaataaaatgccatacaaaaaattagaaagccatgaatttttagttcttaaaacatttgatttaaggcaaaattgaagcaggatatgctgctataagctcataatctaacacccggaaggatagacagactagtgcattcaaggcaatactgggccaaagatgaaaatcttgtctcaagacaaatagcctcagtggttgtgcccttgcctagtatatataacccttatttatgttgcgagtctcacacctcaaatgaacaagagttaaaataaaaataaactaaaataaactaaaaatcaaaatagttttatataaacttaatgagctttatggggtgaatttcttttcttgttttagaatttcatacttgcacatattgcaatttgatcaaattcactgcattgcctcccctccaagtcctccactatcatcccaccacttgtccctcccatcttcaaatgtgctttttataaacacaccaggtgcacatttcactgtctgtatacacaggggtgtgcagctgtcttctggagcatggaaaatccaaacaggttctacattcctgaagaaaacagactcttccacatttcacacctaacattcacaatctatgctaggatcttggctaatctgactttttacaggtcgtgtatggatggtcacagctgtacttatgtacaatgactctgtcgtgttcagaacacagtgtttttaagctatattctgcagccctgccccacccactccagtggtgactacttgacaggcccaaaagcttggacacagtcccgaggtgaaaagttcatggaaatttagtctcctggaaccatggcatcctgtaaaacgaatgctccaaacttgtccttgcattagtcagtgaatggatctgtgtgctttctttcagtgttgttttattataggtgcctccaagcaatgacttgccaaatacctaaagaaaattgaactttgcttcctggccttcaccaatgtcctaggtagtccttgagccaaccctgggcttggaattcagtaagaatattacttattcagtaacattcagaattgcctctagtattgttaagagagatagtgaaagaaatcaggcattgctatctactacatagagttagaaatctcagggcaaacttagcaaagtaagtttagaattcgtattatagttatgtatggcagactacattacttattagtagaaagtcctccatacaatcacttagaataaaagctgaataactcccatatacaggaatttacaatcatttaggaagtagacttggctgtggtttaaagaagaactagtgtattgcattattcatgagaaggttagctagagcggaactccctaattagaactttggcatgggtgtgaaagcccttgccccaggagtgtaaagaactcacacctgacttctaagagtatagctgaccttagatagagctgactttgtcttagttgttttattgcctagttcctgtcagtctttatgtatgcattcctctgttttgtgtaagtgtcattaagcatttggtaacctcaatgtaccttgactgatgtgtcatctaacttccctattttcttctgttttgtttgtttgtttgttttttgttttgttttgtttttgtttttgtttttcgagacagggtttctttgtaaagccctagctgtcctggaactcactttgtagaccaggctggcctcaaactcagaaatccacctgcctctgcctcccaagtgctgggattgaaggcgtgctaatgctcgctttgagaaattacattcagatacagcactctctctgcgtccgtgtctgtttgtcatctgcgccaacttcttcctctcctggttaccaaaatcctggtttcctgcagattgagggacccactgagtccagtccatggcacagatcctggttcttgtaatctttctctcctaaaagtaccttgtagacattctaaaacaaaaatactcagtatattatgcaagttcaacaggatctggcatactaaaatcttaaggtgtagcctcccccagccctgaggaggctgattcagaccgtgctgccactgtggagccttcacacctagatggttttattgttctatcacctgccactgctcttctccaagatgctgctacctgctgagaggttgagtctgccttcctgagaaattcctgagacagctggcaacctggtgattgacaccttactgcaaactagtgctgtcagcctaattgcttacaggctggcaacaggcatcaagagtggatctgcgagacatgagcaagcacagactctttgtaaacattgggctttgatcagaatttatcttggcctcatttctttctcgcctgtctattctctttatagcctcagtctgcctttcaggagggacccggttcctgtggctgtgggcggtcacagtaaggggccatgatttgccaaagaatgataccatggactcagttagtatgtgaacacagagtgttttattctgcagaagtccagcatgctggaatctaccaacatcagaatagagagatacccaagtgatcttgcaggcctgatttaaataaagcacattaggaaatttcaggataggtgacttctgtgttaatctgttgattccatctctatggacatttaattaccagggttgtagctggaatctttctggggatgtctggaaaatattactgaggaagtctggaaacttctgcttacccattgcccttgcttcaggccaggtgactgagaagttgcctggaattgtccagttcttgaaaattgagactaaggcctagtctccttaattgccaatttgaagtctgtcatggaatctgcttaaccttctcaaagtcaatcaatcaatcaatcaatcaatcaatcaatcaatcaaatgaaggtaatgccacatacttacagtctttgagtttttaactccaaggtaagaggttcaccacaaaatcaaggctaacctcttctgcataatgagttctaggccaaataggctacatagtaagactctatctcaaacaaagagatattctaattccatatgtcaatcatcatgctgaaaatatccaacctgatgtggtccatggagaccacagaaaggaaaaggcctgtgggtataaaaatttaataaagggaatgttataaaacattttaaaaataaaaacttaccttcctaagttatatgtaattctcattcccacagaaatactgggtagatgtgatggttaatgttaactgtcaagttgacaggatcactagattctaactgtgtgggagatgggcctctagtcatgcctatggagaaatgtcttaatcaggtatagagacctgactactaaaagggaacaatttcctgggcaggatatagtggactgtatatcatgagaaaagatagatgagcactagcttgtatccatccctctgactcctgcttgttgatgtgatgtgactaactgcatcaagctcctgctacctaaactttacctccataaaggactacaattgaactgtgagctaaaataaacacattcttccttaatttttcggcaaccataagtttaacatacattgcatataaaatgccttatttttcaagttcatcccaccatgttcttatttagttataccatagtatttccttaattgccatgcttacttaactcttggcccactgaactcgcttctttataaggaccctctccattatagacctatggatttccactttccaaggacagccacgtaaaaataggattgctatcagagatcatttaagatttatttatagaatttaactttcaggtctctagagagaggtaaagaagatagaagccactgagaagtagcaaagcttgccagaacctagaccctggtatagataaagccatctgacttttcatacacaatagaaaaaaaaaacaaaacaaagcaaaacaaaacaaaacaaaacaaattcttttatttgaaagcattcatatggatatattctaaaatttccattaattatcattaggtgggtactctagtgttgtggatccataaaaacaattgttaataacatatttctgaattaaggatttagggaataaagttaatttataattcttagaaaacatttaatagtattgtgatgtatctgacctttaacacaccataacaaaaactgggcaagaaggattaagatactatatattttggtaaagcatgactatatcaatgctaatacatggtgtagacatgaggcttccttatattctcttctacccattagtgtatacgtattgaatagtatcttactatcccagatagcagaaaatgtaaaataattgactattttcacaagataccttcctgctctctcaacagttattggttgactttgactaacactgaacaccagtgcacactaacaaatgatcattgaaattaatgacctcatattgtactagcttcaggacagctgttccagaatcttctactgtgaacttattatactccttgctgtggaggctggcgtggcctgtgccattgtaccatctgctcagctctcactcaacagcatctgctattcttgccttggcaccatctgctggccatctccatccccatctgcactgcttagtggtactgctgaaattagaacacatttcacctactcctaagtaaatgtactcctgtgtcctgagtcacttctccttttagatccagaaatgccctttgtggaatatctataagacttactttaagaatcccttcattcttatgaacacacatttcaaattactcatcattcttaaaaactgaaggggactctgtgtggctggctatagtaatatacatgggacttcataattatcattctaaaaatacttgatattcagacaatgtcctgcctttaaaagtatattacatttagaaaaattagaataactttatgcatatttcagaaatattaggtttctaaaatgcatgatgtttgttattttgtcatataagaaatatagtgtgggaaatgttaaattcaaagcacattatttattcaaatttgttactatacaggtatatgaataactggaataaaagacttataattgttcattaaaagtctaacacctttctttatttacctacgccaatgaatgcttctcctgttgaataagggatctgatacatttcagaagttaaacaatgaaagggtttgagtgaagcaaaatgaaacataggatgaagagaagttagcaaggaaaacaacaaaaaattaagactgaattgtgtgacacaaacagttattacacaccagatcagtatactcaaatcctactttccatccctctagaagaccatggctccctaacatttttttttctgccgtttagaaagaggggagacttccataaatttccacttgaagtgtttagactatttaaaatgaaaaaccccagaatttagtaaagattgaatataaatttgtacaacatggttcatgtaaataagtgatagacttagtagatacacacttttattggaaggatcatagaaaggagataaaagaactattataataagaaataaagacaagaaagatgtctgagctcaatcacactgaagctgtgtcaataaaatctgtagtaaaatatagataacttaaaagcattagcaacaaagtcaatatttgtctctgttaattagctttatcaaaaataaaacagaattgtcacacctgtcaaaaggtaattttacaacttggtgcaagaagcttagaaagtggaagaaatgttactatgccccttaaaatagtcttaaatcctctgttttggaggaaacccctactacataagaatattacagatagactaaagaataatgtttcatgttatgggaaagataatgattaattaatcaataaagcaagtatactgagaaatatatattcaaatatatatttctaatatatatggctaataagtcagtgtaacttgattaaataattctaacactaatttttatatacattaaattatcatatttctagaaaacataattactgtactataacaaagtaaatttcatctctcagtaaagacaaaagacacaaatgttatatttctaatactgttattctacccagagattggtttagtactagtagcaatggctaattttaacccaaatctagtatggttatttccttcacaaagagcagtgggtaggccactctttagctacgcagaaaaatgttgttgccttttgaacccacagttgcaagcatgaaataaaaatcctgaaagtttctccagggaaaaaggaggacctggtactgatactgatattcttatatattaaaaaaaaaaaaaaaaaaaaaaaaaaaaaaaaaaaggaaaaaggaaagaaagaaagaaagagagagagagagaaagaaagaaagaaaaagaaagaaagaaagaaagaaagaaagaaagaaagaaagaaaaagaggaaagaatttcatgaagtcattgtttttaatagctgagtagtactccattgtgtaaatgtaccacattttctgtattcattcctctgttgaaggacatgtgggttctttccagcttctggctattataaataaggctgctctgaacatagtggagtatgtgaccttgttatatgttggagcatcttttgggtacatgccctggagtggtatagctgggtcctcaggtaatactatgtccaattttctgaataaccaccaatctcatttccagaatggttgtatcagcttgcaatcccaccaacaatggaggagtgttcctcttttaccaatcctccccagtatctgtggttacctgagtttttttatcttacccattctgactagggtgagggatattttgatttgcatttgcctgatgactaaggatgttgaacatttctttagatgcttcccagccattcgatgtttctcagttgagaattctttgtttagctctggaccccattttaatagggttatttggttctctagcatctaacttcttgagttctctgtatatattggatattaaccctctgtcagatagcatgtagggttgttaaagatctttcccaatctgttggttgcaattttgtcctatgacagtgtcctttgccttacagaatttttaggcaaatggatggaactagaaaatatcatcctgagtgaggtaacccaatcacaaaagaacacacatggtatacacttactgataagtagatattaccccaacatctcagaaaatcaaggtacaattcacagaccaggtgaagctcaagaggatggaagaccaaagtgtgggtgttctggtccttcttaggagtaacaaaatactcacaggatttgcaatttttatgcaaacccacagaacgaataacaatatcaaccagccagacccccctgagctcccagagacaaatccaccaaccaaagagtacacatggagggacccatggctccagctgcatatgtagcagaagatagccttgttggacatcaatgggaggagaggcccttggtcctgggaagacttgatactacagtgtataagaatgccaggatgggaagctggagtgagtgaatggatgtgggaacactgtcagaagcagggcaaggagggatgggatagggtatttccagaggggaaactaggaagggggattacatttgaaatgcaaatagagaaaatactaatgaaaatgttaataaaaaaagaaaaaaaaagagagaagagaagagaagagagaagagaagaggaaaggaaaggaaagaaaggaaatcatgatgttgaatataaaataccaaaatatataacatcaatgcattgtatatgctgctgtaatacctgtgagaccccaaacatactagaatgaaaagaacaggattgtcaatgtgaagagtgcatagatgtatgcatgttctgaagtccagactcctgataggcatactgacaggagccacaagcaatgagtgcaacccactcatggtaccatatatcttcaacgactgaaaggggccttgaagacatttaagaattaccaaaatagagctggccagtggtggtgcatgcctttaatcctaccacctggaaggtagaggctggaggatttctgagttccaggccagcatggtctagagagtgagttccaggacatccagggctacacagagaaaccctgtctcacaaaaaaaaaaaaaaaaaaaaaaaaaaaaaatgagatagagatagagagagagagacagagacagagacagacagagacagagacagagacagagacagagacagagacagagacagacagacagacagagacagagagagaattactaaaataatgacaaataagggaagatttggaagcaatatttgtaactctctgacattccatgactttaagcacctacaaaaagtgatttggggaaacctgggtgtgttgtaaaggtattttgaagagtacaaagcaagtttttttttttttttttattatgaagtgttctaaattccaccttgattaacagatgatttttaaatatttattttttcctttttaaagatttttttattagatattttcttcatttacatttcaaatgctatcctgaaagcaccctatgctccccaacccacttactcctgcttcctggccctggcattcccctgtactggggcatatgatcttcacaagagcaagggcctctcctcccaatgatggctgatgagggctatcttctgctacatatgcagctagggacaggagctctggggatactggtaagtacatattgttgttcctcctatagagttgcagatccctttagctccttgggtattttctctagctccttcactgggggccctgtgttccatccaatagctgactgtgagcatccacttctttgtttgctaggctctggcatagcctcacaggagacaagctggagtgggtgagttggggagcagagcaggaggagggtatacgggactttcagagaggaaactaggaaaggggatagcatttgaattgtaaatgaagaaaatatctaattttttaaaaaagaaatttagcaaagccagtgagactgaaaaatcgggattgcagaattgccattcttgtacctgacaatttctaaagatttattaaaaaaaaaaaaagaaaagaaaatgaaaagaaacaatatgtacacttgcattgaagttctttaactaaaacatttaaaaggcaaataaaatgaagtaaaataagttgaaagcacatgtagagtaaggacatacaaacggaaaaatttcataatattctgaaatacatttgtgttctgacagatatgttaccttaaaaaggttaaaatgttcaaaatcttaaaattatagagcaaaaatataatcataaactagggttgattgttttcaaagaaataattacattataaatttcatgtgttccaaattccacattgtataagtctacactacacacagtcagtcagtcagcccttggccttccctttcattggctagccacagagtcaccatgggaaggttcctgtgatcttcattcagagtattcctctcttacaggtgtaattgttttgtcttttacccagcatatttactaatatttttctatgttgagttaggtaagcacagtatattacagttgtctgttatattcattataatactacagaaaaaatgggttctcctgtggagtctaggatgactgcaatggatctgcaccatccttatctaagttacttttcataatcatacaagtcaactacttagtgatatttctgagaggggttctccatcactaaataatacaggattgcattttaatagtgtgattgtatatatttatatttatttgtggtgtgtgtgtgttggcatggcatgtgtgtagaaaatagaggatatcggatatcctgcaggagtcaattatctctaacacatttgttgctggtatcagtctctgatcttcaggcttgatggcacacacttttacccaggtctatctagccagtctagagtacctgcagctaacccacaagcaacatggaactgagttgctgttgtttatactgcagactgtgttctggtgtttttttttttttttttttttttacttttctctttttttgttttgatttgttttttatttgcagatacctttaaaaaatatttccaaacttggaaaaaattactcaaggtccactttgtattcttagtgaagtatttataaattggttagatatattaccactattgaaaatatgtatatcattattttcatttaattgtattcatccttttaaaaccattatgtgaaaccctagaaaataagggacaacaaattttctaaaactaaagagactgcattccagtttatctgcctacagttgcggagacttgcaattagttgtcagaactatagcctagttgtttgtaaaatgtaggcaaccatagttactattattcaaaatataactgaattcagaaagtttacagataggctaggtggctgaggcagtatggcagcctcaattattatttacttattattaatttgttttgaatatttcttggaagtgaacatcggcctttttacctgataagaatggactttatcagcaaactatagcacttagaaaattttaaggtttaactccaatgtcctggctgacctctaaatctaaggctcaaacattcttctgctttaacttcctactttgtaagatatacaggcatgtgctaaattgtgggtttctatcaatgtttgctgatctttagcatatctggaggcatgctttatgtgtcatgagttgtaaattaaacattatgtggacatgtaattgctttgaaatttgattattattttggttagcttttttttttctagcttgtgttgaagaagatgatcccaaattgatattatggagaatttcttcattttgcttctcctatgaattaaaatttgcttgtagttatcttacatctgcattttcaatgaatacatcattttagacataatgctttctgaaactaccacaaactgaatgccacaaatgctctcttccaatttgatatattttgcttctgaattccttggaattttttaaccagacaacactattgaaccgatagtaacagaataacatgtccacaacaggagagtagatttttctttgctatgtttcaaaaatatctgtaaagaccaaaacctaaggagtgagttgaaaagagatttattccatctacatcacatttagctatatacccataggattctaaatgagcacaccatataaatacatccatattatgcttagcatagtactactcagagtatccaggaataaagtgaatgtatacacccataatcaaaataataataataataataaagactatacatatgactctatctccaagtgatctaatctttcttctgatatctgtgggtacctacaaacatgtggcaaatacactctaagatacatgtacctcatataaataaaaaataaaaataaggtttaaaaaatatattttgtgggtagtagagtatgagagtagggtagctgaggaacacagggataggtaatctacattaaagacatttcaaaagtcatataggaaacttctattgtagaaacttcctaatatatcacatacataaaaacagtttaaaattaacttattaaatagtaatgatgcctcttccagataatgaaggacaataaattaaagacctagggaaagaaatgggtttcctcaatttctactggctagctttcttcggtgctagaagattctatgcacatttttggaaaagcagtattgaacaaactcctagcaacttatccttattccaatagattaggacatctcctaaccctcattagagaatacttcttttgctctagatagtgattatcatgaaaaaaaaaccccacaatccatcatggtataggaaataagagacttcagaatgctcagccttaaacaggacatccatagcatactcctcaccctcatctctcaggaatcattgtaaacagatgtaaaaggagaaacacagccagaattggtcaacaacttcaaggaaacttttcgggacacaggaagctccatatataacaccagttgaggcagcatttataagacttaggcaaactcaagctagacaaatcccagtatggagaagaaaggatctgatttgtttgcttgagtttcagacacttatgggttgaccaagcaacagtgaaaaccacacatctgagcatccaattttatatgaacactagcatctgtccaagagagagataggcagacatagagagactgagagacaaagaaagacataacaacagacaaatacatacacaaaaaacacacacacaaaggagggagagagagggagagggagagggagagggagagagagggggagagggagagggagaatacatattgggtgaggaggaaaagggaaatggttctgggaagcaatggggagttgaatatgatcaaaacacattacaaaacattctcaaaaacttataaaacagataaataaatctagaatatacacatgcttttaatattgatttaaagttatacttatgttgatatcattggcttttggagttcagttgacattcaaagtatttcctgattcttgtgtcccagaaaaagaaaactaacagagatacatggatagtaatagaagcagatccagtttattaacaatacatttcttaggaaaagaagtgagccaggagggttacataatcaaaggtaaaatacaaaaggtttggcttttatcggtcatttgtcttttatcagttttcccaagcacctttgtaccatgcaaggaaggccgggttgtccagtctttctttctgccaagtagtctctttcatatgctaatttttactccttcagacattaacctccacatccatattctttaataaaacctaaaaacaagtgaggaaatttatcatgattttagaatattactgatttgttttgtttccattaaatgaacagatgcaagcatcatctacatttgtcatttgaactaatctctttacttaggagaattctgttaactgagttcatgccactctttgaaacattattgactctgggagaaaaattttaattgtgatctggataatattatgtaagaagccttggtttgtatcagagctctaacatacagtagctgtaccatgttagaaccaccattaattatttcctggacaatggggtttgtaggtataagacctaaggttttaacattccattccctcattaatttcctggctgaatggcattgctttaaatctaagtgtgttttccctcttctgtgccattgaatgaaaaaaaggtattaatgttcctctatagtgcccgtgaagacatgacaaatatatgtgtagttcctaaaccacactatatgcccttatatttcaatagatagtagttatatgttaatgttggcattttattttctatatatcatctaaaatgaatattatattacataattaatgaatgcatattattaaggtggggcagtgggtgtggaggaagaagggggagggtgagagcccgcatctggccagagttcctgtgctctgagcaagcagatgcgggaggactgttggacactttccacgcagccccggttgggtatctggctgtgtgaagccactgaacccacatggtgtggggtggacaaggggcagccccctgtaccaagttctcattctttggcaccccacactggatacagcagaggagctgagagcagaataggggccccagggcgacactctcagccctggagatactggaagagggcagagggagagaggttcccatgcaggcaagagtccttggtcggggccttgatgaacagtaacagtctatggttttagagctttattgtagaaaagcagggggaaagagaaaagatagaaagagatgcatggtggggctggtggagaccaggagaggacatgaggtctcctggatctggagtttctgcgagctactagaggtgtgttctggaaacagaagtcaagtcctctggaagagcaacaagcactcttacccactaagctgtttctccaagctcttaaaatttgatttaaattttaccataagacatgcatgtttgcaaactgcaataatcttcagtattcagtgaaacaatcgagaatgtacccaaagatccccagaacagaacaaaggaaccattcaaattattaatgagttgacttggacaagcacaaagatgataaatgtctgtgatctctgctgacatgtgacttcccttgatgagataccagactcaaatctgagtcactaatacccaaaaactgttgagtgtgcttattgttttaagttattaagaactttgaattattgttataggagtaggcaggaaatacaatttcaatagaaagatgggtgtatggtagcaaaagtataaagtttataaattataattctattataattaaattataaaaattcattcttcactctatgtgggagatatttagatattatagaatttaaattttatggggatagaaccgacttcaccagctaggaggactttggaacataaagcagaaattctctgagtagtagtcaactcccaggacactctcacaaaacaaacaaacaaacaaaaactaaattataataccaaaaaagtcagaaaaagtttttgtttgtttgttttaaatgattttgttttttcaagttgtttttcccatggcctcacacttatgatccactttctcaagatgtgatacccttgcctcttaacttctttttccttggctgaacacttttttgtgagagttttgaacacacaaggcagcctcacagtccccagaggagacaccagttccaggtgctgtaacacacccacaatcttacgatttcaggatcccaggtgtactggctagttttgtgtcaacttgacacagctggggttatcacagagaaaggagcttcagttgaggaaatgcctccatgagatccaactctaaggcattttctcaattagtgatcaagggggaaaggccccttgtgggtgggaccatctctgggctggtagtcttgggttctataagagagcaggctgagcaagccaggggaggcaagccagtaaagaacatccctccatggcctctgcatcagctcctgctttctgacctgcttgagttccagtcctgcatcctttggtgaccaacagcagtatggaaatgtaagccgaataaaccctttcctccccaacttgcttcttggtcatgatgtttgtgcaggaatagaaaccctgactaagacaaattggtaccagcagagtggggtattcctgcgacaacctgaccatgttttggggaggtctgtggaaggactttggaactttgggcttgaagatccattcattgttaagagctctgtcagatgttttggtaggagcttggaagataatgttgagaacagtgcagaagatggaggcctggcttgtgaaattcagagggaaaattaaagacttttcagggccattgctgttttgattgtgaagattctgtagttctggttagctggggctgaagaatcagctgtgattaacaagataccagaactactaaagcaaaaactttgcattactgggactattgatgctggttagctggagctaagaaataagcagtgattaagaagagaccagcatcactgaggtgacatcttctgggaagtgttttctgagagtacagtggctgtgttccagatatagccaaagttgtaccttatgctgtggctggacttggtaatgtgtaagggtcaccccaggtggtactggttttgaaggcatgaaggagttgagcagagcagctgaggcttggcactgtgagaggccatggaaggccattggtgaaagtgcagcctcagttgcaattgatggcccaggactgaaggggtcatgcagtgttttggagatgccagtaccatgagatgaccaccaagagcagcagcagcagtggagtacagacatctggagcctagaggactacgcgtgtactacaaggggcatggctggagaagtgacccaagcccttggaggagcctagaagatcgtgagttggatcccagacattggacggttggagattgatttttgctttgattgtgactgtgccctgatattttccctcttgaaggaagaaactgttttagtggagcccacagttaagagacttttaattgtaaaaagagtttggattttaaaagagatggatattttaaagagattgaaaatttaagaatatgtaaagactgtgggacttttaaagttgtttagatcttggggatgaataagaatgtaagggttgaggcttactagtgatgtgtttgtgtgtcaagttgacaaggggtcaattgtactggctagttttgtgtcaacttgacacagctggggttatcacagagaaaggagcttcagttgaggaaatgcctccatgagatccaactctaaggcattttctcaattagtgatcaagggggaaaggccccttgtgggtgggaccatctctgggctggtagtcttgggttctataagagagcaggctgagcaagccaggggaggcaagccagtaaagaacatccctccatggcctctgcatcagctcctgctttctgacctgcttgagttccagtcctgcatcctttggtgaccaacagcagtatggaaatgtaagccgaataaaccctttcctccccaacttgcttcttggtcatgatgtttgtgcaggaatagaaaccctgactaagacaccaggataccaggagcttagtcacaacaagatctcagggtctcagaggaagcttgactgccaagaactctgaaatatccagaatctcaggtttatgggatcccggaatcacaggatcacagagaaagctggactctgaggagtcctgaattaacccagattataggaaggacaggctccaatcagatatattgaggacaacaagcacttgagataataagatgccaggaagcagcataacagaagaaacagaaaacaagattaattggcatcatcagaaccaaactcccccatcattttaaatcctggatacatgatcccaccagaaaagcaagatatggatataaagtcactcctcatgatgatgatggaggactttaagaaggaaataaataactcccttaaagaaatacagaaaaacacaggtaaacaggtagaagccattaaagaggaaacacaaaaatccctagagagttaaaggaaaacactatcaaataggtgaaagaattgaataaaaccatccaggatcaaaagtggaagtagaaacaataaagaaatcacaaagggggacaactctagagattgaaatcctaggaaagaaatcagggatgatagatgggagaatcagcaagagaataaaagagatggaagagagaatctcaggtgcagaagattccatagaaaacatggacacaacaatcaaagaaaatgcaaaatacaaaaagatcctaaatcaaaacatccaggaaattcaggacacaatgagaagacaaaaccaatggataataggtatagataagaatgaaggctttcaacttaaagggccagtaaatatcatcaacaaatttatagaagaaaacttcactaacctacagggagagatgtccatgaatatagaagaagccgacagaactccaaatagactagaacagaaaagaaattcctcctgacatataataatcagacacaacatatgcactaaataaagatggaatattaaaagcaataagggaaaaaggtcaagtaacatataaaggtagacctattaggattacatgagacttctcaccagagactatgaaagccagaagaccctagacagatgttatacagacaataagagaaagacaaatgccagcccaacaactatacccagcaaaactctcaattaccacagatggagaaacaaaagtattccatgacaaaaccaaattcacacaatatctttccacaaatccagcccttcaaaggatagtaaagggaaaactctaacacaaggatggaaatgacaccctagaaaaagcaagaaactaattcttagacaaacctaaaagaagactgtcacaagaacagaactccaactctaacaacaaaaataaaagaaagtaacaattacttttccttaatatctcttaatgccaatggactcgattcctcaataaaaagacatagactaacagagtgctacataaacaggactcaacattttgctgcatacaggaaaaccaccttagggacaaagacagatactacctcagagtaaaaggctggaaaaccattttccaagaaaacagtctgaagaaacaagctggaggatccgttctaatattgaataaaattgtcttccaacccaagattatcaaaaaagacaaggaggggcacttcatacttgtcaaaggtaaaatcttccaagatgaactctcaattttaaatatctatgctccaaatgcaagggcatccacattcattaaagaaactttagtaaagttcaaagcacacattgcaccacacacaataatagtggtagacttcaagaccgctctctcatcaattgacagattctgcaaacagaaaactaaacagagacacagtgaacctaacagaagttatgaaacaaattgacttaaaaagtatctatataacattttatccttaaaaaaaaggatatacattcttctcagcgcctcatggtaccttctgcaaaattgaccatataattactaacataacatgcctcaacagataaaaaatattgaattcatcccatttatcctatcagatcaccacggactaatgctgatcttcaataacaacataaataatagaaagccaacattgatgtggaaagttaagaacactctactcaatgataccttgagtcaagggaagaaataaagaaagaaattaaaggcttttaaaagtttattgaaaataaagccacaacatatcaaacttatgggacacaatgaaagcagtcctaagaaaactcatagataatgctttaataaagaggcattgctttaaatcttatcttaaattttacttttttaggataggaaccacattaattattatctaaactctgaaatatctttagagacccagcctcttggcctgccttatgccacgtgtcggaaggactctaaacttgagttatcaaatttaacactaaccatctgtatcaatgtaacaattattaatcttaaccaacaacttatgagctgaatgtgaaaatatgcagaacacattttcaataaaaaaggaaccaaatgaagtagttaaatttagaccaatcagagaatattaatttttgtagctacaatcatagaataaaaggcactttaccattgtcaaacacattgatcatgaaccatttatcagcttttttttactctgaaattaagaggctgtgcactcgcccttttttcctaaaacgaacagtttttccagcaggggccctcctgccacgtgtctgattcctggctgaaacacgtggcagctctcggctttgcagataaagttttttttataccatctttattatccaacataaaacatagaacattcattcaaacagactggacacgaacatacatgaactgaacacgagaacagattggtgcaccagacattagacgagacacaaaagggaacagagtactcctgctataaggcccagagagaaatttctctctgctttttgggacattttcctccctcatgatgcagtttttattaactgcagcaatccgcctgattattaatttccttttaataaaccctttcttttctcacacctaaaaatgtagcttcagagagtcacggcctaccgcctgttatagttcccagctcctcttaagcttcactgctgaacctaagtgaaccagcgctcgggtttaacgctgtctcagcttagcccataccttctccagtatgaatttcctttatcctttaattattccatggagctccaaaccagcagtgtctcttccaaaaatcctttgccgtttctcagtcccacgttggttcatcaaatgttgtgtctggccagtagatcacaacatgggttttagcctggaaaggcaatgtggaaacctggaagagaagaggggctgggctgcgcgagataaggaaggaaccaagacaaagctctctgctcaaggttcaatttttactatttcggcactcagttataaaggaagggggaggggcccaattcccgccaaataatcttggagtccagtagcagggtgatcacgtggtggctccaaacagcaaagcggcaggttccagcagtaggagtggcagaaagattgagcgggaagctccacccctgagcaagcaggtttcaggctgggggaggggagactacatctgagtacccccaaaaagatactagagaggagaacacactagcagcttgagagcacacctaaaggctctagaacaaaaggaagcatattcacccaagaagagtatgtggcaggaaatactcaaactcagagctgaaatcaaccaagtggaaacaaaaagtactatacaaaatatcaaccaaaccaggagcttgttcttcgtgaaaatcagcaagatagataaatccttagccagtctaaactagaggacacagggacagtgttctaattaacaaaatgagaaatgaaaagggagacataactacagaacctgaggaaatctaaagcatcatcagatcctactacaaaaggctatactcaaccaaactggaaaacctggatgacatgaacaattttctagacagatatcaggtaccaaatttaaatcaggatcagattaatgatctaatcattcccatatcccctaaagaaatagaagcagtcattaattgtctcccaaccaaaaaacgcacaggaccaggtgggtttagtgcatagttatatcagaccttaaaagaagacctaattctaattctgctcaaactatttcataaaatagaaacagaagttactctacccaattcattctatgaagccacaattactctaatacctaaaccacataaagacccatcaaagaaagagaacttcagaccaatttcccttatgaatatctatgcaaaaatactcaataaaattttggcaaactgaatccaagaacacatcacaagaaccatccatcattatcaaataggcttcatccctgggatgcagggaaggtttaatatatggaaatccatcaatgtagtccactatataaacaaactcaaagacaaaaaccacatgatcatctccttagatgctgagaaagcatttgacaaaatccaacacatattcatgataaaagtcttgggaaagatcagaaacgcaaagcccatacctaaacatagtaaaagcaatatacaacaaatgcgtagccaacatcaaactaaatggagagaaagttgaagcaatcacactaaaatcaggatttagacaaggctgcccactttctccctacctgttcaatatagtacttgaagtcctagccagagcaattagacaacaaaaggagatgaaggcgacacaaattggaaaggaagaagccaaaatatcactatttgcagatgatatgttagttaccctaaaaattccaccagagaactcctaaaactgataatcaacttcagtgctatagctggatataaaattaactcaaataaatcaatgacctttctctacacaaaggataaacaggctgagaaagtaattagggaaacaacagctttcacaatagtcccaaattatataaaatagcttggtgtgactctaactaaggaagaaatcgaagaagatctcagaagatggagagatttcccatgttcatggattggcaggattaatatagcaaaaaatggctatcctaccaaaagcaatctacagatccaatgcaatccccatcaaaattccaactcaattgttcaccgagttagaaagggcaatttgcaaattcctctggaataacaaaaaacctgggagagcaaaaaatttctcaacaataaaagaacctctggcagaatcaccatgcttgacctcaagctgtattacagagcaattgtgataaaacctgcatgttactactacagggacagacaggtagatcaatggaatagaagtgaagacacagaaatgaaccaacatacctatgacgacttgatctttgacaagggagctaaaacaatcccttggaaaaaaagacagcattttcaacaaatggtgctggaacaactggaggttattatgtatcagaatgtgaattgaatcattcttatctccttgttaaaaagctcaaatttaagtagatgaaggaactccacataaaaccagagacactgaaacttatagaggagaaagtagtgaaaagcctggaagatatgggcacaggggaaaaaaatccctgaacagaacagcaatggcttgtgctgtaagatcacgaatcaaccaatggaacctcataaaattgcaaagcttctataagcaaaagacactgtcagtaagacaaaaagacggccaacaaattgggaaaggatttttaccaatcctaaatcctaatggggactaatatccaatatatatatatacatagaactccagaagttagactccagaaaaatcaaataaccctatttaaaaatgtgttacagtgctaaacatagaattctcaactgaggaatactgaatggctgagaagcacctgaaaaaaaaatttcaacatccttagtcatcagggaaatgcaaatcaaaacaacactgagacaccacctcacaccagtcagactggctaagatcaaaaactcaggtgacagcagatgctggcgaggatgtggagaaagaaagaacactccttccattgctggtgggatttcaagttggtacaaaccactctggaaatcagtttggcggttcctcacaaaattggacatagtactactggaagatccagcaatacctctcctgggcatatacccagaagacatcccaacttttaataaggatacatgctccactatgttcatagcagccttatttataatagccagaagctggaaagaacccagatgttcctcaacagaggaattgatacagaaaatgtggtacatttacacaatggattactaatcagttattaaaaacaatgaatttatgaaattcttaggccaatggatggatatgaaggatatcatccggagtgaggtaacctaaacacaaaacaacacccatgaaatgcactcagtaataagtggatattagcccagaaacttagaatacccaagatacaatttgcaaaacacatgagactcaagaaggaagatcaaagtttgcatacttcattccttcttaaaatggggaataaaatacccatggaaggagttacagagacaaagttaggagcagagcatgaaggaatgaccatccagagactgtcccacttggggatctattccataaacaaccaccaaacccagacactggggcagttgccaacaagagcctgctgataggagtttgatatagctgtctcctgtgaggtaccagtgcctgtcaaatacagaagtgaatgctcacagtcatccattggttggagcacaaggtcctgaatgaaagagccagagaaatacccagggagctgaggggtctgaagccccatagaaggaacatcaatatgaactatccaataccctcagagcttcttagaactataccaccaatcaaagaaaacacctggtggaacttgtggctctctctatatatgtagcagaggatggcttgctcagtcatcagtgggaggagaggcccttggtcctatgaaggctctttgccccagtataggggaatgtcaggaccaagaatgggagtgggtgtgttggggagcagggggagggggaggggatagggtattttcggaggcaaaattaggaaaggggataacatttgtaatgtaaatattggatatatctaaaaaagaaaaagtaaaaagaagtgtgagagttgcgtctaatttaaaatcctgtaccatgagtttttcagcaagaatatgtataatattgttgagataaatagaactcttgatcctatttagcacataagtaccgagtctttgtttcattatacatttatgcttcctatatgtttggtagctgatttctgagtgaatgattcattttaaagtttttaaaattcattcattttaatgaacaggatgtgttcacaaacacattagtctttatttatgcctcaatggttttttgggatagctccaatttaaaatgctctttcatgttgttgcatcatatgttaaatcaatgtgtctggattctttgttttaggagaaaagtcattgtagatcattttctaccatgccaaaaagtttaagcagaaagagaacaatttatgaaaatgggagaaagatgagcagaagcagccagctggcaccattttggaggattgcatgcaaatcaagttattctgtgctttccaacactttccaaattctcttgaaaccatacttaaagctggatagcaactctatatgaagtcatcagtgatataaatacagaaagcactgtgtttggtagaataatgccagagactgacaggacaaaaagaaaacaaaaacaaacaaacaaaaaacaagacaattaactttccataaacagttgacttggtgttgagtcaacactcttctttgtaaatagcagtgtagcagggaataaataacaatcatacatttccaatacataaaccaggctggtctactttaagaaacaacagtcttttccagttttcttcttacaacacaggcaaataaattcttcagtaacctgagcactttactttggcaatcaagcaaagttccttgtctacaaggtttaaataagttttacataacttttggtactttaccaacttaatgtacttctgcaaaattctggctacaggatatcatgttttgagggatacttggttttcatttcttgcaggctatttccaggcattcattacaacttccatatcatggccagaaaattctgtattaaaagcctcattttttttcattttaattttcatttaactcctatgtgagtactaacacaaagtaactctttactacctactttgtattttcttcccttattgatagtcaagtcaccctcccttttaaaacatgtataaataaatattttaattaaatgttttattattctacaatggaaaatagaaaaaaggcatggagagaatgttttcaatataatttatgcataaaggtgtacatgtataaatggatatatatatgtatgtattgaaatgttatttgtatataagcagaacatagatcggtagatcaatagatagatagatagatagatagatagatagatagatagatagatagacagacagacagacagatagatgacagggagatataaataaggtcaaataagcatttgtattttttatatccttttctaaagtgtcctggctttaatcatatatgtgttaaatgatgcatagtcaaaagatgatcaataggacaggctccctaaaaattcaattgagagcagtttttctttcttttcttttcttttcttttcttttcttttctttttttttttttttttttttttttttttgtatgcaatgagaaccatgtcatacaattcctggccctcttctttaatgtttctggaatagtaagcaaaattaaaagcaattgtttgacaaactatgagcactcattcaagagtaagttttctatttttatatgtgctttcagtccattggcaagaattgccattcttttcttttaaaagctctttaacttcttatcctcaaactcaagagatatctggattcttagtttttccttactgacatttagtacaaacctacttaatatttccagatttttcaaggcagtgtgtcaactccatacacttctgatttttttttatcagaattttatctttttgtagatatttgttttctgtaatcactgagatctttatgagagtggactagcttcttttgcatctttggatcatcctaagctaactagaataacattgtatcatctcactaatgcactgcaagcactacacagcaggcactaaagaaattcctcagcagatttcactctgtgtgtgtctgtttatgataatcagccaaacatgcagaaaagaacatccttaatactacaattttaatacaagactggagaaatgatgcagtagttaaagtcttttccagaccatattaggaccagaattcaatccaatacagaggacccaaataaataggtaggaactcctttcactctgttttaatctttgctctgcggctgtgagatcaggaggatttctaggatatgctggcccagtagtatatactaggttcagtgagaaaactcatctcaaaaaaaaaaaaaaaatgaggtgaggagcaattaaggacaataccctacattgaccctcgacatctggcctccattcacatataaacctgagtgtacacacatgtgaagacaagtatatagacacataaacataatatttgtaccacacgcatatgaatgcacaaacacacacacatacacacacacacttaaataaacattgatttttaagttagagaaaaaaataaaactgataaaatatttcctttcccactattgagactttcaggaatctttctattcttttgtgagtctatgtatcctgcagctaagttttacagtttgttctcagtgcaaaaatttaacgctgataaagttgtctaggtcattgtatattctagaatatgctcatgatcgtgacttgaaatctaattttgttgtgaacatgtgagaaagcatatctgctgctcttccttgatctgctgaagcagttccccttgtactaattgcctcattgagcatatcctccttttgaaatgtcatttcactcgttagtatgcagtacagacaaagaccagatttgtatcagcacacaattttaagagatacatggatgtgggtaagttatacatcatatttcagttcatttaattgtaagtgaaatgcatatataaaaattcagcagcactgttataaaatcaattacatgcaaaatctattaatgttgacataatctaaaagcaaaggtccaccatttccacaattttagtaggatttatgatcaatacattctgtccaagattatgtcacttttcgagttcattctcataaactactgaagtaagatttaataactccaatttggacatctttacttaagttttaaattttatagatgtatttttgagtatattcccctacttcatgcttctttcccttccctatctctccttttcccttcaattaatctcttttcacaaaagacagttttgatgttacttgtatgccatatatctatacatgattttatgtacctatattatatcaaagaatcacaaaagagagaaaagatgtaacgtttgtcttatgagattgtcttaactcatttaatatctatagttgcttgcattcccatctgaaagacatgactatgttcttctttctgtctggaaaggattagattatgtagttttatatctatgtgtgtatagatagaagaaatatcacacaggtttataaatgtctctgaaatatattgtcttgacactctttgagtaaatactcaggagtggtatagttgtttcatatgataaacctattgttagacttttgagaacccttcatactgacttccagagaggctggactagttcacatccccaccagaagtttaaagcatacaactgtggattagtggagtctggacacattttatttcacatgaatctatttgaaactgtggtcaaaataagcagttagtagagaaacattataatactgtgcatattttacatttgcagtttatcttggggaatcatgaattattttgtggataataggataaacactttactttacataagaaaagcaattcccagaaatcgtagtcttggggaaggagggataattttagctgttcaaatttctttctgcagatcttctagttgctgttctttgatctttcctcatgggagatctcatagtctgaaagatcacacatttcgcagcctttctcagcactgtctcttctctgtgccttctcctttcacatttctcttgtggtgctttctttgttctacttatgtttcctgttgtgtatgctgttcttctgttctctgatttatgaaaatattgtcttcagtacatattctatagtgtttgtgagtacatgtttatttctgtactggacattagtacagaagataatgaagcaaatttaaattaaaagggagtaaaagtaaattaaaagtgaaagtaaaaagatgatcatttatttcaaaagttatgactagacactaatacagtagattattatcaacatatgtgtggatttatgtcaaacaactgtaatctgcagtaaataatacaattactattaattatatggcatgtaaaacataatttttgtctcaaatgtgagacagagttcgaattcattatttttgtattagatacttcactgaattttctttatttgatttcatgttagcctttgtcttaaacgagatgatcatatgtagaagtttattttgtatcctataatattcccttgatccgtttttctaaacttatgacaccaatgctgtccatgcccacaattatagtacattttaatatttagtagtgcaagtgttcaaatttagatgctattccttaatattttatagttttaatcaacttttagaagaaaaatgctttcataatttgtctattcaggtaacaaaaattatcaaacattcatatacacagagacacacaaacacaaaattagctattgtaagtaaaactaaaacctaatatctacttttaaactatttgctaaataagatatagagatatgggtcagctatttcatatacactacttctaaggctagagattattaagaaagagggtgaaataattatatgaataaaggacattgtctataagaaaattatcttctgaagatagcagggatactgcagatatgaacttacagggattgtgacataacacataagatacaggcactaacatgtagtctgctgaatgtaaggattaaggtatttgccaaagcaaagaagattaaatgtgggaaatggtcttttggcaaggtatgtttttatcggttattcagtcaaggcaaggaagagcctgttcctctttttagttgattatgaaaggtcatgttctaacaattcccagatgggggcgttttctctaaggcttgccacagtgtactgcttgatcctcctcagctttttcccacatcctctatctttcctcccacaaaatattaatttttaattgttcatggcccatttaataaagcttaaagttaggacaataagtttatgtagtcggtttttcaagttcaggtactgttcattttttaaaaagaaagttgactaagagtcgcacattatgaacaaaacatgtgtgttcaaacagaaatgctgattttattctcctttgtctctgttttaactgagttcaggaaactagttagcatctcagataaaaggtaagctgtgtttacagatttgcagcctgtggtcaatggagaatcatgcaagatactctggatgctttgccctggtcagtccatggattacatcaagtctcagtgatctagtgaacaaagctcagacaaggcctctaccctgagactatttttcctctaagcctgaatagctgaatagcttcgatttatgctgtctaatatttatctttagaccagcatctttgttgatatcaggctcaattttctgctgaaaccacaacctctttctgatccttctctctacccttgactcctgattttgagttgtgtttacaaacactacccaagagatccaaagtctgtaatctctcatactcatatctagaataatagcaattatatattatatattctggcaattatatggattctattttcattctaaaatccttaatgcattcctggtggtaaggtattaaatagtatcttcctgtttcaacttgattgctctaagtgctatttgagttttaattaacctaaaataatcacctatgttctttgtacttttgattatttaaggaacattaacatgagtggtcacattctacctgctcacaagtcctgaagccaaagcaggattaaggatttgagagatttttttcttagcagatctccttccttctgttcagtcaaactgatctttgctaaaccaaagttcttcgtatgagaccaatgtgctaaattaaccataatctacctgaaaccagtggtcccatgatcttatatcagaggtcagataggagtttttttgtggtttatgtctattgttcacagataaaaagtcacaggcatatattatagtgttcagtcatttatgtatgctacataaaacaaatatttttcacctatttgtgacagaagcctttgtatttattttttacatgaatatgtacatatacatgtttcagttaatctttatgtagttagatatattttccaacaatgcccttatcattaactaaaggcaatatatagtatattatatgccttcacattatgatatgaggcaatctaacagattgtagcattctgggctgcccacttgggttagatgtctactaaagattttggctgtttcttttgatgcaggctgctcctcttatttgagcagatatgtctgaaattttcagaagaccctctgttgtaataaagttttggtggtataatctatttaactaatattctaaactcctggctctatctctactctgtctctttgggctgggtaatatctgcagcaagttactattcagacattagtaaccatgatgcagacacttattaagtactgattaatttatggaataagaggccctcatgtctattattaggttagctattgtagtgataagtagaaaagaactgaagagtaggtaagcaattatagtaagaatagtggttaatatcagattgttagatggaagtaaaggaagggagtatacatgggaataggaataagagaagagaaaaaaacaggatgaatacataggggtttgagttaatgacaaactaaaaagaagagagaaagatgttttagatatcacaaaccaagtctgtctctaggctttagggaaataactgtaagaaaaatgggcagaagctaacagcttctgggacaggcgggagccacagagcttctaaggcagcccccttttccggccccagacatccagccactttcccggccagaggataggtgtccgcccagcccaggagcgctttgcctgagcatcagcagtggacatcttggttccaggactcctccaagtgtattctgcacaggtaagagtgtagaatacagaagctaacagcttctgggacaggctgaaacaactcagcttcggggacaggtcctgttttgggccttcatcttcggccatgagggaggtccaaaagccagatatctgtgcaccttccctgtaagaggagagcttgcctgcagagagtgctctgaccactgaaactcagaggagagagctactctcccaggtctgctgatagaggctaacagaatcacctgaggaacaagctctaaccagagacaactaaatcaactaactccagagattaccagatggagagggttaaacgtaagaatcttactaacagaaaccaagaccactcaccatcagcagaaaccagctctcccacctcacccagtccagggcaccccaacacacccgaaaagctagacccggatttaaaagcatatctcatgatgatggtagaggacatcaagaaggaatcactcaaagaaataaagtagaacatggctaaacaggtataagatcttaaagaggaagcacaaaaaccccttaaagaattacaggaatacatgaccaaacaggtgatggaattgaataaaaccatctaagacctaaagagggaagtaaacacaataaagaaaacccaaagtgatgcaacgctggagatagaaaccatagaaaagaaatctggaaccatagatgcaagcatcagcaacagaatacaagagatagaagagataatctcaggtacagaagattccatagagaacattggaacaccaatcaaagaaaatacaaaatgcaaaacaaccctaactcaaaacatccaggaaatgaaggacaaaatgagaagaccaaacctacagataatagaagtagatgagaatgaagattttcaactcaaaggcccagcaaataacttagacaaacttatagaagaaaacttcccaaacctaaagaatgacatgcccatgaacatacaagaagcctacagaactccaaatagactggaccagaaaagaaattcctcccaacatataataatcagaacaacaaatgcactaattaaagatagaatattaaaagcagaaaggaaaaagggtcaagtaacatataaaggcaagcctatcagaattacaccagatttttcaccagatactaattaagcaagaagagcttggacagaggttatacagacactaagaaaacacaaatgccaacccaggctactatacccagccaaactttcaattaccttagatggagaaaccaaagtattccatgacagaaccaaattcacacattatttttccacaaatctagcccttcaaaggataataacaggaaaaaagaaaatccaatacaaggacagaaaccatgtcctagaaaaagaaagaaggtaatccctcaacaaacctaaaataagacagccacaagaacagaatgccaactttaacaacaaaaaataggaagcaacaattacttttccttaatatctcttaatatcaatggactcaactccccaataaaaaacatagactaacagactggctacaaaaacaggacccaacattctgctgcttacaagaaacacatctcagggaaaaggattgacactaccttaaaatgaaaggctggaaaacaattttccaagcaaatggtctgaagaaacaagctggagtagccattctaatatctaataaaatcgacttccaacccaaggtcatcaaaaaagacaaggaggggcacttcatactcatcaatggtaaaatcttccaagaggaactctcaattctgaatatctatgctccaaatacaagggcagccacattcattaaataaactttagtaaagctcaaagcacacattgcaattcacacaataatagtgggagacttcaacacaccactttctccaatagacagatcatggaaacagaaactaaacagggacacagtgaaactaacagaagttatgaaacaaatggatctaacagatatctatataacatttcatcctaaaacaaaaggatataccttcttctcatcacctcatggtaccttctccaaaattggccacataattggacacaaaacaagtctcaacagatacaataatattgaaattgtcccatgcatcctatcagatcaccatggactaaggccgatcttcaataacaaaataaataatagaaagccaactttcatgtggaaactgaacaatactcttctcaatgataccttggtcaaggaaggaataaagaaaaaaattaaggactttttggagtttaatgaaaatgaagccacaacatacccaaacttatgggacacaatgaaagcattttaagaggaaaactcatagctctgagtgcctccaaaagaaactagagagagcacacattagcagcttgacaacacacctaaaagctctagaacaaaacaaagcaaattcacccaagaggagtagaagacaggaaataatcaaactcaggggtgaaatcaaccaagtggaaacaagaagaactattcagagaatcaaccaaaggaggagctggttcttgagaaaatcaacaagatagataaacccttagccagactcactagagggcacagggaaagcatcctaattaacaaaatcagaaatgaaaagggagacataacaacagatcctgaagaaatccaaaacaccatcagatccttctacaaaaggctatactcaacaaaactggagaacctggatgaaatggacaagtttctagacagataccaggttccaaagttaaatcaagatcaggttaatgatctaaacagtcctatatccctaaagaaatcaaagcagttattaatagtctcccaaccaaaaaaagcccaggactagatgggtttagtgcagagttctatcagaccttcaaagaagatctaattccagtccttcacaaactattccacaaaatagaaacagaaggtaatctacccagctcattctatgaagccacaattactctgatacctaaactacaaaaagacccaacaaagatagagaacttcagaccaatttcccttatgaatatcgatgcaaaagtcctcaataaagttctcactaaccgaatccaagaacacatcaaaacaatcatccatcctgaccaagtagatttcatcccagggatgcagggacggttcaatatacagaaatccatcaaagtactccagtatataaacaaactcaaagacaaaaaccacatgatcatctccttagatgcggagaaaacatttgacaaaatccaacactcattcatgataaaagtcttggaaagatcaggaattcaaggcccatacctaaacatgataaaagcaatctacagcaaaccaatagccaacatcaaagtaaatggtgagaagctggaagaaatcccactaaaatcatgtggttgattgtgaggatgtgggaagcactggagaaaatgagctgtagagagtagaggtgaagaaaaaggcagagcaatgctacatggttcatttaatgaagaatccctcaggttatacttttttttttcagaggacatgggacatctgagagcaatgacaggacagccatgtaactatggaaacaagcataagtactcctgtttactatttaattcttcataaattgattaggttgcttttaaagattagaaatttctgtaaaagttaactagtaggtttatacaaatacacataacaggttgttagaaaaagtgccacctcattagtatcttagaattgactaactgtactaaaaatgaataaagacccattagtatttattttgcataacacaaagacaattattattagccaagtaatggaaatgtacagaatatctttcaattaaatgtatagaaatctttaaattacacacaaacaacagtagtaacatgatcacctgccttctatgtactctttcaaatatacatggctgatggagtctatttcatgttatattctacaaatgttgtctcctgactttttctttcacaattaggcacctttgttattaacttaaatcaatgaagaatatataggataatctcttttaagctttagtcacttggtgagaaacctaaatgctatctgctattttaataccccataaacacacaattgtgtatatttagactctggaagttaaggagtccatatcatttaagttaagtccatatcatctaaatcaactgaatattattttatgaggccccttagctaaggcttaatattgaactttattttcatgtcccattaacaaaaatctaacaaagtaaaaccttgctcaaggcttcttagaaaataattttttacacactaagagtaacctaaatttctgttacttatatttttcaggtatggcagtgaaatttatcttgttattgtttgttgctgcctttgttcctgttgtgactatcagagtatgtaaatattatatgttacataactatattttatttaagggtaggagaagggtgtatcagttattcacatacttcatataatgatgcagttaaacaagtcttattctgttttataaaaagaattatttatttattctatgtatatgattacactatagctatctccagacatatcagaagaggacattggattcctttacagatggttgtgagccatgtggttcctgtgtattgaactcagaacatcttgaagagcaatcattgctcttaactactgagctatttcttctgcccaaacaagttctattcttaatacagcagaatcacgaatgaaaccaaaaggagaaggcctaagtgaactttcaagtttagaagaattgatgaggattaaagttcaactttcactttaactcattggatccatgtctatgtctttttttataaacaataagaaaacaaacaaacaaataaataaaggcaagctagcaaaggatgtgataaggtagagaagggatggaggaagagaaagaaaaaaatccataggcaagaacttgtttacaatttacccatgtgttttaataaccagatcgaagaaattagataaatagtattatttttaattttcctcccacataccctttttgattttaagaatgttataaatcaaaattaaatatgaaattgaaagttacaactgcaaagatgtgtgagagattcagcaatggaatcaaggtatgatgcttggatctaattgctcataaaaataaaacctttgggctagtgatatagctcagtattatagcacttgaattgtgtgtgtgtgtgatgtccagaatagatccccaggatcacaaagtaagataatgatggtaatgctgatgttgttaatgataatgatgacttcaatatttatagaacaaaaccaacatcttcagtggtaaaataggtagagtgtcctacaaatttgtgatcattgagtgacaattcatcattagcaaccaaaataaggcatggaaaactgatcttaatttactttatctaattaaaaaatgacttaatgtcaatagatttttcttttgtagaactagattttttgcgtgactgtgtatgtgtgtttctcatgagtcacagggagatattcacagtgggaaaaattacataatattattgttgaaagattattgtcatcagctactggagtggtggattggtatcagtataccatgatgtccatgtccccatgatcttctaggaagctgaatattgaataccaagctttatggaaggtttcctttaacttccctttgattctctgcttgtttcacatcttttttgtggaagaacatcacaccaccaaaactagagctaacataaagtataggctgcattacaaaggaaaaaggaaagttgggtagtcagtaaccaacatgtgcctttgtgacccggtagtctacgataatttagcccaattttggagcacaaataaaagaaagtatgagcactaccagagttcagagaaaagttcactgaaggaactaaggaactaagatatcagaggtttccagacagatatatggatactagaccactggactgattaaaggaatctagatgtgcaaggtgaatttacataagagatttgatttttgtgaaagtaaatgaacaatctcagaaaaaaaatagacattcctttactgttttattatagaggttgtaaatatttgtggatgaacagagagtcaaatgttatgtttttcattgaaatcaaatgctcaaaggaggaagtctgtgcattcttgtgagcctacaaatttttctttaatataaactagagctgattgcaatgatggaagaaaccagcctgtgagcatgttccaaggaaaggagttggtgtctgtagaggttccatgggaatgtactcttgacctgaatgacttctgttttgtcaaagctccattctggattcttaccattcttaaatttactcaataatccagtagtagctaaggaaaaaacatacaccattcaaattacatattaattttgataactgataaaagaaaccttgggaaatatatgggttttaaaccacacatattaaagactcattatggcaactaatgcgaacacagactattttagaaaactatttttttcttaatgaattacaaagtaaaatgtataactttttaaaaaaggagtcatacattcgaaactacaacggtagtttgaattttacttacacttttaaatgcacccacttgttttaaagcccaccgggtttttaagatataaatataaaagagctataaacctggagagtgaaaagaacatgtagtcacatgctcaaaatacactcttaattttggttcttacacacacatgcacacacacttacatgcacacacacacacttggactcaagtattcattgttcatttgtagagtacaggcattgctttcaacagctttatatagagagaatgagctaaggcagagagtcattccgtgaacaagagatgtcaagccatggcttttgtgattcctattgttcaataacaagaacttctacttgcctctaaatatgaattataaatttgaaaatttcgaagaacagtataatttgtgtactgttttcaacccaaaaatgtagaaatccttaaaacttgaatactaataatttctaaatactaaacataattgttcactgttaagttctaatttcacattgcctataggtctctatgtaatagactttgtgtgacaagggctacttcaaagaagtgatattaatgaataattttctggtagcctctcaaactggatagagctctctataataaactgatcacggaatcacaaactgaaccacaagaggaaattgtcaatacccataacgccttcaggagaaaagtgtccccgccagccaggaacatgctgaaagtggtaagtctagagcacagcatcagtgtagaactggtggcattccctacattgttacttagtcttatgtactgtgactatgccaattgtttgatcatcgcaacaaatctatgtcataacagcaaaagaaactgagtctaagagaatgtgactaactctctcatgtatatgatagaataacaattctgactcagcctcaaatcagtcttaggtgccttcagacatgtgacatgcatttgagaatccggatgttcaaattcatagctaaaaaaatttgacagattttgaaaaaaaaaaactagtagtgaatttcattaaattccatagaaatttagagatctgaccttggcagtgcaaaaactgatggtttctatcacaagaaatatttagggactcaagacagtcaagagtgatgaagttcagtcaacagtgcaggccctgagcagtagttttcaatctataaatatcaaccccttggggggtcacaaagcagttatgcattatggcttataacagtaacaaaattacagttgtgaagtagaaacagaataacttcatggttagtggtcaccacaataggaggaactgtactaaagggtcacagcaccaggaaggttgagaaatactgccctattatcatatgataataactggcagtacactcctgtaaatgatggagaggaagcaacaattgaaaacgaatgtagtttctgaaaaattatctctagaccaccttaaaaattaacagtattttctgatacactattttaaaaccatcagttgtcataagtatccatgtttgtgctactgttaataaatgttctcgagctaaacatagtgatttatatctagactcccagcattcagatagactccgaggctgaggcagaggatccctaggtgttcaaatccaacacaggccacatgctgcgctccagaaaatgtattcttatagtttctaaaagtaatctagtttatttaattttgcactacaatggtttccactgcaaggagtctcaattatatgatcaattttactctaaatccttaattcaagagggacatacccagtaaatctgatcctaatcatattaagataatttccaaattcctatctaccagatgaacaaataactatttctaggaagggctctctactgagcagtctaattatatactgaaagcaattttggtcttttcaattccttggttagacacacacatcaaaaagtccccaaaggcccttatacctttcctactccctgctccccaatccaccctctcctgcttcctggccatggcattctcctgtactggggcatatgatcttcgcaagaccaagggcctctcctcccattgatggccgaatgggccatcctctgctacatatgcaactagagacacagctctggggggtactggctagttcatattgttgttccacctatagggttgcccacccctttagctccttgggtactttctctagcttcttcattaggggccctgtattccatccaatagatgactgtgagcatccacttctgtatttgccaggaattggcatagcctcacaaaagagagctatgtcagggtcctgtcagcaaaatcttgctggcatatgcaatagtgtctgggtttggtggttgaacatgggatggatccctgggtggggcagtctctggatggtccttccttccatctcagctctgaactttgtctctgtaattccttcccatgggtattttgttccccattctaaaaaggagtgaaatatccacactttggtcttccttcttcttgagtttcatgtgttttgcaaatagtgtcttggctataagtttctggcctaatatccacttatcagtgagtgcatatcatgtgggttcttttatgattgggttacctcactcagaatgatatcctccagatgcatccacttgcctaagaatttcatgaattcattgtttttaattgctgagtagtactccattgtgtaaatgtatcacattttctgtatccattcctctgttgaggtacatctgagttctttgatgtccctcaaaagtattatttcttatttgaaaaacggaaacaaataacatgttttggatgcagtcaactagaagagtaattctacacaatagccactagatggtaccgtacacacatacagtaattatcttttcactgtcatgctgagaaaattgacctttactctgaagagtgatggcaataattctaatgttgcctcctacattattactaagtccagtgaacataatgtgttctaaaaatttcgttaaaatgccaatctcaaaccacagaagtagggtaaaacgagtctaaaaagagttgtaactgtctgtacatccttagaaaatttatataaaagtctgggaacataccaaggaaatcatgttattgttgacagtgtaacacacaggctgtgagcacactctgacgatgctgggtgcaggattgtcaggatgtcactgctactcctacatacgtgtgtctgagtggtttattaaatgatgcttcaactgagtatatgctacacttagcccacccaaatgaagagttcaggctaatctcgagtaactgattcaaattctctcttgtatgacccttaaaagctttagaacctggttttgggctggagaaatggctctgcagataagagtactggctgctcttcctgagaatcccggttcaatttccaacatccacagggcagctcacaactgtctgtaattcctgttccaggtgatccaacatgtgctcacagatatacatgcaggcaaaacaccaatgaactttaaaaaataaaatattttttaaaattcacacacagtacactgagttactgcaagagtggaagatttatggttaataacagtgtctaggaatgttaactcctttgctttaatcactacttgaggcagggatctagaactctgctttcattcacagaagccatttgtgtgctaagtgcatattttgagactcaaaatatcacaggttgattgtaaggactgccgactggacaaagcttttggcataacttccatatcatctttacagaagctgtgactcaaagaggtgaaatgacaagccagggttttcttgtttgcatctctgttgcagtgatgaaacactgacccaagggaacatagaggagggggtttatttggtctaaaatgcctgatgacaataaattgttgaggaaaggcaaggcaaggtctagcagaggccatagaagaatgcctcttactggcttattcttcatgacttctcccatctgttttcttatatcacttagaaccacatacaatctctgatcaagaatatgtcaccaaagcatgttcccaagtcactatgatgtagccaattccttagggacagttctttctttccaagtgactctagtttgtatcaaattgacaaaaactagccagcactgagataaaacaaccgataagttgctgggttagttttgtgattgtttgtttcctgaaactctttacataaattataagaaacaagttatggtctgaaggttataacctaattttgaatttgtaatatcacaagaaactgaaatgaattacacacttgttttctttctatgttcctattagaatatttaaggaaaatgtcaccatgtaaaagatcagtttccacaagaaaggacataaggaattgtacatttttttttatatagaacacaagaatcttaggttgagaaagctaatttcattgtgtttctggatattctcttgccttacagagttggagttcagctgctgcagagaatgctagaatcttggcgaggtactgtgacaagtcagacagtgactcacttgagagacgactaccaagtatgtatgggtctgcatgacacaacttgcattaaatagttttcccaacactatagtgaaatgcctgggggtggggctgatcaattcaaagggaggggtttgtttttaaatcatggcttcagaaagttcagcccgtggatttttagcttgaatgatttggaacagaacggaggccacataagatagtggattgaaaagtggaggatgctgttcacctcaaatagaaaagacgtaggagagagaaagagagagaatgccttcagagatccaactctgtcatataggctccaattctcaaagcacctacaacccactcagtcccagtatctagagaacaagccttcaacaggtgagaccttgtgatacagttaaggtgcaaataacttttaaacaaacaaaaatgttaatgaataaaataaatctcaaaaaagaacgtgcctacaaacaactggacaatagtatgctaacagatagaatctacagggaccctcacctatccttgctggcagattgtagcttgatatcatcatttttaatttttaaaatttggatacttttatttttgtagtctaagttctcatcaacaggaggaagctatgatcagcaacattaaagaatgaagcacatcaggctctctaggaggtttctattcaaacatctctcagttgtccacaattttaggatgtgacatttcctcagtttggggacactaggatatgaggaactctagaactaccctaataaacaattttgagtgctcaaaggtaagctcagttttgtcatataaactaaacaagtactacccaatttttagtgaatcctacagaattacgtaacattttaagtaatgctttcttccaaactaactttaagaaatacaaatgtaatttatacttatagattattggaactcttatgccactgtgttaaacaatgcgggtatcatggtattctaaagaaagacagaattattcctcaatatcaaatcctacataaagcatataggacaaatggatccaccatcttttgagcatatgtatcctctttgtaactgattttacctccagagctggtttactttcagctctcatgtgctcaaacagacttttaccatcatcctttacctgataaaagacagagattgatatctgcagtcatgattttaaagctttctcaacaactgtcagtaccatcgcttgccagacttgagcacaacatgatcattcagtgaaacagataaacaactgagtttatttcctgttttaaaacaatgcctaggtgatcatgaaaacatttgggaaataaaatgaggagatcctctgtgttctaacataaaaacatactacacaggctttttcctcatttcagatacattttgtggagaaaatatgcttatggaacattatccctcttcctggtcaaaagttattgaaatctggttcaatgagtccaaatacttcaaatatggagaatggccatccacagatgatgacatcgaaactgatcactacactcaggtaatctgtattcttctagatatacacatgactggatatatgtgtcagtaaaggataactactatttctcttctcaggcctaaggaaaataacccaggaaatcccaaacatctacttcctagattgtggccaggataatgataataccacttgactgacagtgcaaacgggaaattccacctgagcccaccttcttgtctaagaacaagtagtcaataacttctcagctaaagagtgtcagtcttgaacatatttacatatgagtaatcgtaaaggatctcagtaggttcaatacacaacctatatgtgtctagatctatatgtaataataatgattaaagaaggaacaaggagctcacgtttgaaatggaagatgcaaagtgatatagatagggctcatgtatgaagttctcagttaacagagttttttgtttgtatttttttcaaaaacaatttttattagatatttttttcatttacatttcaaatgctatcctgaaagttctctatatgctccccctgctctgcgccccaacccacccacccctgcttctggccctggcattcccctgtactggggcatatgatcttcacaagatcaagggcctctcctcccaatgatggctgactaggccatcttctcttacatatgtaactagagacacaatcaataaacagatttttttttggtttgtatttttatgattaagtggaagcaaaaggcaatcagtaagtttatgggtaatatacctgctggagtcttataaaggatctgaagatctgtactctggtccttatgaagaaaggcaaatattttgtctaatgaggcattttcccagcccacttagcttcattagcatgttttagtttgagtaagttttagttaaagtaagttttattatgagatttcaacatgcaatgtatgtgcagattataatcactgcttttatttgctatttactgtccctgatccatttccacatccatcacctcattgtccctctattgccatgacacatgaaattcatgtgtgacagaaaacctaatccttgacttaaggagactatctatattctctcaataaaaaagatccatagttccgtacattttcctgaaaatggcatattttattgttcttattgtttgaaaaactgtcttttgtgtctttctaacacattcatatggaaacagatacctagactgtcttgtctattgttaatggtacccttgtaaagacaagagcgcatgtcagtattgtatgttgatcaggatttcctcatatatacatccagaaaaaataaaacttaaggaagttctacttctaataatttaagtaactccatacagattttcacaagggattcccagtttatgtttccaccagcaccatgcaggaatccttctctttccacagcatcacaaatatttgtacttttatttttctggagagccatcattctaagagcagtgacacggaatcttgcagaaatgttgattaaatttttctaacagctgaatatgttgataatttttcatgtattaattggacattcatatttcttttaagtacaaccaaccaatagttaaactgttcaaaacctgacactgtcaaggaatagtatcattttcaaaccacatcagatctactttaatttctttcatttaatattctacagatctcaccatgaaatatttaaggctctttgctaaagattctaagtagtttattgccattgtgctttttaagtagaacaattttcttaatttaattttcttattattcatttagttttagtgttatagaaaatttgagagtgattaatgggaatagataaggttttcacaagagagacaaagtgatcatattacccagtataatatttacatttatattaaatgcaggatgtaatcagtggaggaaaatagacaaaggattgacttaatttgattaaaattttgaattatcatttgcctgccacacagagtccaagttaattttaaagaaaaatatccctagaatctctaatgtaacaaaatgtatgctagagaaagagtatttaaatatttaaaatgtaaaacattaaaatatagttattgtccacaaaagagaggactcggaagtgttcactggtgattctcataacttacatgaagtaaattttagttaggattgaccatataagttcccaaagatgtacctcaaataatcagttatgaatgtatgcacagaattattaaataaacaacagcaaattatcttttctttaaggatcagtaccactgaacataaacagcatgtgcaaagaatccatgataccttaaaacttagcaggttaatccaatgatttatgatgccaatggattagaagagagcctgagaaagaaaaacagtgaaaactatgatgatgcaaccccagtcttataacccattcatacaaagtcaatccctttcttatatcatatggctttggttctttcagtcttgtgcagcaatttatccctttgactttctatttatcaaaataattcaagccatatcaaataaatttataaaaataactggtcccatttgccaaaggtgctagaaattgtataacattttgtaaattaataacaagagaggccatgaatttaaaaagtagcaaaattatataacagggtttggagaaagaaaatgaaaatactgtaaaattatcataatcttaaagccaaagaaatattttcaaaaatcaaagaaattggtctttatttgcttaaattttaagagaaaaatactgtcattatcgtacaatgataagaatccattaatattcattttttcacattatttgtactttttcttgaatatggactgaatgaataaatggaacaaatacacaaaatagatgttctaatgtatttttattgtaatttttcaaagtatatgttctatattagtcagaggttaacaaagtttcataaggcaaatgtacgtttaggagaaagaatgagaagagaaacttgcaatcattgagtttttgcctcatccaacgtttacatagtgggtggagccacagatttcatgaaagcgtattcttattccccatagataattaaatgctgtctggtgtggagggaaactgactcctggaaaagttgtgaagagaaagtaatactttacaaaatcagtcagagaaaatcactatgaatagaaagaagaagaaagaggaggaggagaagaggaggagaataataggaaatagggagaagaagggagaaggagaggaggaggaagaggaggaggagaagaaagagaggaaggagaaaaagtagaatagaagaaaaggcaaaagaagaagaaacctgattaaagatttcagtgttggctgagggaaatacaagccaggctcctacccaggtcacagaagaaagtgggcgccattgcatcacccttatttccttctccagcttttttaaacattgtaatatttaaccctcactatacaaatgcatgtactgggcagcattattatagtcgtcttacagatgagaaaaatgaaaccctaataaagaaacttctagatccaatccaaacagaaccaaaccaaaactaaaaccagaacaacaaaaacaaaacaaaacaaaaacaaagtgaacaaacaaacaaaattattacccagcttagaaatacagaggtggaatttaagcttggttcatttgcattaaaatccatttgagtattatactctatgcttgaccagtaattaaacactgttcttgggtttgagtcttatagaaataacctttatcatattagcatattcaccgcataggtttgacatggagagaatgagctgaatagatgatatcaattctgggtttcattgggaattaatataacgagattcttgttccactaaggtgtcctaaacacatgagtttttcatatatctgtcttctggataagacatgtagaatgcaaacttaagacatgatttcctatttaagattaacttttagtgtgtgtgtgtgtgtgtgtgtgtgtgtgtgtgtgtgtgtgtgtgtgtttgtgaacgtgcatgtgtgtttgtttataagtgttatacatataggtaataagaaggccttggatacctggtattggatttagagatggttattggcagtctaggtggatggtggagtcactttcatgtccactaaagaatcaagctcttataaccattgagccatcactccatgacgtgcatgcttacatataataacaataacagtgctgatgttgtcgttagggaacatgaaaatctgtcatcaagtcatctcttatctttacagatggtctgggcctctacctacctcgttggctgtgatgttgcagcttgtcgcagacaaaaggcagctacatatctttatgtgtgtcactattgtcatgagtatgtgatgccagtttattattctttctaaatagcgtatcttcaaaaaataaccttgtctaaggtttcaaatattgtaaattaatgttctagggtttttccccagttattttgttaacaaaatgtcacaaatataccccaaatgtttgatttttcatcatgtttttactaagttaacagagatatacacatttggaaaatttattggtgattctagtcattcactggaaagtgttatcatacagggaagtgaatttgaactgggttaggctaagaaaggagatgcaggcaaatgccactccctgtgctaggctttgggatccctatgttgacttgtgtatttcctttagaaataaaatttatgttactctgagtgacataaagacaatgttttatttttaacagagaaacaattttaaaggcaatatatgtatgcatatatattatgtatgcatatattacttgcatacaaacatatgcatacatacaatgcaaatataaaaaccttttaaaataaattaaatgcacatattctaaatgctaccaacacaggtactagtcacaaaatctagaacatataacccaattatttcagagaaatcagatgtacttccttggaccagtgagtatatttttgtaactttaaaaatggcactaagagggctggtgagatggctcagtgggtaagagcaccgactgctcttctgaaggtcccgagttcaaatctcagcaaacacatggtgactcacaagcacccataatgagattggaagccctcttctggcatgtctgaagacagttacagagtacttatgtataataataaataaatcttttaaaaaagtggcactaagaactgcatattatctaaataaacatttagatattgatagtcaagaagttttgcatttcctgaaacttcaaatatttcatatctatggcttttttctacaatctttcccacacccggtactttctttagttttttttttttcatgtttatgtgcttttatgatttatatactgtaaaatgaggtaaaaagatgctaatgaattgtgtacttttgccactaaactctatgttttctaccacaattttccaagttcttgattaaagcagtacttgccaaatttgactgtacaaatatccagagccctgttagaacatgactgctcaaagatagggagaaatgagtccaagtgtacatacacagaactgctatgggatgagtgcaaaggcagtgctcctagccatgaatctatatcacaaaacccttgtaactagtagctctgtttgcaaatacacttaaacaccatcttttgaattggctgctggcagtagatgtaagttagtattttcccgtgacagttttcactatatctcgacttcttttcttttttaaaggggaaatcatcaagatacactaaatatgccttataaggagggctcaccatgtgacgattgtccaaataactgcgaagatggactgtgcagtgagttgtctttgttgtttaatttaggttgcctgaagttggcataccaaaatggggaacacacatcctttaagtgaagcattgacctgcggtctatatttctgtgactttcatcttaatatatacattatttctgcttcccttctaacacccagagtaatccaacctctttgactttatcactggagggcagaattagaatatctatatatgaaaacttaccttcttgcccaaaagtttaaaattatgaatatagaagcattctaggtaaattattaatatgcatggctacttttcccagacacttcctgagataccttattgctgaccacaaactattcctgtaaggttaccagtctctctgaaatgcttactgctctataaccctcaacttgtctgtaatagatcagtgtggaattttttttattatcacttttattttttttaaagtcaagtcatggcatttccccttcaacagttcctcatcccgtttctcctccccccatcaccaacaggatgtccatccctgtggcaaaccttcccagtccctgggtctcaagtctctggaggtttaggtgcatcttctcccactgaggccagactaggcagtcctctgctgtatatgtgtcgggggccttggaccagctcatgtatggtgcctggatggctcaatctgagagatctcaagggtccagattagttgagactgctggtcttgctatgggaccaccctctacctcagctccatccagccttcccctaattcaaccacggggttccctgacttcaatccaatgtctgttctaagtgtttgcctctgtttcactcagctgcttgtcaggcctctcagaggacagccatgctttgcacaccatagcatcagtaagcgtcaggtcttggaaccttcccataaaacggattctgagttgaaatggtcactggaccttgtttctctgactcttctctgtttttgtccctgcagttcttttagacaggaacagtcctgattagaaattataactgtgggttagtaaccctatccctccacttgatgccctgtctttctgctggaggtggactctacaagttctcttttcccattgtagggcatttcatctaaggtactttcctttgattcctgacagtctctaacctcgcatgtctctggtactttctagatcccccccccccccacctcctacacaccatggtcgcatatttccattcattatgctggtcctcagagtttctcttctctttcccccacctcataccttatcacattcccttttcctctttcccctccttttccacccaggttctctccctttgccatccatggtgattttcatctccctctcaagtgggattgaaacattctcacttggtcccttctgcttgttaacctccttgaattctgtgaattgaattctctacttttttttttaaccaatatccacttattagtgagtacataccatgcatgtttttaggtctgagtttcctcactcaggatgatattttctagttccatccacttgcctgcaaaactcatgatgtccttgttcttaacaactgaatagtattcaattgtgtgcatctggattgtttccagcttctggctattataaataagaccacaatgaacatagtgaagcaagtgtctttgtgctatggtagggcatatttttttgtacattcccagtagtggtatagctgggtcttcaggtagagatatttccaattttctgaggaacctccagattgatatccagagtggttgtaccatttacaatcagtgtggaatttttgtaagctgaccattattcttcttcattagctcagaatctatccaatactggatttttctgatctctcttatattgccatccagatatactactccttagcatatgcctaaggaacttggcatccttctccacatattcttatgtaatagttgtcattttcactccactcacaataactaggaaatgaaaatatgaacctcttcaatagattaatggatagtgaaaatatggtatgtgtatgcactgtggaattctgttcatcctgaaaaaaaaactggaaataaaatatttttcaaagaaattggtgaaactggaaaatactatgctgattaaggtaactcaggcccagaaaataaatgccacatgttctctcctatttgtgaatcttagctccaaagttttggatgcatgtacctcctggggtaacctacagaaataaggaatgtaagaaggaccacagtgtggggctgacagctctagaaaagcgaatagtaggacacaggcacgatgaaggtgaaaatggcaaatatagaaaggagactataactggggataaaggaatatgggcaatatatagggagaaagaggaccgaggaatatataacagtaaggttgtttaaaaaatatgttgaaatattttatgtttagttcaaaatatacacacagtttaaatctaattatatgacttggagtaataatgcttccctcaatgcttctccccaaatcatagactatctaacaaaatcctgagagccaggaatgtgaaaccttccttccaatcgttggtcaggagtgtctaagagactcaaacacaagaaagtctatttccaatgccttgcctgccagccagaactttaagggaagaccctgatgctgaagatagtacagactttggacacaaaaatcaaagaaattaagcttggattgtgaatgtcacaatctgacaaagctccaactctatagacaaaaacctataggcaattagtgattgttttaaaagggagtatagtactgcaacttggagacctctccttttctaaagagaaacaggggagcaaaagtagacgagagattgcagggaaaggaagttgtagagggagagaggaaaaatgggagaagaggaggatagaaagtagaaacattagttgagatgtaaagtaaataataaattaattaaaaatagccaaagcaatcttgagaaaatgtgagggaccaggtcttgcaatactaagctttaatatatcctacaaagctatattagtcaaaacaggacagtattaatagaaagatacgcctctagagcatatgggcagaatccaatgggcagaatacaagtaaacttgtgcagctataaagagaaggtaataaaaaatttaaaaagaaaaaaaaaaagaaaaggaaataaggggaaagggaaggaaagaaggagggaaagagagagagagaatgaatgaatgaatgaatgaatgaggaagaaaatcagtttttttagggatgagacccttaatagattatccaagtcttgacacatatataaaatctaaagaagagtcatcaattaaagagagacagaggaagaaacattggtgggatcctatgaggaagagtaaaggataaaaatgatataaatactatatataagtatcaaattttaaaatattttattattaaatactttattaatttaaattataagttaaaatattataacatataattcatgccacttatgaaactagcactcctaaagagagtcccatttttcccccattctctcggtcattcataaataaagaatgtgtgtttggtctacatcttaaatttactcctctgtttgttgaatttcttcatggacaatatgtccgttgatgacaatggagtgttaaagtcctcatgcatcacttcttggaaagctactaatatttggtttgtgtacttagaagcttttatgcatatgcacttagaatttttattattttacccacttactgtttatcattacacaatgaccttctttgatttttcctgaagtctattttatctgtaattaatggaatgacacttgctttcatctgaattttattaacatggaatatcttgattcattcatatactatctgctagtgtgaatccccagagattttcttgcaaacaacatctatttaaatattgtcttgttttctgtttgttcatttcctatgtctttcaggtggaaaatgaagattaatttacttacattcaaagacatcaactaatgatgtcattgttcaaacttgctttttagttttctttctttttttttaaagatttatttattatttattatatgtaagtacactgtagctgtcttcagacacaccagaagagggcatcagatctcattacagatggttgtgagccaccatgtggttgctgggatttgaactctggacctttgaaagagcagtcgggtactcttacccactgagccatctcaccagcccactttttagttttcaaataacccattttcttcctctcacacagccttcttgcagttagtttacttaatataatagctacattttgatatctgacttacttttttgttttctattaaagatgttttcttagcaataagttgtcaaaggaacttagcaccagatgtgaggtaccccctgttacattttgtattatagaatttagaaataacagaaacttagataaaataataaaaataaatggagaatactttgatttttaaaagactatatttgcagattgttttctcttctgttagattcctattacatgtcaaactttacatctttctaaatctcttacttcagtatattaaagaacctgttattaattttattattttagttttagtatttatactaaagagatgggtagcttatgcattacatttgtaatattggagtatcctgaatttgtgtgtagaattactattttcactgaattttatacttcccaggatttccactgttcatattataggtaaatttgttcagcttaaagaatcacctgcagcatttttttctataggacaggcatagtaaaaattccctttgacctaattttttctgattttgcatgtatctttccttcatttccaaacataacgttggcagggacagaaaccctgcttcacagtttctttccttccacactgaaaaaatgttcaagtccctgttttcctaagccgtggagcatggactatgtcagcacactgctcttaagaatctcctttctaactttccattttgagttgctgattatacaacacatcacgatatatgttgagctaaataaggagggcaaacgtgactttcctatacctggatattattactttgtacagattaaagaatgaacactgtcttttgcgatgtcagagggatggtggagcgtgtttataatgacctgaatgtcacaatgtcacaggtctagaggctcagttcatgaacagtgatttagagggaggagctggacaattcctgatactgaatttctctatgttgagtttctttttttttattaaggcatttttattagatattttcttcatttacatttcaaatgctatccccaaggccccctataccctctcgcgccctgctcccaaacccctccactcctgcttcctggccctgacattcccctgtactggggcatataatcttcacaaaagcaaggtcctctcctcccattgatggccgactacatcatcctctgctacatatgcaactagagacatagctctagggagtaatggttagtccatattgttcctcctctaaggttgcagaccccttcagctccttgggtactttctctagctcctccattcggggcactgtgttccattcaatagatgactgtgagcatccgcttctgtgtttgccaggcattggcatagcctcccaagagagagctatatcagggtcctgtcagcaaaatctttctggcatatgcaataaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntcacgagcaggaggatctctctttaagctgtatttttatgttgaggaagaactatcatggcagtttttcagttagtttgactgctttccctattttcataccataaaaccaagtactgagatctcacagatggtgaatttcttgtgatgatagtttcatgcctgactaggcactgaaatgaacgtgtctttagagagagttggttggagagcactttttcttttttctttttcttggatctatgggagtcatgtggaagcatagggagaagtattgcaggccacaaggggaatagggagtccacaagaagaccaaaagagtcaattcacctggacccttgggggctcccagagactgaaccaccaaccaaagagtgaggatgggctggatctagccccctggaaatatagcaaatgtgtaacttggtcttcatgcaggtccccgaacaactggagcaggggctgtccttgaacctgttgcttggatatagttcccccagcagcgctgccttgtctggcctcagtgggagaagatgagcctaatcctgtagagacttgatctgccagagtgcagtgataccaatgggaggcatctaccctctcatcagagaaagggatgggagaaggaggaggagggaggatctgtggaaaggggatactggcaggagagggggacagatatagggaggtaaagtaaataaagaaatcaattagtggaaaaagagtatatccttcaatgataaacctcaaatacattttaaaaagtgactgaaatgccagtgtcttatcataattgtcatggtaatattgttctcaaaatgacagagaaattgcaaagatcacttatctccagtctgttcaggtgcagtgtcaccaattacaaaactgatattgaataaacatgcttcatagcatgacaagcactttctcctttacttcaaggggatctgattcaacatgtcaattgttttgcattaacctaaaaagctaaccttaatcaagttaatcctttattatatagaacttttctcttgacgtcataaaaagatgatgtctttccttaatggtcacaactgttttaagcacattgttgcataccattctttttgctttccagctaacccctgcatttattatgatgaatacaacaactgtgatacgcaggtaaaactttatgggtgctcacatccagctgtccagccattctgcaaggcttcttgtctgtgtacaactgaaataaaataacctaaagtttgctactatgctctgaagataaagtgtgttttcaggctcaattatttcaccttaatgtaatgtactgtaagattttgtactgaatttcatttttttaaattatgctcaagaacttaatttttaaccatcatttatagacatcaatatattttcatcaattctgtgcccagtgcatgtaataacaaagatttaaatttcattgtttaatgaaagggcaagtcaaatgcttgccccaaatttaatacatgtgatttgtgatatgtagctaaacttaatccagctaatgaatattcttcatctgtaacttgtagtaaaatagcttttacctaacccttttcccaatatctccacttcccaacaaccctaattttttttgttttaatgtttaattttcattctattttaaatcagacattaaaaatattcaacaaacaaacaagagtcaggcttcaacttgaagtatagatatgactcaactaagagtggtcaaaatgtctcccaaactcattagtatctcagcatgcatgtttgtgatgtaggaaatgagagccaggctgtctttgggttccttgtcccttcacctagatttctcctgtgccctcctacacacccctgttgccctgagcaccccattcctagattgtcgtgtatctaaaaccataatgaaatgaccataacaccaggctgccaaattatttgaaatatctaatcctttaaattatattgctttactagaaaaattatgtcctaagatagttctgtatacagttctaataaaaggaaaaaagttctgtatacagttctaatcaaaggaaaaaaagtgtgtgtgtgtgtgtgtgtgtgtgtgtgtgtgtgtgaaaggtgtgtgtttgtatgtnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 42 - DNA, 742 bp, Artificial
  End sequencing M13R 5′ end of 5′ for subclone 5′ arm/3loxP3NwCD confirmation
ataatctacctgaaaccagtggtcccatgatcttatancagaggtcagataggagtttttttgtggtttatgtctattgttcacagataaaaagtcacaggcatatattatagtgttcagtcatttatgtatgctacataaaacaaatatttttcacctatttgtgacagaagcctttgtatttattttttacatgaatatgtacatatacatgtttcagttaatctttgtgtagttagatatattttccaacaatgcccttatcattaactaaaggcaatatatagtatattatatgccttcacattatgatatgaggcaatctaacagattgtagcattctgggctgcccacttgggttagatgtctactaaagattttggctgtttcttttgatgcaggctgctcctcttatttgagcagatatgtctgaaattttcagaagaccctctgttgtaataaagttttggtggtataatctatttaactaatattctaaactcctggctctatctctactctgtctctttgggctgggtaatatctgcagcaagttactattcagacattagtaaccatgatgcagacacttattaagtactgattaatttatggaataagaggccctcatgtctattattangttagctattgtagtgataagtagaaaagaactgaagagtaggtaagcaattatagtaagaatagtggttaatatcagantggttagatgggaagtaaa

SEQ ID NO: 43 - DNA, 431 bp, Artificial
  End sequencing NeoR 3′ end of 5′ for subclone 5′ arm/3loxP3NwCD confirmation
cagccatgtatatttgaaagagtacatagaaggcaggtgatcatgttactactgttgtttgtgtgtaatttaaagatttctatacatttaattgaaagatattctgtacatttccattacttggctaataataattgtctttgtgttatgcaaaataaatactaatgggtctttattcatttttagtacagttagtcaattctaagatactaatgaggtggcactttttctaacaacctgttatgtgtatttgtataaacctactagttaacttttacagaaatttctaatctttaaaagcaacctaatcaatttatgaagaattaaatagtaaacaggagtacttatgcttgtttccatagttacatggctgtcctgtcattgctctcagatgtcccatgtcctctgaaaaaaaaaagtataacctggag

SEQ ID NO: 44 - DNA, 637 bp, Artificial
  End sequencing M13R 5′ end of condition knockout arm for subclone conditional knockout/3loxP3NwCD confirmation
atggagtctatttcatgttatattctacaaatgttgtctcctgactttttctttcacaattaggcacctttgttattaacttaaatcaatgaagaatatataggataatctcttttaagctttagtcacttggtgagaaacctaaatgctatctgctattttaataccccataaacacacaattgtgtatatttagactctggaagttaaggagtccatatcatttaagttaagtccatatcatctaaatcaactgaatattattttatgaggccccttagctaaggcttaatattgaactttattttcatgtcccattaacaaaaatctaacaaagtaaaaccttgctcaaggcttcttagaaaataattttttacacactaagagtaacctaaatttctgttacttatatttttcaggtatggcagtgaaatttatcttgttattgtttgttgctgcctttgttcctgttgtgactatcagagtatgtaaatattatatgttacataactatattttatttaagggtaggagaagggtgtatcagttattcacatacttcatataatgatgcagttaaacaagtcttattctgttttataaaaagaattatttatttattctatgtatatgatta

SEQ ID NO: 45 - DNA, 642 bp, Artificial
  End sequencing NeoR 3′ end of condition knockout arm for subclone conditional knockout/3loxP3NwCD confirmation
aattcttctaaacttgaaagttcacttaggccttctccttttggtttcattcgtgattctgctgtattaagaatagaacttgtttgggcagaagaaatagctcagtagttaagagcaatgattgctcttcaagatgttctgagttcaatacacaggaaccacatggctcacaaccatctgtaaaggaatccaatgtcctcttctgatatgtctggagatagctatagtgtaatcatatacatagaataaataaataattctttttataaaacagaataagacttgtttaactgcatcattatatgaagtatgtgaataactgatacacccttctcctacccttaaataaaatatagttatgtaacatataatatttacatactctgatagtcacaacaggaacaaaggcagcaacaaacaataacaagataaatttcactgccatacctgaaaaatataagtaacagaaatttaggttactcttagtgtgtaaaaaattattttctaagaagccttgagcaaggttttactttgttagatttttgttaatgggacatgaaaataaagttcaatattaagccttagctaaggggcctcataaaataatattcagttgatttagatgatatggacttaacttaa

SEQ ID NO: 46 - DNA, 746 bp, Artificial
  End sequencing M13R 5′ end of 3′ arm for subclone 3′ arm/pCR.21 confirmation
gatgaggattaaagttcaactttcactttaactcattggatccatgtctatgtctttttttataaacaataagaaaacaaacaaacaaataaataaaggcaagctagcaaaggatgtgataaggtagagaagggatggaggaagagaaagaaaaaaatccataggcaagaacttgtttacaatttacccatgtgttttaataaccagatcgaagaaattagataaatagtattatttttaattttcctcccacataccctttttgattttaagaatgttataaatcaaaattaaatatgaaattgaaagttacaactgcaaagatgtgtgagagattcagcaatggaatcaaggtatgatgcttggatctaattgctcataaaaataaaacctttgggctagtgatatagctcagtattatagcacttgaattgtgtgtgtgtgtgatgtccagaatagatccccaggatcacaaagtaagataatgatggtaatgctgatgttgttaatgataatgatgacttcaatatttatagaacaaaaccaacatcttcagtggtaaaataggtagagtgtcctacaaatttgtgatcattgagtgacaattcatcattagcaaccaaaataaggcatggaaaactgatcttaatttactttatctaattaaaaaatgacttaatgtcaatagatttttcttttgtagaactagattttttgcgtgactgtgtatgtgtgtttctcangag

SEQ ID NO: 47 - DNA, 719 bp, Artificial
  End sequencing M13F 3′ end of 3′ arm for subclone 3′ arm/pCR.21 confirmation
ccagcccaaaaccaggttctaaagcttttaagggtcatacaagagagaatttgaatcagttactcgagattagcctgaactcttcatttgggtgggctaagtgtagcatatactcagttgaagcatcatttaataaaccactcagacacacgtatgtaggagtagcagtgacatcctgacaatcctgcacccagcatcgtcagagtgtgctcacagcctgtgtgttacactgtcaacaataacatgatttccttggtatgttcccagacttttatataaattttctaaggatgtacagacagttacaactctttttagactcgttttaccctacttctgtggtttgagattggcattttaacgaaatttttagaacacattatgttcactggacttagtaataatgtaggaggcaacattagaattattgccatcactcttcagagtaaaggtcaattttctcagcatgacagtgaaaagataattactgtatgtgtgtacggtaccatctagtggctattgtgtagaattactcttctagttgactgcatccaaaacatgttatttgtttccgtttttcaaataagaaataatacttttgagggacatcaaagaactcagatgtacctcaacagaggaatggatacaganaatgtgatacatttacacnatggagtactactcagcaattaaaacaatgaantcatgaaattcntagg

SEQ ID NO: 48 - DNA, 386 bp, Artificial
  0.4 kb EcoRI fragment from 5′ probe/pCR2.1-TOPO
gaaatgctgattttattctcctttgtctctgttttaactgagttcaggaaactagttagcatctcagataaaaggtaagctgtgtttacagatttgcagcctgtggtcaatggagaatcatgcaagatactctggatgctttgccctggtcagtccatggattacatcaagtctcagtgatctagtgaacaaagctcagacaaggcctctaccctgagactatttttcctctaagcctgaatagctgaatagcttcgatttatgctgtctaatatttatctttagaccagcatctttgttgatatcaggctcaattttctgctgaaaccacaacctctttctgatccttctctctacccttgactcctgattttgagttgtgttta

SEQ ID NO: 49 - DNA, 251 bp, Artificial
  0.3 kb EcoRI fragment from 3′ probe/pCR2.1-TOPO
ctgcaagagtggaagatttatggttaataacagtgtctaggaatgttaactcctttgctttaatcactacttgaggcagggatctagaactctgctttcattcacagaagccatttgtgtgctaagtgcatattttgagactcaaaatatcacaggttgattgtaaggactgccgactggacaaagcttttggcataacttccatatcatctttacagaagctgtgactcaaagaggtgaaatgacaagcc

(19) United States
(12) Patent Application Publication
(10) Pub. No.: US 2006/0275803 A1
(43) Pub. Date: Dec. 7, 2006

(54) Rat and mouse members of the CRISP family of genes

(75) Inventors: Michael Alexander Nolan, Jamaica Plain, MA (US); Daniel S. Johnston, Trappe, PA (US); Leeying Wu, Lexington, MA (US); Scott Alan Jelinsky, Waltham, MA (US)

Correspondence Address: POTTER ANDERSON & CORROON LLP/WYETH, 1313 NORTH MARKET STREET, HERCULES PLAZA, SIXTH FLOOR, WILMINGTON, DE, 19801, US

(73) Assignee: Wyeth, Madison, NJ (US)

(21) Appl. No.: 11/417,318

(22) Filed: May 3, 2006

Related U.S. Application Data
(60) Provisional application No. 60/677,670, filed on May 4, 2005.

(51) Int. Cl. C12Q 1/68; G01N 33/53; C07H 21/04; C12P 21/06; C07K 14/47; C07K 16/18; A61K 38/17

(52) U.S. Cl. 435/6; 435/69.1; 435/320.1; 435/325; 530/350; 530/388.1; 514/12; 435/7.1; 536/23.2

(57) ABSTRACT

Disclosed herein are nucleic acid and polypeptide sequences of the mouse and rat homologues of human cysteine-rich secretory protein-1. Also disclosed herein are methods related to the use of the aforementioned mouse and rat homologues.

### CROSS-REFERENCE TO RELATED APPLICATION

This application claims priority from U.S. Provisional Application No. 60/677,670, filed May 4, 2005, which is incorporated by reference herein in its entirety.

### FIELD OF THE INVENTION

The present invention relates to compositions and methodologies employing mouse and rat homologues to human cysteine-rich secretory protein-1 (hCRISP1). The compositions and methodologies disclosed herein can be used for diagnosing, prognosing, and treating CRISP1-related conditions and, in particular, conditions associated with the epididymis.

### BACKGROUND OF THE INVENTION

The epididymis is a specialized male reproductive organ in which some of the post-testicular maturation events that are required for male fertility occur. The epididymis consists of a continuous tubule that is densely packed and organized into three anatomically and morphologically distinct regions (caput, corpus, and cauda). These regions can be further subdivided into segments using microdissection techniques and the naturally-occurring boundaries of connective tissue septae and cleavage planes (Turner T T et al., Reproduction 125:871-8 (2003)). The epithelial cells that the line the lumen of the epididymal tubule are responsible for the fluid components of the tubule and exhibit different morphologies and patterns of gene expression in a segment-dependent fashion. As a result, spermatozoa passing through the epididymis are continuously bathed in a series of changing microenvironments within a single tubule (Turner T T, J. Androl. 16:292-98 (1995)).

The dynamic nature of the epididymis is crucial to the post-testicular maturation events that produce progressively motile sperm that are able to undergo the further maturation events in the female reproductive tract (termed capacitation) and eventually fertilize an egg. In the epididymis, spermatozoa experience dramatic changes both to their plasma membrane lipid and protein compositions (Jones R, Oxf. Rev. Reprod. Biol. 11:285-337 (1989); Turner T T, J. Androl. 16:292-98 (1995); Cooper T G, J. Reprod. Fertil. Suppl. 53:119-36 (1998); Jones R, J. Reprod. Fertil. Suppl. 53:73-84 (1998); Toshimori K, Microsc. Res. Tech. 61:1-6 (2003)). Spermatozoa are both transcriptionally and translationally inactive in the epididymis; thus, new proteins must be acquired from the epididymal fluid. The strict regulation of the microenvironments within the tubule suggests that the order in which proteins are acquired is an additional requirement for successful epididymal maturation. This strict regulation is exhibited in the exceptional number of genes encoding secreted proteins that display segment dependent changes along the length of the epididymis (Viger R S et al., J. Androl. 17:27-34 (1996); Hinton B T et al., J. Reprod. Fertil. Suppl. 53:47-57 (1998); Cyr D G et al., J. Androl. 22:124-35 (2001); Roberts R P et al., Biol. Reprod. 67:525-33 (2002)). Similarly, the presence of clusters of genes that follow the same general segment-by-segment pattern (i.e., lowest to highest, highest to lowest, etc.) implies that there are global mechanisms (e.g., testicular androgens) that regulate gene expression to establish these microenvironments.

A promising therapeutic approach to the development of contraceptive agents would be to act upon proteins that are uniquely expressed in the epididymis (CooperT G et al., Hum. Reprod. Update 5:141-52 (1999); Puri C P et al., Asian J. Androl. 2:179-90 (2000); Khole V, Indian J. Exp. Biol. 41:764-72 (2003)). Disrupting the normal expression of these genes or their encoded proteins to alter their required biological function in such a way that the post-testicular maturation of spermatozoa does not occur successfully would result in sperm that are incapable of fertilizing an oocyte. Presently, there are no drugs in use that accomplish this contraceptive approach.

Currently, the CRISP1 (cysteine-rich secretory protein-1) family is the most intensely studied of the mammalian CRISPs in the field of reproductive biology, where rat CRISP1 and mouse CRISP1 are employed as the model systems for understanding the physiological roles of human CRISP1.

A significant need exists to identify the physiological and molecular roles of the mammalian CRISP family members for the screening and development of new drugs, for diagnosis, prognosis, prevention, and treatment of defects in fertility, and for the evaluation of therapies for fertility-related conditions including contraception. The present invention overcomes the problems with the current models for studying CRISP proteins in humans by identifying a novel mouse gene and a novel rat gene that are more similar to human CRISP1 than those identified in the literature.

### SUMMARY OF THE INVENTION

Applicants herein identify the mouse gene 9230112K08Rik (herein termed mCRISPC) and the rat gene #ENSRNOG00000013612 (herein termed rCRISPC) as members of the CRISP family of genes. Applicants further identify the cDNA, mRNA, and protein sequences of the products of the 9230112K08Rik and #ENSRNOG00000013612 genes. Applicants also demonstrate that 9230112K08Rik and #ENSRNOG00000013612 are the closest homologues of hCRISP1 in mouse and rat thus rectifying the disparity between nomenclature and homology for the mouse, rat, and human CRISP family of genes.

A further objective is to utilize the mouse and rat genes and their mRNA and protein products to understand their molecular and physiological properties and apply these findings to the development of therapeutic agents.

One aspect is provides an isolated polynucleotide encoding a CRISPC polypeptide comprising a nucleotide sequence selected from the group consisting of:
(a) a nucleotide sequence encoding an amino acid sequence having at least 65% identity with the amino acid sequence set forth in SEQ ID NO:5;
(b) a nucleotide sequence encoding an amino acid sequence having at least 65% identity with the amino acid sequence set forth in SEQ ID NO:6;
(c) a nucleotide sequence which hybridizes with (a) or (b) under the following conditions: 6×SSC at 45° C. and washed at least once with 0.2×SSC, 0.1% SDS at 50° C.; and
(d) a nucleotide sequence complementary to (a), (b), or (c).

Preferably, the nucleotide sequence encodes an amino acid sequence having at least 70%, more preferably at least 80%, even more preferably at least 90%, and most preferably at least 95% identity with the amino acid sequence of SEQ ID NO:5 or SEQ ID NO:6. Particularly preferred is a nucleotide sequence of nucleotides 97-846 of SEQ ID NO:19 or nucleotides 161-922 of SEQ ID NO:25.

Vectors, transformed host cells, and non-human transgenic animals comprising the isolated polynucleotides are further embodiments.

Another embodiment is for isolated antisense polynucleotides antisense to the isolated polynucleotides. Preferably, the isolated antisense polynucleotides are antisense oligonucleotides, ribozymes, or siRNAs.

Another aspect is for an isolated polynucleotide comprising the polynucleotide sequence of SEQ ID NO:3 or SEQ ID NO:4. A further aspect is for an isolated polynucleotide comprising the polynucleotide sequence of SEQ ID NO:29 or SEQ ID NO:30. Another embodiment is for an isolated polynucleotide fragment comprising at least 12 contiguous nucleotides from the polynucleotide sequence selected from the group consisting of SEQ ID NO:3, SEQ ID NO:4, SEQ ID NO:29, and SEQ ID NO:30.

A further embodiment is for a polynucleotide fragment encoding a biologically active portion of an mCRISPC or rCRISPC protein.

Another aspect is for an isolated CRISPC polypeptide encoded by a nucleotide sequence selected from the group consisting of:
(a) a nucleotide sequence encoding an amino acid sequence having at least 65% identity with the amino acid sequence set forth in SEQ ID NO:5;
(b) a nucleotide sequence encoding an amino acid sequence having at least 65% identity with the amino acid sequence set forth in SEQ ID NO:6; and
(c) a nucleotide sequence that hybridizes with the complement of the nucleotide sequence of (a) or (b) under the following conditions: 6×SSC at 45° C. and washed at least once with 0.2×SSC, 0.1% SDS at 50° C.

Preferably, the isolated CRISPC polypeptides is encoded by nucleotide sequence which encodes an amino acid sequence having at least 70%, more preferably at least 80%, even more preferably at least 90%, and most preferably at least 95% identity with the amino acid sequence of SEQ ID NO:5 or SEQ ID NO:6. Particularly preferred are the isolated CRISPC polypeptides having the amino acid sequences set forth in SEQ ID NO:5 and SEQ ID NO:6.

An additional embodiment is for a fusion protein comprising a first polypeptide consisting of an isolated CRISPC polypeptide operably linked to a second, non-CRISPC polypeptide.

A further embodiment is for an antibody which specifically binds a CRISPC polypeptide comprising SEQ ID NO:5 or SEQ ID NO:6; which specifically binds a CRISPC polypeptide fragment comprising at least 8 contiguous amino acids from SEQ ID NO:5 or SEQ ID NO:6; or which specifically binds a CRISPC polypeptide fragment consisting of SEQ ID NO:13, SEQ ID NO:14, SEQ ID NO:15, or SEQ ID NO:16.

A further aspect is for a method for detecting a CRISPC polypeptide comprising detecting binding of an antibody selected from the group consisting of
(a) an antibody which selectively binds a CRISPC polypeptide comprising SEQ ID NO:5;
(b) an antibody which selectively binds a CRISPC polypeptide fragment comprising at least 8 contiguous amino acids from SEQ ID NO:5;
(c) an antibody which selectively binds a CRISPC polypeptide comprising SEQ ID NO:6; and
(d) an antibody which selectively binds a CRISPC polypeptide fragment comprising at least 8 contiguous amino acids from SEQ ID NO:6;
   to a molecule in a sample suspected of containing a CRISPC polypeptide, wherein the antibody is contacted with the sample under conditions that permit specific binding with any CRISPC polypeptide present in the sample and binding of the antibody to the molecule in the sample indicates the presence of CRISPC.

Another aspect is for a method for detecting expression of CRISPC comprising detecting mRNA encoding CRISPC in a sample from a cell suspected of expressing CRISPC with a probe comprising at least 12 contiguous nucleotides from nucleotides 97-846 of SEQ ID NO:19 or nucleotides 161-922 of SEQ ID NO:25.

A further aspect is for a method for determining whether a CRISPC gene has been mutated or deleted comprising detecting, in a sample of cells from a subject, the presence or absence of a genetic alteration characterized by at least one of an alteration affecting the integrity of a gene encoding a CRISPC protein or the misexpression of a CRISPC gene, wherein the detecting step is performed with at least one of a probe or primer comprising at least 12 contiguous nucleotides from nucleotides 97-846 of SEQ ID NO:19 or nucleotides 161-922 of SEQ ID NO:25.

An additional aspect is for a method of identifying CRISPC variants comprising screening a combinatorial library comprising mCRISPC or rCRISPC mutants for CRISPC agonists or antagonists.

A further aspect is for a method of isolating a CRISPC polypeptide comprising
(a) contacting an mCRISPC or rCRISPC antibody with a sample suspected of containing a CRISPC polypeptide; and
(b) isolating an mCRISPC or rCRISPC antibody-CRISPC polypeptide complex from the sample.

Another aspect is for a method of producing a CRISPC polypeptide comprising
(a) culturing a transformed host cell comprising an expression vector comprising an isolated polynucleotide selected from the group consisting of:
   (i) a nucleotide sequence encoding an amino acid sequence having at least 65% identity with the amino acid sequence set forth in SEQ ID NO:5;
   (ii) a nucleotide sequence encoding an amino acid sequence having at least 65% identity with the amino acid sequence set forth in SEQ ID NO:6;
   (iii) a nucleotide sequence which hybridizes with (i) or (ii) under the following conditions: 6×SSC at 45° C. and washed at least once with 0.2×SSC, 0.1% SDS at 50° C.; and
   (iv) a nucleotide sequence complementary to (i), (ii), or (iii);
in a suitable medium such that a CRISPC polypeptide is produced; and
(b) optionally recovering the CRISPC polypeptide of step (a).

An additional aspect is for a method of screening for compounds which modulate mCRISPC or rCRISPC polypeptide biological activity comprising:
(a) contacting a test compound with a sample containing an mCRISPC or rCRISPC polypeptide; and
(b) determining the ability of the test compound to modulate the biological activity of the mCRISPC or rCRISPC polypeptide.

A further aspect is for a method for the treatment of a mammal in need of increased CRISPC activity comprising administering to the mammal in need thereof a therapeutically effective amount of an mCRISPC or rCRISPC polynucleotide or polypeptide.

Another aspect is for a method for the treatment of a mammal in need of decreased CRISPC activity comprising administering to the mammal in need thereof a therapeutically effective amount of an mCRISPC or rCRISPC antisense polynucleotide. Preferably, the mCRISPC or rCRISPC antisense polynucleotide is an antisense oligonucleotide, a ribozyme, or an siRNA.

An additional aspect is for a method for the treatment of a mammal in need of decreased CRISPC activity comprising administering to the mammal in need thereof a therapeutically effective amount of an mCRISPC or rCRISPC antibody.

Another aspect for a method for obtaining anti-mCRISPC or anti-rCRISPC antibodies comprising:
(a) immunizing an animal with an immunogenic mCRISPC protein, an immunogenic rCRISPC protein, or an immunogenic portion thereof unique to an mCRISPC or rCRISPC polypeptide; and
(b) isolating from the animal antibodies that specifically bind to an mCRISPC or rCRISPC protein.

A further aspect is for a method of developing a sensor cell for determining the activity of a gene comprising:
(a) providing a homogeneous population of cells, wherein each of the cells comprises a signal transduction detection system;
(b) introducing into the population of cells an isolated genomic construct comprising an mCRISPC or rCRISPC promoter operably linked to a targeting sequence, wherein:
   (i) the targeting sequence comprises a region of homology to a target gene sufficient to promote homologous recombination of the isolated genomic construct following introduction into the cells;
   (ii) the mCRISPC or rCRISPC promoter is heterologous to the target gene;
   (iii) following recombination the promoter controls transcription of an mRNA that encodes a polypeptide comprising an activatable domain; and
   (iv) the polypeptide is capable, upon activation of the activatable domain, of altering the signal detected from the signal transduction system;
(c) incubating the population of cells under conditions which cause expression of the polypeptide;
(d) incubating the population of cells under conditions which cause activation of the activatable domain of the polypeptide; and
(e) selecting cells that have altered the signal detected from the signal transduction system.

An additional aspect is for a method for the production of a CRISPC polypeptide comprising:
(a) providing a homogeneous population of cells;
(b) introducing into the population of cells an isolated genomic construct comprising a promoter operably linked to an mCRISPC or rCRISPC targeting sequence, wherein:
   (i) the mCRISPC or rCRISPC targeting sequence comprises a region of homology to a CRISPC target gene sufficient to promote homologous recombination of the isolated genomic construct following introduction into the cells;
   (ii) the promoter is heterologous to the CRISPC target gene; and
   (iii) following recombination the promoter controls transcription of an mRNA that encodes a CRISPC polypeptide; and
(c) incubating the population of cells under conditions which cause expression of the CRISPC polypeptide.

Another embodiment is a kit for detecting CRISPC polypeptide or polynucleotide comprising:
(a) a labeled compound or agent capable of detecting an mCRISPC or rCRISPC polypeptide or polynucleotide in a biological sample;
(b) means for determining the amount of mCRISPC or rCRISPC polypeptide or polynucleotide in the sample;
(c) means for comparing the amount of mCRISPC or rCRISPC polypeptide or polynucleotide in the sample with a standard; and
(d) optionally, instructions for using the kit to detect mCRISPC or rCRISPC polypeptide or polynucleotide.

An additional embodiment is for a kit for identifying modulators of CRISPC activity comprising:
(a) a cell or composition comprising an mCRISPC or rCRISPC polypeptide;
(b) means for determining mCRISPC or rCRISPC polypeptide activity; and
(c) optionally, instructions for using the kit to identify modulators of CRISPC activity.

Another aspect is for a kit for diagnosing a disorder associated with aberrant CRISPC expression and/or activity in a subject comprising:
(a) a reagent for determining expression of mCRISPC or rCRISPC polypeptide or polynucleotide;
(b) a control to which the results of the subject are compared; and
(c) optionally, instructions for using the kit for diagnostic purposes.

A further aspect is for a knockout construct having the sequence set forth in SEQ ID NO:41.

Another aspect is for a transgenic mouse having a homologous disruption in the CRISPC gene, wherein the disruption results in mice having defective sperm function.

Other objects and advantages of the present invention will become apparent to those skilled in the art upon reference to the detailed description that hereinafter follows.

### BRIEF DESCRIPTION OF THE FIGURES

FIG. 1 is a bar graph showing the tissue distribution of the mouse homologue of hCRISP1.

FIG. 2 is a bar graph showing the expression of the mouse homologue of hCRISP1 in epididymis.

FIG. 3 is a bar graph showing the tissue distribution of the rat homologue of hCRISP1.

FIG. 4 is a bar graph showing the expression of the rat homologue of hCRISP1 in epididymis.

FIG. 5 depicts the multiple alignment of the mouse, rat, and human CRISP family of proteins.

FIG. 6 is a phylogenetic tree showing the relationship between CRISP family members using full-length protein sequences.

FIG. 7 is a phylogenetic tree showing the relationship between CRISP family members using the conserved SCP domains.

FIG. 8 is a Western blot analysis of sf9 cells expressing recombinant rCRISPC (rCRISP4).

FIG. 9 is a Western blot analysis of recombinant rCRISPC solubilization conditions.

FIG. 10 is a Western blot analysis of recombinant rCRISPC following anion exchange chromatography.

FIG. 11 is a comparison of rCRISP1 purified +/−DANA in anion exchange chromatography.

FIG. 12 is Western blots of +DANA rCRISPA (rCRISP1) preparation. A. Silver stained SDS-PAGE gel of anion exchange elution fractions. B. CAP-A polyclonal anti-rCRISPA D/E antibody. C. 4E9 monoclonal anti-rCRISPA antibody.

FIG. 13 is a graph showing rCRISPA (rCRISP1) protease activity (assay conditions: 5 μM substrate peptide, 1 μg enzyme, 100 mM NaCl, 25 mM Tris-Cl, pH 7.5). A. Cleaved peptides A04 (RGARARLG), N08 (RKRRAPLA), N12 (AKRAASQI), N17 (RKRRAVLT), B22 (APQRFGKK), B17 (RQRYGKRS), B23 (RVKRYRQS), H03 (RNKRAVQG). B. Cleaved peptides B20 (PQRFGRNT), C16 (RVLGLKAH), J21 (LRNRAQSG), H07 (RVKRQVRS), H11 (RRLLRAIP), N05 (RARRELAP), H01 (RKKRSTKK), O22 (GGVRGPRV), O16 (AATRRQAV), O23 (KNVAMQKG), G24 (RRKRSTRE), A09 (RMRRYADA), M02 (EKQRIIGG), H17 (NGLRADPM). C. Uncleaved peptides L16 (KTPRWGG), N10 (FYRADQPR), B11 (AAGVAPLS), J03 (PQLHSTGG), C01 (GPQGLLGA), K11 (VIPRSGGS), M07 (GLQRALEI), A01 (WQGGRRKF), H02 (RAKRSVHF), A10 (SAARPAPP), M16 (RSRREADL), A05 (SYRKVLGQ), M18 (VHRDMAAR), C23 (RDAQAHPG), M09 (GSQHIRAE), E19 (RLGREGVQ), D06 (ALAEAMKK).

FIG. 14 depicts a vector construction strategy for a conditional knockout of mCRISPC. Neo=neomycin; DTA=diphtheria toxin A; Ap=ApaI; As=AscI; Av=AvrII; B=BamHI; Bgl=Bgl I; Bs=BsiWI; E=EcoRI; H=HindIII; M=MluI; Nr=NruI; Pm=PmeI; RV=EcoRV; S=SacI; Sc=ScaI; SI=Sal I; Sm=SmaI; Sp=SpeI; Sph=SphI; X=XbaI; Xh=XhoI. The triangles indicated loxP sites. NotI can be used for linearization of the final target vector. The lighter shaded region between the first two loxP sites is the condition knockout region.

FIG. 15 shows restriction digests confirming subcloning of the 5′ arm/3loxPNwCD.

FIG. 16 shows restriction digests confirming subcloning of conditional knockout/3loxPNwCD.

FIG. 17 shows restrictions digests confirming subcloning of the 3′ arm/pCR2.1.

FIG. 18 shows a final vector restriction digestion of vector shown in FIG. 14.

FIG. 19 shows a screening strategy for mCRISPC conditional knockout. Neo=neomycin; DTA=diphtheria toxin A; Ap=ApaI; As=AscI; Av=AvrI; B=BamHI; Bgl=Bgl I; Bs=BsiWI; E=EcoRI; H=HindIII; M=MluI; Nr=NruI; Pm=PmeI; RV=EcoRV; S=SacI; Sc=ScaI; Sl=Sal I; Sm=SmaI; Sp=SpeI; Sph=SphI; X=XbaI; Xh=XhoI. The triangles indicated loxP sites. The lighter shaded region between the first two loxP sites is the condition knockout region. Screening strategy: 5′ probe: SphI digestion for genomic DNA; wildtype allele—24 kb; recombinant allele—11 kb. 3′ probe: SphI digestion for genomic DNA; wildtype allele—24 kb; recombinant allele—12 kb. Neo probe: SacI digestion for genomic DNA; recombinant allele—20.6 kb. Exon 1—mouse cDNA nucleotides 1-59; Exon 2—mouse cDNA nucleotides 60-179; Exon 3—mouse cDNA nucleotides 178-241; Exon 4—mouse cDNA nucleotides 242-376; Exon 5—mouse cDNA nucleotides 377467; Exon 6—mouse cDNA nucleotides 468-616; Exon 7—mouse cDNA nucleotides 617-715.

FIG. 20 is a 5′ probe test for 3loxP3NwCD. The 5′ probe is a 0.4 kb EcoRI fragment from 5′ probe/pCR2.1-TOPO. Blot was developed with overnight exposure at −80° C.

FIG. 21 is a 3′ probe test for 3loxP3NwCD. The 3′ probe is a 0.3 kb EcoRI fragment from 3′ probe/pCR2.1-TOPO. Blot was developed with overnight exposure at −80° C.

### BRIEF DESCRIPTION OF SEQUENCES

SEQ ID NO:1 is an cDNA sequence of the mouse homologue of hCRISP1.

SEQ ID NO:2 is an cDNA sequence of the rat homologue of hCRISP1.

SEQ ID NO:3 is a genomic sequence comprising the mouse homologue of hCRISP1.

SEQ ID NO:4 is a genomic sequence comprising the rat homologue of hCRISP1.

SEQ ID NO:5 is an amino acid sequence of the mouse homologue of hCRISP1.

SEQ ID NO:6 is an amino acid sequence of the rat homologue of hCRISP1.

SEQ ID NO:7 is the mouse homologue of hCRISP1 TaqMan® forward primer.

SEQ ID NO:8 is the mouse homologue of hCRISP1 TaqMan® reverse primer.

SEQ ID NO:9 is the mouse homologue of hCRISP1 TaqMan® probe.

SEQ ID NO:10 is the rat homologue of hCRISP1 TaqMan® forward primer.

SEQ ID NO:11 is the rat homologue of hCRISP1 TaqMan® reverse primer.

SEQ ID NO:12 is the rat homologue of hCRISP1 TaqMan® probe.

SEQ ID NO:13 is a mouse homologue of hCRISP1 peptide for polyclonal antibody production.

SEQ ID NO:14 is a mouse homologue of hCRISP1 peptide for polyclonal antibody production.

SEQ ID NO:15 is a rat homologue of hCRISP1 peptide for polyclonal antibody production.

SEQ ID NO:16 is a rat homologue of hCRISP1 peptide for polyclonal antibody production.

SEQ ID NO:17 is a mouse homologue of hCRISP1 mRNA (1 of 6 species identified).

SEQ ID NO:18 is a mouse homologue of hCRISP1 mRNA (2 of 6 species identified).

SEQ ID NO:19 is a mouse homologue of hCRISP1 mRNA (3 of 6 species identified).

SEQ ID NO:20 is a mouse homologue of hCRISP1 mRNA (4 of 6 species identified).

SEQ ID NO:21 is a mouse homologue of hCRISP1 mRNA (5 of 6 species identified).

SEQ ID NO:22 is a mouse homologue of hCRISP1 mRNA (6 of 6 species identified).

SEQ ID NO:23 is a rat homologue of hCRISP1 mRNA (1 of 4 species identified).

SEQ ID NO:24 is a rat homologue of hCRISP1 mRNA (2 of 4 species identified).

SEQ ID NO:25 is a rat homologue of hCRISP1 mRNA (3 of 4 species identified).

SEQ ID NO:26 is a rat homologue of hCRISP1 mRNA (4 of 4 species identified).

SEQ ID NO:27 is a mouse homologue of hCRISP1 deduced amino acid sequence.

SEQ ID NO:28 is a rat homologue of hCRISP1 deduced amino acid sequence.

SEQ ID NO:29 is the promoter region of the mouse homolog of hCRISP1 (first 2000 nt of SEQ ID NO:3).

SEQ ID NO:30 is promoter region of the rat homolog of hCRISP1 (first 2000 nt of SEQ ID NO:4).

SEQ ID NO:31 is an mCRISPC (mCRISP4) RT-PCR cloning forward primer.

SEQ ID NO:32 is an mCRISPC (mCRISP4) RT-PCR cloning full-length reverse primer.

SEQ ID NO:33 is an mCRISPC (mCRISP4) RT-PCR cloning PR-1 domain truncation reverse primer.

SEQ ID NO:34 is an rCRISPC (rCRISP4) RT-PCR cloning forward primer.

SEQ ID NO:35 is an rCRISPC (rCRISP4) RT-PCR cloning full-length reverse primer.

SEQ ID NO:36 is an rCRISPC (rCRISP4) RT-PCR cloning PR-1 domain-only truncation reverse primer.

SEQ ID NO:37 is an mCRISPC (mCRISP4) cDNA for recombinant expression of full-length recombinant protein.

SEQ ID NO:38 is an rCRISPC (rCRISP4) cDNA for recombinant expression of full-length recombinant protein.

SEQ ID NO:39 is an mCRISPC (mCRISP4) cDNA for expression of PR-1 domain-only truncated recombinant protein.

SEQ ID NO:40 is an rCRISPC (rCRISP4) cDNA for expression of PR-1 domain-only truncated recombinant protein.

SEQ ID NO:41 is an mCRISPC (mCRISP4) knock out construct sequence.

SEQ ID NO:42 is an end sequencing M13R 5′ end of 5′ for subclone 5′ arm/3loxP3NwCD confirmation.

SEQ ID NO:43 is an end sequencing NeoR 3′ end of 5′ for subclone 5′ arm/3loxP3NwCD confirmation.

SEQ ID NO:44 is an end sequencing M13R 5′ end of condition knockout arm for subclone conditional knockout/3loxP3NwCD confirmation.

SEQ ID NO:45 is an end sequencing NeoR 3′ end of condition knockout arm for subclone conditional knockout/3loxP3NwCD confirmation.

SEQ ID NO:46 is an end sequencing M13R 5′ end of 3′ arm for subclone 3′ arm/pCR.21 confirmation.

SEQ ID NO:47 is an end sequencing M13F 3′ end of 3′ arm for subclone 3′ arm/pCR.21 confirmation.

SEQ ID NO:48 is a 0.4 kb EcoRI fragment from 5′ probe/pCR2.1-TOPO.

SEQ ID NO:49 is a 0.3 kb EcoRI fragment from 3′ probe/pCR2.1-TOPO.

### DETAILED DESCRIPTION OF THE INVENTION

Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

The NCBI predicted gene 9230112K08Rik was identified as epididymis-specific (Affymetrix MOE430 qualifier 1431468_at) in a transcriptional profiling experiment of the mouse epididymis. For this gene, the invention includes the cDNA/RNA sequence (as supported by mRNAs and EST), the genomic sequence (including intron/exon structure), transcription factors as identified by searching the TRANSFAC® database, amino acid sequence of the protein encoded by the gene, and the tissue-dependent expression profile of 1431468_at (including the epididymal segment-dependent expression levels) that has been confirmed by qRT-PCR analysis.

Furthermore, a homology search identified the closest rat ortholog of 9230112K08Rik as ENSEMBL predicted gene #ENSRNOG00000013612 (78% similarity to 9230112K08Rik at the amino acid level). For this gene, the invention includes the cDNA/RNA sequence (as supported by 5′ and 3′ RACE products), the genomic sequence (including intron/exon structure), transcription factors as identified by searching the TRANSFAC® database (see Tables 3-5), and the amino acid sequence of the protein encoded by the gene (see also Nolan M. A. et al., Biol. Reprod., epublication ahead of print, 2006 doi: 10.1095/biolreprod. 105.048298).

Among human cysteine-rich secretory protein-1 (hCRISP1), 9230112K08Rik, and #ENSRNOG00000013612, there are 149 out of 254 identical amino acid residues by ClustalW alignment (58.66% identity). Between 9230112K08Rik and hCRISP1, there are 152 out of 250 identical amino acids residues (60.8% identity). Between #ENSRNOG00000013612 and hCRISP1, there are 152 out of 254 identical amino acid residues (59.8% identity). Between 9230112K08Rik and #ENSRNOG00000013612, there are 224 out of 254 identical amino acids (88.2% identity).

The genomic structure of the mouse homologue of hCRISP1 (based on NCBI mouse genomic draft; genomic locus 1A3, 18322918-18353664) is as follows:

TABLE 1

| Region in SEQ | Sequence | | Position in |
|---|---|---|---|
| ID NO: 3 | Attribute | Length (bp) | SEQ ID NO: 1 |
| 1-2000 | 5′-sequence | 2000 | — |
| 2001-2059 | Exon #1 | 59 | 1-59 |
| 2060-10850 | Intron #1 | 8791 | — |
| 10851-10915 | Exon #2 | 65 | 60-124 |
| 10916-13729 | Intron #2 | 2814 | — |
| 13730-13864 | Exon #3 | 135 | 125-259 |
| 13865-17654 | Intron #3 | 3790 | — |
| 17655-17745 | Exon #4 | 91 | 260-350 |
| 17746-19113 | Intron #4 | 1368 | — |
| 19114-19262 | Exon #5 | 149 | 351-499 |
| 19263-23576 | Intron #5 | 4314 | — |
| 23577-23674 | Exon #6 | 98 | 500-597 |
| 23675-25079 | Intron #6 | 1405 | — |
| 25080-25168 | Exon #7 | 89 | 598-686 |
| 25169-32187 | Intron #7 | 7019 | — |
| 32188-32315 | Exon #8 | 128 | 687-814 |
| 32316-33201 | 3′-sequence | 886* | — |

*Unable to retrieve 2000-nt 3′-sequence due to gap in available sequence (gap bases represented by “N”).

The genomic structure of the rat homologue of hCRISP1 (based on the NCBI rat genomic draft; genomic locus: 9q12, 16972923-16989513) is as follows:

TABLE 2

| Region in | Sequence | | Position in |
|---|---|---|---|
| SEQ ID NO: 4 | Attribute | Length (bp) | SEQ ID NO: 2 |
| 1-2000 | 5′-sequence | 2000 | — |
| 2001-2075 | Exon #1 | 75 | 1-75 |
| 2076-4333 | Intron #1 | 2258 | — |
| 4334-4547 | Exon #2 | 214 | 76-289 |
| 4548-8424 | Intron #2 | 3877 | — |
| 8425-8573 | Exon #3 | 149 | 290-438 |
| 8574-13503 | Intron #3 | 4930 | — |
| 13504-13601 | Exon #4 | 98 | 439-536 |
| 13602-15089 | Intron #4 | 1488 | — |
| 15090-15178 | Exon #5 | 89 | 537-625 |
| 15179-18463 | Intron #5 | 3285 | — |
| 18464-18591 | Exon #6 | 128 | 626-753 |
| 18592-20591 | 3′-sequence | 2000 | — |

The 1000 bp 5′ to transcriptional start site of hCRISP1, 9230112K08Rik, and #ENROG00000013612 was searched for transcription factors using the TRANSFAC® database (Version 8.3, available from Cognia Corp., New York, N.Y.). TRANSFAC® is a database on transcription factors, their genomic binding sites, and their binding properties.

TABLE 3
(Transcriptional factors at hCRISP1 5′ Upstream Region (1000 bp)

| | | | | Matrix Sequence | |
|---|---|---|---|---|---|
| Matrix | Position | Core | Matrix | (Always the | |
| Identifier | (Strand) | Match | Match | (+)- Strand is Shown) | Factor Name |
| V$GATA4_Q3 | 44 (+) | 1.000 | 0.939 | AGATAcaagaaa | GATA-4 |
| V$PIT1_Q6 | 108 (+) | 1.000 | 0.945 | aaTTCATaattttaaaaa | Pit-1 |
| V$CDX_Q5 | 281 (−) | 0.834 | 0.880 | GATATttctttctttgtt | CDX |
| V$EVI1_04 | 503 (−) | 1.000 | 0.864 | tATCTTttcattccc | Evi-1 |
| V$AFP1_Q6 | 574 (−) | 0.905 | 0.947 | gtgtagTTTAT | AFP1 |
| V$CDX_Q5 | 580 (−) | 1.000 | 0.932 | TTTATatgtttggtgatt | CDX |
| V$EFC_Q6 | 774 (+) | 0.820 | 0.845 | aGTTCCcagggtaa | RFX1 (EF-C) |
| V$RFX1_02 | 817 (−) | 1.000 | 0.977 | atGTTGCcttggttacaa | RFX1 |

Total Sequences Length = 1077
Total Number of Sites Found = 8
Frequency of Sites Per Nucleotide = 0.007428

TABLE 4
(Transcriptional factors at mouse homologue of hCRISP1 5′ Upstream
Region (1000 bp)

| | Position | Core | Matrix | Matrix Sequence (Always | |
|---|---|---|---|---|---|
| Matrix Identifier | (Strand) | Match | Match | the (+)- Strand is Shown) | Factor Name |
| V$CDX_Q5 | 7 (−) | 1.000 | 0.888 | TTTATctttttgtagata | CDX |
| V$HNF3ALPHA_Q6 | 23 (+) | 0.972 | 0.974 | TATTTgttttc | HNF-3alpha |
| V$GATA4_Q3 | 39 (−) | 0.907 | 0.921 | tcactgaGATCT | GATA-4 |
| V$OCT1_Q6 | 69 (−) | 1.000 | 0.918 | ttctTTTGCatcttt | Oct-1 |
| V$SRF_Q5_01 | 292 (−) | 0.990 | 0.904 | ccagaCCATAttagg | SRF |
| V$HNF_Q6_01 | 364 (−) | 1.000 | 0.854 | ctgttTTAATctttgctctgc | HNF-1 |
| V$HNF1_Q6 | 366 (+) | 1.000 | 0.851 | gttTTAATctttgctctg | HNF-1 |
| V$CEBPGAMMA_Q6 | 453 (+) | 0.845 | 0.925 | ctcATCTCaaaaa | C/EBPgamma |
| V$NKX25_02 | 489 (−) | 1.000 | 1.000 | cAATTAag | Nkx2-5 |
| V$DR4_Q2 | 502 (+) | 1.000 | 0.876 | taccctacatTGACCct | LXR, PXR, |
| | | | | | CAR, COUP, |
| | | | | | RAR |
| V$MTATA_B | 538 (+) | 1.000 | 0.942 | acatATAAAcctgagtg | Muscle TATA |
| | | | | | box |
| V$DEC_Q1 | 558 (+) | 0.987 | 0.951 | acaCATGTgaaga | DEC |
| V$FOXJ2_02 | 589 (+) | 1.000 | 0.978 | aacATAATatttgt | FOXJ2 |
| V$FOXJ2_01 | 648 (+) | 1.000 | 0.994 | acttaaatAAACAttgat | FOXJ2 |
| V$HNF3B_01 | 651 (−) | 1.000 | 0.970 | taaatAAACAttgat | HNF-3beta |
| V$FOX_Q2 | 651 (−) | 1.000 | 0.981 | taaatAAACAttg | FOX |
| V$FOXD3_01 | 652 (−) | 0.996 | 0.985 | aaataAACATtg | FOXD3 |
| V$HNF1_Q6_01 | 674 (+) | 0.933 | 0.871 | tagagaaaaaaATAAAactga | HNF1 |
| V$CDX_Q5 | 681 (+) | 1.000 | 0.915 | aaaaataaaactgATAAA | CDX |
| V$PAX3_B | 848 (−) | 1.000 | 0.921 | ctcatgatCGTGActtgaaat | Pax-3 |
| V$CEBPDELTA_Q6 | 945 (+) | 1.000 | 0.990 | aATTGCctcatt | C/EBPdelta |
| V$HNF4_DR1_Q3 | 1005 (−) | 1.000 | 0.882 | cagaCAAAGacca | HNF-4 direct |
| | | | | | repeat 1 |

Total Sequences Length = 1059
Total Number of Sites Found = 22
Frequency of Sites Per Nucleotide = 0.020774

TABLE 5
(Transcriptional factors at rat homologue of hCRISP1 5′ Upstream
Region (1000 bp)

| | | | | Matrix Sequence | |
|---|---|---|---|---|---|
| | Position | Core | Matrix | (Always the (+)- | |
| Matrix Identifier | (Strand) | Match | Match | Strand is Shown) | Factor Name |
| V$OCT1_Q6 | 168 (+) | 1.000 | 0.912 | CagtatGCAAAgtct | Oct-1 |
| V$CEBPGAMMA_Q6 | 347 (+) | 0.845 | 0.925 | ctcATCTCaaaaa | C/EBPgamma |
| V$NKX25_02 | 373 (−) | 1.000 | 1.000 | cAATTAag | Nkx2-5 |
| V$PPAR_DR1_Q2 | 402 (+) | 0.811 | 0.872 | tGACCGctggcct | PPAR direct |
| | | | | | repeat 1 |
| V$HNF3B_01 | 554 (+) | 1.000 | 0.973 | ttaaaTATTTatttt | HNF-3beta |
| V$FOXD3_01 | 556 (+) | 0.944 | 0.954 | aaATATTtattt | FOXD3 |
| V$FOXJ2_02 | 594 (−) | 0.948 | 0.924 | caaaatATTGTcat | FOXJ2 |
| V$CDX2_Q5 | 784 (+) | 1.000 | 0.889 | AaaaaTTTATcact | Cdx-2 |
| V$MRF2_01 | 999 (+) | 1.000 | 0.967 | gtacacAATACaaa | MRF-2 |

Total Sequences Length = 1059
Total Number of Sites Found = 9
Frequency of Sites Per Nucleotide = 0.008499

Each gene is more similar to this human gene than the currently named rat CRISP1 and mouse CRISP1. 9230112K08Rik has a 69% similarity to hCRISP1 at the amino acid level (71% similarity at cDNA level), while #ENSROG00000013612 has a 69% similarity to hCRISP1 at the amino acid level (73% similarity at cDNA level).

The numerical designation following the abbreviation “CRISP” no longer represents the degree of similarity of the proteins. This is demonstrated in Tables 6 and 7.

TABLE 6
(CRISP family similarity - amino acid level)

| % | | | | | | | | | | novel |
|---|---|---|---|---|---|---|---|---|---|---|
| Similarity | hCRISP1 | hCRISP2 | hCRISP3 | mCRISP1 | mCRISP2 | mCRISP3 | novel mCRISP | rCRISP1 | rCRISP2 | rCRISP |
| hCRISP1 | 100 | | | | | | | | | |
| hCRISP2 | 51 | 100 | | | | | | | | |
| hCRISP3 | 50 | 77 | 100 | | | | | | | |
| mCRISP1 | 47 | 65 | 61 | 100 | | | | | | |
| mCRISP2 | 48 | 76 | 66 | 60 | 100 | | | | | |
| mCRISP3 | 46 | 59 | 57 | 80 | 58 | 100 | | | | |
| novel | 69 | 49 | 51 | 49 | 49 | 47 | 100 | | | |
| mCRISP | | | | | | | | | | |
| rCRISP1 | 47 | 64 | 63 | 76 | 63 | 68 | 52 | 100 | | |
| rCRISP2 | 48 | 75 | 67 | 58 | 89 | 57 | 47 | 62 | 100 | |
| novel | 69 | 51 | 55 | 51 | 48 | 48 | 91 | 51 | 48 | 100 |
| rCRISP | | | | | | | | | | |

TABLE 7
(CRISP family similarity - cDNA)

| | | | | | | | novel | | | novel |
|---|---|---|---|---|---|---|---|---|---|---|
| % Similarity | hCRISP1 | hCRISP2 | hCRISP3 | mCRISP1 | mCRISP2 | mCRISP3 | mCRISP | rCRISP1 | rCRISP2 | rCRISP |
| hCRISP1 | 100 | | | | | | | | | |
| hCRISP2 | 55 | 100 | | | | | | | | |
| hCRISP3 | 55 | 79 | 100 | | | | | | | |
| mCRISP1 | 53 | 66 | 66 | 100 | | | | | | |
| mCRISP2 | 54 | 78 | 70 | 64 | 100 | | | | | |
| mCRISP3 | 53 | 63 | 65 | 88 | 63 | 100 | | | | |
| novel mCRISP | 71 | 55 | 57 | 55 | 54 | 55 | 100 | | | |
| rCRISP1 | 52 | 66 | 66 | 82 | 65 | 79 | 57 | 100 | | |
| rCRISP2 | 53 | 76 | 70 | 64 | 90 | 61 | 54 | 64 | 100 | |
| novel rCRISP | 73 | 55 | 55 | 54 | 55 | 52 | 90 | 54 | 55 | 100 |

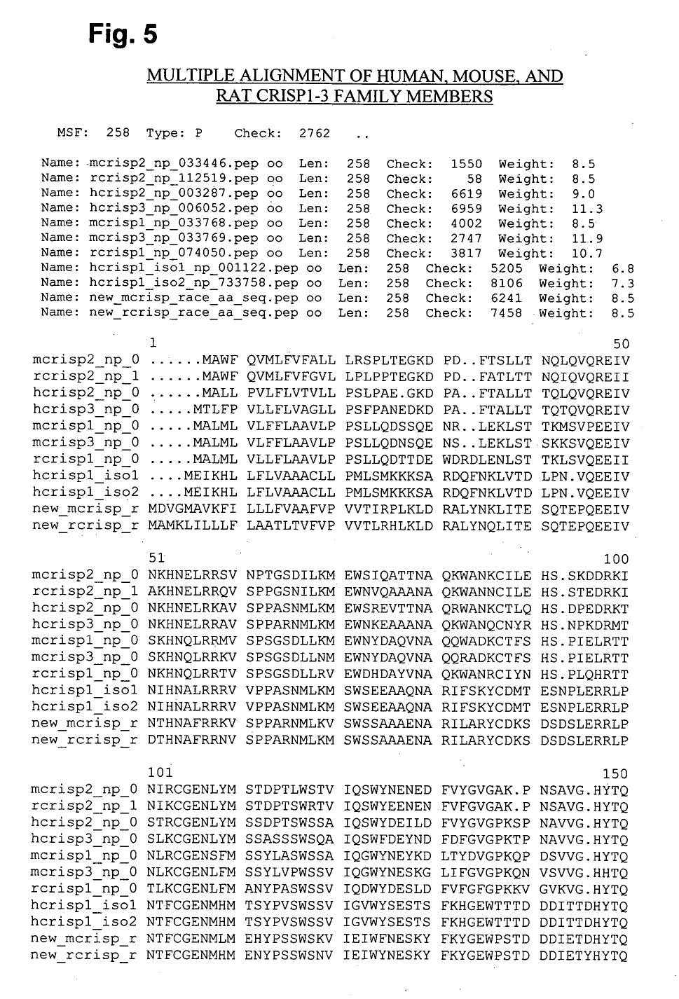

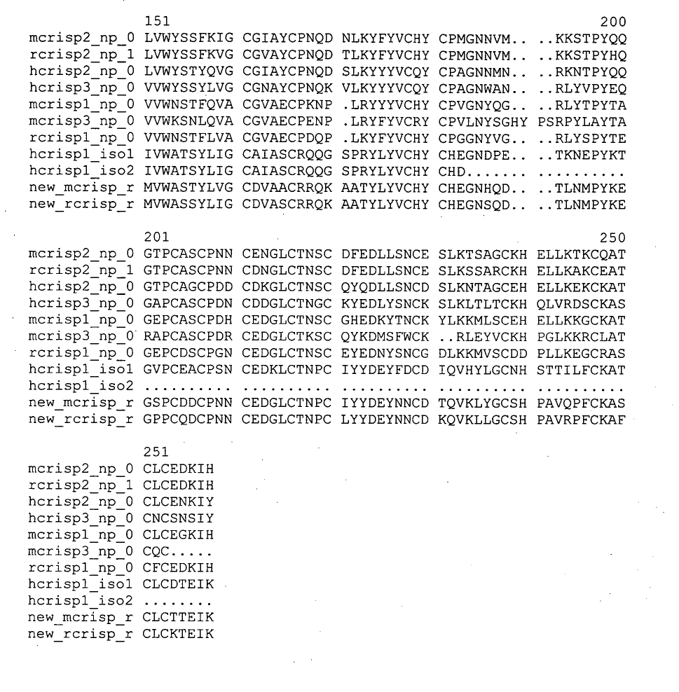

Multiple alignment of the mouse, rat, and human CRISP family of proteins further demonstrates that the mouse and rat homologues disclosed herein have a higher degree of similarity to hCRISP1 than the currently named mCRISP1 and rCRISP1 (see FIG. 5). The human CRISP1 gene encodes for two proteins, termed hCRISP1 isoform 1 and hCRISP1 isoform 2. This is the result of an alternate splicing event that truncates the protein at amino acid 180. The final amino acid in hCRISP1 isoform 2 is also changed from a glutamic acid (E) to an aspartic acid (D).

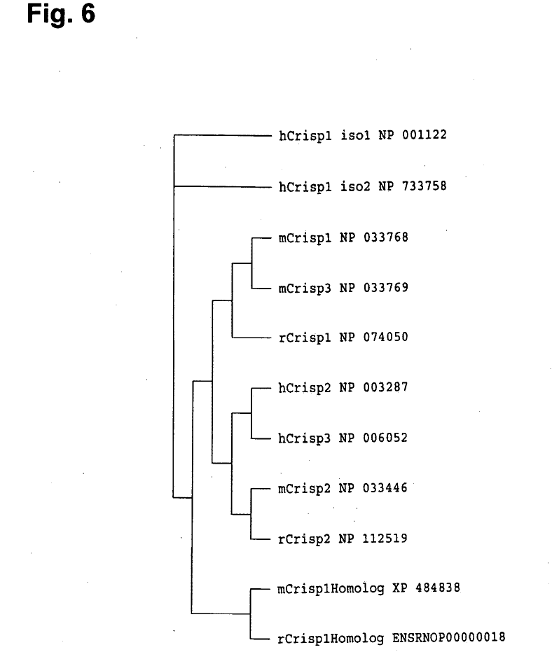

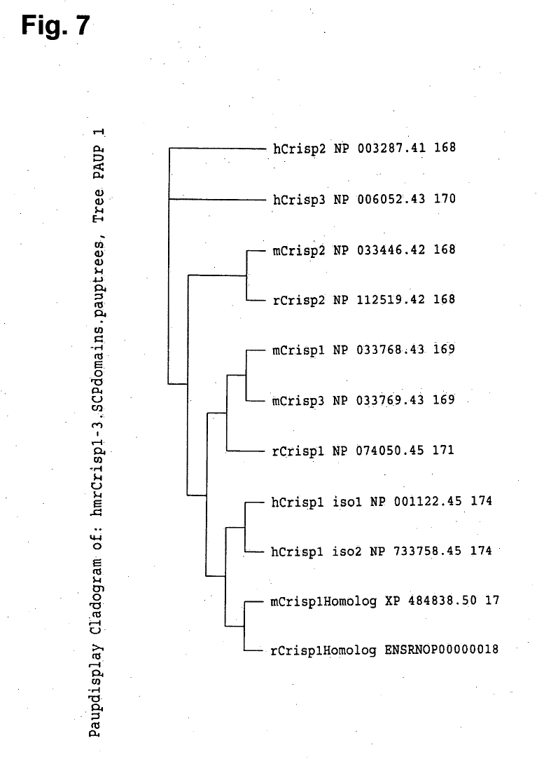

Phylogenetic analysis (using either full-length protein sequences or the conserved SCP domains) generated by paupsearch with bootstrap analysis places these two predicted genes as close homologues of hCRISP1 (see FIG. 6 and FIG. 7).

As a result, Applicants propose the following new nomenclature for the mouse, rat, and human genes and proteins:

TABLE 8

| Prior Name | New Name |
|---|---|
| mCRISP1 | mCRISPA1 |
| mCRISP3 | mCRISPA2 |
| mCRISP2 | mCRISPB |
| Mouse Homologue of | mCRISPC |
| hCRISP1 (mCRISP4) | |
| rCRISP1 | rCRISPA |
| rCRISP2 | rCRISPB |
| Rat Homologue of | rCRISPC |
| hCRISP1 (rCRISP4) | |
| hCRISP3 | hCRISPA |
| hCRISP2 | hCRISPB |
| hCRISP1 | hCRISPC |

I. Definitions

In the context of this disclosure, a number of terms shall be utilized.

As used herein, the term “about” or “approximately” means within 20%, preferably within 10%, and more preferably within 5% of a given value or range.

An “antibody” includes an immunoglobulin molecule capable of binding an epitope present on an antigen. As used herein, the term encompasses not only intact immunoglobulin molecules such as monoclonal and polyclonal antibodies, but also anti-idotypic antibodies, mutants, fragments, fusion proteins, bi-specific antibodies, humanized proteins, and modifications of the immunoglobulin molecule that comprises an antigen recognition site of the required specificity.

The term “cDNAs” includes complementary DNA, that is mRNA molecules present in a cell or organism made into cDNA with an enzyme such as reverse transcriptase. A “cDNA library” includes a collection of mRNA molecules present in a cell or organism, converted into cDNA molecules with the enzyme reverse transcriptase, then inserted into vectors. The library can then be probed for the specific cDNA (and thus mRNA) of interest.

As used herein, an mCRISPC or rCRISPC “chimeric protein” or “fusion protein” comprises an mCRISPC or rCRISPC polypeptide operably linked to a non-mCRISPC or non-rCRISPC polypeptide. An “mCRISPC polypeptide” refers to a polypeptide having an amino acid sequence corresponding to mCRISPC polypeptide, whereas a “non-mCRISPC polypeptide” refers to a polypeptide having an amino acid sequence corresponding to a protein which is not substantially homologous to the mCRISPC protein, for example, a protein which is different from the mCRISPC protein and which is derived from the same or a different organism. An “rCRISPC polypeptide” refers to a polypeptide having an amino acid sequence corresponding to rCRISPC polypeptide, whereas a “non-rCRISPC polypeptide” refers to a polypeptide having an amino acid sequence corresponding to a protein which is not substantially homologous to the rCRISPC protein, e.g., a protein which is different from the rCRISPC protein and which is derived from the same or a different organism. Within an mCRISPC or rCRISPC fusion protein, the mCRISPC or rCRISPC polypeptide can correspond to all or a portion of an mCRISPC or rCRISPC protein. In a preferred embodiment, an mCRISPC or rCRISPC fusion protein comprises at least one biologically active portion of an mCRISPC or rCRISPC protein. Within the fusion protein, the term “operably linked” is intended to indicate that the mCRISPC or rCRISPC polypeptide and the non-mCRISPC or non-rCRISPC polypeptide are fused in-frame to each other. The non-mCRISPC or non-rCRISPC polypeptide can be fused to the N-terminus or C-terminus of the mCRISPC or rCRISPC polypeptide.

A DNA “coding sequence” is a double-stranded DNA sequence which is transcribed and translated into a polypeptide in a cell in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. If the coding sequence is intended for expression in a eukaryotic cell, a polyadenylation signal and transcription termination sequence will usually be located 3′ to the coding sequence.

The terms “cysteine-rich secretory protein” and “CRISP” refer to a family of proteins characterized by sixteen conserved cysteine residues that are found in such diverse systems as plant defense proteins, snake and wasp venom proteins, and lizard toxin proteins (Yamazaki Y et al., Arch. Biochem. Biophys. 412:133-41 (2003)). Non-mammalian CRISP family members have been shown to act on a variety of ion channels: cyclic-nucleotide gated channels in the retinal and olfactory neurons (PsTx and pseudecin) (Brown R L et al., Proc. Natl. Acad. Sci. USA 96:754-59 (1999)) and voltage-gated calcium channels and ryanodine receptors (helothermine) (Morrissette J et al., Biophys. J. 68:2280-88 (1995); Nobile M et al., Exp. Brain Res. 110:15-20 (1996)). Mammalian CRISPs are categorized into three subtypes that have been shown to exhibit differences in tissue-dependent expression and proposed functionality (Kratzschmar J. et al., Eur. J. Biochem. 263:827-36 (1996)). Expression of the CRISP1 (termed “CRISPC” herein) family of proteins is restricted to the epididymis, and these proteins have been implicated in both the inhibition of capacitation (Roberts K P et al., Biol. Reprod. 69:572-81 (2003)) and sperm-egg fusion (Hall J C et al., Prep. Biochem. Biotechnol. 27:239-51 (1997); Cohen D J et al., Mol. Reprod. Dev. 56:180-88 (2000); Evans J P, Hum. Reprod. Update 8:297-311 (2002)). CRISP2 (TPX-1; termed “CRISPB” herein) expression is highest in pachytene spermatocytes and round spermatids in the testis, and may be involved in their interaction with sertoli cells (Maeda T et al., Dev. Growth Differ. 41:715-22 (1999); O'Bryan M K et al., Mol. Reprod. Dev. 58:116-25 (2001); Giese A et al., Gene 299:101-09 (2002)). In mice, CRISP3 (termed “CRISPA” herein) is expressed primarily in the salivary glands, but it is widely expressed in humans (salivary glands, fallopian tubes, seminal vesicles, bone, immune cells, and others). The function of CRISP3 protein is unknown, although it has been shown to bind to the alpha₁B-glycoprotein in human plasma (Schwidetky U et al., Biochem. J. 309:831-36 (1995); Pfisterer P et al., Mol. Cell. Biol. 16:6160-68 (1996); SchambonyA et al., Biochim. Biophys. Acta 1387:206-16 (1998); Udby L et al., J. Leukoc. Biol. 72:462-69 (2002); Udby L et al., Biochemistry 43:12877-86 (2004)).

The terms “effective amount”, “therapeutically effective amount”, and “effective dosage” as used herein, refer to the amount of an effector molecule that, when administered to a mammal in need, is effective to at least partially ameliorate conditions related to, for example, infertility, or is effective to at least partially enhance, for example, contraception.

As used herein, the term “expression” includes the process by which polynucleotides are transcribed into mRNA and translated into peptides, polypeptides, or proteins. If the polynucleotide is derived from genomic DNA, expression may include splicing of the mRNA, if an appropriate eukaryotic host is selected. Regulatory elements required for expression include promoter sequences to bind RNA polymerase and transcription initiation sequences for ribosome binding. For example, a bacterial expression vector includes a promoter such as the lac promoter and for transcription initiation the Shine-Dalgarno sequence and the start codon AUG (Sambrook, J., Fritsh, E. F., and Maniatis, T., Molecular Cloning: A Laboratory Manual, 2nd ed., Cold Spring Harbor Laboratory, Cold Spring Harbor Laboratory Press, Cold Spring Harbor, N.Y., 1989). Similarly, a eukaryotic expression vector includes a heterologous or homologous promoter for RNA polymerase II, a downstream polyadenylation signal, the start codon AUG, and a termination codon for detachment of the ribosome. Such vectors can be obtained commercially or assembled by the sequences described in methods well known in the art, for example, the methods described below for constructing vectors in general.

The term “expression construct” means any double-stranded DNA or double-stranded RNA designed to transcribe an RNA, e.g., a construct that contains at lease one promoter operably linked to a downstream gene or coding region of interest (e.g., a cDNA or genomic DNA fragment that encodes a protein, or any RNA of interest). Transfection or transformation of the expression construct into a recipient cell allows the cell to express RNA or protein encoded by the expression construct. An expression construct may be a genetically engineered plasmid, virus, or an artificial chromosome derived from, for example, a bacteriophage, adenovirus, retrovirus, poxvirus, or herpesvirus, or further embodiments described under “expression vector” below. An expression construct can be replicated in a living cell, or it can be made synthetically. For purposes of this application, the terms “expression construct”, “expression vector”, “vector”, and “plasmid” are used interchangeably to demonstrate the application of the invention in a general, illustrative sense, and are not intended to limit the invention to a particular type of expression construct. Further, the term expression construct or vector is intended to also include instances wherein the cell utilized for the assay already endogenously comprises such DNA sequence.

A “gene” includes a polynucleotide containing at least one open reading frame that is capable of encoding a particular polypeptide or protein after being transcribed and translated. Any of the polynucleotide sequences described herein may be used to identify larger fragments or full-length coding sequences of the gene with which they are associated. Methods of isolating larger fragment sequences are known to those of skill in the art, some of which are described herein.

The term “genetically modified” includes a cell containing and/or expressing a foreign gene or nucleic acid sequence which in turn modifies the genotype or phenotype of the cell or its progeny. This term includes any addition, deletion, or disruption to a cell's endogenous nucleotides.

A “gene product” includes an amino acid (e.g., peptide or polypeptide) generated when a gene is transcribed and translated.

The term “heterologous” refers to a combination of elements not naturally occurring. For example, heterologous DNA refers to DNA not naturally located in the cell, or in a chromosomal site of the cell. Preferably, the heterologous DNA includes a gene foreign to the cell. A heterologous expression regulatory element is such an element operably associated with a different gene than the one it is operably associated with in nature.

The term “homologous” as used herein refers to the sequence similarity between two polymeric molecules, e.g., between two nucleic acid molecules, e.g., two DNA molecules or two RNA molecules, or between two polypeptide molecules. When a nucleotide or amino acid position in both of the two molecules is occupied by the same monomeric nucleotide or amino acid, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGCG5′ share 50% homology. By the term “substantially homologous” as used herein, is meant DNA or RNA which is about 50% homologous, more preferably about 70% homologous, even more preferably about 80% homologous, and most preferably about 90% homologous to the desired nucleic acid.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence. The residues at corresponding positions are then compared and when a position in one sequence is occupied by the same residue as the corresponding position in the other sequence, then the molecules are identical at that position. The percent identity between two sequences, therefore, is a function of the number of identical positions shared by two sequences (i.e., % identity=# of identical positions/total # of positions×100). The percent identity between the two sequences is a function of the number of identical positions shared by the sequences, taking into account the number of gaps, and the length of each gap, which are introduced for optimal alignment of the two sequences.

The comparison of sequences and determination of percent identity between two sequences can be accomplished using a mathematical algorithm. A non-limiting example of a mathematical algorithm utilized for comparison of sequences is the algorithm of Karlin S and Altschul S F, Proc. Natl. Acad. Sci. USA 87:2264-68 (1990), modified as in Karlin S and Altschul S F, Proc. Natl. Acad. Sci. USA 90:5873-77 (1993). Such an algorithm is incorporated into the NBLAST and XBLAST programs (version 2.0) of Altschul S F et al., J. Mol. Biol. 215:403-10 (1990). BLAST nucleotide searches can be performed with the NBLAST program score=100, wordlength=12 to obtain nucleotide sequences homologous to the nucleic acid molecules of the invention. BLAST protein searches can be performed with the XBLAST program, score=50, wordlength=3 to obtain amino acid sequences homologous to the protein molecules of the invention. To obtain gapped alignments for comparison purposes, Gapped BLAST can be utilized as described in Altschul S F et al., Nucleic Acids Res. 25:3389-3402 (1997). When utilizing BLAST and Gapped BLAST programs, the default parameters of the respective programs (e.g., XBLAST and NBLAST) can be used. Another preferred, non-limiting algorithm utilized for the comparison of sequences is the algorithm of Myers E W and Miller W, Comput. Appl. Biosci. 4:11-17 (1988). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

Another non-limiting example of a mathematical algorithm utilized for the alignment of protein sequences is the Lipman-Pearson algorithm (Lipman D J and Pearson W R, Science 227:1435-41 (1985)). When using the Lipman-Pearson algorithm, a PAM250 weight residue table, a gap length penalty of 12, a gap penalty of 4, and a Kutple of 2 can be used. A preferred, non-limiting example of a mathematical algorithm utilized for the alignment of nucleic acid sequences is the Wilbur-Lipman algorithm (Wilbur W J and Lipman D J, Proc. Natl. Acad. Sci. USA 80:726-30 (1983)). When using the Wilbur-Lipman algorithm, a window of 20, gap penalty of 3, Ktuple of 3 can be used. Both the Lipman-Pearson algorithm and the Wilbur-Lipman algorithm are incorporated, for example, into the MEGALIGN program (e.g., version 3.1.7) which is part of the DNASTAR sequence analysis software package.

Additional algorithms for sequence analysis are known in the art, and include ADVANCE and ADAM, described in Torelli A and Robotti C A, Comput. Appl. Biosci. 10:3-5 (1994); and FASTA, described in Pearson W R and Lipman D J, Proc. Natl. Acad. Sci. USA 85:2444-48 (1988).

In a preferred embodiment, the percent identity between two amino acid sequences is determined using the GAP program in the GCG software package, using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using a NWSgapdna. CMP matrix and a gap weight of 40, 50, 60, 70, or 80 and a length weight of 1, 2, 3, 4, 5, or 6.

Protein alignments can also be made using the Geneworks global protein alignment program (e.g., version 2.5.1) with the cost to open gap set at 5, the cost to lengthen gap set at 5, the minimum diagonal length set at 4, the maximum diagonal offset set at 130, the consensus cutoff set at 50% and utilizing the Pam 250 matrix.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul S F et al., J. Mol. Biol. 215:403-10 (1990). BLAST nucleotide searches can be performed with the NBLAST program, score=100, wordlength=12 to obtain nucleotide sequences homologous to mCRISPC or rCRISPC nucleic acid molecules of the invention. BLAST protein searches can be performed with the XBLAST program, score=50, wordlength=3 to obtain amino acid sequences homologous to mCRISPC or rCRISPC protein molecules of the invention. To obtain gapped alignments for comparison purposes, Gapped BLAST can be utilized as described in Altschul S F et al., Nucleic Acids Res. 25:3389-3402 (1997). When utilizing BLAST and Gapped BLAST programs, the default parameters of the respective programs (e.g., XBLAST and NBLAST) can be used. For example, the nucleotide sequences of the invention can be analyzed using the default Blastn matrix 1-3 with gap penalties set at: existence 11 and extension 1. The amino acid sequences of the invention can be analyzed using the default settings: the Blosum62 matrix with gap penalties set at existence 11 and extension 1.

A “host cell” is intended to include any individual cell or cell culture which can be or has been a recipient for vectors or for the incorporation of exogenous nucleic acid molecules, polynucleotides, and/or proteins. It also is intended to include progeny of a single cell. The progeny may not necessarily be completely identical (in morphology or in genomic or total DNA complement) to the original parent cell due to natural, accidental, or deliberate mutation. The cells may be prokaryotic or eukaryotic, and include but are not limited to bacterial cells, yeast cells, insect cells, animal cells, and mammalian cells, e.g., murine, rat, simian, or human cells.

“Hybridization” includes a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under conditions of different “stringency”. The stringency of a hybridization reaction includes the difficulty with which any two nucleic acid molecules will hybridize to one another. Under stringent conditions, nucleic acid molecules at least 65%, 70%, 75% or more identical to each other remain hybridized to each other, whereas molecules with low percent identity cannot remain hybridized. A preferred, non-limiting example of highly stringent hybridization conditions are hybridization in 6× sodium chloride/sodium citrate (SSC) at about 45° C., followed by one or more washes in 0.2×SSC, 0.1% SDS at 50. C, preferably at 55° C., more preferably at 60° C., and even more preferably at 65° C.

When hybridization occurs in an antiparallel configuration between two single-stranded polynucleotides, the reaction is called “annealing” and those polynucleotides are described as “complementary”. A double-stranded polynucleotide can be “complementary” or “homologous” to another polynucleotide if hybridization can occur between one of the strands of the first polynucleotide and the second. “Complementarity” or homology is quantifiable in terms of the proportion of bases in opposing strands that are expected to hydrogen bond with each other, according to generally accepted base-pairing rules.

As used herein, the term “isolated” means that the referenced material is removed from the environment in which it is normally found. Thus, an isolated biological material can be free of cellular components, i.e., components of the cells in which the material is found or produced. In the case of nucleic acid molecules, an isolated nucleic acid includes, for example, a PCR product, an isolated mRNA, a cDNA, or a restriction fragment. In another embodiment, an isolated nucleic acid is preferably excised from the chromosome in which it may be found, and more preferably is no longer joined to non-regulatory, non-coding regions, or to other genes, located upstream or downstream of the gene contained by the isolated nucleic acid molecule when found in the chromosome. In yet another embodiment, the isolated nucleic acid lacks one or more introns. Isolated nucleic acid molecules include sequences inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The term “mammal” refers to a human, a non-human primate, canine, feline, bovine, ovine, porcine, murine, or other veterinary or laboratory mammal. Those skilled in the art recognize that a therapy which reduces the severity of a pathology in one species of mammal is predictive of the effect of the therapy on another species of mammal.

The term “modulate” encompasses either a decrease or an increase in activity depending on the target molecule. For example, an effector molecule is considered to modulate the activity of mCRISPC or rCRISPC if the presence of such effector molecule results in an increase or decrease in mCRISPC or rCRISPC activity.

As used herein, a “naturally-occurring” nucleic acid molecule refers to an RNA or DNA molecule having a nucleotide sequence that occurs in nature (e.g., encodes a natural protein).

The term “operably linked” means that a nucleic acid molecule, i.e., DNA, and one or more regulatory sequences (e.g., a promoter or portion thereof) are connected in such a way as to permit transcription of mRNA from the nucleic acid molecule or permit expression of the product (i.e., a polypeptide) of the nucleic acid molecule when the appropriate molecules are bound to the regulatory sequences. Within a fusion construct, the term “operably linked” is intended to indicate that the mCRISPC or rCRISPC polynucleotide and a non-mCRISPC or non-rCRISPC polynucleotide are fused in-frame to each other. The non-mCRISPC or non-rCRISPC polynucleotide can be fused 3′ or 5′ to the mCRISPC or rCRISPC polynucleotide.

As used herein, the terms “polynucleotide” and “oligonucleotides” are used interchangeably, and include polymeric forms of nucleotides of any length, either deoxyribonucleotides or ribonucleotides, or analogs thereof. Polynucleotides may have any three-dimensional structure, and may perform any function, known or unknown. The following are non-limiting examples of polynucleotides: a gene or gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A), cytosine (C), guanine (G), thymine (T), and uracil (U) for guanine when the polynucleotide is RNA. Thus, the term “polynucleotide sequence” is the alphabetical representation of a polynucleotide molecule.

It is contemplated that where the nucleic acid molecule is RNA, the T (thymine) in non-RNA sequences provided herein is substituted with U (uracil). For example, SEQ ID NO:1 and SEQ ID NO:2 are disclosed herein as cDNA sequences. Thus, It would be obvious to one of ordinary skill in the art that an RNA molecule comprising sequences from these sequences, for example, would have T substituted with U.

The term “polypeptide” includes a compound of two or more subunit amino acids, amino acid analogs, or peptidomimetics. The subunits may be linked by peptide bonds. In another embodiment, the subunit may be linked by other bonds, e.g., ester, ether, etc. As used herein the term “amino acid” includes either natural and/or unnatural or synthetic amino acids, including glycine and both the D or L optical isomers, and amino acid analogs and peptidomimetics. A peptide of three or more amino acids is commonly referred to as an oligopeptide. Peptide chains of greater than three or more amino acids are referred to as a polypeptide or a protein.

A “primer” includes a short polynucleotide, generally with a free 3′-OH group that binds to a target or “template” present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or “set of primers” consisting of an “upstream” and a “downstream” primer, and a catalyst of polymerization, such as a DNA polymerase, and typically a thermally-stable polymerase enzyme. Methods for PCR are well known in the art, and are taught, for example, in MacPherson et al., IRL Press at Oxford University Press (1991). All processes of producing replicate copies of a polynucleotide, such as PCR or gene cloning, are collectively referred to herein as “replication”. A primer can also be used as a probe in hybridization reactions, such as Southern or Northern blot analyses (see, e.g., Sambrook, J., Fritsh, E. F., and Maniatis, T. Molecular Cloning: A Laboratory Manual, 2nd ed., Cold Spring Harbor Laboratory, Cold Spring Harbor Laboratory Press, Cold Spring Harbor, N.Y., 1989).

A “probe” when used in the context of polynucleotide manipulation includes an oligonucleotide that is provided as a reagent to detect a target present in a sample of interest by hybridizing with the target. Usually, a probe will comprise a label or a means by which a label can be attached, either before or subsequent to the hybridization reaction. Suitable labels include, but are not limited to, radioisotopes, fluorochromes, chemiluminescent compounds, dyes, and proteins, including enzymes.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined, for example, by mapping with nuclease S1), as well as protein binding domains (consensus sequences) responsible for the binding of RNA polymerase.

The term “purified” as used herein refers to material that has been isolated under conditions that reduce or eliminate the presence of unrelated materials, i.e., contaminants, including native materials from which the material is obtained. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. As used herein, the term “substantially free” is used operationally, in the context of analytical testing of the material. Preferably, purified material substantially free of contaminants is at least 50% pure; more preferably, at least 90% pure; and more preferably still at least 99% pure. Purity can be evaluated by chromatography, gel electrophoresis, immunoassay, composition analysis, biological assay, and other methods known in the art.

Methods for purification are well-known in the art. For example, nucleic acids can be purified by precipitation, chromatography (including preparative solid phase chromatography, oligonucleotide hybridization, and triple helix chromatography), ultracentrifugation, and other means. Polypeptides and proteins can be purified by various methods including, without limitation, preparative disc-gel electrophoresis, isoelectric focusing, HPLC, reversed-phase HPLC, gel filtration, ion exchange and partition chromatography, precipitation and salting-out chromatography, extraction, and countercurrent distribution. For some purposes, it is preferable to produce the polypeptide in a recombinant system in which the protein contains an additional sequence tag that facilitates purification, such as, but not limited to, a polyhistidine sequence, or a sequence that specifically binds to an antibody, such as FLAG and GST. The polypeptide can then be purified from a crude lysate of the host cell by chromatography on an appropriate solid-phase matrix. Alternatively, antibodies produced against the protein or against peptides derived therefrom can be used as purification reagents. Cells can be purified by various techniques, including, for example, centrifugation, matrix separation (e.g., nylon wool separation), panning and other immunoselection techniques, depletion (e.g., complement depletion of contaminating cells), and cell sorting (e.g., fluorescence activated cell sorting (FACS)). Other purification methods are possible. A purified material may contain less than about 50%, preferably less than about 75%, and most preferably less than about 90%, of the cellular components with which it was originally associated. The “substantially pure” indicates the highest degree of purity which can be achieved using conventional purification techniques known in the art.

The term “test compound” includes compounds with known chemical structure but not necessarily with a known function or biological activity. Test compounds could also have unidentified structures or be mixtures of unknown compounds, for example from crude biological samples such as plant extracts. Large numbers of compounds could be randomly screened from “chemical libraries” which refers to collections of purified chemical compounds or collections of crude extracts from various sources. The chemical libraries may contain compounds that were chemically synthesized or purified from natural products. The compounds may comprise inorganic or organic small molecules or larger organic compounds such as, for example, proteins, peptides, glycoproteins, steroids, lipids, phospholipids, nucleic acids, and lipoproteins. The amount of compound tested can very depending on the chemical library, but, for purified (homogeneous) compound libraries, 10 μM is typically the highest initial dose tested. Methods of introducing test compounds to cells are well known in the art.

II. Isolated Polynucleotides Encoding mCRISPC or rCRISPC or Portions Thereof

In practicing the methods of the invention, various agents can be used to modulate the activity and/or expression of mCRISPC or rCRISPC in a cell. In one embodiment, an agent is a nucleic acid molecule encoding a mCRISPC or rCRISPC polypeptide or a portion thereof. Such nucleic acid molecules are described in more detail below.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid molecule and the amino acid sequence encoded by that nucleic acid molecule, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, |
| | CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, |
| | TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, |
| | TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent because they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA molecule coding for a mCRISPC or rCRISPC polypeptide of the invention (or a portion thereof) can be used to derive the mCRISPC or rCRISPC amino acid sequence, using the genetic code to translate the DNA or RNA molecule into an amino acid sequence. Likewise, for any mCRISPC or rCRISPC amino acid sequence, corresponding polynucleotide sequences that can encode mCRISPC or rCRISPC protein can be deduced from the genetic code (which, because of its redundancy, will produce multiple polynucleotide sequences for any given amino acid sequence). Thus, description and/or disclosure herein of a mCRISPC or rCRISPC polynucleotide sequence should be considered to also include description and/or disclosure of the amino acid sequence encoded by the polynucleotide sequence. Similarly, description and/or disclosure of a mCRISPC or rCRISPC amino acid sequence herein should be considered to also include description and/or disclosure of all possible polynucleotide sequences that can encode the amino acid sequence. One aspect of the invention pertains to isolated nucleic acid molecules that encode mCRISPC or rCRISPC proteins or biologically active portions thereof, as well as nucleic acid fragments sufficient for use as hybridization probes to identify mCRISPC- or rCRISPC-encoding polynucleotides (e.g., mCRISPC or rCRISPC mRNA) and fragments for use as PCR primers for the amplification or mutation of mCRISPC or rCRISPC polynucleotides. The biologically active portions of CRISP proteins remain unknown, but based on the structure of snake venom protein, stecrisp (Gou M et al., J. Biol. Chem. 280:12405-12 (2005)), the mammalian family members are predicted to have three domains: (1) a PR1 domain (pathogenesis-related of group 1, hCRISPC amino acids 1-189, rCRISPC amino acids 1-194, and mCRISPC amino acids 1-190) that may involve endopeptidase activity, (2) a CRD (cysteine-rich domain, hCRISPC amino acids 210-249, rCRISPC amino acids 215-254, and mCRISPC amino acids 211-250) that has two folds similar to two known K⁺ channel blocking sea anemone toxins Shk and BgK, and (3) a hinge region between the PR1 domain and CRD with two disulfide bonds between two pairs of cysteines that are important for the stability of the PR1 domain. It will be understood that in discussing the uses of mCRISPC or rCRISPC nucleic acid molecules, e.g., nucleotides 97-846 of SEQ ID NO:19 or nucleotides 161-922 of SEQ ID NO:25, that fragments of such polynucleotides as well as full length mCRISPC or rCRISPC polynucleotides can be used.

A polynucleotide of the present invention, e.g., having nucleotides 97-846 of SEQ ID NO:19 or nucleotides 161-922 of SEQ ID NO:25, or a portion thereof, can be isolated using standard molecular biology techniques and the sequence information provided herein. For example, using all or portion of the polynucleotide sequence of nucleotides 97-846 of SEQ ID NO:19 or nucleotides 161-922 of SEQ ID NO:25 as a hybridization probe, mCRISPC or rCRISPC polynucleotides can be isolated using standard hybridization and cloning techniques (e.g., as described in Sambrook, J., Fritsh, E. F., and Maniatis, T. Molecular Cloning: A Laboratory Manual, 2nd ed., Cold Spring Harbor Laboratory, Cold Spring Harbor Laboratory Press, Cold Spring Harbor, N.Y., 1989).

Moreover, a polynucleotide encompassing all or a portion of nucleotides 97-846 of SEQ ID NO:19 or nucleotides 161-922 of SEQ ID NO:25 can be isolated by PCR using synthetic oligonucleotide primers designed based upon the sequence of, for example, SEQ ID NO:3 or SEQ ID NO:4, respectively.

A polynucleotide of the invention can be amplified using cDNA, mRNA or alternatively, genomic DNA, as a template and appropriate oligonucleotide primers according to standard PCR amplification techniques. The polynucleotide so amplified can be cloned into an appropriate vector and characterized by DNA sequence analysis. Furthermore, oligonucleotides corresponding to mCRISPC or rCRISPC polynucleotide sequences can be prepared by standard synthetic techniques, e.g., using an automated DNA synthesizer.

In a preferred embodiment, an isolated polynucleotide of the invention comprises the polynucleotide sequence shown in nucleotides 97-846 of SEQ ID NO:19 or nucleotides 161-922 of SEQ ID NO:25.

In another preferred embodiment, an isolated polynucleotide of the invention comprises a polynucleotide which is a complement of the polynucleotide sequence shown in nucleotides 97-846 of SEQ ID NO:19 or nucleotides 161-922 of SEQ ID NO:25 or a portion of any of these polynucleotide sequences. A polynucleotide which is complementary to the polynucleotide sequence shown in nucleotides 97-846 of SEQ ID NO:19 is one which is sufficiently complementary to the polynucleotide sequence shown in nucleotides 97-846 of SEQ ID NO:19, such that it can hybridize to the polynucleotide sequence shown in nucleotides 97-846 of SEQ ID NO:19, thereby forming a stable duplex. A polynucleotide which is complementary to the polynucleotide sequence shown in nucleotides 161-922 of SEQ ID NO:25 is one which is sufficiently complementary to the polynucleotide sequence shown in nucleotides 161-922 of SEQ ID NO:25 respectively, such that it can hybridize to the polynucleotide sequence shown in nucleotides 161-922 of SEQ ID NO:25 respectively, thereby forming a stable duplex.

In still another preferred embodiment, an isolated polynucleotide of the present invention comprises a polynucleotide sequence which is at least about 65%, 70%, 75%, 80%, 85%, 90%, 95%, 98% or more homologous to the polynucleotide sequence (e.g., to the entire length of the nucleotide sequence) shown in nucleotides 97-846 of SEQ ID NO:19 or which is at least about 65%, 70%, 75%, 80%, 85%, 90%, 95%, 98% or more homologous to the polynucleotide sequence (e.g., to the entire length of the nucleotide sequence) shown in nucleotides 161-922 of SEQ ID NO:25 or a portion of any of this nucleotide sequences.

Moreover, a polynucleotide of the invention can comprise only a portion of the polynucleotide sequence of nucleotides 97-846 of SEQ ID NO:19 or nucleotides 161-922 of SEQ ID NO:25; for example, a fragment which can be used as a probe or primer or a fragment encoding a biologically active portion of an mCRISPC or rCRISPC protein. The polynucleotide sequence determined from the cloning of the mCRISPC or rCRISPC genes allows for the generation of probes and primers designed for use in identifying and/or cloning other mCRISPC or rCRISPC family members, as well as mCRISPC or rCRISPC family homologues from other species. The probe/primer typically comprises a substantially purified oligonucleotide. In one embodiment, the oligonucleotide comprises a region of nucleotide sequence that hybridizes under stringent conditions to at least about 12 or 15, preferably about 20 or 25, more preferably about 30, 35, 40, 45, 50, 55, 60, 65, 75, or 100 consecutive polynucleotides of a sense sequence of nucleotides 97-846 of SEQ ID NO:19 or nucleotides 161-922 of SEQ ID NO:25 or of a naturally occurring allelic variant or mutant of nucleotides 97-846 of SEQ ID NO:19 or nucleotides 161-922 of SEQ ID NO:25. In another embodiment, a polynucleotide of the present invention comprises a polynucleotide sequence which is at least about 100, 200, 300, 400, 500, 600, or 700 nucleotides in length and hybridizes under stringent hybridization conditions to a polynucleotides sequence of SEQ ID NO:3 or SEQ ID NO:4 or the complements thereof.

In another embodiment, a polynucleotide of the invention comprises at least about 100, 200, 300, 400, 500, 600, or 700 contiguous nucleotides of SEQ ID NO:3 or SEQ ID NO:4.

In other embodiments, a polynucleotide of the invention has at least 70% identity, more preferably 80% identity, and even more preferably 90% identity with a polynucleotide comprising at least about 100, 200, 300, 400, 500, 600, or 700 polynucleotides of SEQ ID NO:3 or SEQ ID NO:4.

Probes based on the mCRISPC or rCRISPC polynucleotide sequences can be used to detect transcripts or genomic sequences encoding the same or homologous proteins. In preferred embodiments, the probe further comprises a label group attached thereto, for example, the label group can be a radioisotope, a fluorescent compound, an enzyme, or an enzyme co-factor. Such probes can be used as a part of a diagnostic test kit for identifying cells or tissues, particularly the epididymis, which misexpress a mCRISPC or rCRISPC protein, such as by measuring a level of a mCRISPC- or rCRISPC-encoding polynucleotide in a sample of cells from a subject, for example, detecting mCRISPC or rCRISPC mRNA levels or determining whether a genomic mCRISPC or rCRISPC gene has been mutated or deleted.

A nucleic acid fragment encoding a “biologically active portion of an mCRISPC or rCRISPC protein” can be prepared by isolating a portion of the polynucleotide sequence of nucleotides 97-846 of SEQ ID NO:19 or nucleotides 161-922 of SEQ ID NO:25 which encodes a polypeptide having a mCRISPC or rCRISPC biological activity (e.g., inhibition of capacitation and/or modulation of sperm-egg fusion), expressing the encoded portion of the mCRISPC or rCRISPC protein (e.g., by recombinant expression in vitro), and assessing the activity of the encoded portion of the mCRISPC or rCRISPC protein.

Polynucleotides that differ from nucleotides 97-846 of SEQ ID NO:19 or nucleotides 161-922 of SEQ ID NO:25 due to degeneracy of the genetic code, and thus encode the same mCRISPC or rCRISPC proteins as that encoded by nucleotides 97-846 of SEQ ID NO:19 or nucleotides 161-922 of SEQ ID NO:25, are encompassed by the invention. Accordingly, in another embodiment, an isolated polynucleotide of the invention has a polynucleotide sequence encoding a protein having an amino acid sequence shown in SEQ ID NO:5 or SEQ ID NO:6.

In addition to the mCRISPC and rCRISPC polynucleotide sequences shown in nucleotides 97-846 of SEQ ID NO:19 and nucleotides 161-922 of SEQ ID NO:25, it will be appreciated by those skilled in the art that DNA sequence polymorphisms that lead to changes in the amino acid sequences of the mCRISPC or rCRISPC proteins may exist within a population. Such genetic polymorphism in the mCRISPC or rCRISPC genes may exist among individuals within a population due to natural allelic variation. Such natural allelic variations include both functional and non-functional mCRISPC or rCRISPC proteins and can typically result in 1-5% variance in the polynucleotide sequence of a mCRISPC or rCRISPC gene. Any and all such polynucleotide variations and resulting amino acid polymorphisms in mCRISPC or rCRISPC genes that are the result of natural allelic variation and that do not alter the functional activity of an mCRISPC or rCRISPC protein are intended to be within the scope of the invention.

Nucleic acid molecules corresponding to natural allelic variants and homologues of the mCRISPC or rCRISPC molecules of the invention can be isolated, for example, based on their homology to the mCRISPC or rCRISPC polynucleotides disclosed herein using the cDNAs disclosed herein, or portions thereof, as a hybridization probe according to standard hybridization techniques. For example, an mCRISPC or rCRISPC DNA can be isolated from a mouse or rat genomic DNA library using all or portion of SEQ ID NO:3 or SEQ ID NO:4 as a hybridization probe and standard hybridization techniques (e.g., as described in Sambrook, J., et al. Molecular Cloning: A Laboratory Manual, 2nd ed., Cold Spring Harbor Laboratory, Cold Spring Harbor, N.Y., 1989). Moreover, a polynucleotide encompassing all or a portion of an mCRISPC or rCRISPC gene can be isolated by the polymerase chain reaction using oligonucleotide primers designed based upon the sequence of SEQ ID NO:3 or SEQ ID NO:4. For example, mRNA can be isolated from cells (e.g., by the guanidinium-thiocyanate extraction procedure of Chirgwin et al., Biochemistry 18: 5294-99 (1979)) and cDNA can be prepared using reverse transcriptase (e.g., Moloney MLV reverse transcriptase, available from Gibco/BRL, Bethesda, Md.; or AMV reverse transcriptase, available from Seikagaku America, Inc., St. Petersburg, Fla.). Synthetic oligonucleotide primers for PCR amplification can be designed based upon the polynucleotide sequence shown in SEQ ID NO:3 or SEQ ID NO:4. A polynucleotide of the invention can be amplified using cDNA or, alternatively, genomic DNA, as a template and appropriate oligonucleotide primers according to standard PCR amplification techniques. The polynucleotide so amplified can be cloned into an appropriate vector and characterized by DNA sequence analysis. Furthermore, oligonucleotides corresponding to a mCRISPC or rCRISPC polynucleotide sequence can be prepared by standard synthetic techniques, e.g., using an automated DNA synthesizer.

In another embodiment, an isolated polynucleotide of the invention can be identified based on shared nucleotide sequence identity using a mathematical algorithm. Such algorithms are outlined in more detail above (see, e.g., section I).

In another embodiment, an isolated polynucleotide of the invention is at least 15, 20, 25, 30 or more polynucleotides in length and hybridizes under stringent conditions to the nucleic acid molecule comprising the nucleotide sequence of nucleotides 97-846 of SEQ ID NO:19 or nucleotides 161-922 of SEQ ID NO:25 or complements thereof. In other embodiment, the polynucleotide is at least 30, 50, 100, 150, 200, 250, 300, 350, 400, 450, 500, 550, or 600 nucleotides in length. Preferably, the conditions are such that sequences at least 65%, preferably at least about 70%, more preferably at least about 80%, even more preferably at least about 85% or 90% homologous to each other typically remain hybridized to each other. Preferably, an isolated nucleic acid molecule of the invention that hybridizes under stringent conditions to the sequence of nucleotides 97-846 of SEQ ID NO:19 or nucleotides 161-922 of SEQ ID NO:25 or complements thereof corresponds to a naturally-occurring nucleic acid molecule.

In addition to naturally-occurring allelic variants of mCRISPC or rCRISPC sequences that may exist in the population, the skilled artisan will further appreciate that minor changes may be introduced by mutation into polynucleotide sequences, for example, of nucleotides 97-846 of SEQ ID NO:19 or nucleotides 161-922 of SEQ ID NO:25, thereby leading to changes in the amino acid sequence of the encoded protein, without altering the functional activity of an mCRISPC or rCRISPC protein. For example, nucleotide substitutions leading to amino acid substitutions at “non-essential” amino acid residues may be made in the sequence of nucleotides 97-846 of SEQ ID NO:19 or nucleotides 161-922 of SEQ ID NO:25. A “non-essential” amino acid residue is a residue that can be altered from the wild-type sequence of an mCRISPC or rCRISPC polynucleotide (e.g., the sequence of nucleotides 97-846 of SEQ ID NO:19 or nucleotides 161-922 of SEQ ID NO:25) without altering the functional activity of an mCRISPC or rCRISPC molecule. Exemplary residues which are non-essential and, therefore, amenable to substitution can be identified by one of ordinary skill in the art by performing an amino acid alignment of mCRISPC- or rCRISPC-related molecules and determining residues that are not conserved. Such residues, because they have not been conserved, are more likely amenable to substitution.

Accordingly, another aspect of the invention pertains to polynucleotides encoding mCRISPC or rCRISPC proteins that contain changes in amino acid residues that are not essential for an mCRISPC or rCRISPC activity. Such mCRISPC or rCRISPC proteins differ in amino acid sequence from SEQ ID NO:5 or SEQ ID NO:6 yet retain an inherent mCRISPC or rCRISPC activity. An isolated polynucleotide encoding a non-natural variant of an mCRISPC or rCRISPC protein can be created by introducing one or more nucleotide substitutions, additions, or deletions into the polynucleotide sequence of nucleotides 97-846 of SEQ ID NO:19 or nucleotides 161-922 of SEQ ID NO:25 such that one or more amino acid substitutions, additions, or deletions are introduced into the encoded protein. Mutations can be introduced into nucleotides 97-846 of SEQ ID NO:19 or nucleotides 161-922 of SEQ ID NO:25 by standard techniques, such as site-directed mutagenesis and PCR-mediated mutagenesis. Preferably, conservative amino acid substitutions are made at one or more non-essential amino acid residues. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an mCRISPC or rCRISPC polypeptide is preferably replaced with another amino acid residue from the same side chain family.

Alternatively, in another embodiment, mutations can be introduced randomly along all or part of an mCRISPC or rCRISPC coding sequence, such as by saturation mutagenesis, and the resultant mutants can be screened for their ability to bind to DNA and/or activate transcription, or to identify mutants that retain functional activity. Following mutagenesis, the mCRISPC or rCRISPC mutant protein can be expressed recombinantly in a host cell and the functional activity of the mutant protein can be determined using assays available in the art for assessing CRISPC activity.

Yet another aspect of the invention pertains to isolated polynucleotides encoding mCRISPC or rCRISPC fusion proteins. Such polynucleotides, comprising at least a first polynucleotide sequence encoding a full-length mCRISPC or rCRISPC protein, polypeptide, or peptide having CRISPC activity operably linked to a second polynucleotide sequence encoding a non-mCRISPC or non-rCRISPC protein, polypeptide, or peptide can be prepared by standard recombinant DNA techniques.

In a preferred embodiment, a mutant mCRISPC or rCRISPC protein can be assayed for the ability to inhibit capacitation and/or modulation of sperm-egg fusion.

In addition to the polynucleotides encoding mCRISPC or rCRISPC proteins described above, another aspect of the invention pertains to isolated polynucleotides which are antisense thereto. An “antisense” nucleic acid comprises a nucleotide sequence which is complementary to a “sense” nucleic acid encoding a protein, for example, complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire mCRISPC or rCRISPC coding strand, or only to a portion thereof. In one embodiment, an antisense nucleic acid molecule is antisense to a “coding region” of the coding strand of a nucleotide sequence encoding mCRISPC or rCRISPC. The term “coding region” refers to the region of the nucleotide sequence comprising codons which are translated into amino acid residues. In another embodiment, the antisense polynucleotide is antisense to a “noncoding region” of the coding strand of a polynucleotide sequence encoding mCRISPC or rCRISPC. The term “noncoding region” refers to 5′ and 3′ sequences which flank the coding region that are not translated into amino acids (i.e., also referred to as 5′ and 3′ untranslated regions).

Given the coding strand sequences encoding mCRISPC or rCRISPC disclosed herein, antisense nucleic acids of the invention can be designed according to the rules of Watson and Crick base pairing. The antisense polynucleotide can be complementary to the entire coding region of mCRISPC or rCRISPC mRNA, but more preferably is an oligonucleotide which is antisense to only a portion of the coding or noncoding region of mCRISPC or rCRISPC mRNA. For example, the antisense oligonucleotide can be complementary to the region surrounding the translation start site of mCRISPC or rCRISPC mRNA. An antisense oligonucleotide can be, for example, about 5, 10, 15, 20, 25, 30, 35, 40, 45 or 50 nucleotides in length. An antisense polynucleotide of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, for example, phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense polynucleotides of the invention are typically administered to a subject or generated in situ such that they hybridize with or bind to cellular mRNA and/or genomic DNA encoding an mCRISPC or rCRISPC protein to thereby inhibit expression of the protein, for example, by inhibiting transcription and/or translation. The hybridization can be by conventional nucleotide complementarity to form a stable duplex, or, for example, in the case of an antisense polynucleotide which binds to DNA duplexes, through specific interactions in the major groove of the double helix. An example of a route of administration of antisense polynucleotides of the invention include direct injection at a tissue site. Alternatively, antisense polynucleotides can be modified to target selected cells and then administered systemically. For example, for systemic administration, antisense molecules can be modified such that they specifically bind to receptors or antigens expressed on a selected cell surface, for example, by linking the antisense polynucleotides to peptides or antibodies which bind to cell surface receptors or antigens. The antisense polynucleotides can also be delivered to cells using the vectors described herein. To achieve sufficient intracellular concentrations of the antisense molecules, vector constructs in which the antisense polynucleotide is placed under the control of a strong pol II or pol III promoter are preferred.

In yet another embodiment, the antisense polynucleotide of the invention is an α-anomeric nucleic acid molecule. An α-anomeric nucleic acid molecule forms specific double-stranded hybrids with complementary RNA in which, contrary to the usual β-units, the strands run parallel to each other (Gaultier C et al., Nucleic Acids Res. 15:6625-41 (1987)). The antisense nucleic acid molecule can also comprise a 2′-o-methylribonucleotide (Inoue H et al., Nucleic Acids Res. 15:6131-48 (1987)), or a chimeric RNA-DNA analogue (Inoue H et al., FEBS Lett. 215:327-30 (1987)).

In still another embodiment, an antisense polynucleotide of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff J and Gerlach W L, Nature 334:585-91(1988))) can be used to catalytically cleave mCRISPC or rCRISPC mRNA transcripts to thereby inhibit translation of mCRISPC or rCRISPC mRNA. A ribozyme having specificity for an mCRISPC- or rCRISPC-encoding nucleic acid can be designed based upon the nucleotide sequence of nucleotides 97-846 of SEQ ID NO:19 or nucleotides 161-922 of SEQ ID NO:25. For example, a derivative of a Tetrahymena L-19 IVS RNA can be constructed in which the nucleotide sequence of the active site is complementary to the nucleotide sequence to be cleaved in an mCRISPC or rCRISPC-encoding mRNA (see, e.g., U.S. Pat. Nos. 4,987,071 and 5,116,742). Alternatively, mCRISPC or rCRISPC mRNA can be used to select a catalytic RNA having a specific ribonuclease activity from a pool of RNA molecules (see, e.g., Bartel D and Szostak J W, Science 261:1411-18 (1993)).

Alternatively, gene expression can be inhibited by targeting nucleotide sequences complementary to the regulatory region of mCRISPC or rCRISPC (e.g., the mCRISPC or rCRISPC promoter and/or enhancers) to form triple helical structures that prevent transcription of the mCRISPC or rCRISPC gene in target cells (see generally, Helene C, Anticancer Drug Des. 6:569-84 (1991); Helene C et al., Ann. N.Y. Acad Sci. 660:27-36 (1992); Maher L J, Bioassays 14:807-15 (1992)).

In yet another embodiment, the mCRISPC or rCRISPC polynucleotides of the present invention can be modified at the base moiety, sugar moiety, or phosphate backbone to improve, for example, the stability, hybridization, or solubility of the molecule. For example, the deoxyribose phosphate backbone of the polynucleotides can be modified to generate peptide nucleic acids (see Hyrup B et al., Bioorg. Med. Chem. 4:5-23 (1996)). As used herein, the terms “peptide nucleic acids” and “PNAs” refer to nucleic acid mimics, for example, DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B et al., supra; Perry-O'Keefe H et al., Proc. Natl. Acad. Sci. USA 93:14670-75 (1996).

PNAs of mCRISPC or rCRISPC polynucleotides can be used in therapeutic and diagnostic applications. For example, PNAs can be used as antisense or antigene agents for sequence-specific modulation of gene expression by, for example, inducing transcription or translation arrest or inhibiting replication. PNAs of mCRISPC or rCRISPC nucleic acid molecules can also be used in the analysis of single base pair mutations in a gene (e.g., by PNA-directed PCR clamping), as “artificial restriction enzymes” when used in combination with other enzymes, (e.g., S1 nucleases (Hyrup B et al., supra), or as probes or primers for DNA sequencing or hybridization (Hyrup B et al., supra; Perry-O'Keefe H et al., supra).

In another embodiment, PNAs of MCRISPC or rCRISPC can be modified (e.g., to enhance their stability or cellular uptake) by attaching lipophilic or other helper groups to PNA, by the formation of PNA-DNA chimeras, or by the use of liposomes or other techniques of drug delivery known in the art. For example, PNA-DNA chimeras of mCRISPC or rCRISPC polynucleotides can be generated which may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B et al., supra). The synthesis of PNA-DNA chimeras can be performed as described in Hyrup B et al., supra, and Finn P J et al., Nucleic Acids Res. 24:3357-63 (1996). For example, a DNA chain can be synthesized on a solid support using standard phosphoramidite coupling chemistry and modified nucleoside analogs, for example, 5′-(4-methoxytrityl)amino-5′-deoxy-thymidine phosphoramidite, can be used as a between the PNA and the 5′ end of DNA (Mag M et al., Nucleic Acid Res. 17: 5973-88 (1989)). PNA monomers are then coupled in a stepwise manner to produce a chimeric molecule with a 5′ PNA segment and a 3′ DNA segment (Finn P J et al., supra). Alternatively, chimeric molecules can be synthesized with a 5′ DNA segment and a 3′ PNA segment (Petersen K H et al., Bioorg. Med. Chem. Lett. 5:1119-24 (1995)).

In other embodiments, the oligonucleotide may include other appended groups such as peptides (e.g., for targeting host cell receptors in vivo), or agents facilitating transport across the cell membrane (see, e.g., Letsinger R L et al., Proc. Natl. Acad. Sci. USA 86:6553-56 (1989); Lemaitre M et al., Proc. Natl. Acad. Sci. USA 84:648-52 (1987); PCT Publication No. WO88/09810) or the blood-brain barrier (see, e.g., PCT Publication No. WO89/10134). In addition, oligonucleotides can be modified with hybridization-triggered cleavage agents (see, e.g., van der Krol A R et al., Biotechniques 6:958-76 (1988)) or intercalating agents (see, e.g., Zon G, Pharm. Res. 5:539-49 (1988)). To this end, the oligonucleotide may be conjugated to another molecule, (e.g., a peptide, hybridization triggered cross-linking agent, transport agent, or hybridization-triggered cleavage agent).

In one embodiment, mCRISPC or rCRISPC expression can be inhibited by short interfering RNAs (siRNA). The siRNA can be dsRNA having 19-25 nucleotides. siRNAs can be produced endogenously by degradation of longer dsRNA molecules by an RNase III-related nuclease called Dicer. siRNAs can also be introduced into a cell exogenously, or by transcription of an expression construct. Once formed, the siRNAs assemble with protein components into endoribonuclease-containing complexes known as RNA-induced silencing complexes (RISCs). An ATP-generated unwinding of the siRNA activates the RISCs, which in turn target the complementary mRNA transcript by Watson-Crick base-pairing, thereby cleaving and destroying the mRNA. Cleavage of the mRNA takes place near the middle of the region bound by the siRNA strand. This sequence specific mRNA degradation results in gene silencing.

At least two ways can be employed to achieve siRNA-mediated gene silencing. First, siRNAs can be synthesized in vitro and introduced into cells to transiently suppress gene expression. Synthetic siRNA provides an easy and efficient way to achieve RNAi. siRNA are duplexes of short mixed oligonucleotides which can include, for example, 19 RNAs nucleotides with symmetric dinucleotide 3′ overhangs. Using synthetic 21 bp siRNA duplexes (e.g., 19 RNA bases followed by a UU or dTdT 3′ overhang), sequence specific gene silencing can be achieved in mammalian cells. These siRNAs can specifically suppress targeted gene translation in mammalian cells without activation of DNA-dependent protein kinase (PKR) by longer double-stranded RNAs (dsRNA), which may result in non-specific repression of translation of many proteins.

Second, siRNAs can be expressed in vivo from vectors. This approach can be used to stably express siRNAs in cells or transgenic animals. In one embodiment, siRNA expression vectors are engineered to drive siRNA transcription from polymerase III (pol III) transcription units. Pol III transcription units are suitable for hairpin siRNA expression because they deploy a short AT rich transcription termination site that leads to the addition of 2 bp overhangs (e.g., UU) to hairpin siRNAs—a feature that is helpful for siRNA function. The Pol III expression vectors can also be used to create transgenic mice that express siRNA.

In another embodiment, siRNAs can be expressed in a tissue-specific manner. Under this approach, long dsRNAs are first expressed from a promoter (such as CMV (pol II)) in the nuclei of selected cell lines or transgenic mice. The long dsRNAs are processed into siRNAs in the nuclei (e.g., by Dicer). The siRNAs exit from the nuclei and mediate gene-specific silencing. A similar approach can be used in conjunction with tissue-specific (pol II) promoters to create tissue-specific knockdown mice.

Any 3′ dinucleotide overhang, such as UU, can be used for siRNA design. In some cases, G residues in the overhang are avoided because of the potential for the siRNA to be cleaved by RNase at single-stranded G residues.

With regard to the siRNA sequence itself, it has been found that siRNAs with 30-50% GC content can be more active than those with a higher G/C content in certain cases. Moreover, since a 4-6 nucleotide poly(T) tract may act as a termination signal for RNA pol III, stretches of >4 Ts or As in the target sequence may be avoided in certain cases when designing sequences to be expressed from an RNA pol III promoter. In addition, some regions of mRNA may be either highly structured or bound by regulatory proteins. Thus, it may be helpful to select siRNA target sites at different positions along the length of the gene sequence. Finally, the potential target sites can be compared to the appropriate genome database (human, mouse, rat, etc.). Any target sequences with more than 16-17 contiguous base pairs of homology to other coding sequences may be eliminated from consideration in certain cases.

In one embodiment, siRNA can be designed to have two inverted repeats separated by a short spacer sequence and end with a string of Ts that serve as a transcription termination site. This design produces an RNA transcript that is predicted to fold into a short hairpin siRNA. The selection of siRNA target sequence, the length of the inverted repeats that encode the stem of a putative hairpin, the order of the inverted repeats, the length and composition of the spacer sequence that encodes the loop of the hairpin, and the presence or absence of 5′-overhangs, can vary to achieve desirable results.

The siRNA targets can be selected by scanning an mRNA sequence for AA dinucleotides and recording the 19 nucleotides immediately downstream of the AA. Other methods can also been used to select the siRNA targets. In one example, the selection of the siRNA target sequence is purely empirically determined (see, e.g., Sui G et al., Proc. Natl. Acad. Sci. USA 99:5515-20 (2002)), as long as the target sequence starts with GG and does not share significant sequence homology with other genes as analyzed by BLAST search. In another example, a more elaborate method is employed to select the siRNA target sequences. This procedure exploits an observation that any accessible site in endogenous mRNA can be targeted for degradation by synthetic oligodeoxyribonucleotide/RNase H method (see, e.g., Lee N S et al., Nature Biotechnol. 20:500-05 (2002)).

In another embodiment, the hairpin siRNA expression cassette is constructed to contain the sense strand of the target, followed by a short spacer, the antisense strand of the target, and 5-6 Ts as transcription terminator. The order of the sense and antisense strands within the siRNA expression constructs can be altered without affecting the gene silencing activities of the hairpin siRNA. In certain instances, the reversal of the order may cause partial reduction in gene silencing activities.

The length of nucleotide sequence being used as the stem of siRNA expression cassette can range, for instance, from 19 to 29. The loop size can range from 3 to 23 nucleotides. Other lengths and/or loop sizes can also be used.

In yet another embodiment, a 5′ overhang in the hairpin siRNA construct can be used, provided that the hairpin siRNA is functional in gene silencing. In one specific example, the 5′ overhang includes about 6 nucleotide residues.

In still yet another embodiment, the target sequence for RNAi is a 21-mer sequence fragment selected from nucleotides 97-846 of SEQ ID NO:19 or nucleotides 161-922 of SEQ ID NO:25. The 5′ end of the target sequence has dinucleotide “NA,” where “N” can be any base and “A” represents adenine. The remaining 19-mer sequence has a GC content of between 35% and 55%. In addition, the remaining 19-mer sequence does not include any four consecutive A or T (i.e., AAAA or TTTT), three consecutive G or C (i.e., GGG or CCC), or seven “GC” in a row.

Additional criteria can also be used for selecting RNAi target sequences. For instance, the GC content of the remaining 19-mer sequence can be limited to between 45% and 55%. Moreover, any 19-mer sequence having three consecutive identical bases (i.e., GGG, CCC, TTT, or AAA) or a palindrome sequence with 5 or more bases is excluded. Furthermore, the remaining 19-mer sequence can be selected to have low sequence homology to other genes. In one specific example, potential target sequences are searched by BLASTN against NCBI's human UniGene cluster sequence database. The human UniGene database contains non-redundant sets of gene-oriented clusters. Each UniGene cluster includes sequences that represent a unique gene. 19-mer sequences producing no hit to other human genes under the BLASTN search can be selected. During the search, the e-value may be set at a stringent value (such as “1”).

The effectiveness of the siRNA sequences, as well as any other RNAi sequence derived according to the present invention, can be evaluated using various methods known in the art. For instance, an siRNA sequence of the present invention can be introduced into a cell that expresses the mCRISPC or rCRISPC gene. The polypeptide or mRNA level of the mCRISPC or rCRISPC gene in the cell can be detected. A substantial change in the expression level of the mCRISPC or rCRISPC gene before and after the introduction of the siRNA sequence is indicative of the effectiveness of the siRNA sequence in suppressing the expression of the mCRISPC or rCRISPC gene. In one specific example, the expression levels of other genes are also monitored before and after the introduction of the siRNA sequence. An siRNA sequence which has inhibitory effect on mCRISPC or rCRISPC gene expression but does not significantly affect the expression of other genes can be selected. In another specific example, multiple siRNA or other RNAi sequences can be introduced into the same target cell. These siRNA or RNAi sequences specifically inhibit mCRISPC or rCRISPC gene expression but not the expression of other genes. In yet another specific example, siRNA or other RNAi sequences that inhibit the expression of the mCRISPC or rCRISPC gene and other gene or genes can be used.

Antisense polynucleotides may be produced from a heterologous expression cassette in a transfectant cell or transgenic cell. Alternatively, the antisense polynucleotides may comprise soluble oligonucleotides that are administered to the external milieu, either in the culture medium in vitro or in the circulatory system or in interstitial fluid in vivo. Soluble antisense polynucleotides present in the external milieu have been shown to gain access to the cytoplasm and inhibit translation of specific mRNA species.

III. Isolated mCRISPC or rCRISPC Proteins, Fragments Thereof, and Anti-mCRISPC or Anti-rCRISPC Antibodies

Another aspect of the invention pertains to isolated mCRISPC or rCRISPC proteins, and biologically active portions thereof, as well as polypeptide fragments suitable for use as immunogens to raise anti-mCRISPC or anti-rCRISPC antibodies. In one embodiment, native mCRISPC or rCRISPC proteins can be isolated from cells or tissue sources by an appropriate purification scheme using standard protein purification techniques. In another embodiment, mCRISPC or rCRISPC proteins are produced by recombinant DNA techniques. Alternative to recombinant expression, an mCRISPC or rCRISPC protein or polypeptide can be synthesized chemically using standard peptide synthesis techniques. It will be understood that in discussing the uses of mCRISPC or rCRISPC proteins, e.g., as shown in SEQ ID NO:5 or SEQ ID NO:6, that fragments of such proteins that are not full length mCRISPC or rCRISPC polypeptides as well as full length mCRISPC or rCRISPC proteins can be used.

In some embodiments, a CRISPC purification scheme includes the addition of inhibitor(s) of glycosidase activity thereby allowing for purification of glycosylated forms of the protein. In one embodiment, the purification scheme includes the addition of inhibitor(s) of sialidase activity, for example the addition of 2,3-dehydro-2-deoxy-N-acetylneuraminic acid (DANA), thereby allowing for purification of sialylated forms of CRISPC. As discussed in section (V)(F) infra, CRISPC protease activity may depend on whether the protein is sialylated or not. CRISPC glycosylations may be N-linked and/or O-linked and some, none, or all of said N-linked and/or O-linked glycosylations may be sialylated.

Another aspect of the invention pertains to isolated mCRISPC or rCRISPC proteins. Preferably, the mCRISPC or rCRISPC proteins comprise the amino acid sequence encoded by nucleotides 97-846 of SEQ ID NO:19 or nucleotides 161-922 of SEQ ID NO:25 or a portion thereof. In another preferred embodiment, the protein comprises the amino acid sequence of SEQ ID NO:5 or SEQ ID NO:6 or a portion thereof. In other embodiments, the protein has at least 65%, at least 70% amino acid identity, more preferably 80% amino acid identity, more preferably 90%, and even more preferably, 95% amino acid identity with the amino acid sequence shown in SEQ ID NO:5 or a portion thereof. Alternatively, the protein has at least 65%, at least 70% amino acid identity, more preferably 80% amino acid identity, more preferably 90%, and even more preferably, 95% amino acid identity with the amino acid sequence shown in SEQ ID NO:6 or a portion thereof. Preferred portions of mCRISPC or rCRISPC polypeptide molecules are biologically active, for example, a portion of the CRISPC polypeptide having the ability to inhibit capacitation and/or modulate sperm-egg fusion.

Biologically active portions of an mCRISPC or rCRISPC protein include peptides comprising amino acid sequences sufficiently homologous to or derived from the amino acid sequence of the mCRISPC or rCRISPC protein, which include less amino acids than the full length mCRISPC or rCRISPC proteins, and exhibit at least one activity of an mCRISPC or rCRISPC protein.

The invention also provides mCRISPC or rCRISPC chimeric or fusion proteins. For example, in one embodiment, the fusion protein is a GST-mCRISPC or GST-rCRISPC member fusion protein in which the mCRISPC or rCRISPC member sequences are fused to the C-terminus of the GST sequences. In another embodiment, the fusion protein is an mCRISPC- or rCRISPC-HA fusion protein in which the mCRISPC or rCRISPC member nucleotide sequence is inserted in a vector such as pCEP4-HA vector (Herrscher R F et al., Genes Dev. 9:3067-82 (1995)) such that the mCRISPC or rCRISPC member sequences are fused in frame to an influenza hemagglutinin epitope tag. Such fusion proteins can facilitate the purification of a recombinant mCRISPC or rCRISPC member.

Fusion proteins and peptides produced by recombinant techniques may be secreted and isolated from a mixture of cells and medium containing the protein or peptide. Alternatively, the protein or peptide may be retained cytoplasmically and the cells harvested, lysed, and the protein isolated. A cell culture typically includes host cells, media, and other byproducts. Suitable media for cell culture are well known in the art. Protein and peptides can be isolated from cell culture media, host cells, or both using techniques known in the art for purifying proteins and peptides. Techniques for transfecting host cells and purifying proteins and peptides are known in the art.

Preferably, an mCRISPC or rCRISPC fusion protein of the invention is produced by standard recombinant DNA techniques. For example, DNA fragments coding for the different polypeptide sequences are ligated together in-frame in accordance with conventional techniques, for example employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al., John Wiley & Sons: 1992). Moreover, many expression vectors are commercially available that already encode a fusion moiety (e.g., a GST polypeptide or an HA epitope tag). An mCRISPC- or rCRISPC-encoding nucleic acid can be cloned into such an expression vector such that the fusion moiety is linked in-frame to the mCRISPC or rCRISPC protein.

In another embodiment, the fusion protein is an mCRISPC or rCRISPC protein containing a heterologous signal sequence at its N-terminus. In certain host cells (e.g., mammalian host cells), expression and/or secretion of mCRISPC or rCRISPC can be increased through use of a heterologous signal sequence. The mCRISPC or rCRISPC fusion proteins of the invention can be incorporated into pharmaceutical compositions and administered to a subject in vivo. Use of mCRISPC or rCRISPC fusion proteins may be useful therapeutically for the treatment of disorders, for example, conditions related to infertility. Moreover, the mCRISPC or rCRISPC fusion proteins of the invention can be used as immunogens to produce anti-mCRISPC or anti-rCRISPC antibodies in a subject.

The present invention also pertains to variants of the mCRISPC or rCRISPC proteins which function as either mCRISPC or rCRISPC agonists (mimetics) or as mCRISPC or rCRISPC antagonists. Variants of the mCRISPC or rCRISPC proteins can be generated by mutagenesis, for example, discrete point mutation or truncation of an mCRISPC or rCRISPC protein. An agonist of the mCRISPC or rCRISPC proteins can retain substantially the same, or a subset, of the biological activities of the naturally occurring form of an mCRISPC or rCRISPC protein. An antagonist of an mCRISPC or rCRISPC protein can inhibit one or more of the activities of the naturally occurring form of the mCRISPC or rCRISPC protein by, for example, competitively modulating a cellular activity of an mCRISPC or rCRISPC protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the mCRISPC or rCRISPC protein.

In one embodiment, the invention pertains to derivatives of mCRISPC or rCRISPC which may be formed by modifying at least one amino acid residue of mCRISPC or rCRISPC by oxidation, reduction, or other derivatization processes known in the art.

In one embodiment, variants of an mCRISPC or rCRISPC protein which function as either mCRISPC or rCRISPC agonists (mimetics) or as mCRISPC or rCRISPC antagonists can be identified by screening combinatorial libraries of mutants, for example, truncation mutants, of an mCRISPC or rCRISPC protein for mCRISPC or rCRISPC protein agonist or antagonist activity. In one embodiment, a variegated library of mCRISPC or rCRISPC variants is generated by combinatorial mutagenesis at the nucleic acid level and is encoded by a variegated gene library. A variegated library of mCRISPC or rCRISPC variants can be produced by, for example, enzymatically ligating a mixture of synthetic oligonucleotides into gene sequences such that a degenerate set of potential mCRISPC or rCRISPC sequences is expressible as individual polypeptides, or alternatively, as a set of larger fusion proteins (e.g., for phage display) containing the set of mCRISPC or rCRISPC sequences therein. There are a variety of methods which can be used to produce libraries of potential mCRISPC or rCRISPC variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential mCRISPC or rCRISPC sequences. Methods for synthesizing degenerate oligonucleotides are known in the art (see, e.g., Narang S A, Tetrahedron 39:3-22 (1983); Itakura K et al., Annu. Rev. Biochem. 53:323-56 (1984); Itakura K et al., Science 198:1056-63 (1977); Ike Y et al., Nucleic Acids Res. 11:477-88 (1983)).

In addition, libraries of fragments of an mCRISPC or rCRISPC protein coding sequence can be used to generate a variegated population of mCRISPC or rCRISPC fragments for screening and subsequent selection of variants of an mCRISPC or rCRISPC protein. In one embodiment, a library of coding sequence fragments can be generated by treating a double stranded PCR fragment of an mCRISPC or rCRISPC coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal, and internal fragments of various sizes of the mCRISPC or rCRISPC protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of mCRISPC or rCRISPC proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify mCRISPC or rCRISPC variants (Arkin A P and Youvan D C, Proc. Natl. Acad. Sci. USA 89:7811-15 (1992); Delgrave S et al., Protein Eng. 6:327-31 (1993)).

In one embodiment, cell based assays can be exploited to analyze a variegated mCRISPC or rCRISPC library. For example, a library of expression vectors can be transfected into a cell line which ordinarily synthesizes and secretes mCRISPC or rCRISPC. The transfected cells are then cultured such that mCRISPC or rCRISPC and a particular mutant mCRISPC or rCRISPC are secreted and the effect of expression of the mutant on mCRISPC or rCRISPC activity in cell supernatants can be detected, for example, by any of a number of enzymatic assays. Plasmid DNA can then be recovered from the cells which score for inhibition, or alternatively, potentiation of mCRISPC or rCRISPC activity, and the individual clones further characterized.

In addition to mCRISPC or rCRISPC polypeptides consisting only of naturally-occurring amino acids, mCRISPC or rCRISPC peptidomimetics are also provided. Peptide analogs are commonly used in the pharmaceutical industry as non-peptide drugs with properties analogous to those of the template peptide. These types of non-peptide compound are termed “peptide mimetics” or “peptidomimetics” (Fauchere J, Adv. Drug Res. 15:29 (1986); Veber D F and Freidinger R M, Trends Neurosci. 8:392-96 (1985); Evans B E et al., J. Med. Chem 30:1229-39 (1987)) and are usually developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biological or pharmacological activity), such as human CRISPC, but have one or more peptide linkages optionally replaced by a linkage selected from the group consisting of: —CH₂NH—, CH₂S—, —CH₂—CH₂—, —CH═CH— (cis and trans), —COCH₂—, —CH(OH)CH₂—, and —CH₂SO—, by methods known in the art and further described in the following references: Spatola A F in “Chemistry and Biochemistry of Amino Acids, Peptides, and Proteins,” B. Weinstein, ed., Marcel Dekker, New York, p. 267 (1983); Spatola, A F, Vega Data (March 1983), Vol. 1, Issue 3, “Peptide Backbone Modifications” (general review); Morley J S, Trends Pharmcol. Sci. 1:463-68 (1980) (general review); Hudson D et al., Int. J. Pept. Prot. Res. 14:177-85 (1979) (—CH₂NH—, CH₂CH₂—); Spatola A F et al., Life Sci. 38:1243-49 (1986) (—CH₂—S); Hann M M, J. Chem. Soc. Perkin Trans. 1, 307-314 (1982) (—CH—CH—, cis and trans); Almquist R G et al., J. Med. Chem. 23:1392-98 (1980) (—COCH₂—); Jennings-White C et al., Tetrahedron Lett. 23:2533-34 (1982) (—COCH₂—); EP 0 045 665 (—CH(OH)CH₂—); Holladay M W et al., Tetrahedron Lett., 24:4401-04 (1983) (—C(OH)CH₂—); HrubyVJ, Life Sci. 31:189-99 (1982) (—CH₂—S—). A particularly preferred non-peptide linkage is —CH₂NH—. Such peptide mimetics may have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others. Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macromolecules(s) to which the peptidomimetic binds to produce the therapeutic effect. Derivatization (e.g., labeling) of peptidomimetics should not substantially interfere with the desired biological or pharmacological activity of the peptidomimetic.

Systematic substitution of one or more amino acids of an mCRISPC or rCRISPC amino acid sequence with a D-amino acid of the same type (e.g., D-lysine in place of L-lysine) may be used to generate more stable peptides. In addition, constrained peptides comprising an mCRISPC or rCRISPC amino acid sequence or a substantially identical sequence variation may be generated by methods known in the art (Rizo J and Gierasch L M, Ann. Rev. Biochem. 61:387-416 (1992)); for example, by adding internal cysteine residues capable of forming intramolecular disulfide bridges which cyclize the peptide.

The amino acid sequences of mCRISPC or rCRISPC polypeptides identified herein will enable those of skill in the art to produce polypeptides corresponding to mCRISPC or rCRISPC peptide sequences and sequence variants thereof. Such polypeptides may be produced in prokaryotic or eukaryotic host cells by expression of polynucleotides encoding an mCRISPC or rCRISPC peptide sequence, frequently as part of a larger polypeptide. Alternatively, such peptides may be synthesized by chemical methods. Methods for expression of heterologous proteins in recombinant hosts, chemical synthesis of polypeptides, and in vitro translation are well known in the art and are described further in Maniatis et al., Molecular Cloning: A Laboratory Manual (1989), 2nd Ed., Cold Spring Harbor, N.Y.;. Berger and Kimmel, Methods in Enzymology, Volume 152, Guide to Molecular Cloning Techniques (1987), Academic Press, Inc., San Diego, Calif.; Gutte B and Merrifield R B, J. Am. Chem. Soc. 91:501-02 (1969); Chaiken I M, CRC Crit. Rev. Biochem. 11:255-301 (1981); Kaiser E T et al., Science 243:187-92 (1989); Merrifield B, Science 232:341-47 (1986); Kent S B H, Ann. Rev. Biochem. 57:957-89 (1988); Offord, R. E. (1980) Semisynthetic Proteins, Wiley Publishing.

Peptides can be produced, typically by direct chemical synthesis. Peptides can be produced as modified peptides, with nonpeptide moieties attached by covalent linkage to the N-terminus and/or C-terminus. In certain preferred embodiments, either the carboxy-terminus or the amino-terminus, or both, are chemically modified. The most common modifications of the terminal amino and carboxyl groups are acetylation and amidation, respectively. Amino-terminal modifications such as acylation (e.g., acetylation) or alkylation (e.g., methylation) and carboxy-terminal-modifications such as amidation, as well as other terminal modifications, including cyclization, may be incorporated into various embodiments of the invention. Certain amino-terminal and/or carboxy-terminal modifications and/or peptide extensions to the core sequence can provide advantageous physical, chemical, biochemical, and pharmacological properties, such as: enhanced stability, increased potency and/or efficacy, resistance to serum proteases, desirable pharmacokinetic properties, and others. Peptides may be used therapeutically to treat disease.

An isolated mCRISPC or rCRISPC protein, or a portion or fragment thereof, can also be used as an immunogen to generate antibodies that bind mCRISPC or rCRISPC using standard techniques for polyclonal and monoclonal antibody preparation. A full-length mCRISPC or rCRISPC protein can be used or, alternatively, the invention provides antigenic peptide fragments of mCRISPC or rCRISPC for use as immunogens. The antigenic peptide of mCRISPC or rCRISPC comprises at least 8 amino acid residues and encompasses an epitope of mCRISPC or rCRISPC such that an antibody raised against the peptide forms a specific immune complex with mCRISPC or rCRISPC. Preferably, the antigenic peptide comprises at least 10 amino acid residues, more preferably at least 15 amino acid residues, even more preferably at least 20 amino acid residues, and most preferably at least 30 amino acid residues.

Alternatively, an antigenic peptide fragment of an mCRISPC or rCRISPC polypeptide can be used as the immunogen. An antigenic peptide fragment of an mCRISPC or rCRISPC polypeptide typically comprises at least 8 amino acid residues of the amino acid sequence shown in SEQ ID NO:5 or SEQ ID NO:6 and encompasses an epitope of a mCRISPC or rCRISPC polypeptide such that an antibody raised against the peptide forms an immune complex with an mCRISPC or rCRISPC molecule. Preferred epitopes encompassed by the antigenic peptide are regions of mCRISPC or rCRISPC that are located on the surface of the protein, for example, hydrophilic regions. In one embodiment, an antibody binds substantially specifically to an mCRISPC or rCRISPC molecule. In another embodiment, an antibody binds specifically to an mCRISPC or rCRISPC polypeptide.

Preferably, the antigenic peptide comprises at least about 10 amino acid residues, more preferably at least about 15 amino acid residues, even more preferably at least 20 about amino acid residues, and most preferably at least about 30 amino acid residues. Preferred epitopes encompassed by the antigenic peptide are regions of an mCRISPC or rCRISPC polypeptide that are located on the surface of the protein, for example, hydrophilic regions, and that are unique to an mCRISPC or rCRISPC polypeptide. In one embodiment, such epitopes can be specific for an mCRISPC or rCRISPC proteins from one species, such as mouse or human (i.e., an antigenic peptide that spans a region of an mCRISPC or rCRISPC polypeptide that is not conserved across species is used as immunogen; such non-conserved residues can be determined using an alignment such as that provided herein). A standard hydrophobicity analysis of the protein can be performed to identify hydrophilic regions.

In a preferred embodiment, the peptide fragment of mCRISPC comprises SEQ ID NO:13 or SEQ ID NO:14. In another preferred embodiment, the peptide fragment of rCRISPC comprises SEQ ID NO:15 or SEQ ID NO:16.

An mCRISPC or rCRISPC immunogen typically is used to prepare antibodies by immunizing a suitable subject (e.g., rabbit, goat, mouse, or other mammal) with the immunogen. An appropriate immunogenic preparation can contain, for example, a recombinantly expressed mCRISPC or rCRISPC protein or a chemically synthesized mCRISPC or rCRISPC peptide. The preparation can further include an adjuvant, such as Freund's complete or incomplete adjuvant, or similar immunostimulatory agent. Immunization of a suitable subject with an immunogenic mCRISPC or rCRISPC preparation induces a polyclonal anti-mCRISPC or anti-rCRISPC antibody response.

Accordingly, another aspect of the invention pertains to the use of anti-mCRISPC or anti-rCRISPC antibodies. Polyclonal anti-mCRISPC or anti-rCRISPC antibodies can be prepared as described above by immunizing a suitable subject with an mCRISPC or rCRISPC immunogen. The anti-mCRISPC or anti-rCRISPC antibody titer in the immunized subject can be monitored over time by standard techniques, such as with an enzyme linked immunosorbent assay (ELISA) using immobilized an mCRISPC or rCRISPC polypeptide. If desired, the antibody molecules directed against an mCRISPC or rCRISPC polypeptide can be isolated from the mammal (e.g., from the blood) and further purified by well known techniques, such as protein A chromatography to obtain the IgG fraction. At an appropriate time after immunization, for example, when the anti-mCRISPC or ant-rCRISPC antibody titers are highest, antibody-producing cells can be obtained from the subject and used to prepare monoclonal antibodies by standard techniques, such as the hybridoma technique originally described by Kohler G and Milstein C, Nature 256:495-97 (1975) (see also, Brown J P et al., J. Immunol. 127:539-46 (1981); Brown J P et al., J. Biol. Chem. 255:4980-83 (1980); Yeh M Y et al., Proc. Natl. Acad. Sci. USA 76:2927-31 (1979); Yeh M Y et al., Int. J. Cancer 29:269-75 (1982)), the more recent human B cell hybridoma technique (Kozbor D and Roder J C, Immunol. Today 4:72-79 (1983)), the EBV-hybridoma technique (Cole et al. (1985), Monoclonal Antibodies and Cancer Therapy, Alan R. Liss, Inc., pp. 77-96), or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); Lerner E A, Yale J. Biol. Med., 54:387-402 (1981); Gefter M L et al., Somatic Cell Genet. 3:231-36 (1977)). Briefly, an immortal cell line (typically a myeloma) is fused to lymphocytes (typically splenocytes) from a mammal immunized with an mCRISPC or rCRISPC immunogen as described above, and the culture supernatants of the resulting hybridoma cells are screened to identify a hybridoma producing a monoclonal antibody that binds specifically to an mCRISPC or rCRISPC polypeptide.

Any of the many well known protocols used for fusing lymphocytes and immortalized cell lines can be applied for the purpose of generating an anti-mCRISPC or anti-rCRISPC monoclonal antibody (see, e.g., Galfre G et al., Nature 266:550-52 (1977); Geifer M L et al., supra; Lerner E A, supra; Kenneth, Monoclonal Antibodies, cited supra). Moreover, the ordinary skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines may be used as a fusion partner according to standard techniques, for example, the P3-NS1/1-Ag4-1, P3-x63-Ag8.653 or Sp2/O-Ag14 myeloma lines. These myeloma lines are available from the American Type Culture Collection (ATCC), Rockville, Md. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind a mCRISPC or rCRISPC molecule, for example, using a standard ELISA assay.

As an alternative to preparing monoclonal antibody-secreting hybridomas, a monoclonal anti-mCRISPC or anti-rCRISPC antibody can be identified and isolated by screening a recombinant combinatorial immunoglobulin library (e.g., an antibody phage display library) with mCRISPC or rCRISPC to thereby isolate immunoglobulin library members that bind an mCRISPC or rCRISPC polypeptide. Kits for generating and screening phage display libraries are commercially available (e.g., the GE Healthcare Recombinant Phage Antibody System, Catalog No. 27-9400-01). Additionally, examples of methods and reagents particularly amenable for use in generating and screening antibody display library can be found in, for example, U.S. Pat. No. 5,223,409; WO 92/18619; WO 91/17271; WO 92/20791; WO 92/15679; WO 93/01288; WO 92/01047; WO 92/09690; WO 90/02809; Fuchs P et al., Biotechnology (N.Y.) 9:1370-72 (1991); Hay B N et al., Hum. Antibodies Hybridomas 3:81-85 (1992); Huse W D et al., Science 246:1275-81 (1989); Griffiths A D et al., EMBO J. 12:725-34 (1993); Hawkins R E et al., J. Mol. Biol. 226:889-96 (1992); Clarkson T et al., Nature 352:624-28 (1991); Gram H et al., Proc. Natl. Acad. Sci. USA 89:3576-80 (1992); Garrard L J et al., Biotechnology (N.Y.) 9:1373-77 (1991); Hoogenboom H R et al., Nucleic Acids Res. 19:4133-37 (1991); Barbas C F et al., Proc. Natl. Acad. Sci. USA 88:7978-82 (1991); and McCafferty J et al., Nature 348:552-54 (1990).

Additionally, recombinant anti-mCRISPC or anti-rCRISPC antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in WO 87/02671; EP 0 184 187; EP 0 171 496; EP 0 173 494; WO 86/01533; U.S. Pat. No. 4,816,567; EP 0 125 023; Better M et al., Science 240:1041-43 (1988); Liu A Y et al., Proc. Natl. Acad. Sci. USA 84:3439-43 (1987); Liu A Y et al., J. Immunol. 139:3521-26 (1987); Sun L K et al., Proc. Natl. Acad. Sci. USA 84:214-18 (1987); Nishimura Y et al., Cancer Res. 47:999-1005 (1987); Wood C R et al., Nature 314:446-49 (1985); Shaw D R et al., J. Natl. Cancer Inst. 80:1553-59 (1988); Morrison S L, Science 229:1202-07 (1985); U.S. Pat. No. 5,225,539; Verhocyan M et al., Science 239:1534-36 (1988); and Beidler C B et al., J. Immunol. 141:4053-60 (1988).

In addition, humanized antibodies can be made according to standard protocols such as those disclosed in U.S. Pat. No. 5,565,332. In another embodiment, antibody chains or specific binding pair members can be produced by recombination between vectors comprising nucleic acid molecules encoding a fusion of a polypeptide chain of a specific binding pair member and a component of a replicable genetic display package and vectors containing nucleic acid molecules encoding a second polypeptide chain of a single binding pair member using techniques known in the art, for example, as described in U.S. Pat. Nos. 5,565,332; 5,871,907; or 5,733,743.

An anti-mCRISPC or anti-rCRISPC antibody (e.g., monoclonal antibody) can be used to isolate an MCRISPC or rCRISPC polypeptide by standard techniques, such as affinity chromatography or immunoprecipitation. Anti-mCRISPC or anti-rCRISPC antibodies can facilitate the purification of natural mCRISPC or rCRISPC polypeptides from cells and of recombinantly produced mCRISPC or rCRISPC polypeptides expressed in host cells. Moreover, an anti-mCRISPC or rCRISPC antibody can be used to detect an mCRISPC or rCRISPC protein (e.g., in a cellular lysate or cell supernatant). Detection may be facilitated by coupling (i.e., physically linking) the antibody to a detectable substance. Accordingly, in one embodiment, an anti-mCRISPC or anti-rCRISPC antibody of the invention is labeled with a detectable substance. Examples of detectable substances include various enzymes, prosthetic groups, fluorescent materials, luminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride, or phycoerythrin; an example of a luminescent material includes luminol; and examples of suitable radioactive material include ¹²⁵I, ¹³¹I, ³⁵S, or ³H.

Anti-mCRISPC or anti-rCRISPC antibodies are also obtainable by a process comprising:
(a) immunizing an animal with an immunogenic mCRISPC or rCRISPC protein, or an immunogenic portion thereof unique to an mCRISPC or rCRISPC polypeptide; and
(b) isolating from the animal antibodies that specifically bind to an mCRISPC or rCRISPC protein.

Accordingly, in one embodiment, anti-mCRISPC or anti-rCRISPC antibodies can be used, e.g., intracellularly to inhibit protein activity. The use of intracellular antibodies to inhibit protein function in a cell is known in the art (see e.g., Carlson J R, Mol. Cell. Biol. 8:2638-46 (1988); Biocca S et al., EMBO J. 9:101-08 (1990); Werge T M et al., FEBS Lett. 274:193-98 (1990); Carlson J R, Proc. Natl. Acad. Sci. USA 90:7427-28 (1993); Marasco W A et al., Proc. Natl. Acad. Sci. USA 90:7889-93 (1993); Biocca S et al., Biotechnology (N.Y.) 12:396-99 (1994); Chen S-Y et al., Hum. Gene Ther. 5:595-601 (1994); Duan L et al., Proc. Natl. Acad. Sci. USA 91:5075-79 (1994); Chen S-Y et al., Proc. Natl. Acad. Sci. USA 91:5932-36 (1994); Beerli R R et al., J. Biol. Chem. 269:23931-36 (1994); Beerli R R et al., Biochem. Biophys. Res. Commun. 204:666-72 (1994); Mhashilkar A M et al., EMBO J. 14:1542-51 (1995); Richardson J H et al., Proc. Natl. Acad. Sci. USA 92:3137-41 (1995); WO 94/02610; and WO 95/03832).

In one embodiment, a recombinant expression vector is prepared which encodes the antibody chains in a form such that, upon introduction of the vector into a cell, the antibody chains are expressed as a functional antibody in an intracellular compartment of the cell. For inhibition of mCRISPC or rCRISPC activity according to the inhibitory methods of the invention, an intracellular antibody that specifically binds the mCRISPC or rCRISPC protein is expressed in the cytoplasm of the cell. To prepare an intracellular antibody expression vector, antibody light and heavy chain cDNAs encoding antibody chains specific for the target protein of interest, for example, mCRISPC or rCRISPC, are isolated, typically from a hybridoma that secretes a monoclonal antibody specific for the mCRISPC or rCRISPC protein. Hybridomas secreting anti-mCRISPC or anti-rCRISPC monoclonal antibodies, or recombinant anti-mCRISPC or anti-rCRISPC monoclonal antibodies, can be prepared as described above. Once a monoclonal antibody specific for mCRISPC or rCRISPC protein has been identified (e.g., either a hybridoma-derived monoclonal antibody or a recombinant antibody from a combinatorial library), DNAs encoding the light and heavy chains of the monoclonal antibody are isolated by standard molecular biology techniques. For hybridoma derived antibodies, light and heavy chain cDNAs can be obtained, for example, by PCR amplification or cDNA library screening. For recombinant antibodies, such as from a phage display library, cDNA encoding the light and heavy chains can be recovered from the display package (e.g., phage) isolated during the library screening process. Nucleotide sequences of antibody light and heavy chain genes from which PCR primers or cDNA library probes can be prepared are known in the art. For example, many such sequences are disclosed in Kabat E A et al. (1991) Sequences of Proteins of Immunological Interest, Fifth Edition, U.S. Department of Health and Human Services, NIH Publication No. 91-3242 and in the “Vbase” human germline sequence database.

Once obtained, the antibody light and heavy chain sequences are cloned into a recombinant expression vector using standard methods. To allow for cytoplasmic expression of the light and heavy chains, the nucleotide sequences encoding the hydrophobic leaders of the light and heavy chains are removed. An intracellular antibody expression vector can encode an intracellular antibody in one of several different forms. For example, in one embodiment, the vector encodes full-length antibody light and heavy chains such that a full-length antibody is expressed intracellularly. In another embodiment, the vector encodes a full-length light chain but only the VH/CH1 region of the heavy chain such that a Fab fragment is expressed intracellularly. In the most preferred embodiment, the vector encodes a single chain antibody (scFv) wherein the variable regions of the light and heavy chains are linked by a flexible peptide linker (e.g., (Gly₄Ser)₃) and expressed as a single chain molecule. To inhibit mCRISPC or rCRISPC activity in a cell, the expression vector encoding the anti-mCRISPC or anti-rCRISPC intracellular antibody is introduced into the cell by standard transfection methods, as discussed herein.

IV. Recombinant Expression Vectors and Host Cells

Another aspect of the invention pertains to vectors, preferably expression vectors, containing a nucleic acid encoding an mCRISPC or rCRISPC protein (or a portion thereof). The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operably linked to the nucleic acid sequence to be expressed. The term “regulatory sequence” is intended to include promoters, enhancers, and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., mCRISPC or rCRISPC proteins, mutant forms of mCRISPC or rCRISPC proteins, fusion proteins, and the like).

The recombinant expression vectors of the invention can be designed for expression of mCRISPC or rCRISPC proteins or protein fragments in prokaryotic or eukaryotic cells. For example, mCRISPC or rCRISPC proteins can be expressed in bacterial cells such as E. coli, insect cells (using baculovirus expression vectors), yeast cells, or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin, and enterokinase. Typical fusion expression vectors include, for example, pGEX (Pharmacia Biotech Inc; Smith D B and Johnson K S, Gene 67:31-40 (1988)) and pMAL (New England Biolabs, Beverly, Mass.) which fuse glutathione S-transferase (GST) or maltose E binding protein, respectively, to the target recombinant protein.

Purified fusion proteins can be utilized, for example, in mCRISPC or rCRISPC activity assays, (e.g., direct assays or competitive assays described in detail below), or to generate antibodies specific for mCRISPC or rCRISPC proteins.

Examples of suitable inducible non-fusion E. coli expression vectors include pTrc (Amann E et al., Gene 69:301-15 (1988)) and pET 11d (Studier et al., Gene Expression Technology: Methods in Enzymology 185, Academic Press, San Diego, Calif. (1990) pp. 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman S, Gene Expression Technology: Methods in Enzymology 185, Academic Press, San Diego, Calif. (1990) pp. 119-28). Another strategy is to alter the nucleic acid sequence of the nucleic acid to be inserted into an expression vector so that the individual codons for each amino acid are those preferentially utilized in E. coli (Wada K et al., Nucleic Acids Res. 20(Suppl.):2111-18 (1992)). Such alteration of nucleic acid sequences of the invention can be carried out by standard DNA synthesis techniques.

In another embodiment, the mCRISPC or rCRISPC expression vector is a yeast expression vector. Examples of vectors for expression in yeast S. cerivisae include pYepSec1 (Baldari C et al., EMBO J. 6:229-34 (1987)), pMFa (Kurjan J and Herskowitz I, Cell 30:933-43 (1982)), pJRY88 (Schultz L D et al., Gene 54:113-23 (1987)), pYES2 (Invitrogen Corporation, San Diego, Calif.), and picZ (Invitrogen Corp, San Diego, Calif.).

Alternatively, mCRISPC or rCRISPC proteins or polypeptides can be expressed in insect cells using baculovirus expression vectors. Baculovirus vectors available for expression of proteins in cultured insect cells (e.g., Sf 9 cells) include the pAc series (Smith G E et al., Mol. Cell. Biol. 3:2156-65 (1983)) and the pVL series (Lucklow V A and Summers M D, Virology 170:31-39 (1989)).

In yet another embodiment, a nucleic acid of the invention is expressed in mammalian cells using a mammalian expression vector. Examples of mammalian expression vectors include pCDM8 (Seed B, Nature 329:840-41 (1987)) and pMT2PC (Kaufman R J et al., EMBO J. 6:187-95 (1987)). When used in mammalian cells, the expression vector's control functions are often provided by viral regulatory elements. For example, commonly used promoters are derived from polyoma, Adenovirus 2, cytomegalovirus, and Simian Virus 40. For other suitable expression systems for both prokaryotic and eukaryotic cells, see chapters 16 and 17 of Sambrook, J., Fritsh, E. F., and Maniatis, T. Molecular Cloning: A Laboratory Manual, 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert C A et al., Genes Dev. 1:268-77 (1987)), lymphoid-specific promoters (Calame K and Eaton S, Adv. Immunol. 43:235-75 (1988)), in particular promoters of T cell receptors (Winoto A and Baltimore D, EMBO J. 8:729-33 (1989)) and immunoglobulins (Banerji J et al., Cell 33:729-40 (1983); Queen C and Baltimore D, Cell 33:741-48 (1983)), neuron-specific promoters (e.g., the neurofilament promoter; Byrne G W and Ruddle F H, Proc. Natl. Acad. Sci. USA 86:5473-77 (1989)), pancreas-specific promoters (Edlund T et al., Science 230:912-16 (1985)), and mammary gland-specific promoters (e.g., milk whey promoter; U.S. Pat. No. 4,873,316 and EP 0 264 166). Developmentally-regulated promoters are also encompassed, for example the murine hox promoters (Kessel M and Gruss P, Science 249:374-79 (1990)) and the α-fetoprotein promoter (Camper S A and Tilghman S M, Genes Dev. 3:537-46 (1989)).

Moreover, inducible regulatory systems for use in mammalian cells are known in the art, for example systems in which gene expression is regulated by heavy metal ions (see e.g., Mayo K E et al., Cell 29:99-108 (1982); Brinster R L et al., Nature 296:39-42 (1982); Searle P F et al., Mol. Cell. Biol. 5:1480-89 (1985)), heat shock (see e.g., Nouer L et al. (1991) in Heat Shock Response, ed. Nouer L, CRC, Boca Raton, Fla., pp. 167-220), hormones (see e.g., Lee F et al., Nature 294:228-32 (1981); Hynes N E et al., Proc. Natl. Acad. Sci. USA 78:2038-42 (1981); Klock G et al., Nature 329:734-36 (1987); Israel D I and Kaufman R J, Nucleic Acids Res. 17:2589-2604 (1989); WO 93/23431), FK506-related molecules (see e.g., WO 94/18317) or tetracyclines (Gossen M and Bujard H, Proc. Natl. Acad. Sci. USA 89:5547-51 (1992); Gossen M et al., Science 268:1766-69 (1995); WO 94/29442; WO 96/01313). Accordingly, in another embodiment, the invention provides a recombinant expression vector in which an mCRISPC or rCRISPC DNA is operably linked to an inducible eukaryotic promoter, thereby allowing for inducible expression of an mCRISPC or rCRISPC protein in eukaryotic cells.

Also known in the art are methods for expressing endogenous proteins using one-arm homologous recombination (see, e.g., U.S. Published Patent Application No. 2005/0003367; Zeh et al., Assay Drug Dev. Technol. 1:755-65 (2003); Qureshi et al., Assay Drug Dev. Technol. 1:767-76 (2003)). Briefly, an isolated genomic construct comprising a promoter operably linked to an mCRISPC or rCRISPC targeting sequence is introducing into a homogeneous population of cells (such as, for example, a homogeneous population of a human cell line or a homogeneous population of the murine epididymal epithelial cell line as described supra). The promoter is heterologous to the CRISPC target gene. Following recombination, the promoter controls transcription of an mRNA that encodes a CRISPC polypeptide. The population of cells is then incubated under conditions which cause expression of the CRISPC polypeptide.

The inventiori further provides a recombinant expression vector comprising a DNA molecule of the invention cloned into the expression vector in an antisense orientation. That is, the DNA molecule is operably linked to a regulatory sequence in a manner which allows for expression (by transcription of the DNA molecule) of an RNA molecule which is antisense to mCRISPC or rCRISPC mRNA. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific, or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes, see Weintraub H et al., Trends Genet. 1:22-25 (1985).

Another aspect of the invention pertains to host cells into which a recombinant expression vector of the invention has been introduced. For example, an mCRISPC or rCRISPC protein can be expressed in bacterial cells (such as, for example, E. coli), insect cells, yeast, or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms. “transformation” and “transfection” are intended to refer to a variety of art-recognized techniques for introducing foreign nucleic acid (e.g., DNA) into a host cell, including, for example, calcium phosphate or calcium chloride co-precipitation, DEAE-dextran-mediated transfection, lipofection, or electroporation. Suitable methods for transforming or transfecting host cells can be found in Sambrook et al. (Molecular Cloning: A Laboratory Manual, 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin, and methotrexate. Nucleic acid encoding a selectable marker can be introduced into a host cell on the same vector as that encoding an mCRISPC or rCRISPC protein or can be introduced on a separate vector. Cells stably transfected with the introduced nucleic acid can be identified by drug selection (e.g., cells that have incorporated the selectable marker gene will survive, while the other cells die).

In the case of epididymal cells which are stably transfected with mCRISPC or rCRISPC, such lines can be made such that the mCRISPC or rCRISPC gene is inducible, for example, using a murine epididymal epithelial cell line generated from primary epididymal cells infected with the SV40 large T-antigen (growth medium supplemented with D-valine to suppress non-epithelial cell growth, and is either +/−testosterone). For example, the regulation of the expressed gene can be brought about by the double stable expression first of a “regulator” plasmid, which contains the tet-controlled transactivator (tTA) and a second “response” plasmid, which contains mCRISPC or rCRISPC, under the control of a promoter sequence that includes the tetracycline response element (TRE). The commercially available regulator plasmids are in vectors engineered for neomycin selection, necessitating that response vectors be constructed to include a second selectable marker. Using such methods, mCRISPC or rCRISPC expression can be turned off in the presence of an agent, for example, tetracycline or a tetracycline-related compound (e.g., doxycycline) and turned on when the agent, for example, tetracycline, is not added to the culture medium. Construction of this type of cell line permits the stable expression of mCRISPC or rCRISPC in cells in which it is normally toxic.

A host cell of the invention, such as a prokaryotic or eukaryotic host cell in culture, can be used to produce (i.e., express) an mCRISPC or rCRISPC protein. Accordingly, the invention further provides methods for producing an mCRISPC or rCRISPC protein using the host cells of the invention. In one embodiment, the method comprises culturing the host cell of invention (into which a recombinant expression vector encoding an mCRISPC or rCRISPC protein has been introduced) in a suitable medium such that an mCRISPC or rCRISPC protein is produced. In another embodiment, the method further comprises isolating an mCRISPC or rCRISPC protein from the medium or the host cell.

Certain host cells of the invention can also be used to produce non-human transgenic animals. For example, in one embodiment, a host cell of the invention is a fertilized oocyte or an embryonic stem cell into which mCRISPC- or rCRISPC-coding sequences have been introduced. Such host cells can then be used to create non-human transgenic animals in which exogenous mCRISPC or rCRISPC sequences have been introduced into their genome or homologous recombinant animals in which endogenous mCRISPC or rCRISPC sequences have been altered. Such animals are useful for studying the function and/or activity of an mCRISPC or rCRISPC polypeptide and for identifying and/or evaluating modulators of mCRISPC or rCRISPC activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous mCRISPC or rCRISPC gene has been altered by homologous recombination between the endogenous gene and an exogenous DNA molecule introduced into a cell of the animal, for example, an embryonic cell of the animal, prior to development of the animal.

A transgenic animal of the invention can be created by introducing an mCRISPC- or rCRISPC-encoding nucleic acid into the male pronucleus of a fertilized oocyte, e.g., by microinjection, retroviral infection, and allowing the oocyte to develop in a pseudopregnant female foster animal. The mCRISPC or rCRISPC sequence of SEQ ID NO:3 or SEQ ID NO:4 or portion thereof can be introduced as a transgene into the genome of a non-human animal. Alternatively, an mCRISPC or rCRISPC gene homologue, such as another mCRISPC or rCRISPC family member, can be isolated based on hybridization to the mCRISPC or rCRISPC family cDNA sequences of nucleotides 97-846 of SEQ ID NO:19 or nucleotides 161-922 of SEQ ID NO:25 (described further above) and used as a transgene.

Intronic sequences and polyadenylation signals can also be included in the transgene to increase the efficiency of expression of the transgene. A tissue-specific regulatory sequence(s) can be operably linked to an mCRISPC or rCRISPC transgene to direct expression of a mCRISPC or rCRISPC protein to particular cells. Methods for generating transgenic animals via embryo manipulation and microinjection, particularly animals such as mice, have become conventional in the art and are described, for example, in U.S. Pat. Nos. 4,736,866; 4,870,009; 4,873,191; and in Hogan B, Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of an mCRISPC or rCRISPC transgene in its genome and/or expression of mCRISPC or rCRISPC mRNA in tissues or cells of the animals. A transgenic founder animal can then be used to breed additional animals carrying the transgene. Moreover, transgenic animals carrying a transgene encoding an mCRISPC or rCRISPC protein can further be bred to other transgenic animals carrying other transgenes.

To create a homologous recombinant animal, a vector is prepared which contains at least a portion of an mCRISPC or rCRISPC gene into which a deletion, addition, or substitution has been introduced to thereby alter, for example, functionally disrupt, the mCRISPC or rCRISPC gene. In a preferred embodiment, the vector is designed such that, upon homologous recombination, the endogenous mCRISPC or rCRISPC gene is functionally disrupted (i.e., no longer encodes a functional protein; also referred to as a “knock out” vector). Alternatively, the vector can be designed such that, upon homologous recombination, the endogenous mCRISPC or rCRISPC gene is mutated or otherwise altered but still encodes a functional protein (e.g., the upstream regulatory region can be altered to thereby alter the expression of the endogenous mCRISPC or rCRISPC protein). In the homologous recombination vector, the altered portion of the mCRISPC or rCRISPC gene is flanked at its 5′ and 3′ ends by additional nucleic acid sequence of the mCRISPC or rCRISPC gene to allow for homologous recombination to occur between the exogenous mCRISPC or rCRISPC gene carried by the vector and an endogenous mCRISPC or rCRISPC gene in an embryonic stem cell. The additional flanking mCRISPC or rCRISPC nucleic acid sequence is of sufficient length for successful homologous recombination with the endogenous gene. Typically, several kilobases of flanking DNA (both at the 5′ and 3′ ends) are included in the vector (see, e.g., Thomas K R and Capecchi M R, Cell 51:503-12 (1987) for a description of homologous recombination vectors). The vector is introduced into an embryonic stem cell line (e.g., by electroporation) and cells in which the introduced mCRISPC or rCRISPC gene has homologously recombined with the endogenous mCRISPC or rCRISPC gene are selected (see, e.g., Li E et al., Cell 69:915-26 (1992)). The selected cells are then injected into a blastocyst of an animal (e.g., a mouse) to form aggregation chimeras (see, e.g., Bradley A in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal, and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in, for example, Bradley A, Curr. Opin. Biotechnol. 2:823-29 (1991); WO 90/11354; WO 91/01140; WO 92/0968; and WO 93/04169.

In addition to the foregoing, the skilled artisan will appreciate that other approaches known in the art for homologous recombination can be applied to the instant invention. Enzyme-assisted site-specific integration systems are known in the art and can be applied to integrate a DNA molecule at a predetermined location in a second target DNA molecule. Examples of such enzyme-assisted integration systems include the Cre recombinase-lox target system (e.g., as described in Baubonis W and Sauer B, Nucleic Acids Res. 21:2025-29 (1993); and Fukushige S and Sauer B, Proc. Natl. Acad. Sci. USA 89:7905-09 (1992)) and the FLP recombinase-FRT target system (e.g., as described in Dang D T and Perrimon N, Dev. Genet. 13:367-75 (1992); and Fiering S et al., Proc. Natl. Acad. Sci. USA 90:8469-73 (1993)). Tetracycline-regulated inducible homologous recombination systems, such as those described in WO 94/29442 and WO 96/01313, also can be used.

For example, in another embodiment, transgenic non-humans animals can be produced which contain selected systems which allow for regulated expression of the transgene. One example of such a system is the cre/loxP recombinase system of bacteriophage P1. For a description of the cre/loxP recombinase system, see, e.g., Lakso M et al., Proc. Natl. Acad. Sci. USA 89:6232-36 (1992). Another example of a recombinase system is the FLP recombinase system of Saccharomyces cerevisiae (O'Gorman S et al., Science 251:1351-55 (1991)). If a cre/loxP recombinase system is used to regulate expression of the transgene, animals containing transgenes encoding both the Cre recombinase and a selected protein are required. Such animals can be provided through the construction of “double” transgenic animals, for example, by mating two transgenic animals, one containing a transgene encoding a selected protein and the other containing a transgene encoding a recombinase.

Clones of the non-human transgenic animals described herein can also be produced according to the methods described in, for example, Wilmut I et al., Nature 385:810-13 (1997); WO 97/07668; and WO 97/07669. In brief, a cell, for example, a somatic cell, from the transgenic animal can be isolated and induced to exit the growth cycle and enter G_{O} phase. The quiescent cell can then be fused, for example, through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, for example, the somatic cell, is isolated.

V. Uses and Methods of the Invention

The nucleic acid molecules, proteins, protein homologues, and antibodies described herein can be used in one or more of the following methods: a) methods of treatment, preferably in epididymal cells; b) screening assays; c) predictive medicine (e.g., diagnostic assays, prognostic assays, monitoring clinical trials, or pharmacogenetics). The isolated nucleic acid molecules of the invention can be used, for example, to express mCRISPC or rCRISPC protein (e.g., via a recombinant expression vector in a host cell in gene therapy applications), to detect mCRISPC or rCRISPC mRNA (e.g., in a biological sample) or a genetic alteration in an mCRISPC or rCRISPC gene, and to modulate mCRISPC or rCRISPC activity, as described further below. In addition, the mCRISPC or rCRISPC proteins can be used to screen for naturally occurring mCRISPC or rCRISPC binding proteins, to screen for drugs or compounds which modulate mCRISPC or rCRISPC activity, as well as to treat disorders that would benefit from modulation of mCRISPC or rCRISPC, for example, characterized by insufficient or excessive production of mCRISPC or rCRISPC protein or production of mCRISPC or rCRISPC protein forms which have decreased or aberrant activity compared to mCRISPC or rCRISPC wild type protein. Moreover, the anti-mCRISPC or anti-rCRISPC antibodies of the invention can be used to detect and isolate mCRISPC or rCRISPC proteins, regulate the bioavailability of mCRISPC or rCRISPC proteins, and modulate mCRISPC or rCRISPC activity, for example, modulate inhibition of capacitation and/or modulation of sperm-egg fusion. In preferred embodiments the methods of the invention, for example, detection, modulation of mCRISPC or rCRISPC, etc. are performed in isolated sperm.

A. Methods of Modulating CRISPC

The present invention provides for methods of modulating mCRISPC or rCRISPC in a cell, for example, for the purpose of identifying agents that modulate mCRISPC or rCRISPC expression and/or activity, as well as both prophylactic and therapeutic methods of treating a subject at risk of (or susceptible to) a disorder or having a disorder associated with aberrant mCRISPC or rCRISPC expression or activity or a disorder that would benefit from modulation of mCRISPC or rCRISPC activity.

Yet another aspect of the invention pertains to methods of modulating MCRISPC or rCRISPC expression and/or activity in a cell. The modulatory methods of the invention involve contacting the cell with an agent that modulates mCRISPC or rCRISPC expression and/or activity such that MCRISPC or rCRISPC expression and/or activity in the cell is modulated. The agent may act by modulating the activity of mCRISPC or rCRISPC protein in the cell or by modulating transcription of the mCRISPC or rCRISPC gene or translation of the mCRISPC or rCRISPC mRNA.

Accordingly, in one embodiment, the agent inhibits mCRISPC or rCRISPC activity. An inhibitory agent may function, for example, by directly inhibiting mCRISPC or rCRISPC inhibition of capacitation and/or modulation sperm-egg fusion activity or by modulating a signaling pathway which negatively regulates mCRISPC or rCRISPC. In another embodiment, the agent stimulates mCRISPC or rCRISPC activity. A stimulatory agent may function, for example, by directly stimulating mCRISPC or rCRISPC inhibition of capacitation and/or modulation of sperm-egg fusion activity, or by modulating a signaling pathway that leads to stimulation of mCRISPC or rCRISPC activity. Exemplary inhibitory agents include antisense mCRISPC or rCRISPC nucleic acid molecules (e.g., to inhibit translation of mCRISPC or rCRISPC mRNA), intracellular anti-mCRISPC or anti-rCRISPC antibodies (e.g., to inhibit the activity of mCRISPC or rCRISPC protein), and dominant negative mutants of the mCRISPC or rCRISPC protein. Other inhibitory agents that can be used to inhibit the activity of an mCRISPC or rCRISPC protein are chemical compounds that inhibit mCRISPC or rCRISPC inhibition of capacitation and/or modulation of sperm-egg fusion activity. Such compounds can be identified using screening assays that select for such compounds, as described herein. Additionally or alternatively, compounds that inhibit mCRISPC or rCRISPC inhibition of capacitation and/or modulation of sperm-egg fusion activity can be designed using approaches known in the art.

According to another modulatory method of the invention, mCRISPC or rCRISPC activity is stimulated in a cell by contacting the cell with a stimulatory agent. Examples of such stimulatory agents include active mCRISPC or rCRISPC protein and nucleic acid molecules encoding mCRISPC or rCRISPC that are introduced into the cell to increase mCRISPC or rCRISPC activity in the cell. A preferred stimulatory agent is a nucleic acid molecule encoding an mCRISPC or rCRISPC protein, wherein the nucleic acid molecule is introduced into the cell in a form suitable for expression of the active mCRISPC or rCRISPC protein in the cell. To express an mCRISPC or rCRISPC protein in a cell, typically an mCRISPC or rCRISPC cDNA is first introduced into a recombinant expression vector using standard molecular biology techniques, as described herein. An mCRISPC or rCRISPC cDNA can be obtained, for example, by amplification using the PCR or by screening an appropriate cDNA library as described herein. Following isolation or amplification of mCRISPC or rCRISPC cDNA, the DNA fragment is introduced into an expression vector and transfected into target cells by standard methods, as described herein. Other stimulatory agents that can be used to stimulate the activity and/or expression of an mCRISPC or rCRISPC protein are chemical compounds that stimulate CRISPC activity and/or expression in cells, such as compounds that enhance CRISPC inhibition of capacitation and/or modulation of sperm-egg fusion activity. Such compounds can be identified using screening assays that select for such compounds, as described in detail herein.

The modulatory methods of the invention can be performed in vitro (e.g., by culturing the cell with the agent or by introducing the agent into cells in culture) or, alternatively, in vivo (e.g., by administering the agent to a subject or by introducing the agent into cells of a subject, such as by gene therapy). For practicing the modulatory method in vitro, cells can be obtained from a subject by standard methods and incubated (i.e., cultured) in vitro with a modulatory agent of the invention to modulate mCRISPC or rCRISPC activity in the cells.

For stimulatory or inhibitory agents that comprise nucleic acids (including recombinant expression vectors encoding mCRISPC or rCRISPC protein, antisense RNA, intracellular antibodies, or dominant negative inhibitors), the agents can be introduced into cells of the subject using methods known in the art for introducing nucleic acid (e.g., DNA) into cells in vivo. Examples of such methods encompass both non-viral and viral methods, including:

Direct Injection: Naked DNA can be introduced into cells in vivo by directly injecting the DNA into the cells (see, e.g., Acsadi G et al., Nature 332:815-18 (1991); Wolff J A et al., Science 247:1465-68 (1990)). For example, a delivery apparatus (e.g., a “gene gun”) for injecting DNA into cells in vivo can be used. Such an apparatus is commercially available (e.g., from Bio-Rad Laboratories, Hercules, Calif.).

Cationic Lipids: Naked DNA can be introduced into cells in vivo by complexing the DNA with cationic lipids or encapsulating the DNA in cationic liposomes. Examples of suitable cationic lipid formulations include N-[-1-(2,3-dioleoyloxy)propyl]N,N,N-triethylammonium chloride (DOTMA) and a 1:1 molar ratio of 1,2-dimyristyloxy-propyl-3-dimethylhydroxyethylammonium bromide (DMRIE) and dioleoyl phosphatidylethanolamine (DOPE) (see e.g., Logan J J et al., Gene Ther. 2:38-49 (1995); San H et al., Hum. Gene Ther. 4:781-88 (1993)).

Receptor-Mediated DNA Uptake: Naked DNA can also be introduced into cells in vivo by complexing the DNA to a cation, such as polylysine, which is coupled to a ligand for a cell-surface receptor (see, e.g., Wu G Y and Wu C H, J. Biol. Chem. 263:14621-24 (1988); Wilson J M et al., J. Biol. Chem. 267:963-67 (1992); and U.S. Pat. No. 5,166,320). Binding of the DNA-ligand complex to the receptor facilitates uptake of the DNA by receptor-mediated endocytosis. A DNA-ligand complex linked to adenovirus capsids which naturally disrupt endosomes, thereby releasing material into the cytoplasm can be used to avoid degradation of the complex by intracellular lysosomes (see, e.g., Curiel D T et al., Proc. Natl. Acad. Sci. USA 88:8850-54 (1991); Cristiano R J et al., Proc. Natl. Acad. Sci. USA 90:2122-26 (1993)).

Retroviruses: Defective retroviruses are well characterized for use in gene transfer for gene therapy purposes (for a review, see Miller A D, Blood 76:271-78 (1990)). A recombinant retrovirus can be constructed having a nucleotide sequences of interest incorporated into the retroviral genome. Additionally, portions of the retroviral genome can be removed to render the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel F M et al., (eds.) Greene Publishing Associates, (1989), Sections 9.10-9.14 and other standard laboratory manuals. Examples of suitable retroviruses include pLJ, PZIP, pWE, and pEM which are well known to those skilled in the art. Examples of suitable packaging virus lines include ψCrip, ψCre, ψ2 and ψAm. Retroviruses have been used to introduce a variety of genes into many different cell types, including epithelial cells, endothelial cells, lymphocytes, myoblasts, hepatocytes, bone marrow cells, in vitro and/or in vivo (see, e.g., Eglitis M A et al., Science 230:1395-98 (1985); Danos O and Mulligan R C, Proc. Natl. Acad. Sci. USA 85:6460-64 (1988); Wilson J M et al., Proc. Natl. Acad. Sci. USA 85:3014-18 (1988); Armentano D et al., Proc. Natl. Acad. Sci. USA 87:6141-45 (1990); Huber B E et al., Proc. Natl. Acad. Sci. USA 88:8039-43 (1991); Ferry N et al., Proc. Natl. Acad. Sci. USA 88:8377-81 (1991); Chowdhury J R et al., Science 254:1802-05 (1991); van Beusechem V W et al., Proc. Natl. Acad. Sci. USA 89:7640-44 (1992); Kay M A et al., Hum. Gene Ther. 3:641-47 (1992); Dai Y et al., Proc. Natl. Acad. Sci. USA 89:10892-95 (1992); Hwu P et al., J. Immunol. 150:4104-15 (1993); U.S. Pat. No. 4,868,116; U.S. Pat. No. 4,980,286; WO 89/07136; WO 89/02468; WO 89/05345; and WO 92/07573). Retroviral vectors require target cell division in order for the retroviral genome (and foreign nucleic acid inserted into it) to be integrated into the host genome to stably introduce nucleic acid into the cell. Thus, it may be necessary to stimulate replication of the target cell.

Adenoviruses: The genome of an adenovirus can be manipulated such that it encodes and expresses a gene product of interest but is inactivated in terms of its ability to replicate in a normal lytic viral life cycle (see, e.g., Berkner K L, Biotechniques 6:616-29 (1988); Rosenfeld M A et al., Science 252:431-34 (1991); and Rosenfeld M A et al., Cell 68:143-55 (1992)). Suitable adenoviral vectors derived from the adenovirus strain Ad type 5 d1324 or other strains of adenovirus (e.g., Ad2, Ad3, Ad7, etc.) are well known to those skilled in the art. Recombinant adenoviruses are advantageous in that they do not require dividing cells to be effective gene delivery vehicles and can be used to infect a wide variety of cell types, including airway epithelium (Rosenfeld M A et al., Cell 68:143-55 (1992)), endothelial cells (Lemarchand P et al., Proc. Natl. Acad. Sci. USA 89:6482-86 (1992)), hepatocytes (Herz J and Gerard R D, Proc. Natl. Acad. Sci. USA 90:2812-16 (1993)), and muscle cells (Quantin B et al., Proc. Natl. Acad. Sci. USA 89:2581-84 (1992)). Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner K L et al., supra; Haj-Ahmad Y and Graham F L, J. Virol. 57:267-74 (1986)). Most replication-defective adenoviral vectors currently in use are deleted for all or parts of the viral E1 and E3 genes but retain as much as 80% of the adenoviral genetic material.

Adeno-Associated Viruses: Adeno-associated virus (AAV) is a naturally occurring defective virus that requires another virus, such as an adenovirus or a herpes virus, as a helper virus for efficient replication and a productive life cycle (for a review, see Muzyczka N, Curr. Top. Microbiol. Immunol. 158:97-129 (1992)). It is also one of the few viruses that may integrate its DNA into non-dividing cells, and exhibits a high frequency of stable integration (see, e.g., Flotte T R et al., Am. J. Respir. Cell. Mol. Biol. 7:349-56 (1992); Samulski R J et al., J. Virol. 63:3822-28 (1989); and McLaughlin S K et al., J. Virol. 62:1963-73 (1988)). Vectors containing as little as 300 base pairs of AAV can be packaged and can integrate. Space for exogenous DNA is limited to about 4.5 kb. An AAV vector such as that described in Tratschin J D et al., Mol. Cell. Biol. 5:3251-60 (1985), can be used to introduce DNA into cells. A variety of nucleic acids have been introduced into different cell types using AAV vectors (see, e.g., Hermonat P L and Muzyczka N, Proc. Natl. Acad. Sci. USA 81:6466-70 (1984); Tratschin J D et al., Mol. Cell. Biol. 4:2072-81 (1985); Wondisford F E et al., Mol. Endocrinol. 2:32-39 (1988); Tratschin J D et al., J. Virol. 51:611-19 (1984); and Flotte T R et al., J. Biol. Chem. 268:3781-90 (1993)).

The efficacy of a particular expression vector system and method of introducing nucleic acid into a cell can be assessed by standard approaches routinely used in the art. For example, DNA introduced into a cell can be detected by a filter hybridization technique (e.g., Southern blotting) and RNA produced by transcription of introduced DNA can be detected, for example, by Northern blotting, RNase protection, or reverse transcriptase-polymerase chain reaction (RT-PCR). The gene product can be detected by an appropriate assay, for example by immunological detection of a produced protein, such as with a specific antibody, or by a functional assay to detect a functional activity of the gene product.

There are a variety of pathological conditions for which mCRISPC or rCRISPC modulating agents of the present invention can be used in treatment. For example, while the exact activity of CRISPC is not yet know uses for CRISPC may include the treatment or diagnosis of infertility and use as a target of or is itself a contraceptive agent. Based on the protein domains of CRISPC described infra, possible activities biochemical activities include, for example, endopeptidase activity and/or potassium channel blocking activity.

1. Prophylactic Methods

In one aspect, the invention provides a method for preventing in a subject, a disease or condition that would benefit from modulation of CRISPC activity and/or expression, e.g., a disorder associated with an aberrant CRISPC expression or activity, by administering to the subject an mCRISPC or rCRISPC polypeptide or an agent which modulates CRISPC polypeptide expression or at least one CRISPC activity. Subjects at risk for a disease which is caused or contributed to by aberrant CRISPC expression or activity can be identified by, for example, any or a combination of diagnostic or prognostic assays as described herein. Administration of a prophylactic agent can occur prior to the manifestation of symptoms characteristic of CRISPC aberrance, such that a disease or disorder is prevented or, alternatively, delayed in its progression. Depending on the type of CRISPC aberrance or condition, for example, an mCRISPC or rCRISPC polypeptide, mCRISPC or rCRISPC agonist, or mCRISPC or rCRISPC antagonist agent can be used for treating the subject. The appropriate agent can be determined based on screening assays described herein.

2. Therapeutic Methods

Another aspect of the invention pertains to methods of modulating CRISPC expression or activity for therapeutic purposes. Accordingly, in an exemplary embodiment, the modulatory method of the invention involves contacting a cell with an mCRISPC or rCRISPC polypeptide or agent that modulates one or more of the activities of CRISPC protein associated with the cell. An agent that modulates CRISPC protein activity can be an agent as described herein, such as a nucleic acid or a protein, a naturally-occurring target molecule of a CRISPC protein (e.g., a CRISPC binding protein), an mCRISPC or rCRISPC antibody, an mCRISPC or rCRISPC agonist or antagonist, a peptidomimetic of an mCRISPC or rCRISPC agonist or antagonist, or other small molecule. In one embodiment, the agent stimulates one or more CRISPC activities. Examples of such stimulatory agents include active mCRISPC or rCRISPC protein and a nucleic acid molecule encoding mCRISPC or rCRISPC polypeptide that has been introduced into the cell. In another embodiment, the agent inhibits one or more CRISPC activities. Examples of such inhibitory agents include, e.g., antisense mCRISPC or rCRISPC nucleic acid molecules, anti-mCRISPC or anti-rCRISPC antibodies, and mCRISPC or rCRISPC inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder that would benefit from modulation of a CRISPC protein, e.g., infertility, or which is characterized by aberrant expression or activity of a CRISPC protein or nucleic acid molecule. In one embodiment, the method involves administering an agent (e.g., an agent identified by a screening assay described herein), or combination of agents that modulates (e.g., upregulates or downregulates) CRISPC expression or activity. In another embodiment, the method involves administering an mCRISPC or rCRISPC protein or nucleic acid molecule as therapy to compensate for reduced or aberrant CRISPC expression or activity.

Stimulation of CRISPC activity is desirable in situations in which CRISPC is abnormally downregulated and/or in which increased CRISPC activity is likely to have a beneficial effect. Likewise, inhibition of CRISPC activity is desirable in situations in which CRISPC is abnormally upregulated and/or in which decreased CRISPC activity is likely to have a beneficial effect. Exemplary situations in which CRISPC modulation will be desirable are in the treatment of CRISPC associated disorders. For example, epididymis-specific proteins have been shown to be useful in vaccine-based contraceptive approaches, as in the example of the protein eppin in primates (see O'Rand et al., Science 306:1189-90 (2004)). The polypeptides or fragments of those polypeptides resulting from the polynucleotides described herein may also be useful as antigens in immunocontraceptive approaches.

B. Screening Assays:

The invention provides a method (also referred to herein as a “screening assay”) for identifying modulators, that is, candidate or test compounds or agents (e.g., peptides, peptidomimetics, small molecules, or other drugs) which bind to mCRISPC or rCRISPC proteins, have a stimulatory or inhibitory effect on, for example, mCRISPC or rCRISPC expression or mCRISPC or rCRISPC activity.

The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer, or small molecule libraries of compounds (Lam K S, Anticancer Drug Des. 12:145-67 (1997)).

Examples of methods for the synthesis of molecular libraries can be found in the art, for example in: DeWitt S H et al., Proc. Natl. Acad. Sci. USA 90:6909-13 (1993); Erb E et al., Proc. Natl. Acad. Sci. USA 91:11422-26 (1994); Zuckermann R N et al., J. Med. Chem. 37:2678-85 (1994); Cho C Y et al., Science 261:1303-05 (1993); Carrell T et al., Angew. Chem. Int. Ed. Engl. 33:2059-61 (1994); Carrell T et al., Angew. Chem. Int. Ed. Engl. 33:2061-64 (1994); and Gallop M A et al., J. Med. Chem. 37:1233-51 (1994).

Libraries of compounds may be presented in solution (e.g., Houghten R A et al., Biotechniques 13:412-21 (1992)), or on beads (Lam K S et al., Nature 354:82-84 (1991)), chips (Fodor S P A et al., Nature 364:555-56 (1993)), bacteria (U.S. Pat. No. 5,223,409), spores (U.S. Pat. No. 5,223,409), plasmids (Cull M G et al., Proc. Natl. Acad. Sci. USA 89:1865-69 (1992)), or on phage (Scott J K and Smith G P, Science 249:386-90 (1990); Devlin J J et al., Science 249:404-06 (1990); Cwirla S E et al., Proc. Natl. Acad. Sci. 87:6378-82 (1990); Felici F et al., J. Mol. Biol. 222:301-10 (1991); U.S. Pat. No. 5,223,409).

In many drug screening programs which test libraries of modulating agents and natural extracts, high throughput assays are desirable in order to maximize the number of modulating agents surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test modulating agent. Moreover, the effects of cellular toxicity and/or bioavailability of the test modulating agent can be generally ignored in the in vitro system, the assay instead being focused primarily on the effect of the drug on the molecular target as may be manifest in an alteration of binding affinity with upstream or downstream elements.

In one embodiment, the invention provides assays for screening candidate or test compounds which bind to or modulate the activity of a CRISPC protein or polypeptide or biologically active portion thereof, for example, modulate the ability of CRISPC polypeptide to inhibit capacitation and/or modulate sperm-egg fusion.

Assays can be used to screen for modulating agents, including mCRISPC or rCRISPC homologs, which are either agonists or antagonists of the normal cellular function of the subject mCRISPC or rCRISPC polypeptides. For example, the invention provides a method in which an indicator composition is provided which has an mCRISPC or rCRISPC protein having a CRISPC activity. The indicator composition can be contacted with a test compound. The effect of the test compound on CRISPC activity, as measured by a change in the indicator composition, can then be determined to thereby identify a compound that modulates the activity of a CRISPC protein. A statistically significant change, such as a decrease or increase, in the level of CRISPC activity in the presence of the test compound (relative to what is detected in the absence of the test compound) is indicative of the test compound being a CRISPC modulating agent. The indicator composition can be, for example, a cell or a cell extract.

The efficacy of the modulating agent can be assessed by generating dose response curves from data obtained using various concentrations of the test modulating agent. Moreover, a control assay can also be performed to provide a baseline for comparison. In the control assay, isolated and purified mCRISPC or rCRISPC protein is added to a composition containing the CRISPC-binding element, and the formation of a complex is quantitated in the absence of the test modulating agent.

In yet another embodiment, an assay of the present invention is a cell-free assay in which an mCRISPC or rCRISPC protein or biologically active portion thereof is contacted with a test compound and the ability of the test compound to bind to the mCRISPC or rCRISPC protein or biologically active portion thereof is determined. Binding of the test compound to the mCRISPC or rCRISPC protein can be determined either directly or indirectly as described above. In a preferred embodiment, the assay includes contacting the mCRISPC or rCRISPC protein or biologically active portion thereof with a known compound which binds mCRISPC or rCRISPC to form an assay mixture, contacting the assay mixture with a test compound, and determining the ability of the test compound to interact with an mCRISPC or rCRISPC protein, wherein determining the ability of the test compound to interact with an mCRISPC or rCRISPC protein comprises determining the ability of the test compound to preferentially bind to mCRISPC or rCRISPC polypeptide or biologically active portion thereof as compared to the known compound.

In another embodiment, the assay is a cell-free assay in which an mCRISPC or rCRISPC protein or biologically active portion thereof is contacted with a test compound and the ability of the test compound to modulate (e.g., stimulate or inhibit) the activity of the mCRISPC or rCRISPC protein or biologically active portion thereof is determined. The mCRISPC or rCRISPC protein can be provided as a lysate of cells that express mCRISPC or rCRISPC, as a purified or semipurified polypeptide, or as a recombinantly expressed polypeptide. In one embodiment, a cell-free assay system further comprises a cell extract or isolated components of a cell, such as mitochondria. Such cellular components can be isolated using techniques which are known in the art. Preferably, a cell free assay system further comprises at least one target molecule with which mCRISPC or rCRISPC interacts, and the ability of the test compound to modulate the interaction of the mCRISPC or rCRISPC with the target molecule(s) is monitored to thereby identify the test compound as a modulator of mCRISPC or rCRISPC, for example, inhibition of capacitation and/or modulation of sperm-egg fusion activity. Determining the ability of the test compound to modulate the activity of a mCRISPC or rCRISPC protein can be accomplished, for example, by determining the ability of the mCRISPC or rCRISPC protein to bind to a mCRISPC or rCRISPC target molecule by one of the methods described above for determining direct binding. Determining the ability of the mCRISPC or rCRISPC protein to bind to an mCRISPC or rCRISPC target molecule can also be accomplished using a technology such as real-time Biomolecular Interaction Analysis (BIA) (see, e.g., Sjolander S and Urbaniczky C, Anal. Chem. 63:2338-45 (1991) and SzaboA et al., Curr. Opin. Struct. Biol. 5:699-705 (1995)). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In yet another embodiment, the cell-free assay involves contacting an mCRISPC or rCRISPC protein or biologically active portion thereof with a known compound which binds the mCRISPC or rCRISPC protein to form an assay mixture, contacting the assay mixture with a test compound, and determining the ability of the test compound to interact with the mCRISPC or rCRISPC protein, wherein determining the ability of the test compound to interact with the mCRISPC or rCRISPC protein comprises determining the ability of the mCRISPC or rCRISPC protein to preferentially bind to or modulate the activity of an mCRISPC or rCRISPC target molecule.

The cell-free assays of the present invention are amenable to use of both soluble and/or membrane-bound forms of proteins (e.g., mCRISPC or rCRISPC proteins or receptors having intracellular domains to which mCRISPC or rCRISPC binds). In the case of cell-free assays in which a membrane-bound form a protein is used it may be desirable to utilize a solubilizing agent such that the membrane-bound form of the protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

An mCRISPC or rCRISPC target molecule can be, for example, a protein. Suitable assays are known in the art that allow for the detection of protein-protein interactions (e.g., immunoprecipitations, two-hybrid assays, and the like). By performing such assays in the presence and absence of test compounds, these assays can be used to identify compounds that modulate (e.g., inhibit or enhance) the interaction of mCRISPC or rCRISPC with a target molecule(s).

Determining the ability of the mCRISPC or rCRISPC protein to bind to or interact with a ligand of an mCRISPC or rCRISPC molecule can be accomplished, for example, by direct binding. In a direct binding assay, the mCRISPC or rCRISPC protein could be coupled with a radioisotope or enzymatic label such that binding of the mCRISPC or rCRISPC protein to an mCRISPC or rCRISPC target molecule can be determined by detecting the labeled mCRISPC or rCRISPC protein in a complex. For example, mCRISPC or rCRISPC molecules, for example, mCRISPC or rCRISPC proteins, can be labeled with, for example, ¹²⁵I, ³⁵S, ¹⁴C, or ³H, either directly or indirectly, and the radioisotope detected by direct counting of radioemmission or by scintillation counting. Alternatively, mCRISPC or rCRISPC molecules can be enzymatically labeled with, for example, horseradish peroxidase, alkaline phosphatase, or luciferase, and the enzymatic label detected by determination of conversion of an appropriate substrate to product.

Typically, it will be desirable to immobilize mCRISPC or rCRISPC or their respective binding proteins to facilitate separation of complexes from uncomplexed forms of one or both of the proteins, as well as to accommodate automation of the assay. Binding of MCRISPC or rCRISPC to an upstream or downstream binding element, in the presence and absence of a candidate agent, can be accomplished in any vessel suitable for containing the reactants. Examples include microtiter plates, test tubes, and micro-centrifuge tubes. In one embodiment, a fusion protein can be provided which adds a domain that allows the protein to be bound to a matrix. For example, glutathione-S-transferase/mCRISPC (GST/mCRISPC) or glutathione-S-transferase/rCRISPC (GST/rCRISPC) fusion proteins can be adsorbed onto glutathione sepharose beads (Sigma Chemical, St. Louis, Mo.) or glutathione derivatized microtiter plates, which are then combined with the cell lysates and the test modulating agent, and the mixture incubated under conditions conducive to complex formation, for example, at physiological conditions for salt and pH, though slightly more stringent conditions may be desired. Following incubation, the beads are washed to remove any unbound label, and the matrix immobilized and radiolabel determined directly (e.g., beads placed in scintillant), or in the supernatant after the complexes are subsequently dissociated. Alternatively, the complexes can be dissociated from the matrix, separated by SDS-PAGE, and the level of mCRISPC- or rCRISPC-binding protein found in the bead fraction quantitated from the gel using standard electrophoretic techniques.

Other techniques for immobilizing proteins on matrices are also available for use in the subject assay. For instance, mCRISPC or rCRISPC or their cognate binding protein can be immobilized utilizing conjugation of biotin and streptavidin. For instance, biotinylated mCRISPC or rCRISPC molecules can be prepared from biotin-NHS(N-hydroxy-succinimide) using techniques well known in the art (e.g., biotinylation kit, Pierce Biotechnology, Rockford, Ill.), and immobilized in the wells of streptavidin-coated 96 well plates (Pierce Biotechnology). Alternatively, antibodies reactive with mCRISPC or rCRISPC but which do not interfere with binding of upstream or downstream elements can be derivatized to the wells of the plate, and mCRISPC or rCRISPC trapped in the wells by antibody conjugation. As above, preparations of an mCRISPC- or rCRISPC-binding protein (mCRISPC- or rCRISPC-BP) and a test modulating agent are incubated in the mCRISPC- or rCRISPC-presenting wells of the plate, and the amount of complex trapped in the well can be quantitated. Exemplary methods for detecting such complexes, in addition to those described above for the GST-immobilized complexes, include immunodetection of complexes using antibodies reactive with the mCRISPC or rCRISPC binding element, or which are reactive with mCRISPC or rCRISPC protein and compete with the binding element; as well as enzyme-linked assays which rely on detecting an enzymatic activity associated with the binding element, either intrinsic or extrinsic activity. In the instance of the latter, the enzyme can be chemically conjugated or provided as a fusion protein with the mCRISPC binding protein or rCRISPC binding protein. To illustrate, the mCRISPC binding protein or rCRISPC binding protein can be chemically cross-linked or genetically fused with horseradish peroxidase, and the amount of protein trapped in the complex can be assessed with a chromogenic substrate of the enzyme, for example, 3,3′-diamino-benzadine terahydrochloride or 4-chloro-1-napthol. Likewise, a fusion protein comprising the protein and glutathione-S-transferase can be provided, and complex formation quantitated by detecting the GST activity using 1-chloro-2,4-dinitrobenzene (Habig W H et al., J. Biol. Chem. 249:7130-39 (1974)).

For processes which rely on immunodetection for quantitating one of the proteins trapped in the complex, antibodies against the protein, such as anti-mCRISPC or anti-rCRISPC antibodies, can be used. Alternatively, the protein to be detected in the complex can be “epitope tagged” in the form of a fusion protein which includes, in addition to the mCRISPC or rCRISPC sequence, a second protein for which antibodies are readily available (e.g. from commercial sources). For instance, the GST fusion proteins described above can also be used for quantification of binding using antibodies against the GST moiety. Other useful epitope tags include myc-epitopes (see, e.g., Ellison M J and Hochstrasser M, J. Biol. Chem. 266:21150-57 (1991)) which includes a 10-residue sequence from c-myc, as well as the pFLAG® system (SigmaAldrich, St. Louis, Mo.) or the pEZZ-protein A system (GE Healthcare, Piscataway, N.J.).

It is also within the scope of this invention to determine the ability of a compound to modulate the interaction between mCRISPC or rCRISPC and their respective target molecules without the labeling of any of the interactants. For example, a microphysiometer can be used to detect the interaction of mCRISPC or rCRISPC with their respective target molecules without the labeling of mCRISPC or rCRISPC or the target molecules (see, e.g., McConnell H M et al., Science 257:1906-12 (1992)). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between compound and receptor.

In addition to cell-free assays, the readily available source of mCRISPC or rCRISPC proteins provided by the present invention also facilitates the generation of cell-based assays for identifying small molecule agonists/antagonists and the like. For example, cells can be caused to express or overexpress a recombinant mCRISPC or rCRISPC protein in the presence and absence of a test modulating agent of interest, with the assay scoring for modulation in mCRISPC or rCRISPC responses by the target cell mediated by the test agent. For example, as with the cell-free assays, modulating agents which produce a statistically significant change in mCRISPC- or rCRISPC-dependent responses (either an increase or decrease) can be identified.

Recombinant expression vectors that can be used for expression of mCRISPC or rCRISPC are known in the art (see discussions above). In one embodiment, within the expression vector the mCRISPC- or rCRISPC-coding sequences are operably linked to regulatory sequences that allow for constitutive or inducible expression of mCRISPC or rCRISPC in the indicator cell(s). Use of a recombinant expression vector that allows for constitutive or inducible expression of mCRISPC or rCRISPC in a cell is preferred for identification of compounds that enhance or inhibit the activity of mCRISPC or rCRISPC. In an alternate embodiment, within the expression vector, the mCRISPC or rCRISPC coding sequences are operably linked to regulatory sequences of the endogenous mCRISPC or rCRISPC gene (i.e., the promoter regulatory region derived from the endogenous gene). Use of a recombinant expression vector in which mCRISPC or rCRISPC expression is controlled by the endogenous regulatory sequences is preferred for identification of compounds that enhance or inhibit the transcriptional expression of mCRISPC or rCRISPC. In one embodiment, an assay is a cell-based assay comprising contacting a cell expressing an mCRISPC or rCRISPC target molecule (e.g., a CRISPC intracellular interacting molecule) with a test compound and determining the ability of the test compound to modulate (e.g. stimulate or inhibit) the activity of the mCRISPC or rCRISPC target molecule. Determining the ability of the test compound to modulate the activity of an mCRISPC or rCRISPC target molecule can be accomplished, for example, by determining the ability of the mCRISPC or rCRISPC protein to bind to or interact with the mCRISPC or rCRISPC target molecule or its ligand.

In an illustrative embodiment, the expression or activity of mCRISPC or rCRISPC is modulated in cells and the effects of modulating agents of interest on the readout of interest (such as, e.g., inhibition of capacitation and/or modulation of sperm-egg fusion) are measured.

In another embodiment, modulators of mCRISPC or rCRISPC expression are identified in a method wherein a cell is contacted with a candidate compound and the expression of mCRISPC or rCRISPC mRNA or protein in the cell is determined. The level of expression of mCRISPC or rCRISPC mRNA or protein in the presence of the candidate compound is compared to the level of expression of mCRISPC or rCRISPC mRNA or protein in the absence of the candidate compound. The candidate compound can then be identified as a modulator of mCRISPC or rCRISPC expression based on this comparison. For example, when expression of mCRISPC or rCRISPC mRNA or protein is greater (e.g., statistically significantly greater) in the presence of the candidate compound than in its absence, the candidate compound is identified as a stimulator of mCRISPC or rCRISPC mRNA or protein expression. Alternatively, when expression of mCRISPC or rCRISPC mRNA or protein is less (e.g., statistically significantly less) in the presence of the candidate compound than in its absence, the candidate compound is identified as an inhibitor of mCRISPC or rCRISPC mRNA or protein expression. The level of mCRISPC or rCRISPC mRNA or protein expression in the cells can be determined by methods described herein for detecting mCRISPC or rCRISPC mRNA or protein.

In a preferred embodiment, determining the ability of the mCRISPC or rCRISPC protein to bind to or interact with an mCRISPC or rCRISPC target molecule can be accomplished by measuring a read out of the activity of mCRISPC or rCRISPC or of the target molecule. For example, the activity of mCRISPC or rCRISPC or a target molecule can be determined by detecting induction of a cellular second messenger of the target, detecting catalytic/enzymatic activity of the target an appropriate substrate, detecting the induction of a reporter gene (comprising a target-responsive regulatory element operably linked to a nucleic acid encoding a detectable marker, e.g., chloramphenicol acetyl transferase), or detecting a target-regulated cellular response, for example, inhibition of capacitation and/or modulation of sperm-egg fusion.

In yet another aspect of the invention, mCRISPC or rCRISPC proteins or portions thereof can be used as “bait proteins” in a two-hybrid assay or three-hybrid assay (see, e.g., U.S. Pat. No. 5,283,317; Zervos A S et al., Cell 72:223-32 (1993); Madura K et al., J. Biol. Chem. 268:12046-54 (1993); Bartel P et al., Biotechniques 14:920-24 (1993); Iwabuchi K et al., Oncogene 8:1693-96 (1993); and WO 94/10300) to identify other proteins which bind to or interact with mCRISPC or rCRISPC and/or are involved in mCRISPC or rCRISPC activity. Such CRISPC-binding proteins are also likely to be involved in the propagation of signals by the CRISPC proteins or CRISPC targets as, for example, downstream elements of a CRISPC-mediated signaling pathway. Alternatively, such mCRISPC- or rCRISPC-binding proteins may be mCRISPC or rCRISPC inhibitors.

The two-hybrid system is based on the modular nature of most transcription factors, which consist of separable DNA-binding and activation domains. Briefly, the assay utilizes two different DNA constructs. In one construct, the gene that codes for an mCRISPC or rCRISPC protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a mCRISPC- or rCRISPC-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the mCRISPC or rCRISPC protein.

This invention further pertains to novel agents identified by the above-described screening assays. Accordingly, it is within the scope of this invention to further use an agent identified as described herein in an appropriate animal model. For example, an agent identified as described herein (e.g., an mCRISPC or rCRISPC modulating agent, an antisense mCRISPC or rCRISPC polynucleotide, an mCRISPC- or rCRISPC-specific antibody, or an mCRISPC- or rCRISPC-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

In another embodiment, an mCRISPC or rCRISPC promoter can be used in gain-of-function drug discovery applications via one-arm homologous recombination (see, e.g., U.S. Published Patent Application No. 2005/0003367; Zeh et al., Assay Drug Dev. Technol. 1:755-65 (2003); Qureshi et al., Assay Drug Dev. Technol. 1:767-76 (2003)). Briefly, an isolated genomic construct comprising an mCRISPC or rCRISPC promoter operably linked to a targeting sequence is introducing into a homogeneous population of cells (such as, for example, a homogeneous population of a human cell line or a homogeneous population of the murine epididymal epithelial cell line as described supra), wherein each of the cells comprises a signal transduction detection system. The term “targeting sequence” as used herein refers to a DNA sequence that is sufficiently homologous to a portion of the DNA sequence of a target gene to allow homologous recombination to occur within the cell. A sequence is sufficiently homologous if it is capable of binding to a target sequence under highly stringent conditions such as, for example, hybridization to filter bound DNA in 0.5 M NaHPO₄, 7% SDS, 1 mM EDTA at 65° C., and washing in 0.1×SSC/0.1% SDS at 68° C. The mCRISPC or rCRISPC promoter is heterologous to the target gene. Following recombination, the promoter controls transcription of an mRNA that encodes a polypeptide comprising an activatable domain that can alter the signal detected from the signal transduction system. Incubating the the population of cells under conditions which cause expression of the polypeptide and which cause activation of the activatable domain of the polypeptide allow selection of cells that have altered the signal detected from the signal transduction system.

In a preferred embodiment, the mCRISPC or rCRISPC promoter comprises at least 100 contiguous nucleotides from a polynucleotide sequence selected from the group consisting of SEQ ID NO:29 and SEQ ID NO:30, more preferably at least 500 contiguous nucleotides from a polynucleotide sequence selected from the group consisting of SEQ ID NO:29 and SEQ ID NO:30, even more preferably at least 1000 contiguous nucleotides from a polynucleotide sequence selected from the group consisting of SEQ ID NO:29 and SEQ ID NO:30. In a particularly preferred embodiment, the mCRISPC or rCRISPC promoter comprises SEQ ID NO:29 or SEQ ID NO:30.

C. Methods of Rational Drug Design

mCRISPC or rCRISPC and mCRISPC or rCRISPC binding polypeptides can be used for rational drug design of candidate CRISPC-modulating agents. The mCRISPC or rCRISPC polypeptides can be used for protein X-ray crystallography or other structure analysis methods, such as the DOCK program (see, e.g., Kuntz I D et al., J. Mol. Biol. 161: 269-88 (1982); Kuntz I D, Science 257:1078-82 (1992)) and variants thereof. Potential therapeutic drugs may be designed rationally on the basis of structural information thus provided.

D. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome and, thus, locate gene regions associated with genetic disease; (ii) identify an individual from a minute biological sample (tissue typing); and (iii) aid in forensic identification of a biological sample.

E. Predictive Medicine:

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining mCRISPC or rCRISPC protein and/or nucleic acid expression as well as CRISPC activity, in the context of a biological sample (e.g., blood, serum, cells, tissue (preferably the epididymis)) to thereby determine whether an individual is afflicted with a disease or disorder, or is at risk of developing a disorder, associated with aberrant CRISPC expression or activity. The invention also provides for prognostic (or predictive) assays for determining whether an individual is at risk of developing a disorder associated with CRISPC protein, nucleic acid expression, or activity. For example, mutations in a CRISPC gene can be assayed in a biological sample. Such assays can be used for prognostic or predictive purpose to thereby prophylactically treat an individual prior to the onset of a disorder characterized by or associated with CRISPC protein, nucleic acid expression, or activity.

Another aspect of the invention pertains to monitoring the influence of agents (e.g., drugs, compounds) on the expression or activity of CRISPC in clinical trials.

These and other agents are described in further detail in the following sections.

1. Diagnostic Assays

An exemplary method for detecting the presence or absence of CRISPC protein or nucleic acid in a biological sample involves obtaining a biological sample from a test subject and contacting the biological sample with a compound or an agent capable of detecting CRISPC protein or nucleic acid (e.g., mRNA, genomic DNA) that encodes CRISPC protein such that the presence of CRISPC protein or nucleic acid is detected in the biological sample. A preferred agent for detecting CRISPC mRNA to (or genomic DNA is a labeled nucleic acid probe capable of hybridizing to (CRISPC mRNA or genomic DNA. The nucleic acid probe can be, for example, an mCRISPC or rCRISPC nucleic acid, such as the nucleic acid of SEQ ID NO:3 or SEQ ID NO:4, or a portion thereof, such as an oligonucleotide of at least 15, 30, 50, 100, 250, 500 or more nucleotides in length and sufficient to specifically hybridize under stringent conditions to CRISPC mRNA or genomic DNA. Other suitable probes for use in the diagnostic assays of the invention are described herein.

A preferred agent for detecting CRISPC protein is an antibody capable of binding to CRISPC protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells, and biological fluids isolated from a subject, as well as tissues, cells (preferably epididymal cells or tissue), and fluids present within a subject; that is, the detection method of the invention can be used to detect CRISPC mRNA, protein, or genomic DNA in a biological sample in vitro as well as in vivo. For example, in vitro techniques for detection of CRISPC mRNA include Northern hybridizations and in situ hybridizations. In vitro techniques for detection of CRISPC protein include enzyme linked immunosorbent assays (ELISAs), Western blots, immunoprecipitation, and immunofluorescence. In vitro techniques for detection of CRISPC genomic DNA include Southern hybridizations. Furthermore, in vivo techniques for detection of CRISPC protein include introducing into a subject a labeled anti-mCRISPC or anti-rCRISPC antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a sperm sample isolated by conventional means from a subject.

In another embodiment, the methods further involve obtaining a control biological sample from a control subject, contacting the control sample with a compound or agent capable of detecting CRISPC protein, mRNA, or genomic DNA, such that the presence of CRISPC protein, mRNA, or genomic DNA is detected in the biological sample, and comparing the presence of CRISPC protein, mRNA, or genomic DNA in the control sample with the presence of CRISPC protein, mRNA, or genomic DNA in the test sample.

The invention also encompasses kits for detecting the presence of CRISPC in a biological sample. For example, the kit can comprise a labeled compound or agent capable of detecting CRISPC protein or mRNA in a biological sample; means for determining the amount of CRISPC in the sample; and means for comparing the amount of CRISPC in the sample with a standard. The compound or agent can be packaged in a suitable container. The kit can further comprise instructions for using the kit to detect CRISPC protein or nucleic acid.

2. Prognostic Assays

The diagnostic methods described herein can furthermore be utilized to identify subjects having or at risk of developing a disease or disorder associated with aberrant CRISPC expression or activity. For example, the assays described herein, such as the preceding diagnostic assays or the following assays, can be utilized to identify a subject having or at risk of developing a disorder associated with CRISPC protein, nucleic acid expression, or activity. Thus, the present invention provides a method for identifying a disease or disorder associated with aberrant CRISPC expression or activity in which a test sample is obtained from a subject and CRISPC protein or nucleic acid (e.g., mRNA, genomic DNA) is detected, wherein the presence of CRISPC protein or nucleic acid is diagnostic for a subject having or at risk of developing a disease or disorder associated with aberrant CRISPC expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant CRISPC expression or activity. Thus, the present invention provides methods for determining whether a subject can be effectively treated with an agent for a disorder associated with aberrant CRISPC expression or activity in which a test sample is obtained and CRISPC protein or nucleic acid expression or activity is detected (e.g., wherein the abundance of CRISPC protein or nucleic acid expression or activity is diagnostic for a subject that can be administered the agent to treat a disorder associated with aberrant CRISPC expression or activity).

The methods of the invention can also be used to detect genetic alterations in a CRISPC gene, thereby determining if a subject with the altered gene is at risk for a disorder associated with the CRISPC gene. In preferred embodiments, the methods include detecting, in a sample of cells from the subject, the presence or absence of a genetic alteration characterized by at least one of an alteration affecting the integrity of a gene encoding a CRISPC protein or the mis-expression of the CRISPC gene. For example, such genetic alterations can be detected by ascertaining the existence of at least one of 1) a deletion of one or more nucleotides from a CRISPC gene; 2) an addition of one or more nucleotides to a CRISPC gene; 3) a substitution of one or more nucleotides of a CRISPC gene, 4) a chromosomal rearrangement of a CRISPC gene; 5) an alteration in the level of a messenger RNA transcript of a CRISPC gene, 6) aberrant modification of a CRISPC gene, such as of the methylation pattern of the genomic DNA, 7) the presence of a non-wild type splicing pattern of a messenger RNA transcript of a CRISPC gene, 8) a non-wild type level of a CRISPC protein, 9) allelic loss of a CRISPC gene, and 10) inappropriate post-translational modification of a CRISPC protein. As described herein, there are a large number of assay techniques known in the art which can be used for detecting alterations in a CRISPC gene. A preferred biological sample is a tissue or serum sample isolated by conventional means from a subject, for example, an epididymal tissue sample.

In certain embodiments, detection of the alteration involves the use of a probe/primer in a PCR (see, e.g., U.S. Pat. Nos. 4,683,195 and 4,683,202), such as anchor PCR or RACE PCR, or, alternatively, in a ligation chain reaction (LCR) (see, e.g., Landegren U et al., Science 241:1077-80 (1988); Nakazawa H et al., Proc. Natl. Acad. Sci. USA 91:360-64 (1994)), the latter of which can be particularly useful for detecting point mutations in the CRISPC gene (see, e.g., Abravaya K et al., Nucleic Acids Res. 23:675-82 (1995)). This method can include the steps of collecting a sample of cells from a patient, isolating nucleic acid (e.g., genomic, mRNA, or both) from the cells of the sample, contacting the nucleic acid sample with one or more primers which specifically hybridize to a CRISPC gene under conditions such that hybridization and amplification of the CRISPC gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include, for example, self sustained sequence replication (Guatelli J C et al., Proc. Natl. Acad. Sci. USA 87:1874-78 (1990)), transcriptional amplification system (Kwoh D Y et al., Proc. Natl. Acad. Sci. USA 86:1173-77 (1989)), Q-Beta Replicase (Lizardi P M et al., Biotechnology (N.Y.) 6:1197 (1988)), or any other nucleic acid amplification method, followed by the detection of the amplified molecules using techniques well known to those of skill in the art. These detection schemes are especially useful for the detection of nucleic acid molecules if such molecules are present in very low numbers.

In an alternate embodiment, mutations in a CRISPC gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in CRISPC can be identified by hybridizing a sample and control nucleic acids, e.g., DNA or RNA, to high density arrays containing hundreds or thousands of oligonucleotides probes (Cronin M T et al., Hum. Mutat. 7: 244-55 (1996); Kozal M J et al., Nat. Med. 2:753-59 (1996)). For example, genetic mutations in CRISPC can be identified in two dimensional arrays containing light-generated DNA probes as described in Cronin M T et al. (supra). Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the mCRISPC or rCRISPC gene and detect mutations by comparing the sequence of the sample CRISPC with the corresponding wild-type (control) sequence. Examples of sequencing reactions include those based on techniques developed by Maxam A M and Gilbert W, Proc. Natl. Acad. Sci. USA 74:560-64 (1977) or Sanger F et al., Proc. Natl. Acad. Sci. USA 74:5463-67 (1977). It is also contemplated that any of a variety of automated sequencing procedures can be utilized when performing the diagnostic assays (see, e.g., Naeve C W et al., Biotechniques 19:448-53 (1995)), including sequencing by mass spectrometry (see, e.g., WO 94/16101; Cohen A S et al., Adv. Chromatogr. 36:127-62 (1996); and Griffin H G and Griffin A M, Appl. Biochem. Biotechnol. 38:147-59 (1993)).

Other methods for detecting mutations in the mCRISPC or rCRISPC gene include methods in which protection from cleavage agents is used to detect mismatched bases in RNA/RNA or RNA/DNA heteroduplexes (Myers R M et al., Science 230:1242-46 (1985)). In general, the art technique of “mismatch cleavage” starts by providing heteroduplexes formed by hybridizing (labeled) RNA or DNA containing the wild-type mCRISPC or rCRISPC sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation (see, e.g., Cotton R G H et al., Proc. Natl. Acad. Sci. USA 85:4397-4401 (1988); Saleeba J A and Cotton R G H, Meth. Enzymol. 217:286-95 (1993)). In a preferred embodiment, the control DNA or RNA can be labeled for detection. In still another embodiment, the mismatch cleavage reaction employs one or more proteins that recognize mismatched base pairs in double-stranded DNA (so called “DNA mismatch repair” enzymes) in defined systems for detecting and mapping point mutations in CRISPC obtained from samples of cells. For example, the mutY enzyme of E. coli cleaves A at G/A mismatches and the thymidine DNA glycosylase from HeLa cells cleaves T at G/T mismatches (Hsu I C et al., Carcinogenesis 15:1657-62 (1994)). According to an exemplary embodiment, a probe based on an mCRISPC or rCRISPC sequence, for example, a wild-type mCRISPC or rCRISPC sequence, is hybridized to a cDNA or other DNA product from a test cell(s). The duplex is treated with a DNA mismatch repair enzyme, and the cleavage products, if any, can be detected from electrophoresis protocols or the like (see, e.g., U.S. Pat. No. 5,459,039).

In other embodiments, alterations in electrophoretic mobility will be used to identify mutations in CRISPC genes. For example, single strand conformation polymorphism (SSCP) may be used to detect differences in electrophoretic mobility between mutant and wild type nucleic acids (Orita M et al., Proc Natl. Acad. Sci USA: 86:2766-70 (1989); see also, Cotton R G H, Mutat. Res. 285:125-44 (1993); Hayashi K, Genet. Anal. Tech. Appl. 9:73-79 (1992)). Single-stranded DNA fragments of sample and control CRISPC nucleic acids will be denatured and allowed to renature. The secondary structure of single-stranded nucleic acids varies according to sequence; the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen J et al., Trends Genet. 7:5 (1991)).

In yet another embodiment, the movement of mutant or wild-type fragments in polyacrylamide gels containing a gradient of denaturant is assayed using denaturing gradient gel electrophoresis (DGGE) (Myers R M et al., Nature 313:495-98 (1985)). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum V and Riesner D, Biophys. Chem. 26:235-46 (1987)).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki R K et al., Nature 324:163-66 (1986); Saiki R K et al., Proc. Natl. Acad. Sci. USA 86:6230-34 (1989)). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs R A et al., Nucleic Acids Res. 17:2437-48 (1989)) or at the extreme 3′ end of one primer where, under appropriate conditions, mismatch can prevent, or reduce polymerase extension (Prosser J, Trends Biotechnol. 11:238-46 (1993)). In addition, it may be desirable to introduce a novel restriction site in the region of the mutation to create cleavage-based detection (Gasparini P et al., Mol. Cell. Probes 6:1-7 (1992)). It is anticipated that in certain embodiments amplification may also be performed using Taq ligase for amplification (Barany F, Proc. Natl. Acad. Sci USA 88:189-93 (1991)). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, for example, in clinical settings to diagnose patients exhibiting symptoms or family history of a disease or illness involving a CRISPC gene.

Furthermore, any cell type or tissue in which CRISPC is expressed may be utilized in the prognostic assays described herein.

F. CRISPC Protease Activity

The cone snail venom protein Tex31 contains a plant pathogenesis-related type 1 domain (PR-1) that is also conserved in the mammalian CRISP proteins. Tex31 is the only PR-1 domain-containing protein known to have site-specific protease activity (Milne T. J. et al., J. Biol. Chem. 278:31105-10 (2003)). Applicants herein identify protease activity for native rCRISPA (rCRISP1), the first demonstration of protease activity for any mammalian CRISP family member.

Determination of protease activity can be performed by any method as is known in the art. Protease screening methods useful in connection with the present disclosure include, for example, mechanism-based inhibitors which target a protease's active site can be constructed so as to include a biological, chemical, or radiological detection system (see, e.g., Marnett A B & Craik C S, Trends Biotechnol. 23:59-64 (2005)).

The conservation of protease activity between CRISPA and CRISPC is predicted from both the primary amino acid sequence of the PR-1 domain and the overall domain structure conserved among Tex31 and all mammalian CRISPs. The PR-1 domain of Tex31 is hypothesized to contain a basic substrate recognition site with the predicted catalytic residues His130 and Glu115. The mammalian CRISPs have these residues conserved in the PR-1 domain. Sialylation may be important for proteolytic activity because, as Applicants demonstrate herein, purification of rCRISPA with DANA present allows for rCRISPA with protease activity, whereas purification of rCRISPA using known procedures without DANA present results in rCRISPA lacking protease activity. A sialylated glycan on rCRISPA may be influencing the protein's native confirmation and ability to recognize, bind, or cleave its substrate. If rat CRISPC is glycosylated and sialylated, the glycan may also be involved in the protein's confirmation and ability to recognize, bind, or cleave its substrate.

Protease substrate specificity can be determined by any method as is known to one of ordinary skill in the art. For example, substrate phage libraries (see, e.g., Sedlacek R & Chen E, Comb. Chem. High Throughput Screen. 8:197-203 (2005); Ohkubo et al., Comb. Chem. High Throughput Screen. 4:573-83 (2001)) or substrate peptide libraries can be utilized to determine the site specificity of a presumptive protease (available from, for example, JPT Peptide Technologies, Springfield, Va.).

Most of the peptide substrates of rCRISPA contain a sequential arginine-alanine (RA). Most of the cleaved peptides also have at least one dibasic residue pair (KK, RR, KR, RK). These features are similar to a substrates recognized by Tex31, Ac-KLNKR-pNA and Ac-KLNKRWAbuKQSG-NH₂ as described in Milne T. J. et al., J. Biol. Chem. 278:31105-10 (2003). It is predicted, based on sequence conservation noted above, that CRISPC could have similar protease substrate specificity.

CRISPC protease activity may play a role in activation of sperm motility, progression of sperm capacitation, involvement in sperm binding to or releasing from the epithelial cells of the male and/or female reproductive tract, or mediation of sperm-egg binding or fusion. In more general applications, CRISPC protease activity may be used as a tool to cleave specific peptides containing the substrate recognition sequence and/or cleavage site.

VI. Administration of CRISPC Modulating Agents

CRISPC modulating agents of the invention are administered to subjects in a biologically compatible form suitable for pharmaceutical administration in vivo to either enhance or suppress CRISPC inhibition of capacitation and/or modulation of sperm-egg fusion. By “biologically compatible form suitable for administration in vivo” is meant a form of the protein to be administered in which any toxic effects are outweighed by the therapeutic effects of the protein. The term subject is intended to include living organisms in which an immune response can be elicited, for example, mammals. Administration of an agent as described herein can be in any pharmacological form including a therapeutically active amount of an agent alone or in combination with a pharmaceutically acceptable carrier.

Administration of a therapeutically active amount of the therapeutic compositions of the present invention is defined as an amount effective, at dosages and for periods of time necessary to achieve the desired result. For example, a therapeutically active amount of a CRISPC modulating agent may vary according to factors such as the disease state, age, sex, and weight of the individual, and the ability of peptide to elicit a desired response in the individual. Dosage regima may be adjusted to provide the optimum therapeutic response. For example, several divided doses may be administered daily, or the dose may be proportionally reduced as indicated by the exigencies of the therapeutic situation.

The therapeutic or pharmaceutical compositions of the present invention can be administered by any suitable route known in the art including, for example, intravenous, subcutaneous, intramuscular, transdermal, intrathecal, or intracerebral or administration to cells in ex vivo treatment protocols. Administration can be either rapid as by injection or over a period of time as by slow infusion or administration of slow release formulation. For treating the infertility, administration of the therapeutic or pharmaceutical compositions of the present invention can be, for example, after harvesting of epididymal sperm for use in in vitro fertilization (IVF) or intracytoplasmic sperm injection (ICSI). When it is intended that an mCRISPC or rCRISPC polypeptide be administered to ejaculated sperm in the female reproductive tract, administration can be with one or more agents capable of acting as, for example, topical or systemic agents for females.

mCRISPC or rCRISPC can also be linked or conjugated with agents that provide desirable pharmaceutical or pharmacodynamic properties. For example, mCRISPC or rCRISPC can be coupled to any substance known in the art to promote penetration or transport across the blood-brain barrier such as an antibody to the transferrin receptor, and administered by intravenous injection (see, e.g., Friden P M et al., Science 259:373-77 (1993)). Furthermore, mCRISPC or rCRISPC can be stably linked to a polymer such as polyethylene glycol to obtain desirable properties of solubility, stability, half-life, and other pharmaceutically advantageous properties (see, e.g., Davis et al., Enzyme Eng. 4:169-73 (1978); Burnham N L, Am. J. Hosp. Pharm. 51:210-18 (1994)).

Furthermore, the mCRISPC or rCRISPC polypeptide can be in a composition which aids in delivery into the cytosol of a cell. For example, the peptide may be conjugated with a carrier moiety such as a liposome that is capable of delivering the peptide into the cytosol of a cell. Such methods are well known in the art (see, e.g., Amselem S et al., Chem. Phys. Lipids 64:219-37 (1993)). Alternatively, the mCRISPC or rCRISPC polypeptide can be modified to include specific transit peptides or fused to such transit peptides which are capable of delivering the mCRISPC or rCRISPC polypeptide into a cell. In addition, the polypeptide can be delivered directly into a cell by microinjection.

The compositions are usually employed in the form of pharmaceutical preparations. Such preparations are made in a manner well known in the pharmaceutical art. One preferred preparation utilizes a vehicle of physiological saline solution, but it is contemplated that other pharmaceutically acceptable carriers such as physiological concentrations of other non-toxic salts, five percent aqueous glucose solution, sterile water or the like may also be used. As used herein “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. Supplementary active compounds can also be incorporated into the compositions. It may also be desirable that a suitable buffer be present in the composition. Such solutions can, if desired, be lyophilized and stored in a sterile ampoule ready for reconstitution by the addition of sterile water for ready injection. The primary solvent can be aqueous or alternatively non-aqueous. mCRISPC or rCRISPC can also be incorporated into a solid or semi-solid biologically compatible matrix which can be implanted into tissues requiring treatment.

The carrier can also contain other pharmaceutically-acceptable excipients for modifying or maintaining the pH, osmolarity, viscosity, clarity, color, sterility, stability, rate of dissolution, or odor of the formulation. Similarly, the carrier may contain still other pharmaceutically-acceptable excipients for modifying or maintaining release or absorption or penetration across the blood-brain barrier. Such excipients are those substances usually and customarily employed to formulate dosages for parenteral administration in either unit dosage or multi-dose form or for direct infusion by continuous or periodic infusion.

Dose administration can be repeated depending upon the pharmacokinetic parameters of the dosage formulation and the route of administration used.

It is also provided that certain formulations containing the mCRISPC or rCRISPC polypeptide or fragment thereof are to be administered orally. Such formulations are preferably encapsulated and formulated with suitable carriers in solid dosage forms. Some examples of suitable carriers, excipients, and diluents include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methyl cellulose, methyl- and propylhydroxybenzoates, talc, magnesium, stearate, water, mineral oil, and the like. The formulations can additionally include lubricating agents, wetting agents, emulsifying and suspending agents, preserving agents, sweetening agents, or flavoring agents. The compositions may be formulated so as to provide rapid, sustained, or delayed release of the active ingredients after administration to the patient by employing procedures well known in the art. The formulations can also contain substances that diminish proteolytic degradation and/or substances which promote absorption such as, for example, surface active agents.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved and (b) the limitations inherent in the art of compounding such an active compound for the treatment of sensitivity in individuals. The specific dose can be readily calculated by one of ordinary skill in the art, e.g., according to the approximate body weight or body surface area of the patient or the volume of body space to be occupied. The dose will also be calculated dependent upon the particular route of administration selected. Further refinement of the calculations necessary to determine the appropriate dosage for treatment is routinely made by those of ordinary skill in the art. Such calculations can be made without undue experimentation by one skilled in the art in light of the activity disclosed herein in assay preparations of target cells. Exact dosages are determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

Toxicity and therapeutic efficacy of such compounds can be determined by standard pharmaceutical procedures in cell cultures or experimental animals, for example, for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In one embodiment of this invention, an mCRISPC or rCRISPC polypeptide may be therapeutically administered by implanting into patients vectors or cells capable of producing a biologically-active form of mCRISPC or rCRISPC or a precursor of mCRISPC or rCRISPC, that is, a molecule that can be readily converted to a biological-active form of mCRISPC or rCRISPC by the body.

In one approach, cells that secrete mCRISPC or rCRISPC may be encapsulated into semipermeable membranes for implantation into a patient. The cells can be cells that normally express mCRISPC or rCRISPC or a precursor thereof or the cells can be transformed to express mCRISPC or rCRISPC or a biologically active fragment thereof or a precursor thereof. It is preferred that the cell be of human origin. However, the formulations and methods herein can be used for veterinary as well as human applications and the term “patient” or “subject” as used herein is intended to include human and veterinary patients.

Monitoring the influence of agents (e.g., drugs or compounds) on the expression or activity of an mCRISPC or rCRISPC protein can be applied not only in basic drug screening, but also in clinical trials. For example, the effectiveness of an agent determined by a screening assay as described herein to increase CRISPC gene expression, protein levels, or upregulate CRISPC activity, can be monitored in clinical trials of subjects exhibiting decreased CRISPC gene expression, protein levels, or downregulated CRISPC activity. Alternatively, the effectiveness of an agent determined by a screening assay to decrease CRISPC gene expression, protein levels, or downregulate CRISPC activity, can be monitored in clinical trials of subjects exhibiting increased CRISPC gene expression, protein levels, or upregulated CRISPC activity. In such clinical trials, the expression or activity of a CRISPC gene, and preferably, other genes that have been implicated in a disorder can be used as a “read out” or markers of the phenotype of a particular cell.

For example, and not by way of limitation, genes, including CRISPC, that are modulated in cells by treatment with an agent (e.g., compound, drug, or small molecule) which modulates CRISPC activity (e.g., identified in a screening assay as described herein) can be identified. Thus, to study the effect of agents on a CRISPC associated disorder, for example, in a clinical trial, cells can be isolated and RNA prepared and analyzed for the levels of expression of CRISPC and other genes implicated in the CRISPC associated disorder, respectively. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of CRISPC or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the agent; (ii) detecting the level of expression of a CRISPC protein, mRNA, or genomic DNA in the pre-administration sample; (iii) obtaining one or more post-administration samples from the subject; (iv) detecting the level of expression or activity of the CRISPC protein, mRNA, or genomic DNA in the post-administration samples; (v) comparing the level of expression or activity of the CRISPC protein, mRNA, or genomic DNA in the pre-administration sample with the CRISPC protein, mRNA, or genomic DNA in the post administration sample or samples; and (vi) altering the administration of the agent to the subject accordingly. For example, increased administration of the agent may be desirable to increase the expression or activity of CRISPC to higher levels than detected, that is, to increase the effectiveness of the agent. Alternatively, decreased administration of the agent may be desirable to decrease expression or activity of CRISPC to lower levels than detected, that is, to decrease the effectiveness of the agent. According to such an embodiment, CRISPC expression or activity may be used as an indicator of the effectiveness of an agent, even in the absence of an observable phenotypic response.

In a preferred embodiment, the ability of a CRISPC modulating agent to modulate the inhibition of capacitation and/or modulation of sperm-egg fusion in a subject that would benefit from modulation of the expression and/or activity of CRISPC can be measured by detecting an improvement in the condition of the patient after the administration of the agent. Such improvement can be readily measured by one of ordinary skill in the art using indicators appropriate for the specific condition of the patient. Monitoring the response of the patient by measuring changes in the condition of the patient is preferred in situations were the collection of biopsy materials would pose an increased risk and/or detriment to the patient.

It is likely that the level of CRISPC may be altered in a variety of conditions and that quantification of CRISPC levels would provide clinically useful information.

Furthermore, in the treatment of disease conditions, compositions containing mCRISPC or rCRISPC can be administered exogenously and it would likely be desirable to achieve certain target levels of mCRISPC or rCRISPC polypeptide in sera, in any desired tissue compartment, or in the affected tissue. It would, therefore, be advantageous to be able to monitor the levels of mCRISPC or rCRISPC polypeptide in a patient or in a biological sample including a tissue biopsy sample obtained from a patient and, in some cases, also monitoring the levels of native CRISPC. Accordingly, the present invention also provides methods for detecting the presence of CRISPC in a sample from a patient.

VII. Kits of the Invention

Another aspect of the invention pertains to kits for carrying out the screening assays, modulatory methods, or diagnostic assays of the invention. For example, a kit for carrying out a screening assay of the invention can include a cell comprising an mCRISPC or rCRISPC polypeptide, means for determining mCRISPC or rCRISPC polypeptide activity, and instructions for using the kit to identify modulators of mCRISPC or rCRISPC activity. In another embodiment, a kit for carrying out a screening assay of the invention can include an composition comprising an mCRISPC or rCRISPC polypeptide, means for determining mCRISPC or rCRISPC activity, and instructions for using the kit to identify modulators of mCRISPC or rCRISPC activity.

In another embodiment, the invention provides a kit for carrying out a modulatory method of the invention. The kit can include, for example, a modulatory agent of the invention (e.g., an mCRISPC or rCRISPC inhibitory or stimulatory agent) in a suitable carrier and packaged in a suitable container with instructions for use of the modulator to modulate mCRISPC or rCRISPC activity.

Another aspect of the invention pertains to a kit for diagnosing a disorder associated with aberrant CRISPC expression and/or activity in a subject. The kit can include a reagent for determining expression of CRISPC (e.g., a nucleic acid probe(s) for detecting CRISPC mRNA or one or more antibodies for detection of CRISPC proteins), a control to which the results of the subject are compared, and instructions for using the kit for diagnostic purposes.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### EXAMPLES

The present invention is further defined in the following Examples. It should be understood that these Examples, while indicating preferred embodiments of the invention, are given by way of illustration only. From the above discussion and these Examples, one skilled in the art can ascertain the preferred features of this invention, and without departing from the spirit and scope thereof, can make various changes and modification of the invention to adapt it to various uses and conditions.

### Example 1

### Tissue Distribution and Epididymal Expression of 9230112K08Rik

Expression of 9230112K08Rik (mCRISPC) was determined by microarray experiments using MOE430 gene chips, where the mRNA for this gene is represented by Affymetrix Qualifier 1431468_at. Table 9 describes the expression profile of 1431468_at from all tissues available in the Gene Logic Database system, ranked from highest to lowest median expression value. 1431468_expression was determined to be specific to the epididymis by these results, as median expression values below 50 are considered to be below the limits of detection.

TABLE 9

| | | Median | Number of |
|---|---|---|---|
| Rank | Tissue | Expression | Samples |
| 1 | Epididymis_Segment_5 | 3951 | 5 |
| 2 | Epididymis_Segment_4 | 3509 | 5 |
| 3 | Epididymis_Segment_1 | 3338.5 | 12 |
| 4 | Epididymis_Segment_6 | 2898 | 5 |
| 5 | Epididymis_whole | 2779 | 4 |
| 6 | Epididymis_Segment_2 | 1992 | 13 |
| 7 | Epididymis_Segment_7 | 1816 | 3 |
| 8 | Epididymis_Segment_3 | 968 | 5 |
| 9 | Epididymis_Segment_8 | 784 | 5 |
| 10 | Epididymis_Segment_9 | 321 | 4 |
| 11 | Epididymis_Segment_10 | 320 | 5 |
| 12 | Pituitary_gland | 47.5 | 12 |
| 13 | Colon | 45 | 11 |
| 14 | Blood | 35 | 4 |
| 15 | Eye | 26 | 3 |
| 16 | Kidney | 25 | 3 |
| 17 | Glial_cell | 25 | 3 |
| 18 | Cell | 25 | 23 |
| 19 | Mast_cell | 22.5 | 4 |
| 20 | Skeletal_muscle_system_structure | 22 | 3 |
| 21 | Heart | 22 | 3 |
| 22 | Ovary | 21 | 3 |
| 23 | Liver | 19 | 45 |
| 24 | Salivary_gland | 18.5 | 8 |
| 25 | Normal_fat_pad_body_structure | 18 | 38 |
| 26 | Epithelial_cell | 18 | 9 |
| 27 | Small_intestine | 17.5 | 10 |
| 28 | Adrenal_gland | 17 | 7 |
| 29 | Lymph_node | 16 | 13 |
| 30 | Stomach | 15 | 13 |
| 31 | Gastrocnemius_muscle | 14 | 24 |
| 32 | Brain | 14 | 3 |
| 33 | Spleen | 11 | 15 |
| 34 | Bladder | 10 | 3 |
| 35 | Testis | 8 | 3 |
| 36 | Lung | 8 | 77 |
| 37 | Embryo | 4 | 3 |

The below tissues do not meet the minimum
number of samples requirement (3).

| | | | |
|---|---|---|---|
| NA | Breast | 35 | 1 |
| NA | Uterus | 24.5 | 2 |
| NA | Prostate | 16 | 1 |
| NA | vagina | 15 | 1 |
| NA | Oviduct | 10 | 1 |
| NA | Thymus | 0 | 0 |
| NA | Skin | 0 | 0 |
| NA | Monocyte | 0 | 0 |
| NA | Lymphocyte | 0 | 0 |
| NA | Jejunum | 0 | 0 |
| NA | Ileum | 0 | 0 |
| NA | Duodenum | 0 | 0 |
| NA | Corpus_striatum | 0 | 0 |
| NA | Cerebellum | 0 | 0 |
| NA | Adipocyte | 0 | 0 |

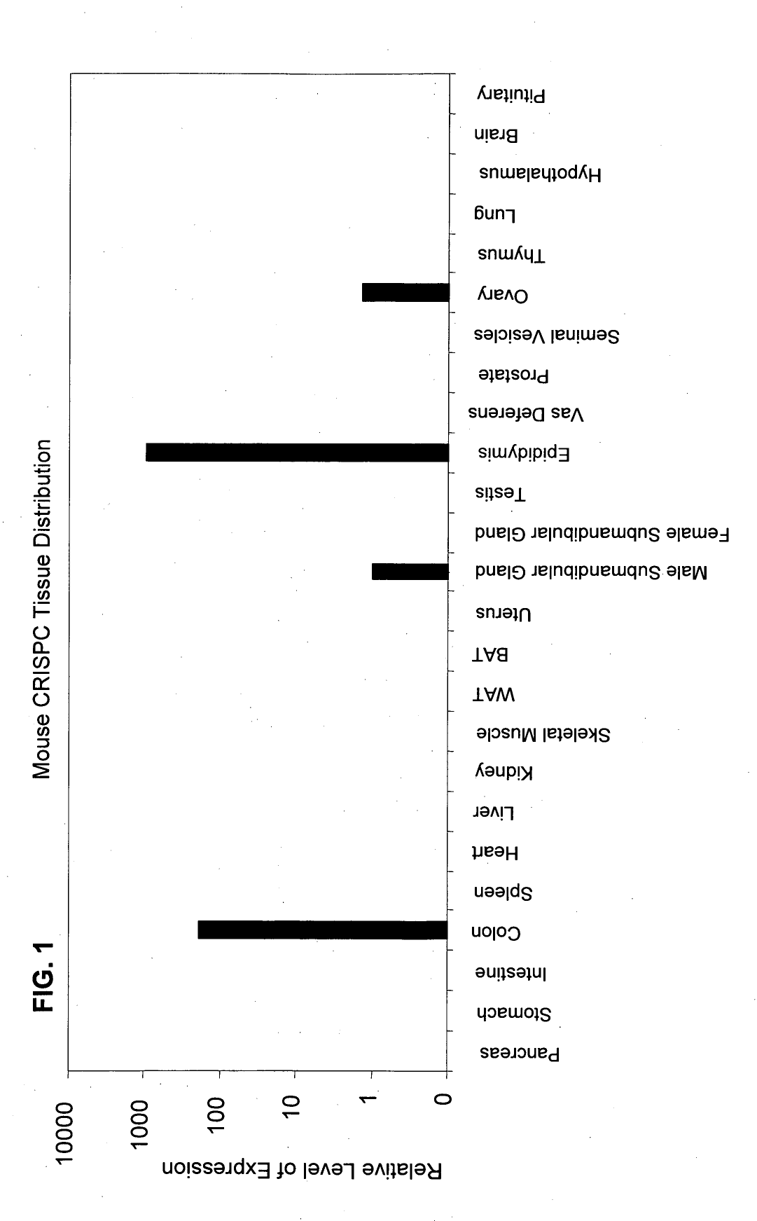

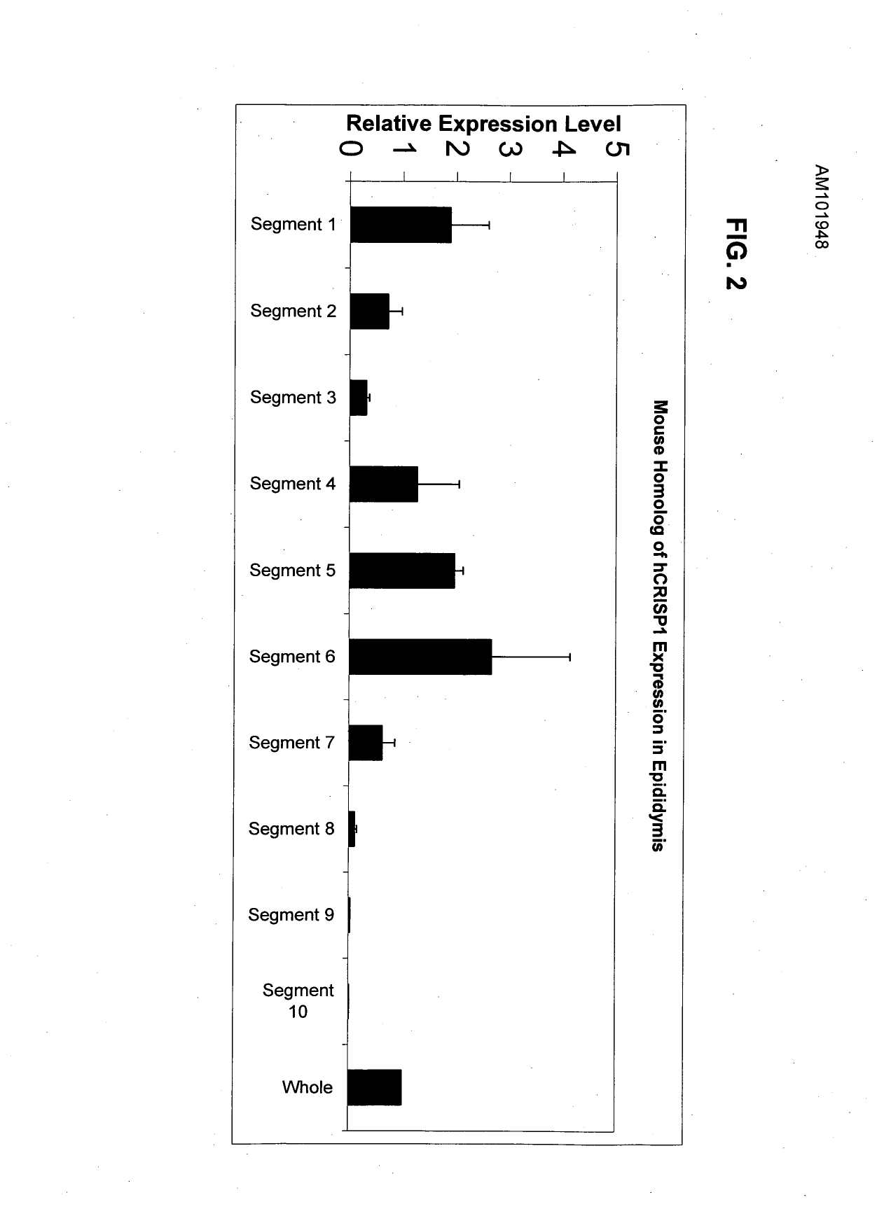

ssion of the mouse homologue of hCRISPC was also determined qRT-PCR analysis using the TaqMan® probe and primer sets described in SEQ ID Nos: 7-9. The tissue distribution of this gene is described in FIG. 1. The epidiymal segment-dependent expression profile of the mouse homologue of hCRISPC is described in FIG. 2.

### Example 2

### Tissue Distribution and Epididymal Expression of the Rat Homologue of hCRISPC

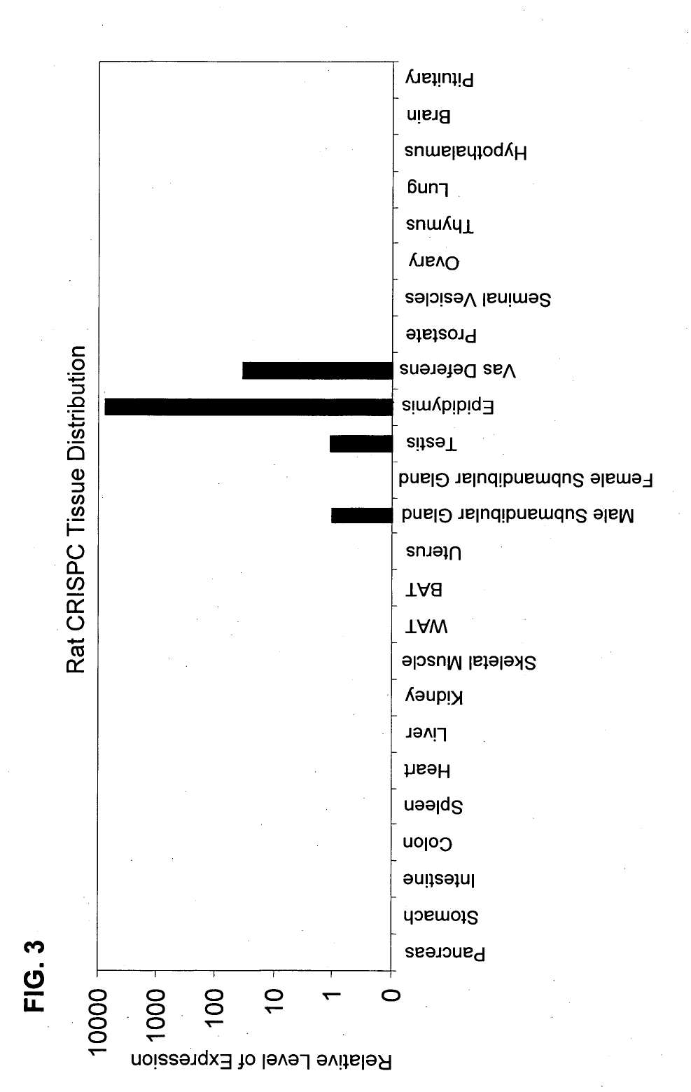

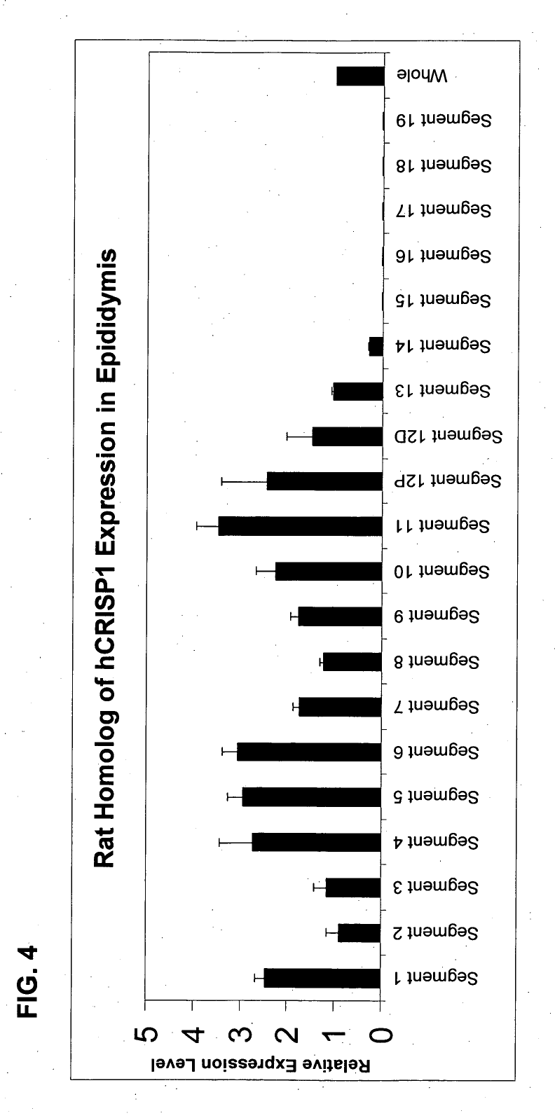

The mRNA for the rat gene #ENSRNOG00000013612 (rCRISPC) is not present as an Affymetrix qualifier in any of the currently available chip sets. Expression of the rat homologue of hCRISPC was determined by qRT-PCR analysis using the TaqMan® probe and primer sets described in SEQ ID NOs: 10-12. The tissue distribution of this gene is described in FIG. 3. The epididymal segment-dependent expression profile of the rat homologue of hCRISPC is described in FIG. 4.

### Example 3

### Recombinant Expression of rCRISPC in an Insect Cell System

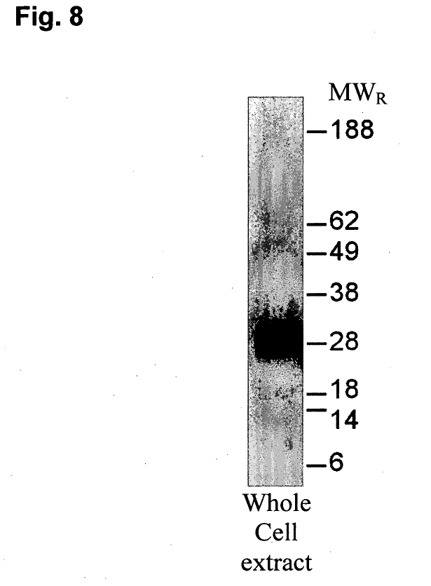

The rat and mouse CRISPC genes were cloned from total RNA extracted from rat and mouse epididymis using RT-PCR to generate and amplify cDNA clones using the primers listed as SEQ ID NOs: 31-36. The cDNAs (full-length=SEQ ID. NOs: 37-38, PR-1 domain-only truncations ═SEQ ID NOs: 39-40) was placed into the Invitrogen pCR8/GW/TOPO Gateway Entry vector. rCRISPC cDNA was successfully integrated into a baculovirus plasmid, and expression of recombinant, full-length protein was verified by Western Blot using the polyclonal antibody generated and affinity purified against SEQ ID NO:15 (FIG. 8).

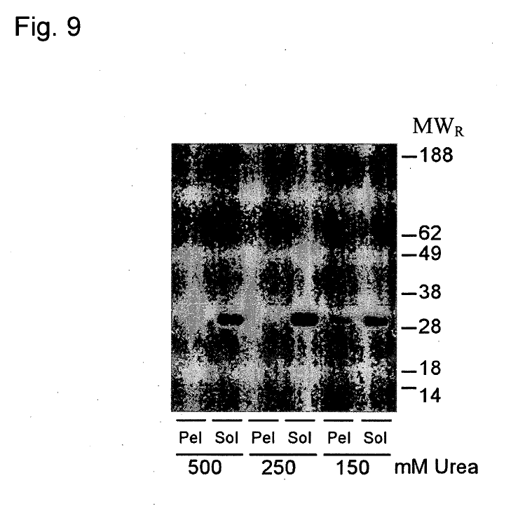

Complete solubilization of this protein from the recombinant rCRISPC-baculovirus infected sf9 cell pellets was tested by sonication in 1% (v/v) Triton X-100, 10% (v/v) glycerol, with increasing concentrations of urea (150-500 mM) detected by Western blotting (FIG. 9). 250 mM urea was the lowest concentration tested to fully solubilize recombinant full-length rCRISPC.

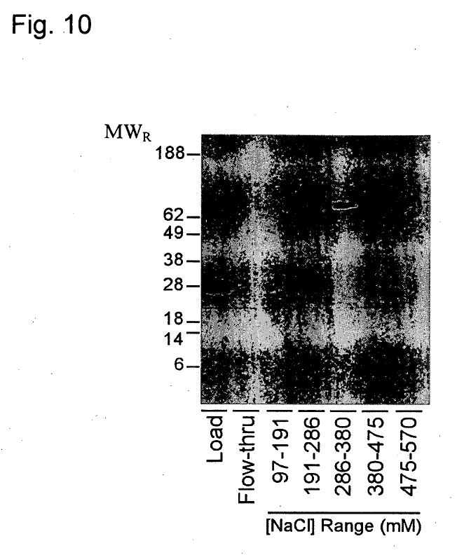

Solubilized recombinant rCRISPC was subjected to anion exchange chromatography as a first step in purification of the protein. rCRISPC-infected sf9 cell pellets were sonicated in 250 mM urea, 1% (v/v) Triton X-100, 10% (v/v) glycerol. Following centrifugation to remove the insoluble material, the supernatant was diluted 5-fold to final concentrations of 50 mM urea, 50 mM NaCl, 25 mM Tris-HCl pH 7.5, 0.2% (v/v) Triton X-100, 10% glycerol and loaded onto a HiTrap® Q FPLC column (GE Healthcare). Elution of bound protein was done by linear gradient from the loading buffer to a final buffer containing 1 M NaCl, 25 mM Tris-HCl, 10% glycerol, and fractions were analyzed for rCRISPC protein by Western blotting (FIG. 10). Recombinant rCRISPC at the appropriate relative molecular weight (˜30 kDa) elutes in two fractions ranging from 286-475 mM NaCl. However, a larger molecular weight (>62 kDa) immunoreactive band elutes between 286-380 mM NaCl. It remains to be determined if this band corresponds to rCRISPC covalently aggregated or bound to other protein(s).

### Example 4

### Purification of rCRISPA (rCRISP1) in the Presence of the Sialidase Inhibitor DANA

A protocol for the purification of rCRISPA (rCRISP1) from the epididymis was published in 1997 (Hall J. C. & Tubbs C. E., Prep. Biochem. Biotechnol. 27:239-51 (1997), incorporated herein by reference). Applicants have modified this protocol to include the following amounts of 2,3-dehydro-2-deoxy-N-acetyineuraminic acid (DANA) as an inhibitor of sialidase activity in each of the following buffers:

Tissue Homogenization Buffer: 25 mM Tris-HCl, 400 μM DANA

Concanavalin-A Affinity FPLC Binding Buffer: 25 mM Tris-HCl, 430 μM DANA, 1 mM MgCl₂, 1 mM MnCl₂, 1 mM CaCl₂

Concanavalin-A Affinity FPLC Elution Buffer: 25 mM Tris-HCl, 430 μM DANA, 200 mM methyl-a-D mannopyranoside

Size Exclusion Chromatography FPLC Buffer: 25 mM Tris-HCl, 43 μM DANA, 150 mM NaCl

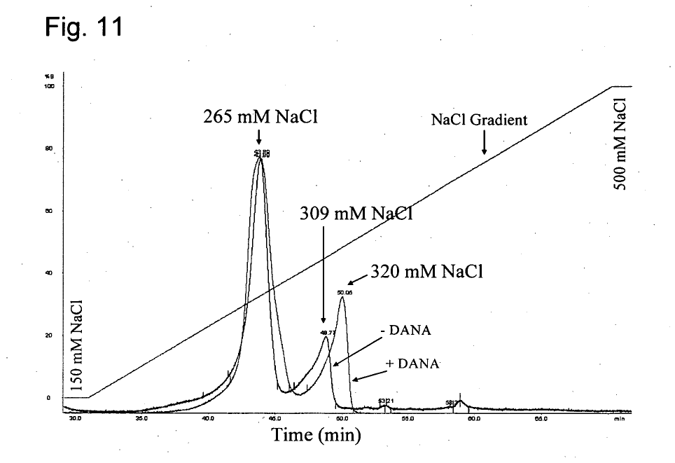

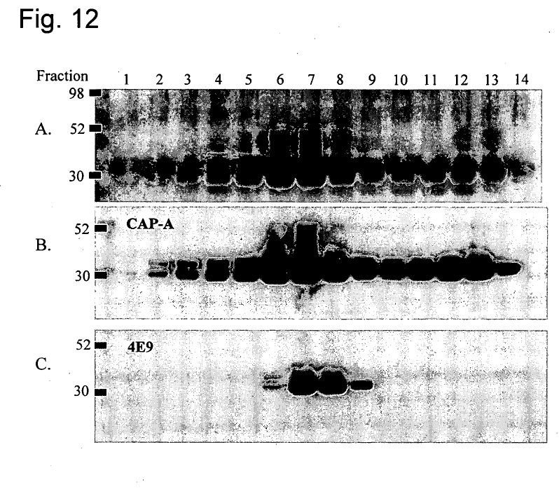

As a result of adding DANA to these buffers, two significant changes occur in the anion exchange profile of rCRISPA: 1) the peak eluting at 265 mM NaCl broadened and 2) the peak eluting at 309 mM NaCl is larger and shifted to elute at a higher (320 mM) concentration of NaCl (FIG. 11). The rCRISPA protein eluting from the anion exchange column was visualized by silver staining and Western blotting using both a polyclonal antibody (CAP-A, recognizing both the D and E forms of rCRISPA) and a monoclonal antibody (4E9, recognizing only the E form of rCRISPA) (FIG. 12). These data improve upon the Hall & Tubbs protocol by demonstrating that the previously published rCRISP1 purification protocol does not allow for purification of sialylated forms of the protein. Including these native glycoforms in the rCRISP1 preparation may be required for the activity of the native protein described herein.

### Example 5

### Identification of Protease Activity in Purified Native rCRISPA (rCRISP1) (+DANA Protocol)

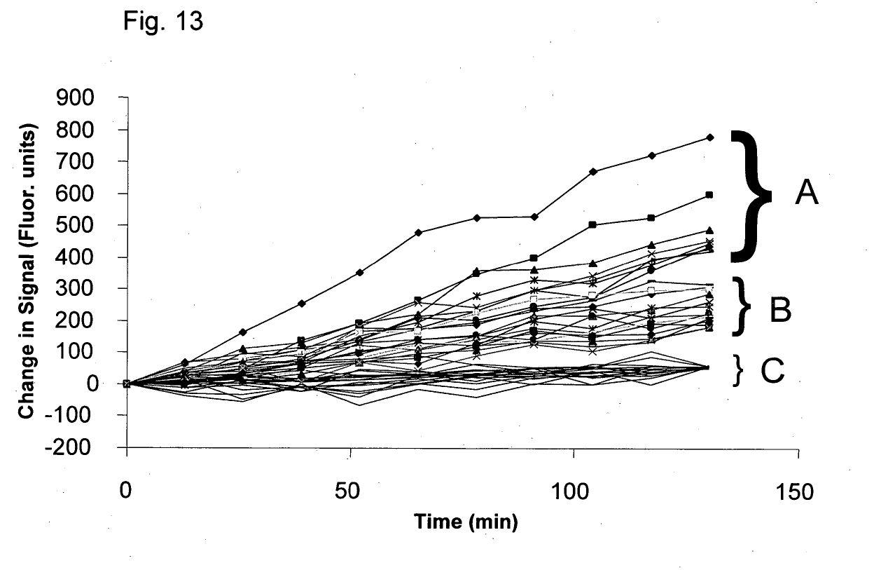

Native rCRISPA (rCRISP1) protein, purified with DANA present (see Example 4), was screened for the ability to cleave 360 unique peptides using the commercially available peptide substrate set (catalog # PrSS-360-75, JPT Peptide Technologies, Springfield, Va.). Protease activity was determined by measuring fluorescence (as per the manufacturer's protocol) at ten minute intervals for 130 minutes in the following assay conditions: 5 mM substrate peptide, 1 mg rCRISPA, 100 mM NaCl, 25 mM Tris-Cl, pH 7.5. The substrate peptides cleaved by rCRISPA in this assay are represented by blue and green traces noted in FIG. 13. Peptides that were not cleaved by rCRISPA are also noted in FIG. 13.

### Example 6

### Generation of CRISPC Knockout Mice

The objective is to create a conditional knockout of the mCRISPC gene in mice using homologous recombination in mouse embryonic stem cells and subsequent blastocyst injection of the appropriate targeted ES cells to create the gene targeted mice.

Mouse chromosome 1 sequence (n.t.# 18,314,000˜18,394,000) was retrieved from the Ensembl database and used as reference in this project. BAC clone RP23-387G12 was used for generating homologous arms and the conditional knockout region for the gene targeting vector, and the Southern probes for screening targeted events. PCR or the RED cloning/gap-repair methods were utilized to clone the appropriate sequences.

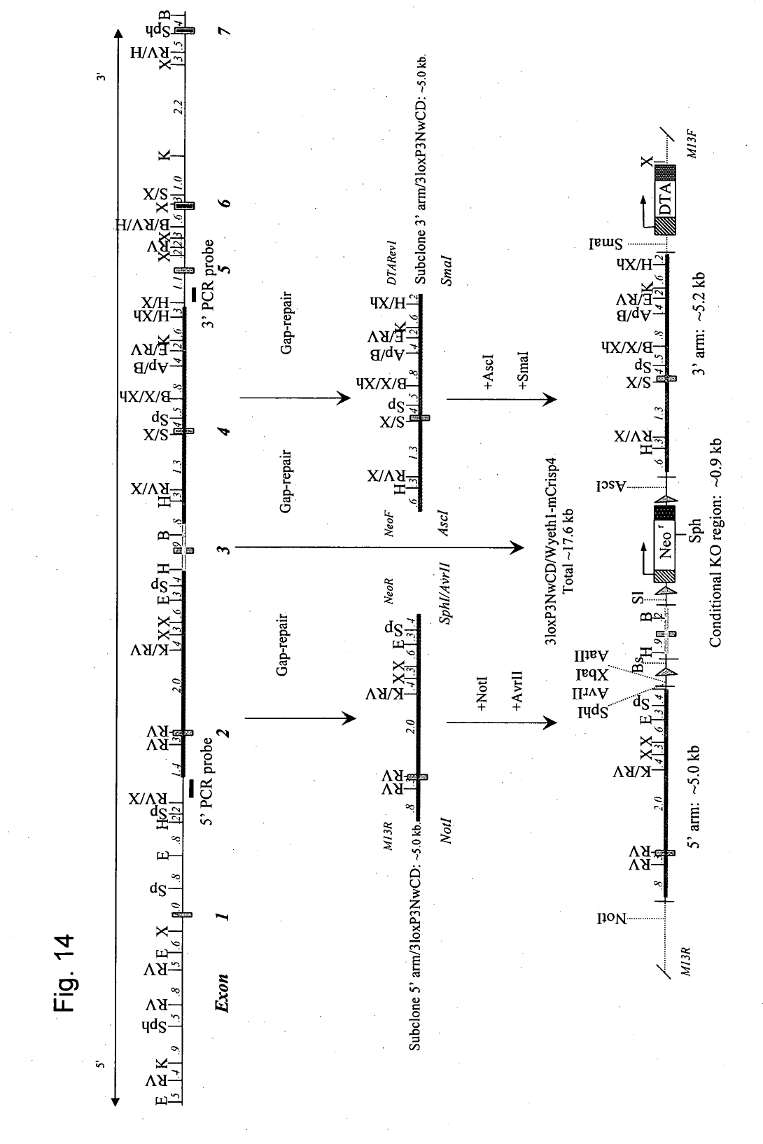

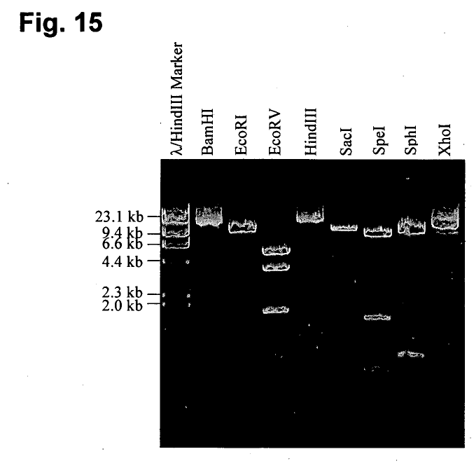

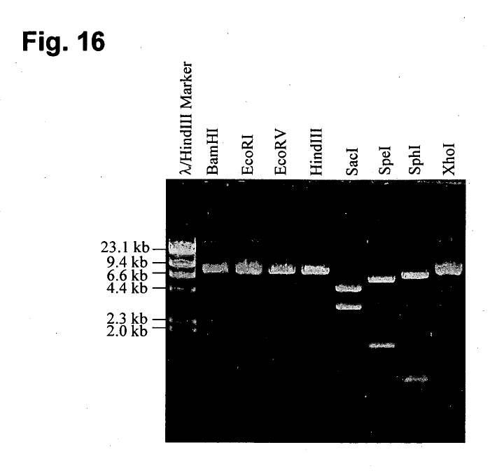

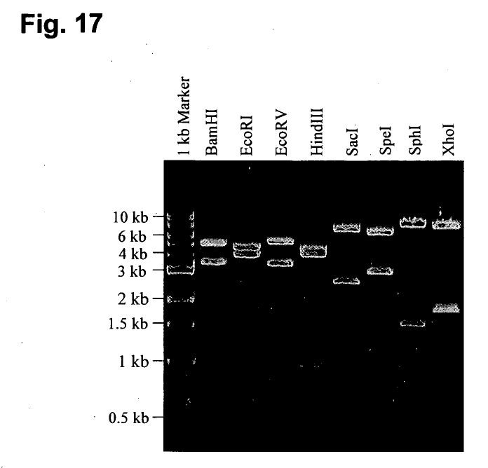

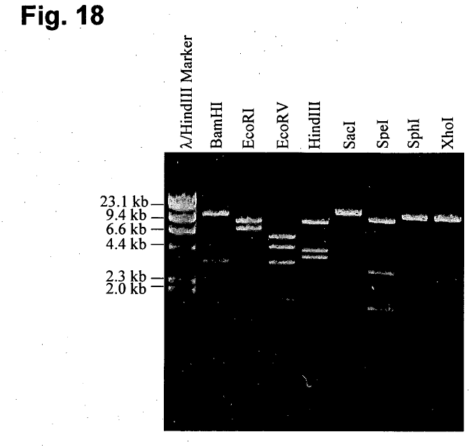

The 5′ homologous arm (5.0 kb), the 3′ homologous arm (5.2 kb), and the conditional knockout region (0.9 kb) were generated by RED cloning/gap repair. They were cloned in 3loxP3NwCD or pCR2.1 vector (see FIG. 14 for vector construction strategy). All of the constructs were confirmed by restriction digestion and end-sequencing (see FIGS. 15-18 and SEQ ID NOs: 42-47).

The final vector was obtained by standard molecular cloning. Aside from the homologous arms, the final vector also contains loxP sequences flanking the conditional knockout region (0.9 kb), loxP sequences flanking a Neo expression cassette (for positive selection of the ES cells), and a DTA expression cassette (for negative selection of the ES cells). The final vector was confirmed by both restriction digestion and end sequencing analysis. Not I was used for linearizing the final vector for electroporation.

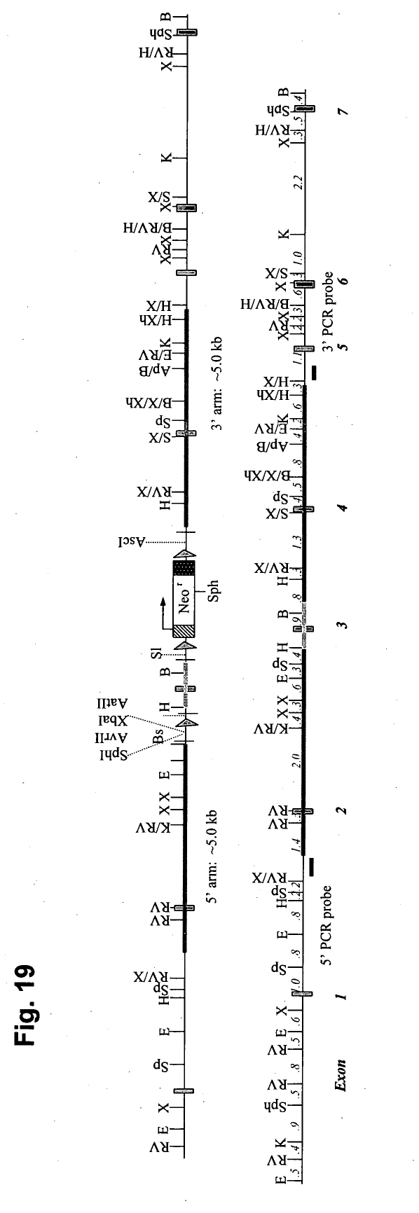

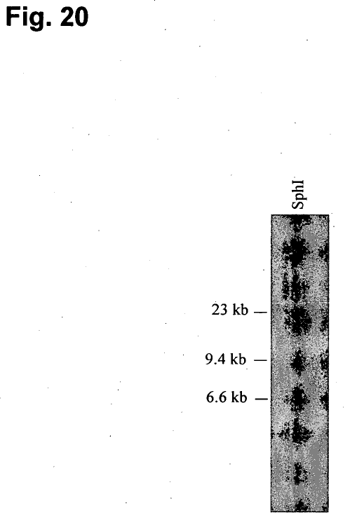

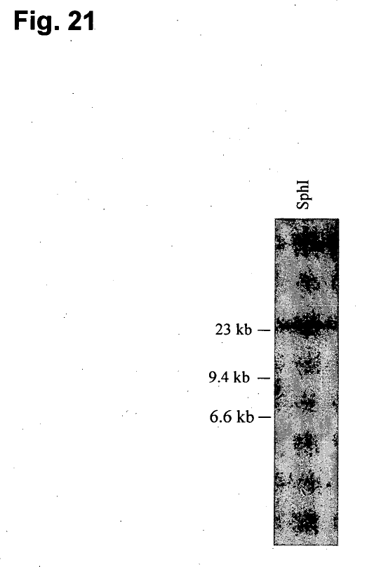

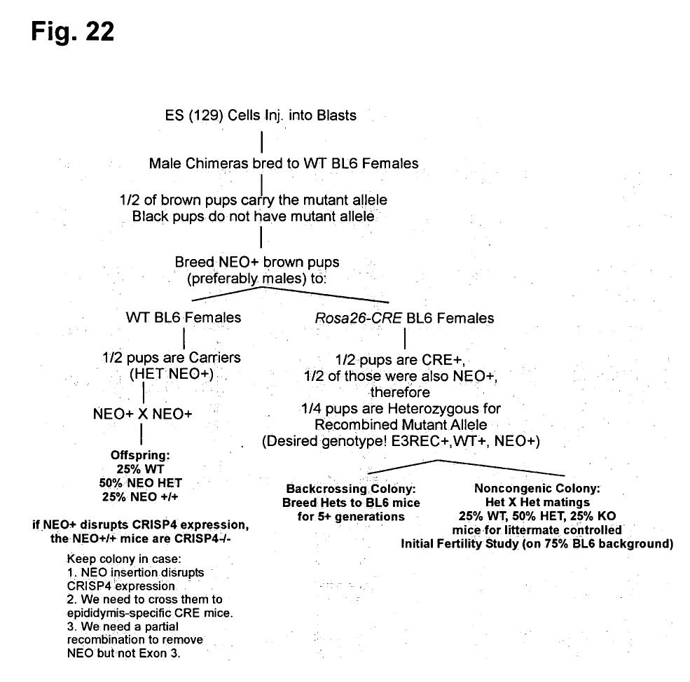

The 5′ and 3′ external probes were generated by PCR reaction using proofreading LA Taq DNA polymerase (Takara), and were tested by genomic Southern analysis for screening of the ES cells (see FIGS. 19-21 and SEQ ID NOs: 48-49). The probes were cloned in the pCR2.1 backbone and confirmed by sequencing.

Mice carrying the targeted allele (loxP-Exon3-loxP-NEO-loxP) will be bred with mice expressing Cre recombinase for either partial (removal of either Exon 3 or NEO) or complete (removal of both Exon 3 and NEO) recombination. Removal of Exon 3 is predicted to disrupt expression of mCRISPC protein. However, the insertion of the NEO may also disrupt mCRISPC expression, and mice homozygous for the non-Cre-recombined allele will be analyzed for the expression of mCRISPC. If these mice lack mCRISPC protein, they are also mCRISPC knockouts.

The breeding scheme for generating CRISPC knockout mice is shown in FIG. 22. Once mice with a complete disruption of mCRISPC expression are generated, their fertility will be tested in a breeding study for their ability to sire or mother offspring. Applicants predict that male CRISPC knockout mice will be infertile and female CRISPC knockout mice will be fertile. Male and female mice will be analyzed for histopathological defects including but not limited to reproductive tissues. Applicants predict that there will be very few histopathological defects, with the highest probability of finding a defect in the epididymis and/or testis. Epididymal spermatozoa from knockout mice will be analyzed for their ability to move in a forward direction, undergo capacitation, fertilize an oocyte in vitro. Applicants predict that mCRISPC null sperm will have defects in sperm function, which may include but are not limited to reduced percent of motile sperm, decreased sperm velocity, lack of capacitated flagellar waveform, loss of tyrosine phosphorylation during capacitation, inability to penetrate the zona pellucida and/or fuse with and fertilize a wildtype oocyte. The knockout mice may also be used to demonstrate an accumulation of the uncleaved endogenous mCRISPC proteolytic substrate in the tissues or cells of the male or female reproductive tracts.

1-96. (canceled)

97. An isolated polynucleotide encoding a CRISPC polypeptide comprising a nucleotide sequence selected from the group consisting of:
(a) a nucleotide sequence encoding an amino acid sequence having at least 65% identity with the amino acid sequence set forth in SEQ ID NO:5;
(b) a nucleotide sequence encoding an amino acid sequence having at least 65% identity with the amino acid sequence set forth in SEQ ID NO:6;
(c) a nucleotide sequence which hybridizes with (a) or (b) under the following conditions: 6×SSC at 45° C. and washed at least once with 0.2×SSC, 0.1% SDS at 50° C.; and
(d) a nucleotide sequence complementary to (a), (b), or (c).

98. The isolated polynucleotide of claim 97, wherein the nucleotide sequence of (a) encodes the amino acid sequence set forth in SEQ ID NO:5.

99. The isolated polynucleotide of claim 98, wherein the nucleotide sequence of (a) is nucleotides 97-846 of SEQ ID NO:19.

100. The isolated polynucleotide of claim 97, wherein the nucleotide sequence of (b) encodes the amino acid sequence set forth in SEQ ID NO:6.

101. The isolated polynucleotide of claim 100, wherein the nucleotide sequence of (b) is nucleotides 161-922 of SEQ ID NO:25.

102. A vector comprising the isolated polynucleotide of claim 97.

103. A transformed host cell comprising the isolated polynucleotide of claim 97.

104. A non-human transgenic animal comprising the isolated polynucleotide of claim 97.

105. An isolated antisense polynucleotide which is antisense to the isolated polynucleotide of claim 97.

106. An isolated polynucleotide comprising the polynucleotide sequence of SEQ ID NO:3 or SEQ ID NO:4.

107. An isolated polynucleotide comprising the polynucleotide sequence of SEQ ID NO:29 or SEQ ID NO:30.

108. An isolated polynucleotide fragment comprising at least 12 contiguous nucleotides from the polynucleotide sequence selected from the group consisting of SEQ ID NO:3, SEQ ID NO:4, SEQ ID NO:29, and SEQ ID NO:30.

109. An isolated polynucleotide fragment encoding a biologically active portion of an mCRISPC or rCRISPC protein.

110. An isolated polynucleotide fragment comprising at least 12 contiguous nucleotides from nucleotides 97-846 of SEQ ID NO:19 or nucleotides 161-922 of SEQ ID NO:25.

111. An isolated CRISPC polypeptide encoded by a nucleotide sequence selected from the group consisting of:
(a) a nucleotide sequence encoding an amino acid sequence having at least 65% identity with the amino acid sequence set forth in SEQ ID NO:5;
(b) a nucleotide sequence encoding an amino acid sequence having at least 65% identity with the amino acid sequence set forth in SEQ ID NO:6; and
(c) a nucleotide sequence that hybridizes with the complement of the nucleotide sequence of (a) or (b) under the following conditions: 6×SSC at 45° C. and washed at least once with 0.2×SSC, 0.1% SDS at 50° C.

112. The isolated CRISPC polypeptide of claim 111, wherein the nucleotide sequence of (a) encodes the amino acid sequence set forth in SEQ ID NO:5.

113. The isolated CRISPC polypeptide of claim 111, wherein the nucleotide sequence of (b) encodes the amino acid sequence set forth in SEQ ID NO:6.

114. The isolated CRISPC polypeptide of claim 111, wherein the isolated CRISPC polypeptide has protease activity.

115. The isolated CRISPC polypeptide of claim 114, wherein the isolated CRISPC polypeptide has site-specific protease activity.

116. The isolated CRISPC polypeptide of claim 115, wherein the CRISPC polypeptide protease activity recognizes an amino acid sequence comprising a sequential arginine-alanine sequence.

117. The isolated CRISPC polypeptide of claim 115, wherein the CRISPC polypeptide protease activity recognizes an amino acid sequence having at least one dibasic amino acid residue pair.

118. The isolated CRISPC polypeptide of claim 114, wherein the CRISPC polypeptide protease activity activates sperm motility, affects progression of sperm capacitation, affects sperm binding to or releasing from epithelial cells of the male and/or female reproductive tract, mediates sperm-egg binding or fusion, or a combination thereof.

119. A fusion protein comprising a first polypeptide consisting of the isolated CRISPC polypeptide of claim 111 operably linked to a second, non-mCRISPC polypeptide.

120. An antibody which specifically binds a CRISPC polypeptide comprising SEQ ID NO:5 or SEQ ID NO:6.

121. An antibody which specifically binds a CRISPC polypeptide fragment comprising at least 8 contiguous amino acids from SEQ ID NO:5 or SEQ ID NO:6.

122. A method for detecting a CRISPC polypeptide comprising detecting binding of an antibody selected from the group consisting of
(a) an antibody which selectively binds a CRISPC polypeptide comprising SEQ ID NO:5;
(b) an antibody which selectively binds a CRISPC polypeptide fragment comprising at least 8 contiguous amino acids from SEQ ID NO:5;
(c) an antibody which selectively binds a CRISPC polypeptide comprising SEQ ID NO:6; and
(d) an antibody which selectively binds a CRISPC polypeptide fragment comprising at least 8 contiguous amino acids from SEQ ID NO:6;
to a molecule in a sample suspected of containing a CRISPC polypeptide, wherein the antibody is contacted with the sample under conditions that permit specific binding with any CRISPC polypeptide present in the sample and binding of the antibody to the molecule in the sample indicates the presence of CRISPC.

123. A method for detecting expression of CRISPC comprising detecting mRNA encoding CRISPC in a sample from a cell suspected of expressing CRISPC with a probe comprising at least 12 contiguous nucleotides from nucleotides 97-846 of SEQ ID NO:19 or nucleotides 161-922 of SEQ ID NO:25.

124. A method for determining whether a CRISPC gene has been mutated or deleted comprising detecting, in a sample of cells from a subject, the presence or absence of a genetic alteration characterized by at least one of an alteration affecting the integrity of a gene encoding a CRISPC protein or the misexpression of a CRISPC gene, wherein the detecting step is performed with at least one of a probe or primer comprising at least 12 contiguous nucleotides from nucleotides 97-846 of SEQ ID NO:19 or nucleotides 161-922 of SEQ ID NO:25.

125. A method of identifying CRISPC variants comprising screening a combinatorial library comprising mCRISPC or rCRISPC mutants for CRISPC agonists or antagonists.

126. A CRISPC variant identified by the method of claim 125.

127. A method of isolating a CRISPC polypeptide comprising
(a) contacting an mCRISPC or rCRISPC antibody with a sample suspected of containing a CRISPC polypeptide; and
(b) isolating an mCRISPC or rCRISPC antibody-CRISPC polypeptide complex from the sample.

128. A method of producing a CRISPC polypeptide comprising
(a) culturing a transformed host cell comprising an expression vector comprising an isolated polynucleotide selected from the group consisting of:
(i) a nucleotide sequence encoding an amino acid sequence having at least 65% identity with the amino acid sequence set forth in SEQ ID NO:5;
(ii) a nucleotide sequence encoding an amino acid sequence having at least 65% identity with the amino acid sequence set forth in SEQ ID NO:6;
(iii) a nucleotide sequence which hybridizes with (i) or (ii) under the following conditions: 6×SSC at 45° C. and washed at least once with 0.2×SSC, 0.1% SDS at 50° C.; and
(iv) a nucleotide sequence complementary to (i), (ii), or (iii);
in a suitable medium such that a CRISPC polypeptide is produced; and
(b) optionally recovering the CRISPC polypeptide of step (a).

129. A method of screening for compounds which modulate mCRISPC or rCRISPC polypeptide biological activity comprising:
(a) contacting a test compound with a sample containing an mCRISPC or rCRISPC polypeptide; and
(b) determining the ability of the test compound to modulate the biological activity of the mCRISPC or rCRISPC polypeptide.

130. A compound identified by the method of claim 129.

131. A method for the treatment of a mammal in need of increased CRISPC activity comprising administering to the mammal in need thereof a therapeutically effective amount of an mCRISPC or rCRISPC polynucleotide or polypeptide.

132. A method for the treatment of a mammal in need of decreased CRISPC activity comprising administering to the mammal in need thereof a therapeutically effective amount of an mCRISPC or rCRISPC antisense polynucleotide.

133. A method for the treatment of a mammal in need of decreased CRISPC activity comprising administering to the mammal in need thereof a therapeutically effective amount of an mCRISPC or rCRISPC antibody.

134. A method for obtaining anti-mCRISPC or anti-rCRISPC antibodies comprising:
(a) immunizing an animal with an immunogenic mCRISPC protein, an immunogenic rCRISPC protein, or an immunogenic portion thereof unique to an mCRISPC or rCRISPC polypeptide; and
(b) isolating from the animal antibodies that specifically bind to an mCRISPC or rCRISPC protein.

135. A method of developing a sensor cell for determining the activity of a gene comprising:
(a) providing a homogeneous population of cells, wherein each of the cells comprises a signal transduction detection system;
(b) introducing into the population of cells an isolated genomic construct comprising an mCRISPC or rCRISPC promoter operably linked to a targeting sequence, wherein:
(i) the targeting sequence comprises a region of homology to a target gene sufficient to promote homologous recombination of the isolated genomic construct following introduction into the cells;
(ii) the mCRISPC or rCRISPC promoter is heterologous to the target gene;
(iii) following recombination the promoter controls transcription of an mRNA that encodes a polypeptide comprising an activatable domain; and
(iv) the polypeptide is capable, upon activation of the activatable domain, of altering the signal detected from the signal transduction system;
(c) incubating the population of cells under conditions which cause expression of the polypeptide;
(d) incubating the population of cells under conditions which cause activation of the activatable domain of the polypeptide; and
(e) selecting cells that have altered the signal detected from the signal transduction system.

136. A method for the production of a CRISPC polypeptide comprising:
(a) providing a homogeneous population of cells;
(b) introducing into the population of cells an isolated genomic construct comprising a promoter operably linked to an mCRISPC or rCRISPC targeting sequence, wherein:
(i) the mCRISPC or rCRISPC targeting sequence comprises a region of homology to a CRISPC target gene sufficient to promote homologous recombination of the isolated genomic construct following introduction into the cells;
(ii) the promoter is heterologous to the CRISPC target gene; and
(iii) following recombination the promoter controls transcription of an mRNA that encodes a CRISPC polypeptide; and
(c) incubating the population of cells under conditions which cause expression of the CRISPC polypeptide.

137. A kit for detecting CRISPC polypeptide or polynucleotide comprising:
(a) a labeled compound or agent capable of detecting an mCRISPC or rCRISPC polypeptide or polynucleotide in a biological sample;
(b) means for determining the amount of mCRISPC or rCRISPC polypeptide or polynucleotide in the sample;
(c) means for comparing the amount of mCRISPC or rCRISPC polypeptide or polynucleotide in the sample with a standard; and
(d) optionally, instructions for using the kit to detect mCRISPC or rCRISPC polypeptide or polynucleotide.

138. A kit for identifying modulators of CRISPC activity comprising:
(a) a cell or composition comprising an mCRISPC or rCRISPC polypeptide;
(b) means for determining mCRISPC or rCRISPC polypeptide activity; and
(c) optionally, instructions for using the kit to identify modulators of CRISPC activity.

139. A kit for diagnosing a disorder associated with aberrant CRISPC expression and/or activity in a subject comprising:
(a) a reagent for determining expression of mCRISPC or rCRISPC polypeptide or polynucleotide;
(b) a control to which the results of the subject are compared; and
(c) optionally, instructions for using the kit for diagnostic purposes.

140. A knockout construct having the sequence set forth in SEQ ID NO:41.

141. A vector comprising the knockout construct of claim 140.

142. A transgenic mouse having a homologous disruption in the CRISPC gene, wherein the disruption results in mice having defective sperm function.

143. The transgenic mouse of claim 142, wherein the defective sperm function is reduced percent of motile sperm, decreased sperm velocity, lack of capacitated flagellar waveform, loss of tyrosine phosphorylation during capacitation, inability to penetrate the zona pellucida and/or fuse with and fertilize a wildtype oocyte, or a combination thereof.
